(19) United States
(12) Patent Application Publication
(10) Pub. No.: US 2021/0283242 A1
(43) Pub. Date: Sep. 16, 2021

(54) IMMUNE-MEDIATED CORONAVIRUS TREATMENTS

(71) Applicants: Heat Biologics, Inc., Morrisville, NC (US); University of Miami, Miami, FL (US)

(72) Inventors: Jeff T. HUTCHINS, Claremore, OK (US); Natasa STRBO, Miami, FL (US); Jeffrey WOLF, Morrisville, NC (US)

(21) Appl. No.: 17/190,098

(22) Filed: Mar. 2, 2021

Related U.S. Application Data
(60) Provisional application No. 63/064,989, filed on Aug. 13, 2020.
(60) Provisional application No. 63/061,390, filed on Aug. 5, 2020.
(60) Provisional application No. 62/991,223, filed on Mar. 18, 2020.
(60) Provisional application No. 62/983,783, filed on Mar. 2, 2020.

(51) Int. Cl. A61K 39/215; C07K 14/005; C07K 14/47; C07K 14/525; A61K 39/39; A61K 39/12; A61P 31/14

(52) U.S. Cl. CPC A61K 39/215; C07K 14/005; C07K 14/47; C07K 14/525; A61K 39/39; A61K 2039/55516; A61P 31/14; C07K 2319/30; C07K 2319/35; C07K 2319/33; A61K 39/12

(57) ABSTRACT

The present invention provides an expression vector, host cells, methods and kits for the treatment or prevention of a coronavirus infection in a subject.

### PRIORITY

The present application claims priority to U.S. Provisional Application No. 62/983,783, filed on Mar. 2, 2020, U.S. Provisional Application No. 62/991,223, filed on Mar. 18, 2020, U.S. Provisional Application No. 63/061,390, filed on Aug. 5, 2020, and U.S. Provisional Application No. 63/064,989, filed on Aug. 13, 2020, the contents of which are herein incorporated by reference in their entireties.

### FIELD

The present invention relates, in part, to compositions and methods useful for immune modulation in connection with, for example, infection by coronavirus.

### INCORPORATION BY REFERENCE OF MATERIAL SUBMITTED ELECTRONICALLY

The contents of the text file named “HTB-035_Sequence Listing_ST25”, which was created on Mar. 2, 2021 and is 361,369 bytes in size, are hereby incorporated herein by reference in their entireties.

### BACKGROUND

The coronavirus (CoV) is a member of the family Coronaviridae, including betacoronavirus and alphacoronavirus respiratory pathogens that have relatively recently become known to invade humans. The Coronaviridae family includes such betacoronavirus as Severe acute respiratory syndrome coronavirus 2 (SARS-CoV-2), SARS-CoV, Middle East Respiratory Syndrome—Corona Virus (MERS-CoV), HCoV-HKU1, and HCoV-OC43. Alphacoronavirus includes, e.g., HCoV-NL63 and HCoV-229E. Coronaviruses invade cells through “spike” surface glycoprotein that is responsible for viral recognition of Angiotensin Converting Enzyme 2 (ACE2), a transmembrane receptor on mammalian hosts that facilitate viral entrance into host cells. Zhou et al., A pneumonia outbreak associated with a new coronavirus of probable bat origin. Nature 2020. A new coronavirus infection 2019 (COVID-19), caused by SARS-CoV-2 (also known as 2019-nCoV) is a new disease thought to be originated from the bat. COVID-19 causes severe respiratory distress and this RNA virus strain has been the cause of the recent outbreak that has been declared a major threat to public health and worldwide emergency. Phylogenetic analysis of the complete genome of 2019-nCoV revealed that the virus was most closely related (89.1% nucleotide similarity) to a group of SARS-like coronaviruses (genus Betacoronavirus, subgenus Sarbecovirus). Wu et al., A new coronavirus associated with human respiratory disease in China. Nature, Feb. 3, 2020. SARS-CoV-2 is thought to spread from person-to-person and the spread may be possible from contact with infected surfaces or objects.

Coronaviruses invade cells through “spike” (S, or Spike) surface glycoprotein that is responsible for viral recognition of Angiotensin Converting Enzyme 2 (ACE2), a transmembrane receptor on mammalian hosts that facilitate viral entrance into host cells. Zhou et al., Nature 579, 270-273 (2020). The trimeric Spike protein of SARS-CoV-2 is heavily glycosylated and it has 22 potential N-glycosylation sites. The Spike protein, a principal target of the humoral immune response, mediates host cell binding and entry. See Watanabe et al., Science, 17 Jul. 2020, Vol. 369, Issue 6501, pp. 330-333. Vaccine development is thus focused on the Spike glycoprotein, and multiple vaccines and antibody approaches are currently being explored. Also, recently there has been some success in development and production of suitable vaccines.

SARS-CoV-2 virus is evolving over time in human populations, as it is passed between hosts, crossing geographical borders. See, e.g., Li et al., Cell. 2020 Sep. 3; 182(5):1284-1294. Thus, new variants of SARS-CoV-2 appear and spread around the world. For example, a D614G variant (i.e. an aspartic acid to glycine amino acid substitution at position 614 in the Spike protein gene) has been dominating the circulating strains in the global pandemic. See Li et al. (2020); Korber et al. Cell. 2020 Aug. 20; 182(4): 812-827.e19. More recently, rapidly spreading variant in the UK (‘VUI-202012/01’ i.e. ‘variant under investigation’) has been reported in the United Kingdom. Tang et al., Journal of Infection, published Dec. 28, 2020. This variant is derived from the SARS-CoV-2 20B/GR clade (lineage B.1.1.7). Id. Efforts worldwide are undertaken to monitor for changes in the Spike protein. See Korber et al. (Cell. 2020). The newly emerging strains pose a risk of the spread of new infections, for which no adequate vaccines or treatments are available.

Accordingly, there is an urgent need for 2019-nCoV vaccines that could prevent and/or mitigate COVID-19 and related infections, and which could target existing and evolving SARS-CoV-2 variants.

### SUMMARY

Accordingly, in various aspects, the present invention relates to use of a cell-based vaccine for treating or preventing coronavirus infection, e.g., COVID-19 infection or a similar disease, which can be caused by various lineages, strains, and variants of SARS-CoV-2. In particular, in embodiments, the present invention relates to compositions and methods that provide vaccine protection from and treatment of infectious diseases including SARS-CoV-2 (2019-nCoV) virus. In embodiments, the cell-based vaccine simultaneously targets multiple variants of a SARS-CoV-2 protein, which provides a great benefit when multiple variants circulate in certain geographical regions and around the world. The compositions and methods are used, in embodiments, for prevention or reduction of symptoms of COVID-19, such as fever, cough, shortness of breath and other breathing difficulties, diarrhea, upper respiratory symptoms (e.g. sneezing, runny nose, dry cough, sore throat), and/or pneumonia.

In various embodiments, the present compositions and methods relate to the use of a SARS-CoV-2 protein and variants thereof as antigens to which an immune response is stimulated. The SARS-CoV-2 is an enveloped, single stranded, RNA virus that encodes a “Spike” protein, also known as the S protein, which is a surface glycoprotein that mediates binding to a cell surface receptor; an integral membrane protein; an envelope protein, and a nucleocapsid protein. The S protein, comprising the 51 subunit and the S2 subunit, is a trimeric class I fusion protein that exists in a prefusion conformation that undergoes a structural rearrangement to fuse the viral membrane with the host-cell membrane. See, e.g., Li, F. Structure, Function, and Evolution of Coronavirus Spike Proteins. Annu. Rev. Virol. 3: 237-261 (2016), which is incorporated herein by reference in its entirety. The structure of the SARS-CoV-2 spike protein in the prefusion conformation has been discovered. See Daniel et al., Cryo-EM structure of the 2019-nCoV spike in the prefusion conformation. Science, 19 Feb. 2020, which is incorporated herein by reference in its entirety. The S protein mediates entry of the virus into host cells by first binding to a host receptor through a receptor-binding domain (RBD) in the 51 subunit and then fusing the viral and host membranes through the S2 subunit. See Tai et al., Cellular & Molecular Immunology volume 17, pages 613-620 (2020). Coronavirus S proteins are extensively glycosylated, encoding around 66-87 N-linked glycosylation sites per trimeric spike. See Watanabe et al., Nature Communications volume 11, Article number: 2688 (2020).

In some embodiments, a cell-based vaccine in accordance with the present disclosure has two or more variants of the Spike proteins. Accordingly, in various embodiments, the cell-based vaccine includes two or more nucleic acids each encoding a respective variant, lineage, or strain of a coronavirus protein. Various variants can be incorporated into an expression vector system in accordance with embodiments of the present disclosure. For example, the variants can include a coronavirus protein having a mutation (e.g., without limitation, a substitution, deletion, or insertion) in any part of the Spike protein, such as in the 51 subunit (e.g., in the RBD of the Spike protein), or in the S2 subunit. In some embodiments, a mutation is in a glycosylation site of the Spike protein.

In various embodiments, the present invention provides an expression vector system comprising (i) a nucleic acid encoding a secretable fusion protein comprising a chaperone protein and an immunoglobulin, or a fragment thereof, and (ii) a nucleic acid encoding a T cell costimulatory fusion protein, wherein the T cell costimulatory fusion protein enhances activation of antigen-specific T cells when administered to a subject; and/or (iii) a nucleic acid encoding a coronavirus protein, or an antigenic portion thereof, wherein each nucleic acid is operably linked to a promoter.

In embodiments, the expression vector system comprises one or more nucleic acid encoding a respective variant of a plurality of variants of a coronavirus protein. In some embodiments, the coronavirus protein is SARS-CoV-2 spike protein.

In some embodiments, the variants (also referred to as lineages) include B.1.1.7, B1.351, B.1, B.1.1.28, B.1.2, CAL.20C, B.6, P.1, and P.2 variants and/or any other variants, or antigenic fragments thereof. In some embodiments, the lineages include A.1, A.2, A.3, A.4, A.5, A.6, A.7, A.8, A.9, B, B.1, B.1.1, B.1.1.1, B.2, B.3, B.4, B.5, B.6, B.7, B.9, B.10, B.11, B.12, B.13, B.14, B.15, B.16, B.17, B.18, B.19, B.20, B.21, B.22, B.23, B.24, B.25, B.26, B.27, C.1, C.2, C.3, D.1, and D2.

In some embodiments, a variant is a SARS-CoV-2 protein having a variation in a glycosylation site of a Spike protein.

In some embodiments, a variant is a Spike protein having one or more of D614G, E484K, N501Y, K417N, S477G, and S477N mutations relative to the amino acid sequence of SEQ ID NO: 37 or an antigenic fragment thereof.

In some embodiments, a variant is a Spike protein having a mutation in the receptor-binding domain (RBD) of the Spike protein. In some embodiments, the mutation in the RBD of the Spike protein is a mutation in a glycosylation site in the RBD.

In some embodiments, a variant is a Spike protein having a mutation outside the RBD of the Spike protein.

Various embodiments also provide related host cell(s) comprising the expression vector system in accordance with the present invention. The nucleic acids encoding the proteins in accordance with embodiments of the present disclosure (e.g., a secretable fusion protein, a T cell costimulatory fusion protein, and a coronavirus protein or an antigenic portion thereof) can be included in one, two, or three expression vectors included in one, two, or three biological cells.

In some embodiments, one, two, or three biological cells are provided that include the nucleic acids in accordance with the present disclosure. In some embodiments, the nucleic acid encoding a secretable fusion protein, the nucleic acid encoding a T cell costimulatory fusion protein, and the nucleic acid encoding a coronavirus protein or an antigenic portion thereof are all present in the same biological cell. In some embodiments, the nucleic acid encoding a secretable fusion protein, the nucleic acid encoding a T cell costimulatory fusion protein, and the nucleic acid encoding a coronavirus protein or an antigenic portion thereof can be included in one, two, or three biological cells (e.g. two biological cells comprise one or two of the nucleic acid encoding a secretable fusion protein, and the nucleic acid encoding a T cell costimulatory fusion protein; e.g. three biological cells each comprise the nucleic acid encoding a secretable fusion protein, and the nucleic acid encoding a T cell costimulatory fusion protein).

In some embodiments, two of the nucleic acid encoding a secretable fusion protein, the nucleic acid encoding a T cell costimulatory fusion protein, and the nucleic acid encoding a coronavirus protein or an antigenic portion thereof are present in the same cell, whereas another one of the three nucleic acids is present on another cell. For example, in some embodiments, the nucleic acid encoding a secretable fusion protein and the nucleic acid encoding a coronavirus protein or an antigenic portion thereof are present in the same cell, whereas the nucleic acid encoding a T cell costimulatory fusion protein is present on another cell that is different from the cell having the nucleic acid encoding a secretable fusion protein and the nucleic acid encoding a coronavirus protein or an antigenic portion thereof. As another example, in some embodiments, the nucleic acid encoding a secretable fusion protein and the nucleic acid encoding a T cell costimulatory fusion protein are present on the same cell. The nucleic acid encoding a secretable fusion protein, the nucleic acid encoding a T cell costimulatory fusion protein, and the nucleic acid encoding a coronavirus protein or an antigenic portion thereof can be included in one, two, or three expression vectors.

In some embodiments, a composition in accordance with the present disclosure having two or more biological cells comprises different biological cells. For example, the two or more biological cells can comprise biological cells that may or may not have a T cell costimulatory fusion protein. Thus, in some embodiments, a biological cell of the two or more biological cells comprises a nucleic acid encoding a secretable fusion protein, a nucleic acid encoding a T cell costimulatory fusion protein (e.g., without limitations, OX40L), and a nucleic acid encoding a coronavirus protein or an antigenic portion thereof. Another biological cell of the two or more biological cells comprises a nucleic acid encoding a secretable fusion protein and a nucleic acid encoding a coronavirus protein or an antigenic portion thereof. For example, in embodiments, a composition is a SARS-CoV-2 cell-based vaccine that comprises a biological cell that expresses gp96 and OX40L, along with a SARS-CoV-2 antigen; and the composition comprises a biological cell that expresses gp96, along with a SARS-CoV-2 antigen.

In some embodiments, a composition comprises a single biological cell that expresses gp96, along with a SARS-CoV-2 antigen.

In some embodiments, a composition comprises a single biological cell that that expresses gp96 and a T cell costimulatory fusion protein (e.g., without limitations, OX40L), along with a SARS-CoV-2 antigen.

In some embodiments, a method of eliciting an immune response against coronavirus in a subject is provided that comprises administering to the subject a composition having a biological cell comprising an expression vector system. The expression vector system comprises (i) a nucleic acid encoding a secretable fusion protein comprising a gp96-Ig, or a fragment thereof; (ii) a nucleic acid encoding a T cell costimulatory fusion protein, optionally OX40L, wherein the T cell costimulatory fusion protein enhances activation of antigen-specific T cells when administered to a subject; and (iii) a nucleic acid encoding a coronavirus protein, or an antigenic portion thereof, wherein each nucleic acid is operably linked to a promoter.

In some embodiments, a composition having a biological cell comprising an expression vector system, the expression vector system comprising (i) a nucleic acid encoding a secretable fusion protein comprising a chaperone protein and an immunoglobulin, or a fragment thereof; and/or (ii) a nucleic acid encoding a T cell costimulatory fusion protein, wherein the T cell costimulatory fusion protein enhances activation of antigen-specific T cells when administered to a subject; and (iii) a nucleic acid encoding a coronavirus protein, or an antigenic portion thereof, wherein each nucleic acid is operably linked to a promoter. In some embodiments, the composition comprises two or more biological cells, wherein a biological cell of the two or more biological cells optionally encodes a nucleic acid encoding a secretable fusion protein comprising a chaperone protein and an immunoglobulin, or a fragment thereof; and a nucleic acid encoding a coronavirus protein, or an antigenic portion thereof.

In some embodiments, the composition comprises a single biological cell.

In some embodiments, an expression vector system in accordance with the present invention includes one or more nucleic acids, each encoding a respective variant of a coronavirus protein or an antigenic portion thereof. In some embodiments, a single nucleic acid encodes more than one variant of a coronavirus protein or an antigenic portion thereof. In some embodiments, the expression vector system comprises a mix of one or more nucleic acids each encoding a respective variant of a coronavirus protein or an antigenic portion thereof, and of one or more nucleic acids each encoding more than one respective variant of a coronavirus protein or an antigenic portion thereof.

In some embodiments, the expression vector system in accordance with the present invention includes two, three, four, five, or more than five nucleic acids encoding a respective variant of a coronavirus protein or an antigenic portion thereof. In some embodiments, each nucleic acid encoding a respective variant of a coronavirus protein or an antigenic portion thereof is included in a respective separate cell. In some embodiments, the nucleic acid encoding a secretable fusion protein, the nucleic acid encoding a T cell costimulatory fusion protein, and the nucleic acid encoding a coronavirus protein or an antigenic portion thereof can each be included in a respective separate cell. In such embodiments, the three cells include three respective expression vectors each having one of the nucleic acid encoding a secretable fusion protein, the nucleic acid encoding a T cell costimulatory fusion protein, and the nucleic acid encoding a coronavirus protein or an antigenic portion thereof.

In some embodiments, a composition is provided that comprises a biological cell comprising an expression vector system comprising one or more: (i) a nucleic acid encoding a secretable fusion protein comprising a chaperone protein and an immunoglobulin, or a fragment thereof, (ii) a nucleic acid encoding a T cell costimulatory fusion protein, wherein the T cell costimulatory fusion protein enhances activation of antigen-specific T cells when administered to a subject; and (iii) a nucleic acid encoding a coronavirus protein, or an antigenic portion thereof, wherein each nucleic acid is operably linked to a promoter. In some embodiments, the composition comprises one or more nucleic acids, each encoding a respective variant of a coronavirus protein or an antigenic portion thereof.

In exemplary embodiments, the coronavirus protein is a betacoronavirus protein (e.g. SARS-CoV-2 (2019-nCoV), SARS-CoV, MERS-CoV, HCoV-HKU1, and HCoV-OC43) or alphacoronavirus protein (e.g. HCoV-NL63 and HCoV-229E). In some embodiments, the coronavirus protein is a betacoronavirus protein such as SARS-CoV-2 (2019-nCoV).

In some embodiments, the spike surface glycoprotein comprises an amino acid sequence having at least one mutation relative to the amino acid sequence of SEQ ID NO: 37.

In some embodiments, the spike surface glycoprotein comprises an amino acid sequence having D614G mutation relative to the amino acid sequence of SEQ ID NO: 37.

In some embodiments, the expression vector system in accordance with the present disclosure leverages gp96 to effectively present one or more SARS-CoV-2 antigens and activate the immune system. The gp96-based expression vector system utilizes natural immune process to induce long-lasting memory responses and can effectively present multiple SARS-CoV-2 antigens and activate the immune system. Thus, the expression vector system or a population of cells transfected with the expression vector is designed to elicit long lasting immune response against SARS-CoV-2 virus. The described methods and compositions aim to trigger mucosal immunity by activating both B and T cell responses at the point of pathogen entry.

In embodiments, the expression vector system in accordance with the present disclosure effectively presents one or more antigens against two or more variants of SARS-CoV-2 virus. The expression vector system can be customized for a certain subject or a population of subjects—e.g., in response to detection of prevalence of certain SARS-CoV-2 variants in a certain region and/or among a certain population. Furthermore, the expression vector system in accordance with the present disclosure can be created to target one or more variants of a coronavirus protein as new variants appear.

The present invention provides a method of eliciting an immune response against a coronavirus in a subject, comprising administering to the subject the expression vector(s) of the present invention or a population of cells transfected with the expression vector(s), in an amount effective to elicit an immune response against coronavirus in the subject. In exemplary embodiments, the coronavirus is a SARS-CoV-2 virus. Accordingly, the present invention further provides a method of eliciting an immune response against SARS-CoV-2 in a subject, comprising administering to the subject the expression vector(s) of the present invention or a population of cells transfected with the expression vector(s), in an amount effective to elicit an immune response against SARS-CoV-2 in the subject.

In various embodiments, the compositions and methods activate an innate, humoral (i.e. antibody response), and/or cellular (i.e. T cell) response in the subject receiving the present compositions. In some embodiments, the activation of cellular or T-cell-driven immunity is more pronounced than the activation of the innate and humoral responses.

In some embodiments, the method is suitable for increasing the subject's T-cell response as compared to the T-cell response of a subject that was not administered the nt compositions. In embodiments, the method is suitable for increasing the subject's antibody response as compared to the antibody response of a subject that was not administered the present compositions. In embodiments, the method is suitable for increasing the subject's innate immune response as compared to the innate immune response of a subject that was not administered the present compositions. In embodiments, the method is suitable for increasing the subject's T-cell response, antibody response, and innate immune response as compared to the T-cell response, antibody response, and innate immune responses of a subject that was not administered the present compositions.

In some embodiments, the method is suitable for increasing and/or restoring the subject's T cell population(s) as compared to the T cell population(s) of a subject that was not administered the present compositions. The subject's T cells include T cells selected from one or more of CD4+ effector T cells, CD8+ effector T cells, CD4+ memory T cells, CD8+ memory T cells, CD4+ central memory T cells, CD8+ central memory T cells, natural killer T cells, CD4+ helper cells, and CD8+ cytotoxic cells. In some embodiments, the method is suitable for increasing and/or restoring the subject's CD4+ helper cells population(s) as compared to the CD4+ helper cells population(s) of a subject that was not administered the present compositions.

In some embodiments, the chaperone protein is the secretable gp96-Ig fusion protein. In some embodiments, the secretable gp96-Ig fusion protein may optionally lack the gp96 KDEL (SEQ ID NO:49) sequence.

In some embodiments, the T cell costimulatory fusion protein comprises one or more agonists of OX40 (e.g., OX40L-Ig), ICOS (e.g., ICOSL-Ig), 4-1BB (e.g., 4-1BBL-Ig), TNFRSF25 (e.g., TL1A-Ig), CD40 (e.g., CD40L-Ig), CD27 (e.g., CD70-Ig), and/or GITR (e.g., GITRL-Ig). In some embodiments, the T cell costimulatory fusion protein is OX40L-Ig, or a portion thereof that binds to OX40. In some embodiments, the T cell costimulatory fusion protein is ICOSL-Ig, or a portion thereof that binds to ICOS. In some embodiments, the T cell costimulatory fusion protein is 4-1BBL-Ig, or a portion thereof that binds to 4-1BBR. In some embodiments, the T cell costimulatory fusion protein is TL1A-Ig, or a portion thereof that binds to TNFRSF25. In some embodiments, the T cell costimulatory fusion protein is GITRL-Ig, or a portion thereof that binds to GITR. In some embodiments, the T cell costimulatory fusion protein is CD40L-Ig, or a portion thereof that binds to CD40. In some embodiments, the T cell costimulatory fusion protein is CD70-Ig, or a portion thereof that binds to CD27. In some embodiments, the Ig tag in the T cell costimulatory fusion protein comprises the Fc region of human IgG1, IgG2, IgG3, IgG4, IgM, IgA, or IgE.

In some embodiments, the materials and methods described herein are advantageous in that, inter alia, they provide a single composition that can achieve both vaccination with, for example, gp96-Ig, and T cell costimulation without the need for independent products. These materials and methods achieve this goal by creating a single vaccine protein (e.g., gp96-Ig) expression vector that has been genetically modified to simultaneously express an costimulatory molecule, including without limitation, fusion proteins such as ICOSL-Ig, 4-1BBL-Ig, TL1A-Ig, OX40L-Ig, CD40L-Ig, CD70-Ig, or GITRL-Ig, to provide T cell costimulation. The vectors, and methods for their use, can provide a costimulatory benefit without the need for an additional antibody therapy to enhance the activation of antigen-specific CD8+ T cells. Thus, combination immunotherapy can be achieved by vector re-engineering to obviate the need for vaccine/antibody/fusion protein regimens, which may reduce both the cost of therapy and the risk of systemic toxicity.

In some embodiments, there is provided a biological cell that comprises an expression vector system comprising: (i) a nucleic acid encoding a secretable fusion protein comprising a chaperone protein and an immunoglobulin, or a fragment thereof, and (ii) a nucleic acid encoding a T cell costimulatory fusion protein, wherein the T cell costimulatory fusion protein enhances activation of antigen-specific T cells when administered to a subject; and/or (iii) a nucleic acid encoding a coronavirus protein, or an antigenic portion thereof, wherein each nucleic acid is operably linked to a promoter. In some embodiments, the expression vector system of the biological cell comprises one or more nucleic acids, each encoding a respective variant of a coronavirus protein or an antigenic portion thereof.

In some embodiments, there is provided (i) a first expression vector system comprising a nucleic acid encoding a fusion protein comprising a chaperone protein and an immunoglobulin, or a fragment thereof, the nucleic acid being operably linked to a promoter, (ii) a second expression vector system comprising a T cell costimulatory fusion protein, wherein the T cell costimulatory fusion protein enhances activation of antigen-specific T cells when administered to a subject; and/or (iii) a third expression vector system comprising a nucleic acid encoding a coronavirus protein, or an antigenic portion thereof, the nucleic acid being operably linked to a promoter.

In some embodiments, there is provided a method of treating or preventing a coronavirus infection with the biological cell. In some embodiments, there is provided a method of treating or preventing a coronavirus infection with two biological cells or three biological cells, wherein the coronavirus infection is caused by one or more variants of a coronavirus protein, or an antigenic portion thereof. Thus, in some embodiments, a method of treating or preventing a coronavirus infection in a subject is provided, comprising administering to the subject the biological cell in accordance with embodiments of the present disclosure.

In some embodiments, there are provided at least two biological cells, the first biological cell comprising an expression vector system comprising a nucleic acid encoding a fusion protein comprising a chaperone protein and an immunoglobulin, or a fragment thereof, the nucleic acid being operably linked to a promoter, and the second biological cell comprising an expression vector system comprising a nucleic acid encoding a coronavirus protein, or an antigenic portion thereof, the nucleic acid being operably linked to a promoter. In some embodiments, there is provided a method of treating or preventing a coronavirus infection with the at least two biological cells. In some embodiments, at least one of the biological cells comprises an expression vector system comprising one or more nucleic acids, each of the one or more nucleic acids encoding a respective variant of a coronavirus protein or an antigenic portion thereof.

In embodiments, various doses of the vaccine in accordance with the present disclosure can be used. In embodiments, a composition in accordance with the present disclosure comprises at least or about 0.5×10⁶ cells transfected with the expression vector system, optionally comprising 0.5×10⁶ cells; and/or an effective amount of cells that express and/or secrete at least or about 500-1000 ng of secretable fusion protein, optionally gp96.

In embodiments, a composition in accordance with the present disclosure comprises at least or about 0.5×10⁶ cells transfected with the expression vector system. In embodiments, the composition optionally comprises 0.5×10⁶ cells. In embodiments, the composition comprises an effective amount of cells that express and/or secrete at least or about 500-1000 ng of secretable fusion protein, optionally gp96.

The present invention also provides a composition comprising an expression vector system, a host cell, or a population of cells, as presently disclosed herein, and an excipient, carrier, or diluent. In exemplary aspects, the composition is a pharmaceutical composition.

Additionally, the present invention provides a kit comprising an expression vector system, a host cell, a population of cells, or a composition, in accordance with embodiments of the present disclosure.

The present inventions furthermore provide a method of treating or preventing a coronavirus infection in a subject, comprising administering to the subject the expression vector of the present invention or a population of cells transfected with the expression vector, in an amount effective to treat or prevent the coronavirus infection. In exemplary embodiments, the coronavirus infection is a SARS-CoV-2 infection. Accordingly, the present inventions furthermore provide a method of treating or preventing a coronavirus (e.g., SARS-CoV-2) infection in a subject, comprising administering to the subject the expression vector of the present invention or a population of cells transfected with the expression vector, in an amount effective to treat or prevent the SARS-CoV-2 infection. The coronavirus infection can be caused by any one or more variants of a coronavirus protein,

### BRIEF DESCRIPTION OF THE DRAWINGS

FIG. 1 is a schematic illustration of an exemplary expression vector of the present invention.

FIG. 2 is a schematic representation of the re-engineering of an gp96-Ig vector to generate a cell-based combination product that encodes the gp96-Ig fusion protein in a first cassette, and a T cell costimulatory fusion protein in a second cassette. ICOS-Fc, 4-1BBL-Fc, and OX40L-Fc are shown for illustration.

FIG. 3 is a schematic representation of a mammalian expression vector (B45) encoding a secretable gp96-Ig fusion protein in one expression cassette and a T cell costimulatory fusion protein (by way of non-limiting illustration, ICOSL-IgG4 Fc) in a second cassette.

FIGS. 4A-4E show a schematic illustration of gp96-Ig and SARS-CoV-2 protein S constructs used to generate vaccine cells HEK-293-gp96-Ig-S and AD-100-gp96-Ig-S, and graphs and images related to the expression of protein S by the vaccine cells. In FIG. 4A, each panel presents the protein expressed by the DNA (black outline) for the gp96-Ig and SARS-CoV-2 protein S vaccine antigen. N=amino terminus; C=carboxy terminus; TM=transmembrane domain: KDEL=retention signal; CH2 CH3 gamma 1=heavy chain of IgG1. Gp96-Ig and SARS-CoV2-S DNA were cloned into the mammalian expression vectors B45 and pcDNA3.1 which are transfected into HEK-293 and AD100. Stable transfection vaccine cells were generated after selection with Zeocyn and Neomycin. In FIG. 4B, one million of 293-gp96-Ig-S and AD100-gp96-Ig-S cells were plated in 1 ml for 24 hours (h) and gp96-Ig production in the supernatant was determined by ELISA using anti-human IgG antibody for detection with mouse IgG1 (0.5 ug/ml) as a standard. In FIGS. 4C and 4D, cell lysates were analyzed under reduced conditions by SDS-PAGE and Western blotting using anti-protein S antibody and recombinant protein 51 as a positive control. FIG. 4E shows immunofluorescence (IF) for protein S (in green) expressed in AD100-gp96-Ig-S cells using rabbit anti-SARS-CoV2 S1 antibody (FIG. 4E, panel “A”, left) and anti-rabbit Ig-AF488 as secondary antibody (FIG. 4E, panel “B”, right). AD100 was used as a negative control and beta-actin for protein quantification. Original magnification 40× with DAPI nuclear staining shown in blue.

FIGS. 5A-5C are a series of graphs showing how secreted gp96-Ig-S vaccine induces CD8 T cell effector memory (TEM) and resident memory (TRM) responses in the lungs. Equivalent number of AD100-gp96-Ig-S vaccine cells that produce 200 ng/ml gp96-Ig or PBS were injected by s.c. route in C56Bl/6 mice. Five days later mice were sacrificed and spleens (SPL), lungs and bronchoalveolar lavage (BAL) was isolated and frequency of CD4 and CD8 T cells (FIG. 5A); naive (N) CD44−CD62L+, central memory (CM) CD44+CD62L+ and effector memory (EM) CD44+CD62L− CD8 T cells (FIG. 5B); and resident memory (TRM) CD69+ cells (FIG. 5C), were determined by flow cytometry after staining the cells with antibodies against following surface markers: CD45, CD3, CD4, CD8, CD44, CD62L and CD69 antibodies. Bar graph shows percentage of CD4+ and CD8+ cells within CD3+ cells or CD8 T cell memory subset within CD8+ T cells. Data represent at least two technical replicates with 3-6 independent biological replicates per group. *p<0.05, **p<0.01, ***p<0.001 (a-b, Mann-Whitney tests were used to compare 2 experimental groups. To compare >2 experimental groups, Kruskal-Wallis ANOVA with Dunn's multiple comparisons tests were applied.

FIGS. 6A-6F are a series of graphs showing how secreted gp96-Ig-S vaccine induces protein S specific CD8+ and CD4+ T cells in the spleen and lung tissue. Five days after the vaccination of C57Bl6 mice, splenocytes and lung cells were isolated form vaccinated and control mice (PBS) and re-stimulated in vitro with 51 and S2 overlapping peptides from SARS-CoV-2 protein in the presence of protein transport inhibitor, brefeldin A for the last 5 h of culture. After 20 h of culture, intracellular cytokine staining (ICS) was preform to quantify protein S specific CD8+ and CD4+ T cell responses. Cytokine expression in the presence of no peptides was considered background and it was subtracted from the responses measured from peptide pool stimulated samples for each individual mouse. FIG. 6A and FIG. 6B show CD8+ T cell form spleen and lungs expressing IFNγ, TNFα and IL-2 in responses to S1 and S2 peptide pool. FIG. 6C and FIG. 6D show CD4+ T cells form spleen and lungs expressing IFNγ, TNFα and IL-2 in responses to S1 and S2 peptide pool. FIG. 6E shows the proportion of antigen (protein S)-experienced CD8+ and CD4+ T cells isolated from spleen and lung tissue expressing IFN-γ, TNF-α or IL-2 after o/n stimulation with S1+S2 peptides. Pie charts corresponding to cytokine profiles of CD8+ and CD4+ T cells T cells isolated from spleen and lung tissue. FIG. 6F shows polyfunctional profiles of antigen experienced CD8+ and CD4+ T cells. Pie charts corresponding to polyfunctional profiles of CD8+CD4+ T cells isolated from spleen and lung tissue after o/n stimulation with S1+S2 peptides. Assessment of the mean proportion of cells making any combination of 1-3 cytokines (IFN-γ, TNFα, IL-2). Data represent at least two technical replicates with 3-6 independent biological replicates per group. *p<0.05, **p<0.01, ***p<0.001. Kruskal-Wallis ANOVA with Dunn's multiple comparisons tests were applied. Asterisks (*) above or inside the column denote significant differences between indicated T cell producing cytokine in vaccine and control (PBS) at 0.05 alpha level.

FIGS. 7A and 7B are a series of graphs showing secreted Gp96-Ig-S vaccine induces S1 and S2 specific CD8+ T cells in the spleen, lung tissue and BAL. Five days after the vaccination of HLA-A2 transgenic mice, splenocytes and lung cells were isolated from vaccinated and control mice (PBS). Cell were stained with HLA-A2 02-01 pentamers containing FIAGLIAIV (SEQ ID NO: 96) and YLQPRTFLL (SEQ ID NO: 97) peptides, followed by surface for CD45, CD3, CD4, CD8 and CD19. FIG. 7A are bar graphs representing percentage of the pentamer positive cells within S1 (FIG. 7A, left panel) and S2 (FIG. 7A, right panel) specific CD8+ T cells. FIG. 7B are representative zebra plots of gated CD8 T cells expressing indicated peptide specific TCR+ CD8 T cells in vaccinated and non-vaccinated HLA-A2 mice. Data represent at least two technical replicates with 3-6 independent biological replicates per group. *p<0.05, **p<0.01, ***p<0.001. Kruskal-Wallis ANOVA with Dunn's multiple comparisons tests were applied. Asterisks (*) above or inside the column denote significant differences between indicated pentamer positive(+) CD8+ T cells in the vaccinated group and control (PBS) at 0.05 alpha level.

FIG. 8 is a graph showing how the secreted Gp96-Ig-S vaccine induces CD69+CXCR6+ S− specific (YQL) CD8+ T cells in the spleen, lung tissue and BAL. Five days after the vaccination of HLA-A2 transgenic mice, splenocytes and lung cells were isolated from vaccinated and control mice (PBS) and re-stimulated in vitro with S1 and S2 overlapping peptides from SARS-CoV-2 protein in the presence of protein transport inhibitor, brefeldin A for the last 5 h of culture. After 20 h of culture, cell were stained with an HLA-A2 02-01 pentamer containing FIAGLIAIV (SEQ ID NO: 96) and YLQPRTFLL (SEQ ID NO: 97) peptides, followed by surface for CD45, CD3, CD4, CD8, CD69, CXCR6. Bar graphs represent percentage of the pentamer positive cells within CD8+ T cells. Representative zebra plots of gated CD8 T cells expressing indicated peptide specific TCR+ CD8 T cells in vaccinated and non-vaccinated HLA-A2 mice. Data represent at least two technical replicates with 3-6 independent biological replicates per group. *p<0.05, **p<0.01, ***p<0.001. Kruskal-Wallis ANOVA with Dunn's multiple comparisons tests were applied.

FIGS. 9A-9F show results of comparing frequency of HLA-A02.1 pentamer+ cells (YLQ+) within CD8+ T cells after vaccination with different number of ZVX-60 and ZVX-55 vaccine cells. Bar graphs represent percentage of pentamer positive (YLQ+) cells within CD8+ T cells, as follows: ZVX-60 in spleen (“SPL”) (FIG. 9A), ZVX-55 in spleen (“SPL”) (FIG. 9B), ZVX-60 in lungs (FIG. 9C), ZVX-55 in lungs (FIG. 9D), ZVX-60 in BAL (FIG. 9E), and ZVX-55 in BAL (FIG. 9F). In FIGS. 9A, 9C, and 9E, the x-axis shows control (“CTRL”), 0.25×10⁶, 0.5×10⁶, 1×10⁶, and 2×10⁶ injected cells for ZVX-60. In FIGS. 9B, 9D, and 9F, the x-axis shows control (“CTRL”), 0.2×10⁶, 0.5×10⁶, and 1×10⁶ injected cells for ZVX-55. The data represents at least 2 technical replicates with 3-5 independent biologic replicates per group.

FIG. 10 illustrates results of the study of CD69 and CXCR6 marker expression on CD8+ T cells after ZVX-60 vaccination. Bar graphs represent percentage of marker positive cells within total CD8+ T cells for CD69 (0.25×10⁶ injected cells), CD69 (0.5×10⁶ injected cells), CD69 (1×10⁶ injected cells), CXCR6 (0.25×10⁶ injected cells), CXCR6 (0.5×10⁶ injected cells), and CXCR6 (1×10⁶ injected cells) for each of the spleen (“SPL”), lungs, and BAL. Data represent at least 2 technical replicates with 3 independent biologic replicates per group.

FIGS. 11A-11F illustrate results of comparison of frequency of different CD8+ and CD4+ T cell subsets after several different doses of ZVX-60. In FIGS. 11A-11F, bar graphs represent percentage of positive cells of CD8+ T and CD4+ T cell subsets: effector memory (“EM,” CD44+CD62L−), central memory (“CM,” CD44+CD62L+), naïve (“Naïve,” CD44−CD62L−); and effector (“EFF,” CD44−CD62L−) cells, within total CD8+ T cells or CD4+ T cells. FIG. 11A shows percentage of positive cells within CD8+ T cells in the spleen (“SPL”). FIG. 11B shows percentage of positive cells within CD4+ T cells in the spleen (“SPL”). FIG. 11C shows percentage of positive cells within CD8+ T cells in lungs. FIG. 11D shows percentage of positive cells within CD4+ T cells in lungs. FIG. 11E shows percentage of positive cells within CD8+ T cells in the BAL. FIG. 11F shows percentage of positive cells within CD4+ T cells in the BAL. Data represent at least 2 technical replicates with 3-5 independent biologic replicates per group. For each of the EM, CM, Naïve, and EFF subsets, in FIGS. 11A-11F, the following doses of ZVX-60 vaccine cells are shown, in this order: control (“CTRL), 0.25×10⁶, 0.5×10⁶, 1×10⁶, and 2×10⁶ vaccine cells.

### DETAILED DESCRIPTION

The present invention provides an expression vector system, a composition, or various biologicals cells, and methods that use them, which are able to stimulate, without limitation, innate and adaptive immune responses in the host cell thereby providing direct protection against SARS-CoV-2 infection. Further, the present invention provides a gp96-based SARS-CoV-2 vaccine that demonstrates a significant and robust T cell mediated immune response. As disclosed herein, the gp96-based SARS-CoV-2 vaccine induces the expansion of both “killer” CD8 T cells that destroy virus infected cells, and “helper” CD4 T cells that help antibody production and release antiviral cytokines (e.g., IFNγ, TNF-α, and IL-2) that amplifies the immune response. Upon vaccination, memory CD8 T cells migrated to the lungs and airway passages, which are the tissue-specific site of interest for SARS-CoV-2 infection.

In embodiments, the expression vector system, composition, or biological cells provide protection against two or more different variants of SARS-CoV-2 protein (e.g., a Spike (or “S”) protein) or an antigenic portion thereof. Accordingly, the present disclosure allows preventing or mitigating a SARS-CoV-2 infection that can be caused by more than one variant of a coronavirus protein.

As the SARS-CoV-2 coronavirus continues to spread around the world, it evolves such that new variants, resulting from one or more mutations, continuously appear. For example, mutations in the gene encoding Spike protein are being continuously reported. See, e.g., Dawood. New Microbes New Infect. 2020; 35:100673; Korber et al., Cell. 2020 doi: 10.1016/j.cell.2020.06.043. Published online Jul. 3, 2020; Saha et al., Biosci. Rep. 2020; Sheikh et al., Infect. Genet. Evol. 2020; 84:104330; and van Dorp et al., Infect. Genet. Evol. 2020; 83:104351.

Today, no consistent nomenclature has been established for SARS-CoV-2. See WHO Headquarters (8 Jan. 2021). “3.6 Considerations for virus naming and nomenclature.” SARS-CoV-2 genomic sequencing for public health goals: Interim guidance, 8 Jan. 2021. World Health Organization. The WHO is currently working on the standard nomenclature. While there are many thousands of variants of SARS-CoV-2 (see Koyama et al., June 2020, “Variant analysis of SARS-CoV-2 genomes.” Bulletin of the World Health Organization. 98 (7): 495-504), subtypes of the virus can be put into larger groupings such as lineages or clades. As of today, three main general nomenclatures have been used: GISAID (Global Initiative on Sharing All Influenza Data) which has identified eight global clades (S, O, L, V, G, GH, GR, and GV) (Shu & McCauley. “GISAID: Global initiative on sharing all influenza data—from vision to reality.” Euro Surveil. 2017; 22(13):30494); Nextstrain which, as of January 2021, has identified 11 major clades (19A, 19B, and 20A-20I) (Hadfield et al., “Nextstrain: real-time tracking of pathogen evolution.” Bioinformatics. 2018; 34(23):4121-3; Nextstrain, available from www://nextstrain.org/); and PANGOLIN (Phylogenetic Assignment of Named Global Outbreak Lineages) software that, as of February 2021, has identified multiple PANGO lineages and six major lineages (A, B, B.1, B.1.1, B.1.177, B.1.1.7) (Rambaut et al., “A dynamic nomenclature proposal for SARS-CoV-2 lineages to assist genomic epidemiology.” Nat Microbiol 5, 1403-1407 (2020) doi:10.1038/s41564-020-0770-5). A PANGO lineage is “a cluster of sequences that are associated with an epidemiological event, for instance an introduction of the virus into a distinct geographic area with evidence of onward spread.” Rambaut et al., (2020).

Recently, a SARS-CoV-2 virus variant, referred to as B.1.1.7 (also referred to as lineage B.1.1.7 or 201/501Y.V1/B.1.1.7), or, in the UK, as SARS-CoV-2 VUI 202012/01, has been uncovered, which is defined by multiple spike protein mutations (deletion 69-70, deletion 144, N501Y, A570D, D614G, P681H, T716I, S982A, D1118H) present, as well as mutations in other genomic regions. The variant belongs to GISAID clade GR. One of the mutations (N501Y) is located within the receptor binding domain. See “Rapid increase of a SARS-CoV-2 variant with multiple spike protein mutations observed in the United Kingdom.” published online 20 Dec. 2020. European Center for Disease Prevention and Control (ECDC).

A new SARS-CoV-2 variant 501Y.V2, also known as B.1.351 lineage (or 501.V2, 20H/501Y.V2), has been recently detected in South Africa, and it is characterized by eight lineage-defining mutations in the spike protein, including three at residues in the receptor-binding domain (RBD) (K417N, E484K, and N501Y) that may have functional significance. See Tegally et al., “Emergence and rapid spread of a new severe acute respiratory syndrome-related coronavirus 2 (SARS-CoV-2) lineage with multiple spike mutations in South Africa,” published online Dec. 22, 2020, medRxiv.

The variant B.1.1.28 (501Y.V3) has been initially discovered in South Africa and Brazil, and recently found in Japan. This variant has 12 mutations in the Spike protein, and includes three mutations (K417T, E484K, and N501Y) at the same RBD residues as B.1.351. See Faria et al., “Genomic characterisation of an emergent SARS-CoV-2 lineage in Manaus: preliminary findings,” published online Jan. 12, 2021, COVID-19 Genomics Consortium UK (CoG-UK); see also Naveca et al., “Phylogenetic relationship of SARS-CoV-2 sequences from Amazonas with emerging Brazilian variants harboring mutations E484K and N501Y in the Spike protein,” published online Jan. 11, 2021, available from www://virological.org/.

Another recently identified lineage is SARS-CoV-2 P2, originated from B.1.1.28, distinguished by five single-nucleotide variants (SNVs): C100U, C28253U, G28628U, G28975U, and C29754U. The SNV G23012A (E484K), in the receptor-binding domain of Spike protein, was widely spread across the samples. See Voloch et al., “Genomic characterization of a novel SARS-CoV-2 lineage from Rio de Janeiro, Brazil,” published online Dec. 26, 2020. medRxiv.

Another known variant (or lineage) is P.1 (20J/501Y.V3), which is also a branch of the B.1.1.28 lineage, and that was first reported by the National Institute of Infectious Diseases (NIID) in Japan. The P.1 variant contains three mutations in the spike protein receptor binding domain: K417T, E484K, and N501Y. The full set of spike protein changes for the variant are amino acid change L18F, T20N, P26S, D138Y, R1905, K417T, E484K, N501Y, H655Y, T10271, and V1176F. See Faria et al., published online Jan. 12, 2021; see also “Risk related to the spread of new SARS-CoV-2 variants of concern in the EU/EEA—first update,” published online Jan. 21, 2021, The European Centre for Disease Prevention and Control.

Independent genomic surveillance programs based in New Mexico and Louisiana simultaneously detected a rapid rise of numerous clade 20 G (lineage B.1.2) infections carrying a Q677P substitution in the Spike protein. Hodcroft et al., medRxiv, BMJ Yale, published online Feb. 14, 2021, doi: doi.org/10.1101/2021.02.12.21251658. The variant Q677P cases have been detected predominantly in the south central and southwest United States; as of Feb. 3, 2021, GISAID data showed 499 viral sequences of this variant from the USA. Id.

Most recently, a new SARS-CoV-2 variant, CAL.20C, has been detected in Southern California after a surge in local infections in October 2020. See Zhang et al., “Emergence of a Novel SARS-CoV-2 Variant in Southern California.” JAMA. Published online Feb. 11, 2021. doi:10.1001/jama.2021.1612. This novel variant has descended from cluster 20C, is defined by 5 mutations (ORF1a: 14205V, ORF1b: D1183Y, S: S131; W152C; and L452R), and designated CAL.20C (20C/S:452R; /B.1.429). Id. The S131, W152C, and L452R mutations are in the S-protein, characterizing this strain as a subclade of 20 C. The S protein L452R mutation is within a known receptor binding domain that has been found to be resistant to certain spike (S) protein monoclonal antibodies. Li et al., Cell. 2020; 182(5):1284-1294.e9

One of the most dominant variants is D614G (i.e. an aspartic acid to glycine amino acid substitution at position 614 in the viral S gene), which is suspected to have increased infectivity and transmission. See Korber et al., Cell. 2020; Tang et al., “Emergence of a new SARS-CoV-2 variant in the UK,” published online Dec. 28, 2020, Journal of Infection. Other notable mutations are E484K, which has been reported to be an escape mutation (i.e., a mutation that improves a virus's ability to evade the host's immune system (See Wise, Feb. 5, 2021. “Covid-19: The E484K mutation and the risks it poses.” The BMJ. 372: n359. doi:10.1136/bmj.n359)); N501Y; K417N, and S477G/N (see Singh et al., Feb. 22, 2021. “Serine 477 plays a crucial role in the interaction of the SARS-CoV-2 spike protein with the human receptor ACE2.” Scientific Reports. 11 (1): 4320. doi:10.1038/s41598-021-83761-5; Schrörs et al., Feb. 4, 2021. “Large-scale analysis of SARS-CoV-2 spike-glycoprotein mutants demonstrates the need for continuous screening of virus isolates.” bioRxiv: 2021.02.04.429765).

Most current SARS-CoV-2 vaccines deliver immunogens based on the Spike protein sequence of the Wuhan reference sequence (GenBank accession no. MN908947). See Korber et al., Cell. 2020; Wang et al., J. Med. Virol. 2020; 92: 667-674. Accordingly, as new variants, particularly variants having one or more mutations in the Spike protein, emerge, existing vaccines and other treatments may not keep up. For example, a E484K amino acid mutation in the receptor-binding-domain (RBD) of the B.1.351 variant was reported to be “associated with escape from neutralising antibodies,” which can adversely affect the efficacy of COVID vaccines directed to the Spike protein. Callaway (7 Jan. 2021). “Could new COVID variants undermine vaccines? Labs scramble to find out.” Nature. Different other changes in the Spike protein, as well as in other parts of the coronavirus, can affect availability and efficacy of vaccines.

Accordingly, embodiments of the present disclosure provide a cell-based vaccine that allows simultaneously targeting one or more variants of a coronavirus protein, such as, without limitation, the Spike protein.

In embodiments, a “variant” refers to any one or more mutations in a coronavirus protein, wherein the coronavirus protein variant can be naturally occurring or an engineered protein.

For purposes of the present disclosure, the variant can be interchangeably referred to as a lineage or strain. It should be appreciated however that there may be differences between a variant, a strain, and a lineage. For example, a variant can be defined to be a strain once that variant has a certain frequency of occurrence in a population. Because the coronavirus causing SARS-CoV-2 is evolving, the definition of the coronavirus variants is also changing as more data is being collected. For example, GISAID (Global Initiative on Sharing All Influenza Data) currently includes over 30,000 of coronavirus sequences. Since the public release of the first reference sequence (GenBank Accession No.: MN908947) on Jan. 12, 2020, the number of sequences available has increased exponentially, at a current rate of approximately 70 new sequences per day. Rouchka et al., Variant analysis of 1,040 SARS-CoV-2 genomes. PLOS ONE, Published: Nov. 5, 2020; see also Shu & McCauley. GISAID: Global initiative on sharing all influenza data—from vision to reality. Euro Surveill. 2017; 22(13):30494. Furthermore, as mentioned above, a nomenclature for SARS-CoV-2 lineages is being developed, such that it is suggested to use a “dynamic” nomenclature that evolves as viral lineages appear and disappear through time. See Rambaut et al., “A dynamic nomenclature proposal for SARS-CoV-2 lineages to assist genomic epidemiology.” Nat Microbiol 5, 1403-1407 (2020).

In various embodiments, a cell-based vaccine is provided that is capable of targeting a coronavirus protein, such as, e.g., a SARS-CoV-2 protein, that belongs to any variant, strain, lineage, and/or clade of coronavirus. In various embodiments, a cell-based vaccine is provided that is capable of targeting a “cocktail” of coronavirus proteins, such as, e.g., one or more SARS-CoV-2 proteins, that belong to any variant, strain, lineage, and/or clade of coronavirus. In some embodiments, the cell-based vaccine includes a T cell costimulatory fusion protein such as, for example, OX40L.

In embodiments, various doses of the vaccine in accordance with the present disclosure can be used. In some embodiments, the composition comprises at least or about 0.5×10⁶ cells transfected with the expression vector system, optionally comprising 0.5×10⁶ cells; and/or an effective amount of cells that express and/or secrete at least or about 500-1000 ng of secretable fusion protein, optionally gp96.

In embodiments, the composition comprises at least 0.5×10⁶ cells transfected with the expression vector system. In embodiments, the composition comprises about 0.5×10⁶ cells transfected with the expression vector system.

In embodiments, the composition comprises from about 0.25×10⁶ cells to about 1×10⁶ cells transfected with the expression vector system. In embodiments, the composition comprises from about 0.25×10⁶ cells to about 0.5×10⁶ cells transfected with the expression vector system. In embodiments, the composition comprises at least 0.25×10⁶ cells transfected with the expression vector system. In embodiments, the composition comprises about 0.25×10⁶ cells transfected with the expression vector system.

In embodiments, the composition comprises an effective amount of cells that express and/or secrete at least or about 500-1000 ng of secretable fusion protein, optionally gp96.

In embodiments, the composition comprises an effective amount of cells that express and/or secrete at least 500 ng of secretable fusion protein, optionally gp96. In embodiments, the composition comprises an effective amount of cells that express and/or secrete from about 500 ng to about 1000 ng of secretable fusion protein, optionally gp96. In embodiments, the composition comprises an effective amount of cells that express and/or secrete about 500 ng of secretable fusion protein, optionally gp96.

In embodiments, the composition comprises an effective amount of cells that express at least 500 ng of secretable fusion protein, optionally gp96. In embodiments, the composition comprises an effective amount of cells that express from about 500 ng to about 1000 ng of secretable fusion protein, optionally gp96. In embodiments, the composition comprises an effective amount of cells that express about 500 ng of secretable fusion protein, optionally gp96.

In embodiments, the composition comprises an effective amount of cells that secrete at least 500 ng of secretable fusion protein, optionally gp96. In embodiments, the composition comprises an effective amount of cells that secrete from about 500 ng to about 1000 ng of secretable fusion protein, optionally gp96. In embodiments, the composition comprises an effective amount of cells that secrete about 500 ng of secretable fusion protein, optionally gp96.

In embodiments, the composition comprises an effective amount of cells (e.g., without limitation, of a vaccine including OX40L) that is from about 500 ng to about 1000 ng of a secretable fusion protein, optionally gp96, or about 1000 ng of the secretable fusion protein. In some embodiments, a dose of the vaccine is from about 500 ng to about 2000 ng, or from about 500 ng to about 1500 ng, or from about 500 ng to about 1400 ng, or from about 500 ng to about 1300 ng, or from about 500 ng to about 1200 ng, or from about 500 ng to about 1100 ng, or from about 500 ng to about 1000 ng, or from about 500 ng to about 800 ng of the secretable fusion protein.

In some embodiments, a lower dose of OX40L is used (based on receptor occupancy), since, at higher doses/receptor occupancy, OX40L expression is reduced. Accordingly, higher doses of OX40L can surprisingly be less efficient than lower doses (e.g., can lead to a loss of OX40L receptor expression).

In some embodiments, a dose of a vaccine including OX40L of from about 500 ng to about 1000 ng of the vaccine cells induces central memory CD8+ T cells, whereas the doses of 2000 ng and higher induce primarily effector memory and effector CD8+ T cell phenotype. Similarly, a low dose of a vaccine induces central memory CD4+ T cells, while a high dose induces effector CD4+ T cell phenotype.

It was observed that, for solid tumors treated with OX40 mAb, OX40 receptor occupancy between 20% and 50% both in vivo and in vitro was associated with maximum enhancement of T-cell effector function by anti-OX40 treatment, whereas a receptor occupancy >40% led to a profound loss in OX40 receptor expression. See Wang et al., Clin Cancer Res. 2019 Nov. 15; 25(22):6709-6720. It was also observed that, a high dose OX40 agonist mAb reduced rather than enhanced immune response in monkeys. See Gamse et al., Toxicology and Applied Pharmacology, Volume 409, 2020, 115285, ISSN 0041-008X. These findings suggest that, at higher doses/receptor occupancy, OX40 expression is reduced. Also, repeat dosing can be used.

Furthermore, in embodiments, targeting receptor occupancy between approximately 20% and 50% results in maximal potentiation of T-cell responses by a therapeutic OX40 agonist antibody.

Vaccine Proteins

Vaccine proteins can induce immune responses, including long-lasting immune responses, that find use in the present invention.

In embodiments, the expression vector system, compositions, and cells are capable of activating subject's innate immune response, as well as humoral response (i.e. antibody response) and cellular response (i.e. T cell response). In some embodiments, the expression vector system, composition, and cells are able to activate the immune response, antibody response, and/or the T-cell-driven cellular immune in a subject.

In various embodiments, the present invention provides expression vectors comprising a first nucleotide sequence encoding a secretable vaccine protein, a second nucleotide sequence encoding a T cell costimulatory fusion protein, and/or a third nucleotide sequence encoding a coronavirus protein, or an antigenic portion thereof. In embodiments, a third nucleotide sequence is in the form of one, two, or more than two nucleic acids, each encoding a respective variant of a coronavirus protein or an antigenic portion thereof. Compositions comprising the expression vectors of the present invention are also provided. In various embodiments, such compositions are utilized in methods of treating subjects to stimulate immune responses in the subject affected by a coronavirus or at risk to be affected by a coronavirus (e.g., without limitation during an outbreak), including enhancing the activation of antigen-specific T cells in the subject. The present compositions find use in the treating or preventing a coronavirus infection in a subject.

In some embodiments, the secretable vaccine protein is a heat shock protein (hsp) gp96 that is localized in the endoplasmic reticulum (ER) and serves as a chaperone for peptides on their way to MHC class I and II molecules. Gp96 obtained from tumor cells and used as a vaccine can induce specific tumor immunity, presumably through the transport of tumor-specific peptides to antigen-presenting cells (APCs) (J Immunol 1999, 163(10):5178-5182). For example, gp96-associated peptides are cross-presented to CD8 cells by dendritic cells (DCs). Gp96-based vaccination modality has also been shown to provide protection against mucosal infection caused by simian immunodeficiency virus. Strbo et al., J Immunol. 2013; 190(6):2495-2499.

In embodiments in accordance with the present disclosure, an expression vector system or a population of cells transfected with the expression vector system is designed to use gp96 so as to trigger mucosal immunity by activating both B and T cell responses at the point of pathogen entry. The gp96-based expression vector system effectively presents multiple SARS-CoV-2 antigens and activates the immune system thereby. The gp96-based expression vector system utilizes natural and adaptive immune process to induce long-lasting memory responses against SARS-CoV-2 virus.

In some embodiments, the present compositions stimulate, promote, or increase one or more of a T-cell response, antibody response, and activation of innate immunity. In some embodiments, the present compositions stimulate, promote, or increase all three of the T-cell response, antibody response, and activation of innate immunity, thereby activating all three arms of the subject's immune system.

In some embodiments, the present compositions activate innate immunity via Toll-Like Receptor (TLRs), as, without wishing to be bound by the theory, gp96 activates Toll-Like Receptor 4/2 (TLR4 and TLR2) on macrophages and dendritic cells.

Furthermore, the present compositions, adapted to present multiple SARS-CoV-2 antigens, in accordance with embodiments of the present disclosure, stimulate, promote, or increase a prominent cellular immune response via CD4 and CD8 T cells, in addition to the humoral immune response, via neutralizing IgG antibody.

The present invention addresses the problem that antibody responses in patients who recovered from SARS-CoV-2 may weaken or disappear, which may be due to the lack of optimal activation of T-cell immunity. For example, without limitation, CD4 T helper cells may not have been activated in response to SARS-CoV-2 infection, which can be a mechanism by which the virus suppresses host immunity and escapes immunosurveillance. In embodiments, this issue is addressed by providing an expression vector system, a composition, or various biologicals cells that are capable of activating robust T-cell immunity.

In embodiments, the method that uses the present compositions that present SARS-CoV-2 antigens is suitable for increasing the subject's T-cell response as compared to the T-cell response of a subject that was not administered the compositions. In embodiments, the method is suitable for increasing the subject's antibody response as compared to the antibody response of a subject that was not administered the compositions. In embodiments, the method is suitable for increasing the subject's innate immune response as compared to the innate immune response of a subject that was not administered the compositions. In embodiments, the method is suitable for increasing the subject's T-cell response, antibody response, and innate immune response as compared to the T-cell response, antibody response, and innate immune responses of a subject that was not administered the compositions.

In embodiments, the method is suitable for increasing the subject's innate immune response as compared to the innate immune response of a subject that was not administered the present compositions. In embodiments, the method is suitable for increasing the subject's adaptive immune response as compared to the adaptive immune response of a subject that was not administered the compositions. In embodiments, the method is suitable for increasing the subject's innate immune response and adaptive immune response as compared to the innate and adaptive immune responses of a subject that was not administered the compositions.

In some embodiments, methods and compositions of the present invention are for improving and/or increasing vaccine efficacy in a patient and include maintaining and/or increasing the patient's T cell populations (e.g., CD4+ and/or CD8+ T cell populations). In some embodiments, methods and compositions of the present invention are for improving and/or increasing vaccine efficacy in a patient and include maintaining and/or increasing the patient's antigen-specific antibody titers (e.g., IgG, IgM and IgA). In further embodiments, methods of the present invention provide for mitigation of age-related immunosenescence as measured by an increase or restoration of a patient's antigen-specific antibody titers (e.g., IgG, IgM and IgA).

In embodiments, the method is suitable for increasing and/or restoring the subject's T cell population(s) as compared to the T cell populations of a subject that was not administered the present compositions. In embodiments, the subject's T cells, including T cells selected from one or more of CD4+ effector T cells, CD8+ effector T cells, CD4+ memory T cells, CD8+ memory T cells, CD4+ central memory T cells, CD8+ central memory T cells, natural killer T cells, CD4+ helper cells, and CD8+ cytotoxic cells, are increased and/or restored as compared to the T cell populations of a subject that was not administered the compositions.

In embodiments, the method is suitable for increasing and/or restoring the subject's T cell population(s) as compared to the T cell populations of a subject that was administered another vaccine (e.g., without limitation, another coronavirus vaccine). In embodiments, the subject's T cells, including T cells selected from one or more of CD4+ effector T cells, CD8+ effector T cells, CD4+ memory T cells, CD8+ memory T cells, CD4+ central memory T cells, CD8+ central memory T cells, natural killer T cells, CD4+ helper cells, and CD8+ cytotoxic cells, are increased and/or restored as compared to the T cell populations of a subject that was administered another vaccine.

In embodiments, the subject's CD4+ helper cells population(s) are increased and/or restored as compared to the CD4+ helper cells populations of a subject that was not administered the present compositions. In some embodiments, without wishing to be bound by the theory, OX40L co-stimulation expands CD4 helper T cells that promote B-cell differentiation and IgG/IgA antibody class switching.

More specifically, in some embodiments, the present invention provides methods for improving and/or increasing vaccine efficacy in a patient, as measured by an increase and/or restoration of the patient's T cell subsets. In some embodiments, the T cells are T helper cells (e.g., Tₕ cells). In further embodiments, T helper cells secrete cytokines that attract one or more of macrophages, neutrophils, other lymphocytes, and other cytokines to further direct these cells. In some embodiments, CD4+ T helper cells are one of several subsets, including, Th1, Th2, Th17, Th9, and Tfh, with each subset having a different function.

In some embodiments, T cells are cytotoxic cells that optionally produce IL-2 and IFNγ cytokines. In further embodiments, these T cells are cytotoxic CD8+ T cells (also known as Tc cells or T-killer cells).

In some embodiments, memory T cells elicited by compositions and methods of the present invention are long-lived and can expand to large numbers of effector T cells when re-exposed to their cognate antigen. For example, the memory T cells elicited by methods of the present invention can persist in a subject for at least about 1 year, or at least about 10 years, or at least about 20 years, or at least about 30 years, or at least about 40 years, or at least about 50 years, or at least about 60 years, or at least about 70 years, or at least about 80 years. In some embodiments, memory T cells elicited by the compositions and methods of the present invention can last for the entire lifespan of a subject.

In some embodiments, memory T cells provide a patient's immune system with memory against previously encountered pathogens. In further embodiments, memory T cell populations include, but are not limited to, tissue-resident memory T (Trm) cells, stem memory TSCM cells, and virtual memory T cells. In some embodiments, memory T cells are classified as CD4+ or CD8+ and express CD45RO. In some embodiments, memory T cells are further differentiated into various subsets. For example, in some embodiments, memory T cell subsets include: Central memory T cells (T_{CM} cells), which can express CD45RO, C—C chemokine receptor type 7 (CCR7), L-selectin (CD62L), and CD44; Effector memory T cells (TEM cells and T_{EMRA} cells), which express CD45RO and CD44 but lack expression of CCR7 and CD62L; Tissue resident memory T cells (TRM), which is associated with the integrin aeβ7; and Virtual memory T cells.

When a cell abnormally dies through necrosis or infection, gp96 is naturally released into the surrounding microenvironment. Thus, gp96 becomes a Danger Associated Molecular Protein or “DAMP,” a molecular warning signal for localized innate activation of the immune system. In this context, gp96 serves as a potent adjuvant, or immune stimulator, via TLR4 and TLR2 signaling which serves to activate APCs to specialized dendritic cells that upregulate T-cell costimulatory ligands, MHC and immune activating cytokine. It is the powerful adjuvant that shows specificity to CD8+“killer” T-cells through cross-presentation of the gp96-chaperoned tumor associated peptide antigens directly to MHC class I molecules for direct activation and expansion of CD8+ T-cells.

A vaccination system was developed for antitumor therapy by transfecting a gp96-Ig G1-Fc fusion protein into tumor cells, resulting in secretion of gp96-Ig in complex with chaperoned tumor peptides (see, J Immunother 2008, 31(4):394-401, and references cited therein). Parenteral administration of gp96-Ig secreting tumor cells triggers robust, antigen-specific CD8 cytotoxic T lymphocyte (CTL) expansion, combined with activation of the innate immune system.

The expression vectors provided herein contain a first nucleotide sequence that encodes a gp96-Ig fusion protein. The coding region of human gp96 is 2,412 bases in length (SEQ ID NO:47), and encodes an 803 amino acid protein (SEQ ID NO:48) that includes a 21 amino acid signal peptide at the amino terminus, a potential transmembrane region rich in hydrophobic residues, and an ER retention peptide sequence at the carboxyl terminus (GENBANK® Accession No. X15187; see, Maki et al., Proc Natl Acad Sci USA 1990, 87:5658-5562). The DNA and protein sequences of human gp96 follow:

(SEQ ID NO: 47)
atgagggccctgtgggtgctgggcctctgctgcgtcctgctgaccttcgg
gtcggtcagagctgacgatgaagttgatgtggatggtacagtagaagagg
atctgggtaaaagtagagaaggatcaaggacggatgatgaagtagtacag
agagaggaagaagctattcagttggatggattaaatgcatcacaaataag
agaacttagagagaagtcggaaaagtttgccttccaagccgaagttaaca
gaatgatgaaacttatcatcaattcattgtataaaaataaagagattttc
ctgagagaactgatttcaaatgcttctgatgctttagataagataaggct
aatatcactgactgatgaaaatgctctttctggaaatgaggaactaacag
tcaaaattaagtgtgataaggagaagaacctgctgcatgtcacagacacc
ggtgtaggaatgaccagagaagagttggttaaaaaccttggtaccatagc
caaatctgggacaagcgagtttttaaacaaaatgactgaagcacaggaag
atggccagtcaacttctgaattgattggccagtttggtgtcggtttctat
tccgccttccttgtagcagataaggttattgtcacttcaaaacacaacaa
cgatacccagcacatctgggagtctgactccaatgaattttctgtaattg
ctgacccaagaggaaacactctaggacggggaacgacaattacccttgtc
ttaaaagaagaagcatctgattaccttgaattggatacaattaaaaatct
cgtcaaaaaatattcacagttcataaactttcctatttatgtatggagca
gcaagactgaaactgttgaggagcccatggaggaagaagaagcagccaaa
gaagagaaagaagaatctgatgatgaagctgcagtagaggaagaagaaga
agaaaagaaaccaaagactaaaaaagttgaaaaaactgtctgggactggg
aacttatgaatgatatcaaaccaatatggcagagaccatcaaaagaagta
gaagaagatgaatacaaagctttctacaaatcattttcaaaggaaagtga
tgaccccatggcttatattcactttactgctgaaggggaagttaccttca
aatcaattttatttgtacccacatctgctccacgtggtctgtttgacgaa
tatggatctaaaaagagcgattacattaagctctatgtgcgccgtgtatt
catcacagacgacttccatgatatgatgcctaaatacctcaattttgtca
agggtgtggtggactcagatgatctccccttgaatgtttcccgcgagact
cttcagcaacataaactgcttaaggtgattaggaagaagcttgttcgtaa
aacgctggacatgatcaagaagattgctgatgataaatacaatgatactt
tttggaaagaatttggtaccaacatcaagcttggtgtgattgaagaccac
tcgaatcgaacacgtcttgctaaacttcttaggttccagtcttctcatca
tccaactgacattactagcctagaccagtatgtggaaagaatgaaggaaa
aacaagacaaaatctacttcatggctgggtccagcagaaaagaggctgaa
tcttctccatttgttgagcgacttctgaaaaagggctatgaagttattta
cctcacagaacctgtggatgaatactgtattcaggcccttcccgaatttg
atgggaagaggttccagaatgttgccaaggaaggagtgaagttcgatgaa
agtgagaaaactaaggagagtcgtgaagcagttgagaaagaatttgagcc
tctgctgaattggatgaaagataaagcccttaaggacaagattgaaaagg
ctgtggtgtctcagcgcctgacagaatctccgtgtgctttggtggccagc
cagtacggatggtctggcaacatggagagaatcatgaaagcacaagcgta
ccaaacgggcaaggacatctctacaaattactatgcgagtcagaagaaaa
catttgaaattaatcccagacacccgctgatcagagacatgcttcgacga
attaaggaagatgaagatgataaaacagttttggatcttgctgtggtttt
gtttgaaacagcaacgcttcggtcagggtatcttttaccagacactaaag
catatggagatagaatagaaagaatgcttcgcctcagtttgaacattgac
cctgatgcaaaggtggaagaagagcccgaagaagaacctgaagagacagc
agaagacacaacagaagacacagagcaagacgaagatgaagaaatggatg
tgggaacagatgaagaagaagaaacagcaaaggaatctacagctgaaaaa
gatgaattgtaa
(SEQ ID NO: 48)
MRALWVLGLCCVLLTFGSVRADDEVDVDGTVEEDLGKSREGSRTDDEVVQ
REEEAIQLDGLNASQIRELREKSEKFAFQAEVNRMMKLIINSLYKNKEIF
LRELISNASDALDKIRLISLTDENALSGNEELTVKIKCDKEKNLLHVTDT
GVGMTREELVKNLGTIAKSGTSEFLNKMTEAQEDGQSTSELIGQFGVGFY
SAFLVADKVIVTSKHNNDTQHIWESDSNEFSVIADPRGNTLGRGTTITLV
LKEEASDYLELDTIKNLVKKYSQFINFPIYVWSSKTETVEEPMEEEEAAK
EEKEESDDEAAVEEEEEEKKPKTKKVEKTVWDWELMNDIKPIWQRPSKEV
EEDEYKAFYKSFSKESDDPMAYIHFTAEGEVTFKSILFVPTSAPRGLFDE
YGSKKSDYIKLYVRRVFITDDFHDMMPKYLNFVKGVVDSDDLPLNVSRET
LQQHKLLKVIRKKLVRKTLDMIKKIADDKYNDTFWKEFGTNIKLGVIEDH
SNRTRLAKLLRFQSSHHPTDITSLDQYVERMKEKQDKIYFMAGSSRKEAE
SSPFVERLLKKGYEVIYLTEPVDEYCIQALPEFDGKRFQNVAKEGVKFDE
SEKTKESREAVEKEFEPLLNWMKDKALKDKIEKAVVSQRLTESPCALVAS
QYGWSGNMERIMKAQAYQTGKDISTNYYASQKKTFEINPRHPLIRDMLRR
IKEDEDDKTVLDLAVVLFETATLRSGYLLPDTKAYGDRIERMLRLSLNID
PDAKVEEEPEEEPEETAEDTTEDTEQDEDEEMDVGTDEEEETAKESTAEK
DEL.

A nucleic acid encoding a gp96-Ig fusion sequence can be produced using, for example, methods described in U.S. Pat. No. 8,685,384, which is incorporated herein by reference in its entirety. In some embodiments, the gp96 portion of a gp96-Ig fusion protein can contain all or a portion of a wild type gp96 sequence (e.g., the human sequence set forth in SEQ ID NO:48). For example, a secretable gp96-Ig fusion protein can include the first 799 amino acids of SEQ ID NO:48, such that it lacks the C-terminal KDEL (SEQ ID NO:49) sequence. Alternatively, the gp96 portion of the fusion protein can have an amino acid sequence that contains one or more substitutions, deletions, or additions as compared to the first 799 amino acids of the wild type gp96 sequence, such that it has at least 90% (e.g., at least 90%, at least 91%, at least 92%, at least 93%, at least 94%, at least 95%, at least 96%, at least 97%, at least 98%, or at least 99%) sequence identity to the wild type polypeptide.

In various embodiments, the gp96-Ig fusion protein and/or the costimulatory molecule fusions, comprise a linker. In various embodiments, the linker may be derived from naturally-occurring multi-domain proteins or are empirical linkers as described, for example, in Chichili et al., (2013), Protein Sci. 22(2):153-167, Chen et al., (2013), Adv Drug Deliv Rev. 65(10):1357-1369, the entire contents of which are hereby incorporated by reference. In some embodiments, the linker may be designed using linker designing databases and computer programs such as those described in Chen et al., (2013), Adv Drug Deliv Rev. 65(10):1357-1369 and Crasto et. al., (2000), Protein Eng. 13(5):309-312, the entire contents of which are hereby incorporated by reference.

In some embodiments, the linker is a synthetic linker such as PEG.

In other embodiments, the linker is a polypeptide. In some embodiments, the linker is less than about 100 amino acids long. For example, the linker may be less than about 100, about 95, about 90, about 85, about 80, about 75, about 70, about 65, about 60, about 55, about 50, about 45, about 40, about 35, about 30, about 25, about 20, about 19, about 18, about 17, about 16, about 15, about 14, about 13, about 12, about 11, about 10, about 9, about 8, about 7, about 6, about 5, about 4, about 3, or about 2 amino acids long. In some embodiments, the linker is flexible. In another embodiment, the linker is rigid. In various embodiments, the linker is substantially comprised of glycine and serine residues (e.g. about 30%, or about 40%, or about 50%, or about 60%, or about 70%, or about 80%, or about 90%, or about 95%, or about 97% glycines and serines).

In various embodiments, the linker is a hinge region of an antibody (e.g., of IgG, IgA, IgD, and IgE, inclusive of subclasses (e.g. IgG1, IgG2, IgG3, and IgG4, and IgA1 and IgA2)). The hinge region, found in IgG, IgA, IgD, and IgE class antibodies, acts as a flexible spacer, allowing the Fab portion to move freely in space. In contrast to the constant regions, the hinge domains are structurally diverse, varying in both sequence and length among immunoglobulin classes and subclasses. For example, the length and flexibility of the hinge region varies among the IgG subclasses. The hinge region of IgG1 encompasses amino acids 216-231 and, because it is freely flexible, the Fab fragments can rotate about their axes of symmetry and move within a sphere centered at the first of two inter-heavy chain disulfide bridges. IgG2 has a shorter hinge than IgG1, with 12 amino acid residues and four disulfide bridges. The hinge region of IgG2 lacks a glycine residue, is relatively short, and contains a rigid poly-proline double helix, stabilized by extra inter-heavy chain disulfide bridges. These properties restrict the flexibility of the IgG2 molecule. IgG3 differs from the other subclasses by its unique extended hinge region (about four times as long as the IgG1 hinge), containing 62 amino acids (including 21 prolines and 11 cysteines), forming an inflexible poly-proline double helix. In IgG3, the Fab fragments are relatively far away from the Fc fragment, giving the molecule a greater flexibility. The elongated hinge in IgG3 is also responsible for its higher molecular weight compared to the other subclasses. The hinge region of IgG4 is shorter than that of IgG1 and its flexibility is intermediate between that of IgG1 and IgG2. The flexibility of the hinge regions reportedly decreases in the order IgG3>IgG1>IgG4>IgG2.

Additional illustrative linkers include, but are not limited to, linkers having the sequence LE, GGGGS (SEQ ID NO:72), (GGGGS)ₙ (n=1-4) (SEQ ID NO: 73), (Gly)₈ (SEQ ID NO:74), (Gly)₆ (SEQ ID NO:75), (EAAAK)ₙ (n=1-3) (SEQ ID NO: 76), A(EAAAK)ₙA (n=2-5) (SEQ ID NO: 77), AEAAAKEAAAKA (SEQ ID NO: 78), A(EAAAK)₄ALEA(EAAAK)₄A (SEQ ID NO: 79), PAPAP (SEQ ID NO: 80), KESGSVSSEQLAQFRSLD (SEQ ID NO: 81), EGKSSGSGSESKST (SEQ ID NO: 82), GSAGSAAGSGEF (SEQ ID NO: 83), and (XP)ₙ, with X designating any amino acid, e.g., Ala, Lys, or Glu.

In various embodiments, the linker may be functional. For example, without limitation, the linker may function to improve the folding and/or stability, improve the expression, improve the pharmacokinetics, and/or improve the bioactivity of the present compositions. In another example, the linker may function to target the compositions to a particular cell type or location.

In some embodiments, a gp96 peptide can be fused to the hinge, CH2 and CH3 domains of murine IgG1 (Bowen et al., J Immunol 1996, 156:442-449). This region of the IgG1 molecule contains three cysteine residues that normally are involved in disulfide bonding with other cysteines in the Ig molecule. Since none of the cysteines are required for the peptide to function as a tag, one or more of these cysteine residues can be substituted by another amino acid residue, such as, for example, serine.

Various leader sequences known in the art also can be used for efficient secretion of gp96-Ig fusion proteins from bacterial and mammalian cells (see, von Heijne, J Mol Biol 1985, 184:99-105). Leader peptides can be selected based on the intended host cell, and may include bacterial, yeast, viral, animal, and mammalian sequences. For example, the herpes virus glycoprotein D leader peptide is suitable for use in a variety of mammalian cells. Another leader peptide for use in mammalian cells can be obtained from the V-J2-C region of the mouse immunoglobulin kappa chain (Bernard et al., Proc Natl Acad Sci USA 1981, 78:5812-5816). DNA sequences encoding peptide tags or leader peptides are known or readily available from libraries or commercial suppliers, and are suitable in the fusion proteins described herein.

Furthermore, in various embodiments, one may substitute the gp96 of the present disclosure with one or more vaccine proteins. For instance, various heat shock proteins are among the vaccine proteins. In various embodiments, the heat shock protein is one or more of a small hsp, hsp40, hsp60, hsp70, hsp90, and hsp110 family member, inclusive of fragments, variants, mutants, derivatives or combinations thereof (Hickey, et al., 1989, Mol. Cell. Biol. 9:2615-2626; Jindal, 1989, Mol. Cell. Biol. 9:2279-2283).

Expression Vectors and Host Cells

The present invention provides an expression vector system comprising (i) a nucleic acid encoding a fusion protein comprising a chaperone protein and an immunoglobulin, or a fragment thereof, (ii) a nucleic acid encoding a T cell costimulatory fusion protein, wherein the T cell costimulatory fusion protein enhances activation of antigen-specific T cells when administered to a subject; and/or (iii) a nucleic acid encoding a coronavirus protein, or an antigenic portion thereof, wherein each nucleic acid is operably linked to a promoter. In embodiments, the expression vector system comprises one, two, or more than two nucleic acids, each encoding a respective variant of a coronavirus protein or an antigenic portion thereof.

In some embodiments, the coronavirus is a betacoronavirus protein or an alphacoronavirus protein, optionally wherein the betacoronavirus protein is selected from a SARS-CoV-2, SARS-CoV, MERS-CoV, HCoV-HKU1, and HCoV-OC43 protein, or an antigenic fragment thereof, or the alphacoronavirus protein is selected from an HCoV-NL63 and HCoV-229E protein, or an antigenic fragment thereof. In some embodiments, the betacoronavirus protein is SARS-CoV-2 protein. In some embodiments, the SARS-CoV-2 protein is a variant of the SARS-CoV-2 protein, optionally a variant of a spike surface glycoprotein.

In embodiments, the coronavirus protein can be any protein recorded in the Global Initiative for Sharing All Influenza Data (GISAID) database (www.gisaid.org/).

In some embodiments, the coronavirus protein can be any protein included in any of the PANGO lineages (found in www.cov-lineages.org). Rambaut et al., (2020). In some embodiments, the coronavirus protein can be an engineered protein. For example, a bioinformatics analysis can be applied to “predict” one or more coronavirus protein variants to be targeted, e.g., in a certain geographical region, for an outbreak, etc.

In some embodiments, the present invention provides the expression vector system in which the nucleic acid encoding one or more the fusion proteins is operably linked to a promoter which is different from the promoter which is operably linked to the nucleic acid encoding the coronavirus protein, or an antigenic portion thereof.

In some embodiments, an expression vector system is provided that comprises (i) a nucleic acid encoding a secretable fusion protein comprising a chaperone protein and an immunoglobulin, or a fragment thereof, and (ii) a nucleic acid encoding a T cell costimulatory fusion protein, wherein the T cell costimulatory fusion protein enhances activation of antigen-specific T cells when administered to a subject; and/or (iii) a nucleic acid encoding a coronavirus protein, or an antigenic portion thereof. Each nucleic acid can be operably linked to a promoter. In embodiments, the expression vector system comprises one or more nucleic acids, each encoding a respective variant of a coronavirus protein or an antigenic portion thereof.

In some embodiments, the expression vector system comprises (i) a nucleic acid encoding a secretable fusion protein comprising a chaperone protein and an immunoglobulin, or a fragment thereof, (ii) a nucleic acid encoding a T cell costimulatory fusion protein; and/or (iii) a nucleic acid encoding a coronavirus protein, or an antigenic portion thereof. In some embodiments, the expression vector system comprises (i) a nucleic acid encoding a secretable fusion protein comprising a chaperone protein and an immunoglobulin, or a fragment thereof, and (ii) a nucleic acid encoding a T cell costimulatory fusion protein. In some embodiments, the expression vector system comprises a nucleic acid encoding a secretable fusion protein comprising a chaperone protein and an immunoglobulin, and a nucleic acid encoding a coronavirus protein, or an antigenic portion thereof.

In some embodiments, the T cell costimulatory protein can be an agonist of OX40 (e.g., an OX40 ligand-Ig (OX40L-Ig) fusion, or a fragment thereof that binds OX40), an agonist of inducible T-cell costimulator (ICOS) (e.g., an ICOS ligand-Ig (ICOSL-Ig) fusion, or a fragment thereof that binds ICOS), an agonist of CD40 (e.g., a CD40L-Ig fusion protein, or fragment thereof), an agonist of CD27 (e.g. a CD70-Ig fusion protein or fragment thereof), or an agonist of 4-1BB (e.g., a 4-1BB ligand-Ig (4-1BBL-Ig) fusion, or a fragment thereof that binds 4-1BB). In some embodiments, the expression vector system can encode an agonist of TNFRSF25 (e.g., a TL1A-Ig fusion, or a fragment thereof that binds TNFRSF25), or an agonist of glucocorticoid-induced tumor necrosis factor receptor (GITR) (e.g., a GITR ligand-Ig (GITRL-Ig) fusion, or a fragment thereof that binds GITR), or an agonist of CD40 (e.g., a CD40 ligand-Ig (CD40L-Ig) fusion, or a fragment thereof that binds CD40); or an agonist of CD27 (e.g., a CD27 ligand-Ig (e.g. CD70L-Ig) fusion, or a fragment thereof that binds CD40).

Additional costimulatory molecules that may be utilized in the present invention include, but are not limited to, HVEM, CD28, CD30, CD30L, CD40, CD70, LIGHT (CD258), B7-1, and B7-2.

In some embodiments, there is provided a biological cell comprising a first recombinant protein having an amino acid sequence of at least 95% sequence identity with SEQ ID NO: 2 and a second recombinant protein having an amino acid sequence of at least 95% sequence identity with the amino acid sequence of SEQ ID NO: 37, the amino acid sequence of SEQ ID NO: 40, the amino acid sequence of SEQ ID NO: 39, or the amino acid sequence of SEQ ID NO: 44, or an antigenic fragment of any of the foregoing. In some embodiments, the first recombinant protein has at least 97% sequence identity with SEQ ID NO: 2 and the second recombinant protein having an amino acid sequence of at least 97% sequence identity with the amino acid sequence of SEQ ID NO: 37, the amino acid sequence of SEQ ID NO: 40, the amino acid sequence of SEQ ID NO: 39, or the amino acid sequence of SEQ ID NO: 44, or an antigenic fragment of any of the foregoing. In some embodiments, the first recombinant protein has at least 98% sequence identity with SEQ ID NO: 2 and the second recombinant protein having an amino acid sequence of at least 98% sequence identity with the amino acid sequence of SEQ ID NO: 37, the amino acid sequence of SEQ ID NO: 40, the amino acid sequence of SEQ ID NO: 39, or the amino acid sequence of SEQ ID NO: 44, or an antigenic fragment of any of the foregoing. In any of the embodiments described herein, or combination of the embodiments, SEQ ID NO: 2 can lack the terminal KDEL sequence.

In some embodiments, there are provided at least two biological cells, the first biological cell comprising an expression vector system comprising a nucleic acid encoding a fusion protein comprising a chaperone protein and an immunoglobulin, or a fragment thereof, the nucleic acid being operably linked to a promoter, the second biological cell comprising an expression vector system comprising a nucleic acid encoding a T cell costimulatory fusion protein, wherein the T cell costimulatory fusion protein enhances activation of antigen-specific T cells when administered to a subject; and/or the third biological cell comprising an expression vector system comprising a nucleic acid encoding a coronavirus protein, or an antigenic portion thereof, the nucleic acid being operably linked to a promoter.

In some embodiments, the third biological cell comprises more than one expression vector system, such that two or more expression vector systems each comprise a respective nucleic acid encoding a respective variant of a coronavirus protein.

As another variation, in some embodiments, more than one biological cell comprises a nucleic acid encoding a respective variant of a coronavirus protein. Thus, the third biological cell can comprise more than one biological cell, each comprising an expression vector system comprising a nucleic acid encoding a respective variant of a coronavirus protein, or an antigenic portion thereof, whereby such biological cell comprises respective different variants of a coronavirus protein.

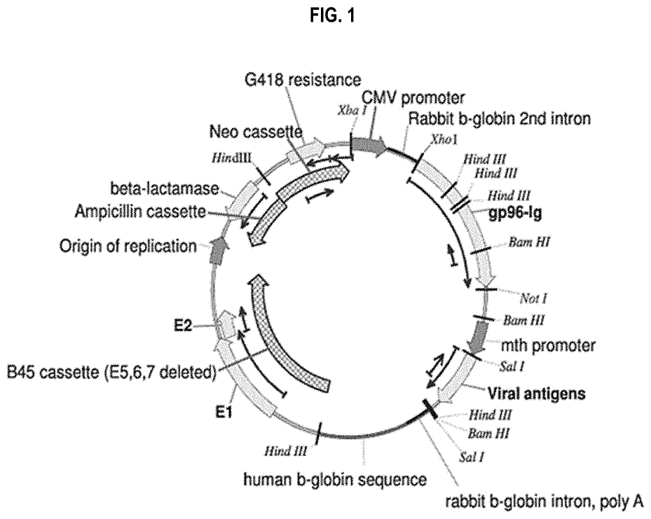

As used herein, the term “expression vector system” refers to one expression vector comprising all components or a set of two or more expression vectors designed to function together. For purposes herein, the term “expression vector” means a genetically-modified oligonucleotide or polynucleotide construct that permits the expression of an mRNA, protein, polypeptide, or peptide by a host cell, when the construct comprises a nucleotide sequence encoding the mRNA, protein, polypeptide, or peptide, and the expression vector is contacted with the cell under conditions sufficient to have the mRNA, protein, polypeptide, or peptide expressed within the cell. The expression vector(s) of the disclosure are not naturally-occurring as a whole. However, parts of the vectors can be naturally-occurring. Examples of expression vectors are shown in FIGS. 1-3.

The expression vectors of the present invention comprise any type of nucleotides, including, but not limited to DNA and RNA, which may be single-stranded or double-stranded, synthesized or obtained in part from natural sources, and which in exemplary aspects contain natural, non-natural or altered nucleotides. In exemplary aspects, the altered nucleotides or non-naturally occurring internucleotide linkages do not hinder the transcription or replication of the vector. In exemplary aspects, the expression vector system comprises one or more modified or non-natural nucleotides selected from the group consisting of: 5-fluorouracil, 5-bromouracil, 5-chlorouracil, 5-iodouracil, hypoxanthine, xanthine, 4-acetylcytosine, 5-(carboxyhydroxymethyl) uracil, 5-carboxymethylaminomethyl-2-thiouridine, 5-carboxymethylaminomethyluracil, dihydrouracil, beta-D-galactosyl queuosine, inosine, N6-isopentenyladenine, 1-methylguanine, 1-methylinosine, 2,2-dimethylguanine, 2-methyladenine, 2-methylguanine, 3-methylcytosine, 5-methylcytosine, N-substituted adenine, 7-methylguanine, 5-methylammomethyluracil, 5-methoxyaminomethyl-2-thiouracil, beta-D-mannosyl queuosine, 5′-methoxycarboxymethyluracil, 5-methoxyuracil, 2-methylthio-N6-isopentenyladenine, uracil-5-oxyacetic acid (v), wybutoxosine, pseudouracil, queuosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, and 2,6-diaminopurine.

The expression vectors disclosed herein in illustrative aspects comprise naturally-occurring or non-naturally-occurring internucleotide linkages, or both types of linkages. In exemplary aspects, the expression vector system comprises one or more modified inter-nucleotide linkages such as phosphoroamidate linkages and phosphorothioate linkages.

The expression vector system of the present invention may comprise any one or more suitable expression vectors, and may include one or more expression vectors used to transform or transfect any suitable host. Suitable expression vectors include those designed for propagation and expansion or for expression or both, such as plasmids and viruses. In various embodiments, the expression vector system in exemplary aspects comprises one or more expression vectors such as those from the pUC series (Fermentas Life Sciences), the pBluescript series (Stratagene, LaJolla, Calif.), the pET series (Novagen, Madison, Wis.), the pGEX series (Pharmacia Biotech, Uppsala, Sweden), and the pEX series (Clontech, Palo Alto, Calif.). Bacteriophage vectors, such as λGTIO, λGTI 1, λZapII (Stratagene), λEMBL4, and λNMI 149, also can be used. Examples of plant expression vectors include pBlOI, pBI101.2, pBI101.3, pBI121 and pBIN19 (Clontech). Examples of animal expression vectors include pEUK-CI, pMAM and pMAMneo (Clontech). In exemplary aspects, the expression vector system comprises a pBCMGSNeo expression vector and/or a pBCMGHis expression vector, as described in Yamazaki et al., 1999, supra. In exemplary aspects, the expression vector system comprises a viral vector, e.g., a retroviral vector, an adenovirus vector, an adeno-associated virus (AAV) vector, or a lentivirus vector.

The expression vectors and systems comprising the expression vectors of the present invention can be prepared using standard recombinant DNA techniques described in, for example, Sambrook et al., Molecular Cloning: A Laboratory Manual, and Ausubel et al., Current Protocols in Molecular Biology (1994). Constructs of expression vectors, which are circular or linear, can be prepared to contain a replication system functional in a prokaryotic or eukaryotic host cell. Replication systems can be derived, e.g., from ColEI, 2μ plasmid, λ, SV40, bovine papilloma virus, and the like.

The expression vector system may be designed for either transient expression, for stable expression, or for both. In exemplary aspects, the recombinant expression vector system comprises elements necessary for integration into the host genome. Also, the recombinant expression vectors can be made for constitutive expression or for inducible expression. For example, the recombinant expression vector system may comprise one or more suicide genes and/or one or more constitutive or inducible promoters.

In exemplary aspects, the expression vector system comprises regulatory sequences, such as transcription and translation initiation and termination codons, which are specific to the type of host (e.g., bacterium, fungus, or animal) into which the vector is to be introduced, as appropriate and taking into consideration whether the vector is DNA- or RNA-based.

The expression vector system in exemplary aspects comprises a native promoter operably linked to the nucleic acid comprising a nucleotide sequence encoding the fusion protein or the coronavirus (e.g., SARS-CoV-2) protein, or an antigenic portion thereof, or the nucleotide sequence which is complementary to or which hybridizes to the nucleotide sequence encoding the fusion protein or the coronavirus protein, or an antigenic portion thereof. The selection of promoters, e.g., strong, weak, inducible, tissue-specific and developmental-specific, is within the ordinary skill of the artisan. Similarly, the combining of a nucleotide sequence with a promoter is also within the skill of the artisan. The promoter can be a non-viral promoter or a viral promoter, e.g., a cytomegalovirus (CMV) promoter, an SV40 promoter, an RSV promoter, metallothionein (Mth) promoter, or a promoter found in the long-terminal repeat of the murine stem cell virus.

An expression vector also can include transcription enhancer elements, such as those found in SV40 virus, Hepatitis B virus, cytomegalovirus, immunoglobulin genes, metallothionein, and β-actin (see, Bittner et al., Meth Enzymol 1987, 153:516-544; and Gorman, Curr Op Biotechnol 1990, 1:36-47). In addition, an expression vector can contain sequences that permit maintenance and replication of the vector in more than one type of host cell, or integration of the vector into the host chromosome. Such sequences include, without limitation, to replication origins, autonomously replicating sequences (ARS), centromere DNA, and telomere DNA.

In exemplary aspects, the nucleic acid encoding a secretable fusion protein and the nucleic acid encoding a T cell costimulatory fusion protein are operably linked to the same promoter which is also operably linked to the nucleic acid encoding the coronavirus (e.g., 2019-nCoV) protein or an antigenic portion thereof. In some embodiments, one or more of the nucleic acid encoding a secretable fusion protein, the nucleic acid encoding a T cell costimulatory fusion protein, and the nucleic acid encoding the coronavirus (e.g., 2019-nCoV) protein, or an antigenic portion thereof, are operably linked to different promoters. For example, in exemplary aspects, the nucleic acid encoding the secretable fusion protein is operably linked to a promoter which is different from the promoter which is operably linked to the nucleic acid encoding the coronavirus (e.g., 2019-nCoV) protein, or an antigenic portion thereof. In exemplary aspects, the nucleic acid encoding the fusion protein is operably linked to a CMV promoter. In exemplary aspects, the nucleic acid encoding the coronavirus (e.g., 2019-nCoV) protein, or an antigenic portion thereof, is operably linked to an Mth promoter.

In some embodiments, the nucleic acid encoding the fusion protein and the nucleic acid encoding the coronavirus (e.g., 2019-nCoV) protein, or antigenic portion thereof, are present on the same expression vector. In some embodiments, the nucleic acid encoding the fusion protein is present on an expression vector which is different from the expression vector comprising the nucleic acid encoding the coronavirus protein, or antigenic portion thereof. In some embodiments, the expression vector system comprises two or more nucleic acids each encoding a different coronavirus protein, or an antigenic portion thereof. In some embodiments, the expression vector system comprises one, two, or more than two nucleic acids each encoding a different variant of a coronavirus protein, or an antigenic portion thereof. In some embodiments, a single nucleic acid encodes more than one variant of a coronavirus protein, or an antigenic portion thereof. In some embodiments, each nucleic acid encodes a respective variant of a coronavirus protein, or an antigenic portion thereof.

In some embodiments, the expression vector system of the present invention comprises only one recombinant expression vector. Alternatively, in some embodiments, the expression vector system comprises more than one expression vector. In exemplary aspects, the expression vector system comprises one expression vector comprising the nucleic acid encoding the fusion protein, one expression vector encoding a T cell costimulatory fusion protein, and one expression vector per number of different coronavirus (e.g., 2019-nCoV) proteins, or antigenic portion, encoded by the system. In exemplary aspects, the expression vector system comprises a nucleic acid encoding the fusion protein, a nucleic acid encoding a T cell costimulatory fusion protein, and one or two different coronavirus (e.g., 2019-nCoV) protein, or antigenic portion, and thereby comprises three expression vectors. In exemplary aspects, the recombinant expression vector system comprises two, three, four, five, or more recombinant expression vectors. In exemplary aspects, the expression vector system comprises at least two expression vectors and the nucleic acid encoding the fusion protein is present on an expression vector which is different from the expression vector comprising the nucleic acid encoding the coronavirus (e.g., 2019-nCoV) protein, or antigenic portion thereof. The expression vectors can be included in one, two, or more biological cells.

The expression vector system of the present invention in exemplary aspects comprises additional components. For example, in exemplary aspects, each vector of the recombinant expression vector system comprises a selectable marker. In exemplary aspects, the selectable marker is a gene product which confers resistance to an antibiotic, including but not limited to ampicillin, kanamycin, neomycin/G418, tetracycline, geneticin, triclosan, puromycin, zeocin, and hygromycin. In exemplary aspects, the selectable marker is one or more of kanamycin resistance genes, puromycin resistance genes, zeocin resistance genes, neomycin/G418 resistance genes, hygromycin resistance genes, histidinol resistance genes, tetracycline resistance genes, geneticin resistance genes, triclosan resistance genes, R-fluroorotic acid resistance genes, 5-fluorouracil resistance genes and ampicillin resistance genes. Combination of any of the selectable markers described herein is contemplated. In exemplary aspects, when the system comprises more than one recombinant expression vector, each vector comprises a selectable marker. In exemplary aspects, each vector has the same selectable marker. Alternatively, each vector within the system comprises a different selectable marker.

In some embodiments, the expression vector system further comprises a nucleic acid encoding a bovine papilloma virus (BPV) protein. The BPV early region encodes nonstructural proteins E1 to E7. E1 and E2 are nonstructural proteins derived from bovine papilloma virus (BPV). E5, E6 and E7 are viral oncoproteins derived from BPV and have the Gene Accession ID Numbers 1489021, 3783667 and 3783668, respectively. In exemplary aspects, the expression vector system further comprises a nucleotide sequence which encodes a BPV E1 and/or a BPV E2. In exemplary aspects, the expression vector system further comprises a nucleic acid encoding an E1 amino acid sequence of SEQ ID NO: 19 and/or an E2 amino acid sequence of SEQ ID NO: 22. In exemplary aspects, the expression vector system does not comprise a nucleic acid encoding a BPV viral oncoprotein. In exemplary aspects, the expression vector system does not comprise a nucleic acid encoding E5, E6, and/or E7. In exemplary aspects, the expression vector system does not comprise nucleotides 3878 to 4012 of GenBank Accession No. NC_001522.1 encoding E5, nucleotides 91 to 519 of GenBank Accession No. NC_007612.1 encoding E6, and/or nucleotides 522 to 836 of GenBank Accession No. NC_007612.1 encoding E7. In exemplary aspects, the expression vector system does not comprise any one of SEQ ID NOs: 32-34.

In some embodiments, the expression vector system comprises the vector, or one or more elements thereof, as shown in FIG. 1. In exemplary aspects, the expression vector system of the present invention comprises the sequence of SEQ ID NOS: 24 and/or 25.

In some embodiments, the expression vector system comprises the sequence of SEQ ID NO: 24 or SEQ ID NO: 25.

In some embodiments, the expression vector system comprises one or more nucleic acids encoding one or more variants of a coronavirus protein or antigenic portion thereof. In embodiments, the variants are selected from a plurality of variants of a coronavirus protein comprising, without limitation, B.1.1.7, B.1.351 (501Y.V2), B.1, B.1.1.28, B.1.2, CAL.20C, B.6, P.1, and P.2 variants, or antigenic fragments thereof. In some embodiments, the lineages include A.1, A.2, A.3, A.4, A.5, A.6, A.7, A.8, A.9, B, B.1, B.1.1, B.1.1.1, B.2, B.3, B.4, B.5, B.6, B.7, B.9, B.10, B.11, B.12, B.13, B.14, B.15, B.16, B.17, B.18, B.19, B.20, B.21, B.22, B.23, B.24, B.25, B.26, B.27, C.1, C.2, C.3, D.1, and D.2 variants, or antigenic fragments thereof. See Rambaut et al., (2020),In various embodiments, the expression vector system of the present invention encodes proteins that can be expressed in prokaryotic and eukaryotic cells. In various embodiments, expression vectors can be introduced into host cells for producing the fusion protein and the SARS-CoV-2 proteins, including variants of SARS-CoV-2 proteins. There are a variety of techniques available for introducing nucleic acids into viable cells. Techniques suitable for the transfer of nucleic acid into mammalian cells in vitro include the use of liposomes, electroporation, microinjection, cell fusion, polymer-based systems, DEAE-dextran, viral transduction, the calcium phosphate precipitation method, etc. For in vivo gene transfer, a number of techniques and reagents may also be used, including electroporation, liposomes; natural polymer-based delivery vehicles, such as chitosan and gelatin; viral vectors are also suitable for in vivo transduction.

The present invention further provides a cell (e.g., a host cell) comprising the expression vector system described herein. Cells (e.g., host cells) may be cultured in vitro or genetically engineered, for example. Host cells can be obtained from normal or affected subjects, including healthy humans, patients infected with the SARS-CoV-2 virus, private laboratory deposits, public culture collections such as the American Type Culture Collection, or from commercial suppliers.

In some embodiments, a host cell a mammalian host cell. The mammalian host cell can be a human host cell. In some embodiments, the host cell is an NIH 3T3 cell or an HEK 293 cell.

Cells that can be used include, without limitation, epithelial cells, endothelial cells, keratinocytes, fibroblasts, muscle cells, hepatocytes; blood cells such as T lymphocytes, B lymphocytes, monocytes, macrophages, neutrophils, eosinophils, megakaryocytes, or granulocytes, various stem or progenitor cells, such as hematopoietic stem or progenitor cells (e.g., as obtained from bone marrow), umbilical cord blood, peripheral blood, fetal liver, etc., and tumor cells (e.g., human tumor cells). The choice of cell type can be determined by one of skill in the art. In various embodiments, the cells are irradiated.

In some embodiments, the gp96-Ig fusion protein, SARS-CoV-2 spike protein, and/or T cell costimulatory fusion protein-Ig secretes into culture supernatants at rate of about 50 ng/mL/24 h/10⁶ vaccine cells to about 500 ng/mL/24 h/10⁶ vaccine cells. In some embodiments, the gp96-Ig fusion protein, SARS-CoV-2 spike protein, and/or T cell costimulatory fusion protein-Ig secretes into culture supernatants at rate of about 50 ng/mL/24 h/10⁶ vaccine cells, about 100 ng/mL/24 h/10⁶ vaccine cells, about 125 ng/mL/24 h/10⁶ vaccine cells, about 150 ng/mL/24 h/10⁶ vaccine cells, about 175 ng/mL/24 h/10⁶ vaccine cells, about 200 ng/mL/24 h/10⁶ vaccine cells, about 250 ng/mL/24 h/10⁶ vaccine cells, about 300 ng/mL/24 h/10⁶ vaccine cells, about 350 ng/mL/24 h/10⁶ vaccine cells, about 400 ng/mL/24 h/10⁶ vaccine cells, about 450 ng/mL/24 h/10⁶ vaccine cells, or about 500 ng/mL/24 h/10⁶ vaccine cells. In some embodiments, the gp96-Ig fusion protein, SARS-CoV-2 spike protein, and/or T cell costimulatory fusion protein-Ig secretes into culture supernatants at rate of about 125 ng/mL/24 h/10⁶ vaccine cells.

T-Cell Co-Stimulation

In addition to a gp96-Ig fusion protein and a nucleic acid encoding a coronavirus protein, the expression vectors provided herein can encode one or more biological response modifiers. In various embodiments, the present expression vectors can encode one or more T cell costimultory molecules.

In various embodiments, the present expression vector encode an agonist of OX40 (e.g., an OX40 ligand-Ig (OX40L-Ig) fusion, or a fragment thereof that binds OX40), an agonist of inducible T-cell costimulator (ICOS) (e.g., an ICOS ligand-Ig (ICOSL-Ig) fusion, or a fragment thereof that binds ICOS), an agonist of CD40 (e.g., a CD40L-Ig fusion protein, or fragment thereof), an agonist of CD27 (e.g. a CD70-Ig fusion protein or fragment thereof), or an agonist of 4-1BB (e.g., a 4-1BB ligand-Ig (4-1BBL-Ig) fusion, or a fragment thereof that binds 4-1BB). In some embodiments, a vector can encode an agonist of TNFRSF25 (e.g., a TL1A-Ig fusion, or a fragment thereof that binds TNFRSF25), or an agonist of glucocorticoid-induced tumor necrosis factor receptor (GITR) (e.g., a GITR ligand-Ig (GITRL-Ig) fusion, or a fragment thereof that binds GITR), or an agonist of CD40 (e.g., a CD40 ligand-Ig (CD40L-Ig) fusion, or a fragment thereof that binds CD40); or an agonist of CD27 (e.g., a CD27 ligand-Ig (e.g. CD70L-Ig) fusion, or a fragment thereof that binds CD40).

ICOS is an inducible T cell costimulatory receptor molecule that displays some homology to CD28 and CTLA-4, and interacts with B7-H2 expressed on the surface of antigen-presenting cells. ICOS has been implicated in the regulation of cell-mediated and humoral immune responses.

4-1BB is a type 2 transmembrane glycoprotein belonging to the TNF superfamily, and is expressed on activated T Lymphocytes.

OX40 (also referred to as CD134 or TNFRSF4) is a T cell costimulatory molecule that is engaged by OX40L, and frequently is induced in antigen presenting cells and other cell types. OX40 is known to enhance cytokine expression and survival of effector T cells.

GITR (TNFRSF18) is a T cell costimulatory molecule that is engaged by GITRL and is preferentially expressed in FoxP3+ regulatory T cells. GITR plays a significant role in the maintenance and function of Treg within the tumor microenvironment.

TNFRSF25 is a T cell costimulatory molecule that is preferentially expressed in CD4+ and CD8+ T cells following antigen stimulation. Signaling through TNFRSF25 is provided by TL1A, and functions to enhance T cell sensitivity to IL-2 receptor mediated proliferation in a cognate antigen dependent manner.

CD40 is a costimulatory protein found on various antigen presenting cells which plays a role in their activation. The binding of CD40L (CD154) on TH cells to CD40 activates antigen presenting cells and induces a variety of downstream effects.

CD27 a T cell costimulatory molecule belonging to the TNF superfamily which plays a role in the generation and long-term maintenance of T cell immunity. It binds to a ligand CD70 in various immunological processes.

Additional costimulatory molecules that may be utilized in the present invention include, but are not limited to, HVEM, CD28, CD30, CD30L, CD40, CD70, LIGHT (CD258), B7-1, and B7-2.

As for the gp96-Ig fusions, the Ig portion (“tag”) of the T cell costimulatory fusion protein can include a non-variable portion of an immunoglobulin molecule or domain (e.g., an IgG1, IgG2, IgG3, IgG4, IgM, IgA, or IgE molecule). Such portions typically include at least functional CH2 and CH3 domains of the constant region of an immunoglobulin heavy chain. In some embodiments, a T cell costimulatory peptide can be fused to the hinge, CH2 and CH3 domains of murine IgG1 (Bowen et al., J Immunol 1996, 156:442-449). The Ig tag can be from a mammalian (e.g., human, mouse, monkey, or rat) immunoglobulin, but human immunoglobulin can be particularly useful when the fusion protein is intended for in vivo use for humans. Again, DNAs encoding immunoglobulin light or heavy chain constant regions are known or readily available from cDNA libraries. Various leader sequences as described above also can be used for secretion of T cell costimulatory fusion proteins from bacterial and mammalian cells.

In some embodiments, the heat shock protein gp96, genetically fused to an immunoglobulin domain (e.g., an IgG1, IgG2, IgG3, IgG4, IgM, IgA, or IgE molecule), acts as a potent adjuvant that activates TLR2 and TLR4 on professional antigen-presenting cells (APCs).

A representative nucleotide optimized sequence (SEQ ID NO:50) encoding the extracellular domain of human ICOSL fused to Ig, and the amino acid sequence of the encoded fusion (SEQ ID NO:51) are provided:

(SEQ ID NO: 50)
ATGAGACTGGGAAGCCCTGGCCTGCTGTTTCTGCTGTTCAGCAGCCTGAG
AGCCGACACCCAGGAAAAAGAAGTGCGGGCCATGGTGGGAAGCGACGTGG
AACTGAGCTGCGCCTGTCCTGAGGGCAGCAGATTCGACCTGAACGACGTG
TACGTGTACTGGCAGACCAGCGAGAGCAAGACCGTCGTGACCTACCACAT
CCCCCAGAACAGCTCCCTGGAAAACGTGGACAGCCGGTACAGAAACCGGG
CCCTGATGTCTCCTGCCGGCATGCTGAGAGGCGACTTCAGCCTGCGGCTG
TTCAACGTGACCCCCCAGGACGAGCAGAAATTCCACTGCCTGGTGCTGAG
CCAGAGCCTGGGCTTCCAGGAAGTGCTGAGCGTGGAAGTGACCCTGCACG
TGGCCGCCAATTTCAGCGTGCCAGTGGTGTCTGCCCCCCACAGCCCTTCT
CAGGATGAGCTGACCTTCACCTGTACCAGCATCAACGGCTACCCCAGACC
CAATGTGTACTGGATCAACAAGACCGACAACAGCCTGCTGGACCAGGCCC
TGCAGAACGATACCGTGTTCCTGAACATGCGGGGCCTGTACGACGTGGTG
TCCGTGCTGAGAATCGCCAGAACCCCCAGCGTGAACATCGGCTGCTGCAT
CGAGAACGTGCTGCTGCAGCAGAACCTGACCGTGGGCAGCCAGACCGGCA
ACGACATCGGCGAGAGAGACAAGATCACCGAGAACCCCGTGTCCACCGGC
GAGAAGAATGCCGCCACCTCTAAGTACGGCCCTCCCTGCCCTTCTTGCCC
AGCCCCTGAATTTCTGGGCGGACCCTCCGTGTTTCTGTTCCCCCCAAAGC
CCAAGGACACCCTGATGATCAGCCGGACCCCCGAAGTGACCTGCGTGGTG
GTGGATGTGTCCCAGGAAGATCCCGAGGTGCAGTTCAATTGGTACGTGGA
CGGGGTGGAAGTGCACAACGCCAAGACCAAGCCCAGAGAGGAACAGTTCA
ACAGCACCTACCGGGTGGTGTCTGTGCTGACCGTGCTGCACCAGGATTGG
CTGAGCGGCAAAGAGTACAAGTGCAAGGTGTCCAGCAAGGGCCTGCCCAG
CAGCATCGAAAAGACCATCAGCAACGCCACCGGCCAGCCCAGGGAACCCC
AGGTGTACACACTGCCCCCTAGCCAGGAAGAGATGACCAAGAACCAGGTG
TCCCTGACCTGTCTCGTGAAGGGCTTCTACCCCTCCGATATCGCCGTGGA
ATGGGAGAGCAACGGCCAGCCAGAGAACAACTACAAGACCACCCCCCCAG
TGCTGGACAGCGACGGCTCATTCTTCCTGTACTCCCGGCTGACAGTGGAC
AAGAGCAGCTGGCAGGAAGGCAACGTGTTCAGCTGCAGCGTGATGCACGA
AGCCCTGCACAACCACTACACCCAGAAGTCCCTGTCTCTGTCCCTGGGCA
AATGA.
(SEQ ID NO: 51)
MRLGSPGLLFLLFSSLRADTQEKEVRAMVGSDVELSCACPEGSRFDLNDV
YVYWQTSESKTVVTYHIPQNSSLENVDSRYRNRALMSPAGMLRGDFSLRL
FNVTPQDEQKFHCLVLSQSLGFQEVLSVEVTLHVAANFSVPVVSAPHSPS
QDELTFTCTSINGYPRPNVYWINKTDNSLLDQALQNDTVFLNMRGLYDVV
SVLRIARTPSVNIGCCIENVLLQQNLTVGSQTGNDIGERDKITENPVSTG
EKNAATSKYGPPCPSCPAPEFLGGPSVFLFPPKPKDTLMISRTPEVTCVV
VDVSQEDPEVQFNWYVDGVEVHNAKTKPREEQFNSTYRVVSVLTVLHQDW
LSGKEYKCKVSSKGLPSSIEKTISNATGQPREPQVYTLPPSQEEMTKNQV
SLTCLVKGFYPSDIAVEWESNGQPENNYKTTPPVLDSDGSFFLYSRLTVD
KSSWQEGNVFSCSVMHEALHNHYTQKSLSLSLGK.

A representative nucleotide optimized sequence (SEQ ID NO:52) encoding the extracellular domain of human 4-1BBL fused to Ig, and the encoded amino acid sequence (SEQ ID NO:53) are provided:

(SEQ ID NO: 52)
ATGTCTAAGTACGGCCCTCCCTGCCCTAGCTGCCCTGCCCCTGAATTTCT
GGGCGGACCCAGCGTGTTCCTGTTCCCCCCAAAGCCCAAGGACACCCTGA
TGATCAGCCGGACCCCCGAAGTGACCTGCGTGGTGGTGGATGTGTCCCAG
GAAGATCCCGAGGTGCAGTTCAATTGGTACGTGGACGGCGTGGAAGTGCA
CAACGCCAAGACCAAGCCCAGAGAGGAACAGTTCAACAGCACCTACCGGG
TGGTGTCCGTGCTGACCGTGCTGCACCAGGATTGGCTGAGCGGCAAAGAG
TACAAGTGCAAGGTGTCCAGCAAGGGCCTGCCCAGCAGCATCGAGAAAAC
CATCAGCAACGCCACCGGCCAGCCCAGGGAACCCCAGGTGTACACACTGC
CCCCTAGCCAGGAAGAGATGACCAAGAACCAGGTGTCCCTGACCTGTCTC
GTGAAGGGCTTCTACCCCTCCGATATCGCCGTGGAATGGGAGAGCAACGG
CCAGCCTGAGAACAACTACAAGACCACCCCCCCAGTGCTGGACAGCGACG
GCTCATTCTTCCTGTACAGCAGACTGACCGTGGACAAGAGCAGCTGGCAG
GAAGGCAACGTGTTCAGCTGCAGCGTGATGCACGAGGCCCTGCACAACCA
CTACACCCAGAAGTCCCTGTCTCTGAGCCTGGGCAAGGCCTGTCCATGGG
CTGTGTCTGGCGCTAGAGCCTCTCCTGGATCTGCCGCCAGCCCCAGACTG
AGAGAGGGACCTGAGCTGAGCCCCGATGATCCTGCCGGACTGCTGGATCT
GAGACAGGGCATGTTCGCCCAGCTGGTGGCCCAGAACGTGCTGCTGATCG
ATGGCCCCCTGAGCTGGTACAGCGATCCTGGACTGGCTGGCGTGTCACTG
ACAGGCGGCCTGAGCTACAAAGAGGACACCAAAGAACTGGTGGTGGCCAA
GGCCGGCGTGTACTACGTGTTCTTTCAGCTGGAACTGCGGAGAGTGGTGG
CCGGCGAAGGATCCGGCTCTGTGTCTCTGGCTCTGCATCTGCAGCCCCTG
AGATCTGCTGCTGGCGCTGCTGCTCTGGCCCTGACAGTGGACCTGCCTCC
TGCCTCTAGCGAGGCCAGAAACAGCGCATTCGGGTTTCAAGGCAGACTGC
TGCACCTGTCTGCCGGCCAGAGACTGGGAGTGCATCTGCACACAGAGGCC
AGAGCCAGGCACGCCTGGCAGCTGACTCAGGGCGCTACAGTGCTGGGCCT
GTTCAGAGTGACCCCCGAGATTCCAGCCGGCCTGCCTAGCCCCAGATCCG
AATGA.
(SEQ ID NO: 53)
MSKYGPPCPSCPAPEFLGGPSVFLFPPKPKDTLMISRTPEVTCVVVDVSQ
EDPEVQFNWYVDGVEVHNAKTKPREEQFNSTYRVVSVLTVLHQDWLSGKE
YKCKVSSKGLPSSIEKTISNATGQPREPQVYTLPPSQEEMTKNQVSLTCL
VKGFYPSDIAVEWESNGQPENNYKTTPPVLDSDGSFFLYSRLTVDKSSWQ
EGNVFSCSVMHEALHNHYTQKSLSLSLGKACPWAVSGARASPGSAASPRL
REGPELSPDDPAGLLDLRQGMFAQLVAQNVLLIDGPLSWYSDPGLAGVSL
TGGLSYKEDTKELVVAKAGVYYVFFQLELRRVVAGEGSGSVSLALHLQPL
RSAAGAAALALTVDLPPASSEARNSAFGFQGRLLHLSAGQRLGVHLHTEA
RARHAWQLTQGATVLGLFRVTPEIPAGLPSPRSE.

A representative nucleotide optimized sequence (SEQ ID NO:54) encoding the extracellular domain of human TL1A fused to Ig, and the encoded amino acid sequence (SEQ ID NO:55) are provided:

(SEQ ID NO: 54)
ATGTCTAAGTACGGCCCTCCCTGCCCTAGCTGCCCTGCCCCTGAATTTCT
GGGCGGACCCAGCGTGTTCCTGTTCCCCCCAAAGCCCAAGGACACCCTGA
TGATCAGCCGGACCCCCGAAGTGACCTGCGTGGTGGTGGATGTGTCCCAG
GAAGATCCCGAGGTGCAGTTCAATTGGTACGTGGACGGCGTGGAAGTGCA
CAACGCCAAGACCAAGCCCAGAGAGGAACAGTTCAACAGCACCTACCGGG
TGGTGTCCGTGCTGACCGTGCTGCACCAGGATTGGCTGAGCGGCAAAGAG
TACAAGTGCAAGGTGTCCAGCAAGGGCCTGCCCAGCAGCATCGAGAAAAC
CATCAGCAACGCCACCGGCCAGCCCAGGGAACCCCAGGTGTACACACTGC
CCCCTAGCCAGGAAGAGATGACCAAGAACCAGGTGTCCCTGACCTGTCTC
GTGAAGGGCTTCTACCCCTCCGATATCGCCGTGGAATGGGAGAGCAACGG
CCAGCCTGAGAACAACTACAAGACCACCCCCCCAGTGCTGGACAGCGACG
GCTCATTCTTCCTGTACAGCAGACTGACCGTGGACAAGAGCAGCTGGCAG
GAAGGCAACGTGTTCAGCTGCAGCGTGATGCACGAGGCCCTGCACAACCA
CTACACCCAGAAGTCCCTGTCTCTGAGCCTGGGCAAGATCGAGGGCCGGA
TGGATAGAGCCCAGGGCGAAGCCTGCGTGCAGTTCCAGGCTCTGAAGGGC
CAGGAATTCGCCCCCAGCCACCAGCAGGTGTACGCCCCTCTGAGAGCCGA
CGGCGATAAGCCTAGAGCCCACCTGACAGTCGTGCGGCAGACCCCTACCC
AGCACTTCAAGAATCAGTTCCCCGCCCTGCACTGGGAGCACGAACTGGGC
CTGGCCTTCACCAAGAACAGAATGAACTACACCAACAAGTTTCTGCTGAT
CCCCGAGAGCGGCGACTACTTCATCTACAGCCAAGTGACCTTCCGGGGCA
TGACCAGCGAGTGCAGCGAGATCAGACAGGCCGGCAGACCTAACAAGCCC
GACAGCATCACCGTCGTGATCACCAAAGTGACCGACAGCTACCCCGAGCC
CACCCAGCTGCTGATGGGCACCAAGAGCGTGTGCGAAGTGGGCAGCAACT
GGTTCCAGCCCATCTACCTGGGCGCCATGTTTAGTCTGCAAGAGGGCGAC
AAGCTGATGGTCAACGTGTCCGACATCAGCCTGGTGGATTACACCAAAGA
GGACAAGACCTTCTTCGGCGCCTTTCTGCTCTGA
(SEQ ID NO: 55)
MSKYGPPCPSCPAPEFLGGPSVFLFPPKPKDTLMISRTPEVTCVVVDVSQ
EDPEVQFNWYVDGVEVHNAKTKPREEQFNSTYRVVSVLTVLHQDWLSGKE
YKCKVSSKGLPSSIEKTISNATGQPREPQVYTLPPSQEEMTKNQVSLTCL
VKGFYPSDIAVEWESNGQPENNYKTTPPVLDSDGSFFLYSRLTVDKSSWQ
EGNVFSCSVMHEALHNHYTQKSLSLSLGKIEGRMDRAQGEACVQFQALKG
QEFAPSHQQVYAPLRADGDKPRAHLTVVRQTPTQHFKNQFPALHWEHELG
LAFTKNRMNYTNKFLLIPESGDYFIYSQVTFRGMTSECSEIRQAGRPNKP
DSITVVITKVTDSYPEPTQLLMGTKSVCEVGSNWFQPIYLGAMFSLQEGD
KLMVNVSDISLVDYTKEDKTFFGAFLL.

A representative nucleotide optimized sequence (SEQ ID NO:56) encoding human OX40L-Ig, and the encoded amino acid sequence (SEQ ID NO:57) are provided:

(SEQ ID NO: 56)
ATGTCTAAGTACGGCCCTCCCTGCCCTAGCTGCCCTGCCCCTGAATTTCT
GGGCGGACCCAGCGTGTTCCTGTTCCCCCCAAAGCCCAAGGACACCCTGA
TGATCAGCCGGACCCCCGAAGTGACCTGCGTGGTGGTGGATGTGTCCCAG
GAAGATCCCGAGGTGCAGTTCAATTGGTACGTGGACGGCGTGGAAGTGCA
CAACGCCAAGACCAAGCCCAGAGAGGAACAGTTCAACAGCACCTACCGGG
TGGTGTCCGTGCTGACCGTGCTGCACCAGGATTGGCTGAGCGGCAAAGAG
TACAAGTGCAAGGTGTCCAGCAAGGGCCTGCCCAGCAGCATCGAGAAAAC
CATCAGCAACGCCACCGGCCAGCCCAGGGAACCCCAGGTGTACACACTGC
CCCCTAGCCAGGAAGAGATGACCAAGAACCAGGTGTCCCTGACCTGTCTC
GTGAAGGGCTTCTACCCCTCCGATATCGCCGTGGAATGGGAGAGCAACGG
CCAGCCTGAGAACAACTACAAGACCACCCCCCCAGTGCTGGACAGCGACG
GCTCATTCTTCCTGTACAGCAGACTGACCGTGGACAAGAGCAGCTGGCAG
GAAGGCAACGTGTTCAGCTGCAGCGTGATGCACGAGGCCCTGCACAACCA
CTACACCCAGAAGTCCCTGTCTCTGAGCCTGGGCAAGATCGAGGGCCGGA
TGGATCAGGTGTCACACAGATACCCCCGGATCCAGAGCATCAAAGTGCAG
TTTACCGAGTACAAGAAAGAGAAGGGCTTTATCCTGACCAGCCAGAAAGA
GGACGAGATCATGAAGGTGCAGAACAACAGCGTGATCATCAACTGCGACG
GGTTCTACCTGATCAGCCTGAAGGGCTACTTCAGTCAGGAAGTGAACATC
AGCCTGCACTACCAGAAGGACGAGGAACCCCTGTTCCAGCTGAAGAAAGT
GCGGAGCGTGAACAGCCTGATGGTGGCCTCTCTGACCTACAAGGACAAGG
TGTACCTGAACGTGACCACCGACAACACCAGCCTGGACGACTTCCACGTG
AACGGCGGCGAGCTGATCCTGATTCACCAGAACCCCGGCGAGTTCTGCGT
GCTCTGA.
(SEQ ID NO: 57)
MSKYGPPCPSCPAPEFLGGPSVFLFPPKPKDTLMISRTPEVICVVVDVSQ
EDPEVQFNWYVDGVEVHNAKTKPREEQFNSTYRVVSVLTVLHQDWLSGKE
YKCKVSSKGLPSSIEKTISNATGQPREPQVYTLPPSQEEMTKNQVSLTCL
VKGFYPSDIAVEWESNGQPENNYKTTPPVLDSDGSFFLYSRLTVDKSSWQ
EGNVFSCSVMHEALHNHYTQKSLSLSLGKIEGRMDQVSHRYPRIQSIKVQ
FTEYKKEKGFILTSQKEDEIMKVQNNSVIINCDGFYLISLKGYFSQEVNI
SLHYQKDEEPLFQLKKVRSVNSLMVASLTYKDKVYLNVTTDNTSLDDFHV
NGGELILIHQNPGEFCVL.

Representative nucleotide and amino acid sequences for human TL1A are set forth in SEQ ID NO:58 and SEQ ID NO:59, respectively:

(SEQ ID NO: 58)
TCCCAAGTAGCTGGGACTACAGGAGCCCACCACCACCCCCGGCTAATTTT
TTGTATTTTTAGTAGAGACGGGGTTTCACCGTGTTAGCCAAGATGGTCTT
GATCACCTGACCTCGTGATCCACCCGCCTTGGCCTCCCAAAGTGCTGGGA
TTACAGGCATGAGCCACCGCGCCCGGCCTCCATTCAAGTCTTTATTGAAT
ATCTGCTATGTTCTACACACTGTTCTAGGTGCTGGGGATGCAACAGGGGA
CAAAATAGGCAAAATCCCTGTCCTTTTGGGGTTGACATTCTAGTGACTCT
TCATGTAGTCTAGAAGAAGCTCAGTGAATAGTGTCTGTGGTTGTTACCAG
GGACACAATGACAGGAACATTCTTGGGTAGAGTGAGAGGCCTGGGGAGGG
AAGGGTCTCTAGGATGGAGCAGATGCTGGGCAGTCTTAGGGAGCCCCTCC
TGGCATGCACCCCCTCATCCCTCAGGCCACCCCCGTCCCTTGCAGGAGCA
CCCTGGGGAGCTGTCCAGAGCGCTGTGCCGCTGTCTGTGGCTGGAGGCAG
AGTAGGTGGTGTGCTGGGAATGCGAGTGGGAGAACTGGGATGGACCGAGG
GGAGGCGGGTGAGGAGGGGGGCAACCACCCAACACCCACCAGCTGCTTTC
AGTGTTCTGGGTCCAGGTGCTCCTGGCTGGCCTTGTGGTCCCCCTCCTGC
TTGGGGCCACCCTGACCTACACATACCGCCACTGCTGGCCTCACAAGCCC
CTGGTTACTGCAGATGAAGCTGGGATGGAGGCTCTGACCCCACCACCGGC
CACCCATCTGTCACCCTTGGACAGCGCCCACACCCTTCTAGCACCTCCTG
ACAGCAGTGAGAAGATCTGCACCGTCCAGTTGGTGGGTAACAGCTGGACC
CCTGGCTACCCCGAGACCCAGGAGGCGCTCTGCCCGCAGGTGACATGGTC
CTGGGACCAGTTGCCCAGCAGAGCTCTTGGCCCCGCTGCTGCGCCCACAC
TCTCGCCAGAGTCCCCAGCCGGCTCGCCAGCCATGATGCTGCAGCCGGGC
CCGCAGCTCTACGACGTGATGGACGCGGTCCCAGCGCGGCGCTGGAAGGA
GTTCGTGCGCACGCTGGGGCTGCGCGAGGCAGAGATCGAAGCCGTGGAGG
TGGAGATCGGCCGCTTCCGAGACCAGCAGTACGAGATGCTCAAGCGCTGG
CGCCAGCAGCAGCCCGCGGGCCTCGGAGCCGTTTACGCGGCCCTGGAGCG
CATGGGGCTGGACGGCTGCGTGGAAGACTTGCGCAGCCGCCTGCAGCGCG
GCCCGTGACACGGCGCCCACTTGCCACCTAGGCGCTCTGGTGGCCCTTGC
AGAAGCCCTAAGTACGGTTACTTATGCGTGTAGACATTTTATGTCACTTA
TTAAGCCGCTGGCACGGCCCTGCGTAGCAGCACCAGCCGGCCCCACCCCT
GCTCGCCCCTATCGCTCCAGCCAAGGCGAAGAAGCACGAACGAATGTCGA
GAGGGGGTGAAGACATTTCTCAACTTCTCGGCCGGAGTTTGGCTGAGATC
GCGGTATTAAATCTGTGAAAGAAAACAAAACAAAACAA.
(SEQ ID NO: 59)
MEQRPRGCAAVAAALLLVLLGARAQGGTRSPRCDCAGDFHKKIGLFCCRG
CPAGHYLKAPCTEPCGNSTCLVCPQDTFLAWENHHNSECARCQACDEQAS
QVALENCSAVADTRCGCKPGWFVECQVSQCVSSSPFYCQPCLDCGALHRH
TRLLCSRRDTDCGTCLPGFYEHGDGCVSCPTPPPSLAGAPWGAVQSAVPL
SVAGGRVGVFWVQVLLAGLVVPLLLGATLTYTYRHCWPHKPLVTADEAGM
EALTPPPATHLSPLDSAHTLLAPPDSSEKICTVQLVGNSWTPGYPETQEA
LCPQVTWSWDQLPSRALGPAAAPTLSPESPAGSPAMMLQPGPQLYDVMDA
VPARRWKEFVRTLGLREAEIEAVEVEIGRFRDQQYEMLKRWRQQQPAGLG
AVYAALERMGLDGCVEDLRSRLQRGP.

Representative nucleotide and amino acid sequences for human HVEM are set forth in SEQ ID NO:84 (accession no. CR456909) and SEQ ID NO:85, respectively (accession no. CR456909):

(SEQ ID NO: 84)
ATGGAGCCTCCTGGAGACTGGGGGCCTCCTCCCTGGAGATCCACCCCCAA
AACCGACGTCTTGAGGCTGGTGCTGTATCTCACCTTCCTGGGAGCCCCCT
GCTACGCCCCAGCTCTGCCGTCCTGCAAGGAGGACGAGTACCCAGTGGGC
TCCGAGTGCTGCCCCAAGTGCAGTCCAGGTTATCGTGTGAAGGAGGCCTG
CGGGGAGCTGACGGGCACAGTGTGTGAACCCTGCCCTCCAGGCACCTACA
TTGCCCACCTCAATGGCCTAAGCAAGTGTCTGCAGTGCCAAATGTGTGAC
CCAGCCATGGGCCTGCGCGCGAGCCGGAACTGCTCCAGGACAGAGAACGC
CGTGTGTGGCTGCAGCCCAGGCCACTTCTGCATCGTCCAGGACGGGGACC
ACTGCGCCGCGTGCCGCGCTTACGCCACCTCCAGCCCGGGCCAGAGGGTG
CAGAAGGGAGGCACCGAGAGTCAGGACACCCTGTGTCAGAACTGCCCCCC
GGGGACCTTCTCTCCCAATGGGACCCTGGAGGAATGTCAGCACCAGACCA
AGTGCAGCTGGCTGGTGACGAAGGCCGGAGCTGGGACCAGCAGCTCCCAC
TGGGTATGGTGGTTTCTCTCAGGGAGCCTCGTCATCGTCATTGTTTGCTC
CACAGTTGGCCTAATCATATGTGTGAAAAGAAGAAAGCCAAGGGGTGATG
TAGTCAAGGTGATCGTCTCCGTCCAGCGGAAAAGACAGGAGGCAGAAGGT
GAGGCCACAGTCATTGAGGCCCTGCAGGCCCCTCCGGACGTCACCACGGT
GGCCGTGGAGGAGACAATACCCTCATTCACGGGGAGGAGCCCAAACCATT
AA.
(SEQ ID NO: 85)
MEPPGDWGPPPWRSTPKTDVLRLVLYLTFLGAPCYAPALPSCKEDEYPVG
SECCPKCSPGYRVKEACGELTGTVCEPCPPGTYIAHLNGLSKCLQCQMCD
PAMGLRASRNCSRTENAVCGCSPGHFCIVQDGDHCAACRAYATSSPGQRV
QKGGTESQDTLCQNCPPGTFSPNGTLEECQHQTKCSWLVTKAGAGTSSSH
WVWWFLSGSLVIVIVCSTVGLIICVKRRKPRGDVVKVIVSVQRKRQEAEG
EATVIEALQAPPDVTTVAVEETIPSFTGRSPNH.

Representative nucleotide and amino acid sequences for human CD28 are set forth in SEQ ID NO:86 (accession no. NM_006139) and SEQ ID NO:87, respectively:

(SEQ ID NO: 86)
TAAAGTCATCAAAACAACGTTATATCCTGTGTGAAATGCTGCAGTCAGGA
TGCCTTGTGGTTTGAGTGCCTTGATCATGTGCCCTAAGGGGATGGTGGCG
GTGGTGGTGGCCGTGGATGACGGAGACTCTCAGGCCTTGGCAGGTGCGTC
TTTCAGTTCCCCTCACACTTCGGGTTCCTCGGGGAGGAGGGGCTGGAACC
CTAGCCCATCGTCAGGACAAAGATGCTCAGGCTGCTCTTGGCTCTCAACT
TATTCCCTTCAATTCAAGTAACAGGAAACAAGATTTTGGTGAAGCAGTCG
CCCATGCTTGTAGCGTACGACAATGCGGTCAACCTTAGCTGCAAGTATTC
CTACAATCTCTTCTCAAGGGAGTTCCGGGCATCCCTTCACAAAGGACTGG
ATAGTGCTGTGGAAGTCTGTGTTGTATATGGGAATTACTCCCAGCAGCTT
CAGGTTTACTCAAAAACGGGGTTCAACTGTGATGGGAAATTGGGCAATGA
ATCAGTGACATTCTACCTCCAGAATTTGTATGTTAACCAAACAGATATTT
ACTTCTGCAAAATTGAAGTTATGTATCCTCCTCCTTACCTAGACAATGAG
AAGAGCAATGGAACCATTATCCATGTGAAAGGGAAACACCTTTGTCCAAG
TCCCCTATTTCCCGGACCTTCTAAGCCCTTTTGGGTGCTGGTGGTGGTTG
GTGGAGTCCTGGCTTGCTATAGCTTGCTAGTAACAGTGGCCTTTATTATT
TTCTGGGTGAGGAGTAAGAGGAGCAGGCTCCTGCACAGTGACTACATGAA
CATGACTCCCCGCCGCCCCGGGCCCACCCGCAAGCATTACCAGCCCTATG
CCCCACCACGCGACTTCGCAGCCTATCGCTCCTGACACGGACGCCTATCC
AGAAGCCAGCCGGCTGGCAGCCCCCATCTGCTCAATATCACTGCTCTGGA
TAGGAAATGACCGCCATCTCCAGCCGGCCACCTCAGGCCCCTGTTGGGCC
ACCAATGCCAATTTTTCTCGAGTGACTAGACCAAATATCAAGATCATTTT
GAGACTCTGAAATGAAGTAAAAGAGATTTCCTGTGACAGGCCAAGTCTTA
CAGTGCCATGGCCCACATTCCAACTTACCATGTACTTAGTGACTTGACTG
AGAAGTTAGGGTAGAAAACAAAAAGGGAGTGGATTCTGGGAGCCTCTTCC
CTTTCTCACTCACCTGCACATCTCAGTCAAGCAAAGTGTGGTATCCACAG
ACATTTTAGTTGCAGAAGAAAGGCTAGGAAATCATTCCTTTTGGTTAAAT
GGGTGTTTAATCTTTTGGTTAGTGGGTTAAACGGGGTAAGTTAGAGTAGG
GGGAGGGATAGGAAGACATATTTAAAAACCATTAAAACACTGTCTCCCAC
TCATGAAATGAGCCACGTAGTTCCTATTTAATGCTGTTTTCCTTTAGTTT
AGAAATACATAGACATTGTCTTTTATGAATTCTGATCATATTTAGTCATT
TTGACCAAATGAGGGATTTGGTCAAATGAGGGATTCCCTCAAAGCAATAT
CAGGTAAACCAAGTTGCTTTCCTCACTCCCTGTCATGAGACTTCAGTGTT
AATGTTCACAATATACTTTCGAAAGAATAAAATAGTTCTCCTACATGAAG
AAAGAATATGTCAGGAAATAAGGTCACTTTATGTCAAAATTATTTGAGTA
CTATGGGACCTGGCGCAGTGGCTCATGCTTGTAATCCCAGCACTTTGGGA
GGCCGAGGTGGGCAGATCACTTGAGATCAGGACCAGCCTGGTCAAGATGG
TGAAACTCCGTCTGTACTAAAAATACAAAATTTAGCTTGGCCTGGTGGCA
GGCACCTGTAATCCCAGCTGCCCAAGAGGCTGAGGCATGAGAATCGCTTG
AACCTGGCAGGCGGAGGTTGCAGTGAGCCGAGATAGTGCCACAGCTCTCC
AGCCTGGGCGACAGAGTGAGACTCCATCTCAAACAACAACAACAACAACA
ACAACAACAACAAACCACAAAATTATTTGAGTACTGTGAAGGATTATTTG
TCTAACAGTTCATTCCAATCAGACCAGGTAGGAGCTTTCCTGTTTCATAT
GTTTCAGGGTTGCACAGTTGGTCTCTTTAATGTCGGTGTGGAGATCCAAA
GTGGGTTGTGGAAAGAGCGTCCATAGGAGAAGTGAGAATACTGTGAAAAA
GGGATGTTAGCATTCATTAGAGTATGAGGATGAGTCCCAAGAAGGTTCTT
TGGAAGGAGGACGAATAGAATGGAGTAATGAAATTCTTGCCATGTGCTGA
GGAGATAGCCAGCATTAGGTGACAATCTTCCAGAAGTGGTCAGGCAGAAG
GTGCCCTGGTGAGAGCTCCTTTACAGGGACTTTATGTGGTTTAGGGCTCA
GAGCTCCAAAACTCTGGGCTCAGCTGCTCCTGTACCTTGGAGGTCCATTC
ACATGGGAAAGTATTTTGGAATGTGTCTTTTGAAGAGAGCATCAGAGTTC
TTAAGGGACTGGGTAAGGCCTGACCCTGAAATGACCATGGATATTTTTCT
ACCTACAGTTTGAGTCAACTAGAATATGCCTGGGGACCTTGAAGAATGGC
CCTTCAGTGGCCCTCACCATTTGTTCATGCTTCAGTTAATTCAGGTGTTG
AAGGAGCTTAGGTTTTAGAGGCACGTAGACTTGGTTCAAGTCTCGTTAGT
AGTTGAATAGCCTCAGGCAAGTCACTGCCCACCTAAGATGATGGTTCTTC
AACTATAAAATGGAGATAATGGTTACAAATGTCTCTTCCTATAGTATAAT
CTCCATAAGGGCATGGCCCAAGTCTGTCTTTGACTCTGCCTATCCCTGAC
ATTTAGTAGCATGCCCGACATACAATGTTAGCTATTGGTATTATTGCCAT
ATAGATAAATTATGTATAAAAATTAAACTGGGCAATAGCCTAAGAAGGGG
GGAATATTGTAACACAAATTTAAACCCACTACGCAGGGATGAGGTGCTAT
AATATGAGGACCTTTTAACTTCCATCATTTTCCTGTTTCTTGAAATAGTT
TATCTTGTAATGAAATATAAGGCACCTCCCACTTTTATGTATAGAAAGAG
GTCTTTTAATTTTTTTTTAATGTGAGAAGGAAGGGAGGAGTAGGAATCTT
GAGATTCCAGATCGAAAATACTGTACTTTGGTTGATTTTTAAGTGGGCTT
CCATTCCATGGATTTAATCAGTCCCAAGAAGATCAAACTCAGCAGTACTT
GGGTGCTGAAGAACTGTTGGATTTACCCTGGCACGTGTGCCACTTGCCAG
CTTCTTGGGCACACAGAGTTCTTCAATCCAAGTTATCAGATTGTATTTGA
AAATGACAGAGCTGGAGAGTTTTTTGAAATGGCAGTGGCAAATAAATAAA
TACTTTTTTTTAAATGGAAAGACTTGATCTATGGTAATAAATGATTTTGT
TTTCTGACTGGAAAAATAGGCCTACTAAAGATGAATCACACTTGAGATGT
TTCTTACTCACTCTGCACAGAAACAAAGAAGAAATGTTATACAGGGAAGT
CCGTTTTCACTATTAGTATGAACCAAGAAATGGTTCAAAAACAGTGGTAG
GAGCAATGCTTTCATAGTTTCAGATATGGTAGTTATGAAGAAAACAATGT
CATTTGCTGCTATTATTGTAAGAGTCTTATAATTAATGGTACTCCTATAA
TTTTTGATTGTGAGCTCACCTATTTGGGTTAAGCATGCCAATTTAAAGAG
ACCAAGTGTATGTACATTATGTTCTACATATTCAGTGATAAAATTACTAA
ACTACTATATGTCTGCTTTAAATTTGTACTTTAATATTGTCTTTTGGTAT
TAAGAAAGATATGCTTTCAGAATAGATATGCTTCGCTTTGGCAAGGAATT
TGGATAGAACTTGCTATTTAAAAGAGGTGTGGGGTAAATCCTTGTATAAA
TCTCCAGTTTAGCCTTTTTTGAAAAAGCTAGACTTTCAAATACTAATTTC
ACTTCAAGCAGGGTACGTTTCTGGTTTGTTTGCTTGACTTCAGTCACAAT
TTCTTATCAGACCAATGGCTGACCTCTTTGAGATGTCAGGCTAGGCTTAC
CTATGTGTTCTGTGTCATGTGAATGCTGAGAAGTTTGACAGAGATCCAAC
TTCAGCCTTGACCCCATCAGTCCCTCGGGTTAACTAACTGAGCCACCGGT
CCTCATGGCTATTTTAATGAGGGTATTGATGGTTAAATGCATGTCTGATC
CCTTATCCCAGCCATTTGCACTGCCAGCTGGGAACTATACCAGACCTGGA
TACTGATCCCAAAGTGTTAAATTCAACTACATGCTGGAGATTAGAGATGG
TGCCAATAAAGGACCCAGAACCAGGATCTTGATTGCTATAGACTTATTAA
TAATCCAGGTCAAAGAGAGTGACACACACTCTCTCAAGACCTGGGGTGAG
GGAGTCTGTGTTATCTGCAAGGCCATTTGAGGCTCAGAAAGTCTCTCTTT
CCTATAGATATATGCATACTTTCTGACATATAGGAATGTATCAGGAATAC
TCAACCATCACAGGCATGTTCCTACCTCAGGGCCTTTACATGTCCTGTTT
ACTCTGTCTAGAATGTCCTTCTGTAGATGACCTGGCTTGCCTCGTCACCC
TTCAGGTCCTTGCTCAAGTGTCATCTTCTCCCCTAGTTAAACTACCCCAC
ACCCTGTCTGCTTTCCTTGCTTATTTTTCTCCATAGCATTTTACCATCTC
TTACATTAGACATTTTTCTTATTTATTTGTAGTTTATAAGCTTCATGAGG
CAAGTAACTTTGCTTTGTTTCTTGCTGTATCTCCAGTGCCCAGAGCAGTG
CCTGGTATATAATAAATATTTATTGACTGAGTGAAAAAAAAAAAAAAAAA.
(SEQ ID NO: 87)
MLRLLLALNLFPSIQVTGNKILVKQSPMLVAYDNAVNLSCKYSYNLFSRE
FRASLHKGLDSAVEVCVVYGNYSQQLQVYSKTGFNCDGKLGNESVTFYLQ
NLYVNQTDIYFCKIEVMYPPPYLDNEKSNGTIIHVKGKHLCPSPLFPGPS
KPFWVLVVVGGVLACYSLLVTVAFIIFWVRSKRSRLLHSDYMNMTPRRPG
PTRKHYQPYAPPRDFAAYRS.

Representative nucleotide and amino acid sequences for human CD30L are set forth in SEQ ID NO:88 (accession no. L09753) and SEQ ID NO:89, respectively:

(SEQ ID NO: 88)
CCAAGTCACATGATTCAGGATTCAGGGGGAGAATCCTTCTTGGAACAGAG
ATGGGCCCAGAACTGAATCAGATGAAGAGAGATAAGGTGTGATGTGGGGA
AGACTATATAAAGAATGGACCCAGGGCTGCAGCAAGCACTCAACGGAATG
GCCCCTCCTGGAGACACAGCCATGCATGTGCCGGCGGGCTCCGTGGCCAG
CCACCTGGGGACCACGAGCCGCAGCTATTTCTATTTGACCACAGCCACTC
TGGCTCTGTGCCTTGTCTTCACGGTGGCCACTATTATGGTGTTGGTCGTT
CAGAGGACGGACTCCATTCCCAACTCACCTGACAACGTCCCCCTCAAAGG
AGGAAATTGCTCAGAAGACCTCTTATGTATCCTGAAAAGAGCTCCATTCA
AGAAGTCATGGGCCTACCTCCAAGTGGCAAAGCATCTAAACAAAACCAAG
TTGTCTTGGAACAAAGATGGCATTCTCCATGGAGTCAGATATCAGGATGG
GAATCTGGTGATCCAATTCCCTGGTTTGTACTTCATCATTTGCCAACTGC
AGTTTCTTGTACAATGCCCAAATAATTCTGTCGATCTGAAGTTGGAGCTT
CTCATCAACAAGCATATCAAAAAACAGGCCCTGGTGACAGTGTGTGAGTC
TGGAATGCAAACGAAACACGTATACCAGAATCTCTCTCAATTCTTGCTGG
ATTACCTGCAGGTCAACACCACCATATCAGTCAATGTGGATACATTCCAG
TACATAGATACAAGCACCTTTCCTCTTGAGAATGTGTTGTCCATCTTCTT
ATACAGTAATTCAGACTGAACAGTTTCTCTTGGCCTTCAGGAAGAAAGCG
CCTCTCTACCATACAGTATTTCATCCCTCCAAACACTTGGGCAAAAAGAA
AACTTTAGACCAAGACAAACTACACAGGGTATTAAATAGTATACTTCTCC
TTCTGTCTCTTGGAAAGATACAGCTCCAGGGTTAAAAAGAGAGTTTTTAG
TGAAGTATCTTTCAGATAGCAGGCAGGGAAGCAATGTAGTGTGGTGGGCA
GAGCCCCACACAGAATCAGAAGGGATGAATGGATGTCCCAGCCCAACCAC
TAATTCACTGTATGGTCTTGATCTATTTCTTCTGTTTTGAGAGCCTCCAG
TTAAAATGGGGCTTCAGTACCAGAGCAGCTAGCAACTCTGCCCTAATGGG
AAATGAAGGGGAGCTGGGTGTGAGTGTTTACACTGTGCCCTTCACGGGAT
ACTTCTTTTATCTGCAGATGGCCTAATGCTTAGTTGTCCAAGTCGCGATC
AAGGACTCTCTCACACAGGAAACTTCCCTATACTGGCAGATACACTTGTG
ACTGAACCATGCCCAGTTTATGCCTGTCTGACTGTCACTCTGGCACTAGG
AGGCTGATCTTGTACTCCATATGACCCCACCCCTAGGAACCCCCAGGGAA
AACCAGGCTCGGACAGCCCCCTGTTCCTGAGATGGAAAGCACAAATTTAA
TACACCACCACAATGGAAAACAAGTTCAAAGACTTTTACTTACAGATCCT
GGACAGAAAGGGCATAATGAGTCTGAAGGGCAGTCCTCCTTCTCCAGGTT
ACATGAGGCAGGAATAAGAAGTCAGACAGAGACAGCAAGACAGTTAACAA
CGTAGGTAAAGAAATAGGGTGTGGTCACTCTCAATTCACTGGCAAATGCC
TGAATGGTCTGTCTGAAGGAAGCAACAGAGAAGTGGGGAATCCAGTCTGC
TAGGCAGGAAAGATGCCTCTAAGTTCTTGTCTCTGGCCAGAGGTGTGGTA
TAGAACCAGAAACCCATATCAAGGGTGACTAAGCCCGGCTTCCGGTATGA
GAAATTAAACTTGTATACAAAATGGTTGCCAAGGCAACATAAAATTATAA
GAATTC.
(SEQ ID NO: 89)
MDPGLQQALNGMAPPGDTAMHVPAGSVASHLGTTSRSYFYLTTATLALCL
VFTVATIMVLVVQRTDSIPNSPDNVPLKGGNCSEDLLCILKRAPFKKSWA
YLQVAKHLNKTKLSWNKDGILHGVRYQDGNLVIQFPGLYFIICQLQFLVQ
CPNNSVDLKLELLINKHIKKQALVTVCESGMQTKHVYQNLSQFLLDYLQV
NTTISVNVDTFQYIDTSTFPLENVLSIFLYSNSD.

Representative nucleotide and amino acid sequences for human CD40 are set forth in SEQ ID NO:90 (accession no. NM_001250) and SEQ ID NO:91, respectively:

(SEQ ID NO: 90)
TTTCCTGGGCGGGGCCAAGGCTGGGGCAGGGGAGTCAGCAGAGGCCTCGC
TCGGGCGCCCAGTGGTCCTGCCGCCTGGTCTCACCTCGCTATGGTTCGTC
TGCCTCTGCAGTGCGTCCTCTGGGGCTGCTTGCTGACCGCTGTCCATCCA
GAACCACCCACTGCATGCAGAGAAAAACAGTACCTAATAAACAGTCAGTG
CTGTTCTTTGTGCCAGCCAGGACAGAAACTGGTGAGTGACTGCACAGAGT
TCACTGAAACGGAATGCCTTCCTTGCGGTGAAAGCGAATTCCTAGACACC
TGGAACAGAGAGACACACTGCCACCAGCACAAATACTGCGACCCCAACCT
AGGGCTTCGGGTCCAGCAGAAGGGCACCTCAGAAACAGACACCATCTGCA
CCTGTGAAGAAGGCTGGCACTGTACGAGTGAGGCCTGTGAGAGCTGTGTC
CTGCACCGCTCATGCTCGCCCGGCTTTGGGGTCAAGCAGATTGCTACAGG
GGTTTCTGATACCATCTGCGAGCCCTGCCCAGTCGGCTTCTTCTCCAATG
TGTCATCTGCTTTCGAAAAATGTCACCCTTGGACAAGCTGTGAGACCAAA
GACCTGGTTGTGCAACAGGCAGGCACAAACAAGACTGATGTTGTCTGTGG
TCCCCAGGATCGGCTGAGAGCCCTGGTGGTGATCCCCATCATCTTCGGGA
TCCTGTTTGCCATCCTCTTGGTGCTGGTCTTTATCAAAAAGGTGGCCAAG
AAGCCAACCAATAAGGCCCCCCACCCCAAGCAGGAACCCCAGGAGATCAA
TTTTCCCGACGATCTTCCTGGCTCCAACACTGCTGCTCCAGTGCAGGAGA
CTTTACATGGATGCCAACCGGTCACCCAGGAGGATGGCAAAGAGAGTCGC
ATCTCAGTGCAGGAGAGACAGTGAGGCTGCACCCACCCAGGAGTGTGGCC
ACGTGGGCAAACAGGCAGTTGGCCAGAGAGCCTGGTGCTGCTGCTGCTGT
GGCGTGAGGGTGAGGGGCTGGCACTGACTGGGCATAGCTCCCCGCTTCTG
CCTGCACCCCTGCAGTTTGAGACAGGAGACCTGGCACTGGATGCAGAAAC
AGTTCACCTTGAAGAACCTCTCACTTCACCCTGGAGCCCATCCAGTCTCC
CAACTTGTATTAAAGACAGAGGCAGAAGTTTGGTGGTGGTGGTGTTGGGG
TATGGTTTAGTAATATCCACCAGACCTTCCGATCCAGCAGTTTGGTGCCC
AGAGAGGCATCATGGTGGCTTCCCTGCGCCCAGGAAGCCATATACACAGA
TGCCCATTGCAGCATTGTTTGTGATAGTGAACAACTGGAAGCTGCTTAAC
TGTCCATCAGCAGGAGACTGGCTAAATAAAATTAGAATATATTTATACAA
CAGAATCTCAAAAACACTGTTGAGTAAGGAAAAAAAGGCATGCTGCTGAA
TGATGGGTATGGAACTTTTTAAAAAAGTACATGCTTTTATGTATGTATAT
TGCCTATGGATATATGTATAAATACAATATGCATCATATATTGATATAAC
AAGGGTTCTGGAAGGGTACACAGAAAACCCACAGCTCGAAGAGTGGTGAC
GTCTGGGGTGGGGAAGAAGGGTCTGGGGG.
(SEQ ID NO: 91)
MVRLPLQCVLWGCLLTAVHPEPPTACREKQYLINSQCCSLCQPGQKLVSD
CTEFTETECLPCGESEFLDTWNRETHCHQHKYCDPNLGLRVQQKGTSETD
TICTCEEGWHCTSEACESCVLHRSCSPGFGVKQIATGVSDTICEPCPVGF
FSNVSSAFEKCHPWTSCETKDLVVQQAGTNKTDVVCGPQDRLRALVVIPI
IFGILFAILLVLVFIKKVAKKPTNKAPHPKQEPQEINFPDDLPGSNTAAP
VQETLHGCQPVTQEDGKESRISVQERQ.

Representative nucleotide and amino acid sequences for human CD70 are set forth in SEQ ID NO:92 (accession no. NM_001252) and SEQ ID NO:93, respectively:

(SEQ ID NO: 92)
CCAGAGAGGGGCAGGCTGGTCCCCTGACAGGTTGAAGCAAGTAGACGCCC
AGGAGCCCCGGGAGGGGGCTGCAGTTTCCTTCCTTCCTTCTCGGCAGCGC
TCCGCGCCCCCATCGCCCCTCCTGCGCTAGCGGAGGTGATCGCCGCGGCG
ATGCCGGAGGAGGGTTCGGGCTGCTCGGTGCGGCGCAGGCCCTATGGGTG
CGTCCTGCGGGCTGCTTTGGTCCCATTGGTCGCGGGCTTGGTGATCTGCC
TCGTGGTGTGCATCCAGCGCTTCGCACAGGCTCAGCAGCAGCTGCCGCTC
GAGTCACTTGGGTGGGACGTAGCTGAGCTGCAGCTGAATCACACAGGACC
TCAGCAGGACCCCAGGCTATACTGGCAGGGGGGCCCAGCACTGGGCCGCT
CCTTCCTGCATGGACCAGAGCTGGACAAGGGGCAGCTACGTATCCATCGT
GATGGCATCTACATGGTACACATCCAGGTGACGCTGGCCATCTGCTCCTC
CACGACGGCCTCCAGGCACCACCCCACCACCCTGGCCGTGGGAATCTGCT
CTCCCGCCTCCCGTAGCATCAGCCTGCTGCGTCTCAGCTTCCACCAAGGT
TGTACCATTGCCTCCCAGCGCCTGACGCCCCTGGCCCGAGGGGACACACT
CTGCACCAACCTCACTGGGACACTTTTGCCTTCCCGAAACACTGATGAGA
CCTTCTTTGGAGTGCAGTGGGTGCGCCCCTGACCACTGCTGCTGATTAGG
GTTTTTTAAATTTTATTTTATTTTATTTAAGTTCAAGAGAAAAAGTGTAC
ACACAGGGGCCACCCGGGGTTGGGGTGGGAGTGTGGTGGGGGGTAGTGGT
GGCAGGACAAGAGAAGGCATTGAGCTTTTTCTTTCATTTTCCTATTAAAA
AATACAAAAATCA.
(SEQ ID NO: 93)
MPEEGSGCSVRRRPYGCVLRAALVPLVAGLVICLVVCIQRFAQAQQQLPL
ESLGWDVAELQLNHTGPQQDPRLYWQGGPALGRSFLHGPELDKGQLRIHR
DGIYMVHIQVTLAICSSTTASRHHPTTLAVGICSPASRSISLLRLSFHQG
CTIASQRLTPLARGDTLCTNLTGTLLPSRNTDETFFGVQWVRP.

Representative nucleotide and amino acid sequences for human LIGHT are set forth in SEQ ID NO:94 (accession no. CR541854) and SEQ ID NO:95, respectively:

(SEQ ID NO: 94)
ATGGAGGAGAGTGTCGTACGGCCCTCAGTGTTTGTGGTGGATGGACAGAC
CGACATCCCATTCACGAGGCTGGGACGAAGCCACCGGAGACAGTCGTGCA
GTGTGGCCCGGGTGGGTCTGGGTCTCTTGCTGTTGCTGATGGGGGCCGGG
CTGGCCGTCCAAGGCTGGTTCCTCCTGCAGCTGCACTGGCGTCTAGGAGA
GATGGTCACCCGCCTGCCTGACGGACCTGCAGGCTCCTGGGAGCAGCTGA
TACAAGAGCGAAGGTCTCACGAGGTCAACCCAGCAGCGCATCTCACAGGG
GCCAACTCCAGCTTGACCGGCAGCGGGGGGCCGCTGTTATGGGAGACTCA
GCTGGGCCTGGCCTTCCTGAGGGGCCTCAGCTACCACGATGGGGCCCTTG
TGGTCACCAAAGCTGGCTACTACTACATCTACTCCAAGGTGCAGCTGGGC
GGTGTGGGCTGCCCGCTGGGCCTGGCCAGCACCATCACCCACGGCCTCTA
CAAGCGCACACCCCGCTACCCCGAGGAGCTGGAGCTGTTGGTCAGCCAGC
AGTCACCCTGCGGACGGGCCACCAGCAGCTCCCGGGTCTGGTGGGACAGC
AGCTTCCTGGGTGGTGTGGTACACCTGGAGGCTGGGGAGGAGGTGGTCGT
CCGTGTGCTGGATGAACGCCTGGTTCGACTGCGTGATGGTACCCGGTCTT
ACTTCGGGGCTTTCATGGTGTGA.
(SEQ ID NO: 95)
MEESVVRPSVFVVDGQTDIPFTRLGRSHRRQSCSVARVGLGLLLLLMGAG
LAVQGWFLLQLHWRLGEMVTRLPDGPAGSWEQLIQERRSHEVNPAAHLTG
ANSSLTGSGGPLLWETQLGLAFLRGLSYHDGALVVTKAGYYYIYSKVQLG
GVGCPLGLASTITHGLYKRTPRYPEELELLVSQQSPCGRATSSSRVWWDS
SFLGGVVHLEAGEEVVVRVLDERLVRLRDGTRSYFGAFMV.

In various embodiments, the present invention provides for variants comprising any of the sequences described herein, for instance, a sequence having at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99%) sequence identity with any of the sequences disclosed herein (for example, SEQ ID NOS: 47-59 and 84-95).

In various embodiments, the present invention provides for an amino acid sequence having one or more amino acid mutations relative any of the protein sequences described herein. In some embodiments, the one or more amino acid mutations may be independently selected from conservative or non-conservative substitutions, insertions, deletions, and truncations as described herein.

Coronavirus

As used herein, the term “coronavirus” refers to any one of the genus of viruses in the family Coronaviridae, including, but not limited to the betacoronavirus (e.g. SARS-CoV-2 (2019-nCoV), SARS-CoV, MERS-CoV, HCoV-HKU1, and HCoV-OC43) and alphacoronavirus (e.g. HCoV-NL63 and HCoV-229E). In exemplary aspects, the coronavirus is SARS-CoV-2 virus. Phylogenetic analysis of the complete genome of SARS-CoV-2 (GenBank Accession No.: MN908947) revealed that the virus was most closely related (89.1% nucleotide similarity) to a group of SARS-like coronaviruses (genus Betacoronavirus, subgenus Sarbecovirus). Wu et al., A new coronavirus associated with human respiratory disease in China. Nature, Feb. 3, 2020, which is incorporated herein by reference in its entirety. In various embodiments, the coronavirus is a variant of a SARS-CoV-2 protein, such as, without limitation, a protein (or an antigenic fragment thereof) having one or more mutations relative to the sequence of SARS-CoV-2 (GenBank Accession No.: MN908947).

Coronavirus Proteins

In various embodiments, the expression vector system of the present invention comprises one, two, or more variants of a coronavirus protein, or an antigenic portion thereof. In embodiments, the expression vector system of the present invention comprises a nucleic acid encoding a coronavirus protein, or an antigenic portion thereof. The coronavirus protein is a betacoronavirus protein or an alphacoronavirus protein, optionally wherein the betacoronavirus protein is selected from a SARS-CoV-2, SARS-CoV, MERS-CoV, HCoV-HKU1, and HCoV-OC43 protein, or an antigenic fragment thereof or the alphacoronavirus protein is selected from an HCoV-NL63 and HCoV-229E protein, or an antigenic fragment thereof.

In some embodiments, the betacoronavirus protein is a SARS-CoV-2 protein or a variant thereof.

In some embodiments, wherein the SARS-CoV-2 protein comprises an amino acid encoded by a nucleic acid having a nucleotide sequence of SEQ ID NO: 46, or an antigenic fragment thereof. In some embodiments, the SARS-CoV-2 protein comprises the amino acid that encompasses an amino acid of sequence of SEQ ID NO: 36, an amino acid of sequence of SEQ ID NO: 37, an amino acid of sequence of SEQ ID NO: 38, an amino acid of sequence of SEQ ID NO: 39, an amino acid of sequence of SEQ ID NO: 40, an amino acid of sequence of SEQ ID NO: 41, an amino acid of sequence of SEQ ID NO: 42, an amino acid of sequence of SEQ ID NO: 43, and an amino acid of sequence of SEQ ID NO: 44, or an antigenic fragment thereof.

In some embodiments, the coronavirus protein is a SARS-CoV-2 protein, or an antigenic fragment thereof, selected from the spike surface glycoprotein, membrane glycoprotein M, envelope protein E, and nucleocapsid phosphoprotein N. The coronavirus protein can include one or more mutations. In some embodiments, the spike surface glycoprotein comprises the amino acid sequence of SEQ ID NO: 37, membrane glycoprotein precursor M comprises the amino acid sequence of SEQ ID NO: 40, the envelope protein E comprises the amino acid sequence of SEQ ID NO: 39, and the nucleocapsid phosphoprotein N comprises the amino acid sequence of SEQ ID NO: 44, or an amino acid sequence having at least about 90%, or at least about 95%, or at least about 97%, or at least about 98%, or at least about 99% identity with any of the foregoing, or an antigenic fragment of any of the foregoing, or a variant of any of the foregoing.

In embodiments, the coronavirus includes one or more mutations in any one or more of the spike surface glycoprotein, membrane glycoprotein M, envelope protein E, and nucleocapsid phosphoprotein N.

In embodiments, the coronavirus includes one or more mutations in the spike surface glycoprotein (Spike protein).

In some embodiments, the spike surface glycoprotein comprises an amino acid sequence having at least one mutation relative to the amino acid sequence of SEQ ID NO: 37.

In some embodiments, the spike surface glycoprotein comprises an amino acid sequence having D614G mutation relative to the amino acid sequence of SEQ ID NO: 37.

In some embodiments, the spike surface glycoprotein comprises an amino acid sequence having E484K mutation relative to the amino acid sequence of SEQ ID NO: 37.

In some embodiments, the spike surface glycoprotein comprises an amino acid sequence having N501Y mutation relative to the amino acid sequence of SEQ ID NO: 37.

In some embodiments, the spike surface glycoprotein comprises an amino acid sequence having K417N mutation relative to the amino acid sequence of SEQ ID NO: 37.

In some embodiments, the spike surface glycoprotein comprises an amino acid sequence having S477G or S477N mutation relative to the amino acid sequence of SEQ ID NO: 37.

In some embodiments, the spike surface glycoprotein comprises one or more of D614G, E484K, N501Y, K417N, S477G, and S477N mutations relative to the amino acid sequence of SEQ ID NO: 37.

In some embodiments, the expression vector comprises two or more nucleic acids each encoding a different coronavirus protein, or an antigenic portion thereof.

In some embodiments, the coronavirus is betacoronavirus such as SARS-CoV-2 (2019-nCoV) or another betacoronavirus, and the complete genome of the SARS-CoV-2 coronavirus (29903 nucleotides, single-stranded RNA) is described in the NCBI database as GenBank Reference Sequence: MN908947. The coronavirus protein can be selected from the group consisting of: coronavirus spike protein (GenBank Reference Sequence: QHD43416), coronavirus membrane glycoprotein M (GenBank Reference Sequence: QHD43419), coronavirus envelope protein E (GenBank Reference Sequence: QHD43418), and coronavirus nucleocapsid phosphoprotein E (GenBank Reference Sequence: QHD43423), or any variant thereof.

In various embodiments, the coronavirus is SARS-CoV-2 (2019-nCoV). In some embodiments, the expression vector system of the present invention comprises a nucleic acid encoding a SARS-CoV-2 virus protein, or an antigenic portion thereof. In exemplary aspects, the expression vector comprises two or more nucleic acids each encoding a different coronavirus protein, or an antigenic portion thereof. The nucleic acid sequence of the SARS-CoV-2 (2019-nCoV) virus has recently been identified. Wu et al., A new coronavirus associated with human respiratory disease in China. Nature, Feb. 3, 2020; see also GenBank Accession Number: MN908947.3, the contents of which are hereby incorporated by reference. In various embodiments, the expression vector system of the invention comprises a nucleic acid encoding any of the known coronavirus protein or an antigenic portion, fragments, or variants thereof. In various embodiments, the SARS-CoV-2 protein is one or more of a spike protein, membrane glycoprotein M, envelope protein E, and nucleocapsid phosphoprotein E, or antigenic portions, fragments, or variants thereof. The trimeric spike (S) protein comprises subunits S1 and S2. See Daniel et al., Cryo-EM structure of the 2019-nCoV spike in the prefusion conformation. Science, 19 Feb. 2020, which is incorporated herein by reference in its entirety.

In some embodiments, the spike protein comprises the following amino acid sequence:

(SEQ ID NO: 37)
MFVFLVLLPLVSSQCVNLTTRTQLPPAYTNSFTRGVYYPDKVFRSSVLHS
TQDLFLPFFSNVTWFHAIHVSGTNGTKRFDNPVLPFNDGVYFASTEKSNI
IRGWIFGTTLDSKTQSLLIVNNATNVVIKVCEFQFCNDPFLGVYYHKNNK
SWMESEFRVYSSANNCTFEYVSQPFLMDLEGKQGNFKNLREFVFKNIDGY
FKIYSKHIPINLVRDLPQGFSALEPLVDLPIGINITRFQTLLALHRSYLT
PGDSSSGWTAGAAAYYVGYLQPRTFLLKYNENGTITDAVDCALDPLSETK
CTLKSFTVEKGIYQTSNFRVQPTESIVRFPNITNLCPFGEVFNATRFASV
YAWNRKRISNCVADYSVLYNSASFSTFKCYGVSPTKLNDLCFTNVYADSF
VIRGDEVRQIAPGQTGKIADYNYKLPDDFTGCVIAWNSNNLDSKVGGNYN
YLYRLFRKSNLKPFERDISTEIYQAGSTPCNGVEGFNCYFPLQSYGFQPT
NGVGYQPYRVVVLSFELLHAPATVCGPKKSTNLVKNKCVNFNFNGLTGTG
VLTESNKKFLPFQQFGRDIADTTDAVRDPQTLEILDITPCSFGGVSVITP
GTNTSNQVAVLYQDVNCTEVPVAIHADQLTPTWRVYSTGSNVFQTRAGCL
IGAEHVNNSYECDIPIGAGICASYQTQTNSPRRARSVASQSIIAYTMSLG
AENSVAYSNNSIAIPTNFTISVTTEILPVSMTKTSVDCTMYICGDSTECS
NLLLQYGSFCTQLNRALTGIAVEQDKNTQEVFAQVKQIYKTPPIKDFGGF
NFSQILPDPSKPSKRSFIEDLLFNKVTLADAGFIKQYGDCLGDIAARDLI
CAQKFNGLTVLPPLLTDEMIAQYTSALLAGTITSGWTFGAGAALQIPFAM
QMAYRFNGIGVTQNVLYENQKLIANQFNSAIGKIQDSLSSTASALGKLQD
VVNQNAQALNTLVKQLSSNFGAISSVLNDILSRLDKVEAEVQIDRLITGR
LQSLQTYVTQQLIRAAEIRASANLAATKMSECVLGQSKRVDFCGKGYHLM
SFPQSAPHGVVFLHVTYVPAQEKNFTTAPAICHDGKAHFPREGVFVSNGT
HWFVTQRNFYEPQIITTDNTFVSGNCDVVIGIVNNTVYDPLQPELDSFKE
ELDKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCCSC
GSCCKFDEDDSEPVLKGVKLHYT.

In some embodiments, the envelope protein comprises the following amino acid sequence:

(SEQ ID NO: 39)
MYSFVSEETGTLIVNSVLLFLAFVVFLLVTLAILTALRLCAYCCNIVNV
SLVKPSFYVYSRVKNLNSSRVPDLLV.

In some embodiments, the membrane protein comprises the following amino acid sequence:

(SEQ ID NO: 40)
MADSNGTITVEELKKLLEQWNLVIGFLFLTWICLLQFAYANRNRFLYII
KLIFLWLLWPVTLACFVLAAVYRINWITGGIAIAMACLVGLMWLSYFIA
SFRLFARTRSMWSFNPETNILLNVPLHGTILTRPLLESELVIGAVILRG
HLRIAGHHLGRCDIKDLPKEITVATSRTLSYYKLGASQRVAGDSGFAAY
SRYRIGNYKLNTDHSSSSDNIALLVQ.

In some embodiments, the nucleocapsid phosphoprotein comprises the following amino acid sequence:

(SEQ ID NO: 44)
MSDNGPQNQRNAPRITFGGPSDSTGSNQNGERSGARSKQRRPQGLPNNT
ASWFTALTQHGKEDLKFPRGQGVPINTNSSPDDQIGYYRRATRRIRGGD
GKMKDLSPRWYFYYLGTGPEAGLPYGANKDGIIWVATEGALNTPKDHIG
TRNPANNAAIVLQLPQGTTLPKGFYAEGSRGGSQASSRSSSRSRNSSRN
STPGSSRGTSPARMAGNGGDAALALLLLDRLNQLESKMSGKGQQQQGQT
VTKKSAAEASKKPRQKRTATKAYNVTQAFGRRGPEQTQGNFGDQELIRQ
GTDYKHWPQIAQFAPSASAFFGMSRIGMEVTPSGTWLTYTGAIKLDDKD
PNFKDQVILLNKHIDAYKTFPPTEPKKDKKKKADETQALPQRQKKQQTV
TLLPAADLDDFSKQLQQSMSSADSTQA.

In some embodiments, the expression vector system comprises a nucleic acid encoding the SARS-CoV-2 protein comprising a nucleic acid encoding the SARS-CoV-2 protein surface glycoprotein protein, a nucleic acid encoding the SARS-CoV-2 protein membrane glycoprotein, a nucleic acid encoding the SARS-CoV-2 protein envelope protein E, and/or a nucleic acid encoding the SARS-CoV-2 protein Nucleocapsid protein E, or antigenic portions, fragments, or variants thereof. In some embodiments, the expression vector system comprises a nucleic acid encoding the amino acid sequence of SEQ ID NO: 37, a nucleic acid encoding the amino acid sequence of SEQ ID NO: 39, a nucleic acid encoding the amino acid sequence of SEQ ID NO: 40, a nucleic acid encoding the amino acid sequence of SEQ ID NO: 44. In some embodiments, the expression vector system comprises a nucleic acid encoding the amino acid sequence of SEQ ID NO: 37.

In some embodiments, the expression vector system comprises a nucleic acid encoding the amino acid sequence of SEQ ID NO: 37 or a variant thereof having one or more mutations, a nucleic acid encoding the amino acid sequence of SEQ ID NO: 39 or a variant thereof having one or more mutations, a nucleic acid encoding the amino acid sequence of SEQ ID NO: 40 or a variant thereof having one or more mutations, a nucleic acid encoding the amino acid sequence of SEQ ID NO: 44 or a variant thereof having one or more mutations. In some embodiments, the expression vector system comprises a nucleic acid encoding the amino acid sequence of SEQ ID NO: 37 or a variant thereof having one or more mutations.

Alternatively, in some embodiments, the expression vector system of the present invention may comprise a nucleic acid encoding a SARS-CoV-2 (2019-nCoV) protein variant that contains one or more substitutions, deletions, or additions as compared to any known wild type amino acid sequence of the 2019-nCoV protein or a 2019-nCoV amino acid sequence disclosed herein.

In various embodiments, the 2019-nCoV protein may comprise an amino acid sequence that has at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with any known wild type amino acid sequence of the 2019-nCoV protein or a 2019-nCoV amino acid sequence disclosed herein (e.g. about 60%, or about 61%, or about 62%, or about 63%, or about 64%, or about 65%, or about 66%, or about 67%, or about 68%, or about 69%, or about 70%, or about 71%, or about 72%, or about 73%, or about 74%, or about 75%, or about 76%, or about 77%, or about 78%, or about 79%, or about 80%, or about 81%, or about 82%, or about 83%, or about 84%, or about 85%, or about 86%, or about 87%, or about 88%, or about 89%, or about 90%, or about 91%, or about 92%, or about 93%, or about 94%, or about 95%, or about 96%, or about 97%, or about 98%, or about 99% sequence identity), e.g. relative to an amino acid encoded by a nucleic acid having a nucleotide sequence of SEQ ID NO: 46, or an antigenic portion thereof.

In various embodiments, the 2019-nCoV protein may comprise an amino acid sequence that has at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with any known wild type amino acid sequence of the 2019-nCoV protein or a 2019-nCoV amino acid sequence disclosed herein (e.g. about 60%, or about 61%, or about 62%, or about 63%, or about 64%, or about 65%, or about 66%, or about 67%, or about 68%, or about 69%, or about 70%, or about 71%, or about 72%, or about 73%, or about 74%, or about 75%, or about 76%, or about 77%, or about 78%, or about 79%, or about 80%, or about 81%, or about 82%, or about 83%, or about 84%, or about 85%, or about 86%, or about 87%, or about 88%, or about 89%, or about 90%, or about 91%, or about 92%, or about 93%, or about 94%, or about 95%, or about 96%, or about 97%, or about 98%, or about 99% sequence identity), e.g. relative to any one of SEQ ID NOs: 37, 39, 40, 44, or an antigenic fragment thereof.

In various embodiments, the 2019-nCoV protein may comprise an amino acid sequence that has one or more mutations relative to an amino acid sequence having at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with any known wild type amino acid sequence of the 2019-nCoV protein or a 2019-nCoV amino acid sequence disclosed herein (e.g. about 60%, or about 61%, or about 62%, or about 63%, or about 64%, or about 65%, or about 66%, or about 67%, or about 68%, or about 69%, or about 70%, or about 71%, or about 72%, or about 73%, or about 74%, or about 75%, or about 76%, or about 77%, or about 78%, or about 79%, or about 80%, or about 81%, or about 82%, or about 83%, or about 84%, or about 85%, or about 86%, or about 87%, or about 88%, or about 89%, or about 90%, or about 91%, or about 92%, or about 93%, or about 94%, or about 95%, or about 96%, or about 97%, or about 98%, or about 99% sequence identity), e.g. relative to an amino acid encoded by a nucleic acid having a nucleotide sequence of SEQ ID NO: 46, or an antigenic portion thereof.

In various embodiments, the 2019-nCoV protein may comprise an amino acid sequence that has one or more mutations relative to an amino acid sequence having at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with any known wild type amino acid sequence of the 2019-nCoV protein or a 2019-nCoV amino acid sequence disclosed herein (e.g. about 60%, or about 61%, or about 62%, or about 63%, or about 64%, or about 65%, or about 66%, or about 67%, or about 68%, or about 69%, or about 70%, or about 71%, or about 72%, or about 73%, or about 74%, or about 75%, or about 76%, or about 77%, or about 78%, or about 79%, or about 80%, or about 81%, or about 82%, or about 83%, or about 84%, or about 85%, or about 86%, or about 87%, or about 88%, or about 89%, or about 90%, or about 91%, or about 92%, or about 93%, or about 94%, or about 95%, or about 96%, or about 97%, or about 98%, or about 99% sequence identity), e.g. relative to any one of SEQ ID NOs: 37, 39, 40, 44, or an antigenic fragment thereof.

In various embodiments, the SARS-CoV-2 protein portion of the nucleic acid can encode an amino acid sequence that differs from any known wild type amino acid sequence of the SARS-CoV-2 protein or a SARS-CoV-2 amino acid sequence disclosed herein, or from any variant of SARS-CoV-2 protein, at one or more amino acid positions, such that it contains one or more conservative substitutions, non-conservative substitutions, splice variants, isoforms, homologues from other species, and polymorphisms.

In some embodiments, present invention provides an expression vector system comprising (i) a nucleic acid encoding the amino acid sequence of SEQ ID NO: 2, optionally lacking the terminal KDEL sequence and (ii) a nucleic acid encoding the amino acid sequence of any one or more of SEQ ID NO: 37, SEQ ID NO: 39, SEQ ID NO: 40, or SEQ ID NO: 44, wherein each nucleic acid is operably linked to a promoter. In some embodiments, present invention provides an expression vector system comprising (i) a nucleic acid encoding the amino acid sequence of SEQ ID NO: 2, optionally lacking the terminal KDEL sequence and (ii) a nucleic acid encoding the amino acid sequence of SEQ ID NO: 37, wherein each nucleic acid is operably linked to a promoter. In some embodiments, present invention provides a method of treating or preventing a coronavirus infection in a subject, comprising administering to the subject this expression vector.

In some embodiments, present invention provides a biological cell comprising a first recombinant protein having an amino acid sequence of at least about 90%, or at least about 95% or at least about 97%, or at least about 98%, or at least about 99% sequence identity with SEQ ID NO: 2, optionally lacking the terminal KDEL sequence and a second recombinant protein having an amino acid sequence of at least about 90%, or at least about 95% or at least about 97%, or at least about 98%, or at least about 99% sequence identity with SEQ ID NO: 37. In some embodiments, present invention provides a method of treating or preventing a coronavirus infection in a subject, comprising administering to the subject the biological cell.

In some embodiments, present invention provides a biological cell comprising a first recombinant protein having an amino acid sequence of at least about 90%, or at least about 95% or at least about 97%, or at least about 98%, or at least about 99% sequence identity with SEQ ID NO: 2, optionally lacking the terminal KDEL sequence and a second recombinant protein having an amino acid sequence of at least about 90%, or at least about 95% or at least about 97%, or at least about 98%, or at least about 99% sequence identity with of any one or more of SEQ ID NO: 37, SEQ ID NO: 39, SEQ ID NO: 40, or SEQ ID NO: 44. In some embodiments, present invention provides a method of treating or preventing a coronavirus infection in a subject, comprising administering to the subject the biological cell.

As defined herein, a “conservative substitution” denotes the replacement of an amino acid residue by another, biologically similar, residue. Typically, biological similarity, as referred to above, reflects substitutions on the wild type sequence with conserved amino acids. For example, conservative amino acid substitutions would be expected to have little or no effect on biological activity, particularly if they represent less than 10% of the total number of residues in the polypeptide or protein. Conservative substitutions may be made, for instance, on the basis of similarity in polarity, charge, size, solubility, hydrophobicity, hydrophilicity, and/or the amphipathic nature of the amino acid residues involved. The 20 naturally occurring amino acids can be grouped into the following six standard amino acid groups: (1) hydrophobic: Met, Ala, Val, Leu, Ile; (2) neutral hydrophilic: Cys, Ser, Thr; Asn, Gln; (3) acidic: Asp, Glu; (4) basic: His, Lys, Arg; (5) residues that influence chain orientation: Gly, Pro; and (6) aromatic: Trp, Tyr, Phe. Accordingly, conservative substitutions may be effected by exchanging an amino acid by another amino acid listed within the same group of the six standard amino acid groups shown above. For example, the exchange of Asp by Glu retains one negative charge in the so modified polypeptide. In addition, glycine and proline may be substituted for one another based on their ability to disrupt α-helices. Additional examples of conserved amino acid substitutions, include, without limitation, the substitution of one hydrophobic residue for another, such as isoleucine, valine, leucine, or methionine, or the substitution of one polar residue for another, such as the substitution of arginine for lysine, glutamic for aspartic acid, or glutamine for asparagine, and the like. The term “conservative substitution” also includes the use of a substituted amino acid residue in place of an un-substituted parent amino acid residue, provided that antibodies raised to the substituted polypeptide also immunoreact with the un-substituted polypeptide.

As used herein, “non-conservative substitutions” are defined as exchanges of an amino acid by another amino acid listed in a different group of the six standard amino acid groups (1) to (6) shown above.

In various embodiments, the substitutions may also include non-classical amino acids (e.g. selenocysteine, pyrrolysine, N-formylmethionine 3-alanine, GABA and 6-Aminolevulinic acid, 4-aminobenzoic acid (PABA), D-isomers of the common amino acids, 2,4-diaminobutyric acid, α-amino isobutyric acid, 4-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxyproline, sarcosme, citrulline, homocitrulline, cysteic acid, t-butylglycine, t-butylalanine, phenylglycine, cyclohexylalanine, β-alanine, fluoro-amino acids, designer amino acids such as β methyl amino acids, C α-methyl amino acids, N α-methyl amino acids, and amino acid analogs in general).

Mutations may also be made to the nucleotide sequences of the present 2019-nCoV protein sequence by reference to the genetic code, including taking into account codon degeneracy. Any of the nucleic acid sequences described herein may be codon optimized.

In some embodiments, a COVID-19 vaccine in accordance with the present disclosure induces antigen-specific CD8+ T lymphocytes in epithelial tissues, including lungs. Fisher et al., Frontiers in Immunology, 11, 26 Jan. 2021; 3740, which is incorporated by reference herein in its entirety.

Tissue-resident memory (TRM) T cells have been recognized as a distinct population of memory cells that are capable of rapidly responding to infection in the tissue, without requiring priming in the lymph nodes. See Beura et al., Nat Immunol (2018) 19(2):173-82; Park et al., Nat Immunol (2018) 19(2):183-91; Wakim et al., Science (2008) 319(5860):198-202; Wein et al., J Exp Med (2019) 216(12):2748-62. Several key molecules important for CD8+ T cell entry and retention in the lung have been identified. See Agostini et al., Am J Respir Crit Care Med (2005) 172(10):1290-8; Freeman et al., Am J Pathol (2007) 171(3):767-76; Galkina et al., J Clin Invest (2005) 115(12):3473-83; Kohlmeier et al., Immunity (2008) 29(1):101-13; Ray et al., Immunity (2004) 20(2):167-79; Slutter et al., Immunity (2013) 39(5):939-48. Recently, CD69 and CXCR6 have been confirmed as core markers that define TRM cells in the lungs. See Wein et al., J Exp Med (2019) 216(12):2748-62; Hombrink et al., Nat Immunol (2016) 17(12):1467-78; Kumar et al., Cell Rep (2017) 20(12):2921-34; Mackay et al., Nat Immunol (2013) 14(12):1294-301. Furthermore, it was confirmed that CXCR6-CXCL16 interactions control the localization and maintenance of virus-specific CD8+ TRM cells in the lungs. Wein et al., J Exp Med (2019) 216(12):2748-62. It has also been shown that, in heterosubtypic influenza challenge studies), TRM were required for effective clearance of the virus. See Hogan et al., J Immunol (2001) 166(3):1813-22; Wu et al., J Leukoc Biol (2014) 95(2):215-24; Zens et al., JCI Insight (2016) 1(10):e85832. Therefore, vaccination strategies targeting generation of TRM and their persistence may provide enhanced immunity, compared with vaccines that rely on circulating responses. See Zens et al. (2016). The advantage provided by the gp96-based technology platform in accordance with embodiments of the present disclosure is that any antigen (such as SARS-CoV-2 S peptides) in the complex with gp96 can drive a potent and long-standing immune response.

In some embodiments, a SARS-CoV-2 cell-based vaccine induces protein S (Spike)-specific CD8+ and CD4+ T lymphocytes in epithelial tissues, including lungs and airways. The secreted gp96-Ig-COVID-19 vaccine can elicit robust long-term memory T-cell responses against multiple SARS-CoV-2 antigens and is designed to work cohesively with other treatments/vaccines (as boosters or as second-line defense) with large-scale manufacturing potential.

In some embodiments, a SARS-CoV-2 cell-based vaccine is capable of induction of cellular immune responses in epithelial tissues such as the lungs.

In some embodiments, a SARS-CoV-2 cell-based vaccine induces S1-specific CD8+ T cells in the spleen, lung tissue, and BAL.

In some embodiments, a SARS-CoV-2 cell-based vaccine upregulates CD69 and CXCR6 markers on CD8+ T cells.

In some embodiments, a SARS-CoV-2 cell-based vaccine is capable of inducing CD8+ and CD4+ effector cells in a dose-dependent manner.

Chaperones/Fusion Proteins

In various embodiments, the expression vector system of the present invention comprises a nucleic acid encoding a fusion protein comprising a chaperone protein and an immunoglobulin, or a fragment thereof. In some embodiments, the chaperone protein is selected from the group consisting of: gp96, Hsp70, BiP, and Grp78. In some embodiments, the chaperone protein is gp96. In some embodiments, the chaperone protein comprises an amino acid sequence of any one of SEQ ID NOs: 2, 29,30, and 31, or an amino acid sequence having at least about 90%, or at least about 95%, or at least about 97%, or at least about 98%, or at least about 99% identity thereto. In some embodiments, the chaperone protein is gp96 comprising the amino acid sequence of SEQ ID NO: 2.

In some embodiments, gp96, genetically fused to an immunoglobulin domain (e.g., an IgG1, IgG2, IgG3, IgG4, IgM, IgA, or IgE molecule), activates TLR2 and TLR4 on professional antigen-presenting cells (APCs).

In some embodiments, the fusion protein comprises an Fc fragment of an immunoglobulin. In some embodiments, the immunoglobulin is an IgG1 immunoglobulin. In some embodiments, the Fc fragment comprises the amino acid sequence of SEQ ID NO: 5, or an amino acid sequence having at least about 90%, or at least about 95%, or at least about 97%, or at least about 98%, or at least about 99% identity thereto.

In some embodiments, the fusion protein of the expression vector system comprises the amino acid sequence of SEQ ID NO: 8, or an amino acid sequence having at least about 90%, or at least about 95%, or at least about 97%, or at least about 98%, or at least about 99% identity thereto.

The amino acid sequences of an Fc fragment of an IgG1 antibody (SEQ ID NO: 5) and of gp96 fused to an Fc fragment of an IgG1 antibody (SEQ ID NO: 8) are provided below:

(SEQ ID NO: 5)
VPRDSGSKPSISTVPEVSSVFIFPPKPKDVLTITLTPKVICVVVDISKD
DPEVQFSWFVDDVEVHTAQTKPREEQFNSTFRSVSELPIMHQDWLNGKE
FKCRVNSAAFPAPIEKTISKTKGRPKAPQVYTIPPPKEQMAKDKVSLTC
MITDFFPEDITVEWQWNGQPAENYKNTQPIMDTDGSYFVYSKLNVQKSN
WEAGNTFTCSVLHEGLHNHHTEKSLSHSPGK
(SEQ ID NO: 8)
MMKLIINSLYKNKEIFLRELISNASDALDKIRLISLTDENALSGNEELT
VKIKCDKEKNLLHVTDTGVGMTREELVKNLGTIAKSGTSEFLNKMTEAQ
EDGQSTSELIGQFGVGFYSAFLVADKVIVTSKHNNDTQHIWESDSNEFS
VIADPRGNTLGRGTTITLVLKEEASDYLELDTIKNLVKKYSQFINFPIY
VWSSKTETVEEPMEEEEAAKEEKEESDDEAAVEEEEEEKKPKTKKVEKT
VWDWELMNDIKPIWQRPSKEVEEDEYKAFYKSFSKESDDPMAYIHFTAE
GEVTFKSILFVPTSAPRGLFDEYGSKKSDYIKLYVRRVFITDDFHDMMP
KYLNFVKGVVDSDDLPLNVSRETLQQHKLLKVIRKKLVRKTLDMIKKIA
DDKYNDTFWKEFGTNIKLGVIEDHSNRTRLAKLLRFQSSHHPTDITSLD
QYVERMKEKQDKIYFMAGSSRKEAESSPFVERLLKKGYEVIYLTEPVDE
YCIQALPEFDGKRFQNVAKEGVKFDESEKTKESREAVEKEFEPLLNWMK
DKALKDKIEKAVVSQRLTESPCALVASQYGWSGNMERIMKAQAYQTGKD
ISTNYYASQKKTFEINPRHPLIRDMLRRIKEDEDDKTVLDLAVVLFETA
TLRSGYLLPDTKAYGDRIERMLRLSLNIDPDAKVEEEPEEEPEETAEDT
TEDTEQDEDEEMDVGTDEEEETAKESTAEGSVPRDSGSKPSISTVPEVS
SVFIFPPKPKDVLTITLTPKVTCVVVDISKDDPEVQFSWFVDDVEVHTA
QTKPREEQFNSTFRSVSELPIMHQDWLNGKEFKCRVNSAAFPAPIEKTI
SKTKGRPKAPQVYTIPPPKEQMAKDKVSLTCMITDFFPEDITVEWQWNG
QPAENYKNTQPIMDTDGSYFVYSKLNVQKSNWEAGNTFTCSVLHEGLHN
HHTEKSLSHSPGK

In some aspects, the chaperone protein is gp96. The coding region of human gp96 is 2,412 bases in length, and encodes an 803 amino acid protein that includes a 21 amino acid signal peptide at the amino terminus, a potential transmembrane region rich in hydrophobic residues, and an ER retention peptide sequence at the carboxyl terminus (GENBANK® Accession No. X15187; see, Maki et al., Proc Natl Acad Sci USA 1990, 87:5658-5562). The DNA sequence (SEQ ID NO: 1) and protein sequence (SEQ ID NO: 2) of human gp96 are provided below:

(SEQ ID NO: 1)
atgagggccctgtgggtgctgggcctctgctgcgtcctgctgaccttcg
ggtcggtcagagctgacgatgaagttgatgtggatggtacagtagaaga
ggatctgggtaaaagtagagaaggatcaaggacggatgatgaagtagta
cagagagaggaagaagctattcagttggatggattaaatgcatcacaaa
taagagaacttagagagaagtcggaaaagtttgccttccaagccgaagt
taacagaatgatgaaacttatcatcaattcattgtataaaaataaagag
attttcctgagagaactgatttcaaatgcttctgatgctttagataaga
taaggctaatatcactgactgatgaaaatgctctttctggaaatgagga
actaacagtcaaaattaagtgtgataaggagaagaacctgctgcatgtc
acagacaccggtgtaggaatgaccagagaagagttggttaaaaaccttg
gtaccatagccaaatctgggacaagcgagtttttaaacaaaatgactga
agcacaggaagatggccagtcaacttctgaattgattggccagtttggt
gtcggtttctattccgccttccttgtagcagataaggttattgtcactt
caaaacacaacaacgatacccagcacatctgggagtctgactccaatga
attttctgtaattgctgacccaagaggaaacactctaggacggggaacg
acaattacccttgtcttaaaagaagaagcatctgattaccttgaattgg
atacaattaaaaatctcgtcaaaaaatattcacagttcataaactttcc
tatttatgtatggagcagcaagactgaaactgttgaggagcccatggag
gaagaagaagcagccaaagaagagaaagaagaatctgatgatgaagctg
cagtagaggaagaagaagaagaaaagaaaccaaagactaaaaaagttga
aaaaactgtctgggactgggaacttatgaatgatatcaaaccaatatgg
cagagaccatcaaaagaagtagaagaagatgaatacaaagctttctaca
aatcattttcaaaggaaagtgatgaccccatggcttatattcactttac
tgctgaaggggaagttaccttcaaatcaattttatttgtacccacatct
gctccacgtggtctgtttgacgaatatggatctaaaaagagcgattaca
ttaagctctatgtgcgccgtgtattcatcacagacgacttccatgatat
gatgcctaaatacctcaattttgtcaagggtgtggtggactcagatgat
ctccccttgaatgtttcccgcgagactcttcagcaacataaactgctta
aggtgattaggaagaagcttgttcgtaaaacgctggacatgatcaagaa
gattgctgatgataaatacaatgatactttttggaaagaatttggtacc
aacatcaagcttggtgtgattgaagaccactcgaatcgaacacgtcttg
ctaaacttcttaggttccagtcttctcatcatccaactgacattactag
cctagaccagtatgtggaaagaatgaaggaaaaacaagacaaaatctac
ttcatggctgggtccagcagaaaagaggctgaatcttctccatttgttg
agcgacttctgaaaaagggctatgaagttatttacctcacagaacctgt
ggatgaatactgtattcaggcccttcccgaatttgatgggaagaggttc
cagaatgttgccaaggaaggagtgaagttcgatgaaagtgagaaaacta
aggagagtcgtgaagcagttgagaaagaatttgagcctctgctgaattg
gatgaaagataaagcccttaaggacaagattgaaaaggctgtggtgtct
cagcgcctgacagaatctccgtgtgctttggtggccagccagtacggat
ggtctggcaacatggagagaatcatgaaagcacaagcgtaccaaacggg
caaggacatctctacaaattactatgcgagtcagaagaaaacatttgaa
attaatcccagacacccgctgatcagagacatgcttcgacgaattaagg
aagatgaagatgataaaacagttttggatcttgctgtggttttgtttga
aacagcaacgcttcggtcagggtatcttttaccagacactaaagcatat
ggagatagaatagaaagaatgcttcgcctcagtttgaacattgaccctg
atgcaaaggtggaagaagagcccgaagaagaacctgaagagacagcaga
agacacaacagaagacacagagcaagacgaagatgaagaaatggatgtg
ggaacagatgaagaagaagaaacagcaaaggaatctacagctgaaaaag
atgaattgtaa.
(SEQ ID NO: 2)
MRALWVLGLCCVLLTFGSVRADDEVDVDGTVEEDLGKSREGSRTDDEVV
QREEEAIQLDGLNASQIRELREKSEKFAFQAEVNRMMKLIINSLYKNKE
IFLRELISNASDALDKIRLISLTDENALSGNEELTVKIKCDKEKNLLHV
TDTGVGMTREELVKNLGTIAKSGTSEFLNKMTEAQEDGQSTSELIGQFG
VGFYSAFLVADKVIVTSKHNNDTQHIWESDSNEFSVIADPRGNTLGRGT
TITLVLKEEASDYLELDTIKNLVKKYSQFINFPIYVWSSKTETVEEPME
EEEAAKEEKEESDDEAAVEEEEEEKKPKTKKVEKTVWDWELMNDIKPIW
QRPSKEVEEDEYKAFYKSFSKESDDPMAYIHFTAEGEVTFKSILFVPTS
APRGLFDEYGSKKSDYIKLYVRRVFITDDFHDMMPKYLNFVKGVVDSDD
LPLNVSRETLQQHKLLKVIRKKLVRKTLDMIKKIADDKYNDTFWKEFGT
NIKLGVIEDHSNRTRLAKLLRFQSSHHPTDITSLDQYVERMKEKQDKIY
FMAGSSRKEAESSPFVERLLKKGYEVIYLTEPVDEYCIQALPEFDGKRF
QNVAKEGVKFDESEKTKESREAVEKEFEPLLNWMKDKALKDKIEKAVVS
QRLTESPCALVASQYGWSGNMERIMKAQAYQTGKDISTNYYASQKKTFE
INPRHPLIRDMLRRIKEDEDDKTVLDLAVVLFETATLRSGYLLPDTKAY
GDRIERMLRLSLNIDPDAKVEEEPEEEPEETAEDTTEDTEQDEDEEMDV
GTDEEEETAKESTAEKDEL.

In exemplary aspects, the gp96 comprises the amino acid sequence of SEQ ID NO: 2. In exemplary aspects, the gp96 comprises the amino acid sequence of SEQ ID NO: 2 but without the terminal KDEL sequence.

In various embodiments, the gp96 portion of the fusion protein comprises an amino acid sequence that has at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with any known wild type amino acid sequences of gp96 or a gp96 amino acid sequence disclosed herein (e.g. about 60%, or about 61%, or about 62%, or about 63%, or about 64%, or about 65%, or about 66%, or about 67%, or about 68%, or about 69%, or about 70%, or about 71%, or about 72%, or about 73%, or about 74%, or about 75%, or about 76%, or about 77%, or about 78%, or about 79%, or about 80%, or about 81%, or about 82%, or about 83%, or about 84%, or about 85%, or about 86%, or about 87%, or about 88%, or about 89%, or about 90%, or about 91%, or about 92%, or about 93%, or about 94%, or about 95%, or about 96%, or about 97%, or about 98%, or about 99% sequence identity).

Thus, in some embodiments, the gp96 portion of nucleic acid encoding a gp96-Ig fusion polypeptide can encode an amino acid sequence that differs from the wild type gp96 polypeptide at one or more amino acid positions, such that it contains one or more conservative substitutions, non-conservative substitutions, splice variants, isoforms, homologues from other species, and polymorphisms as described previously.

Mutations may also be made to the nucleotide sequences of the present fusion proteins by reference to the genetic code, including taking into account codon degeneracy.

In some embodiments, the chaperone protein may be a heat shock protein. In various embodiments, the heat shock protein is one or more of hsp40, hsp60, hsp70, hsp90, and hsp110 family members, inclusive of fragments, variants, mutants, derivatives or combinations thereof (Hickey, et al., 1989, Mol. Cell. Biol. 9:2615-2626; Jindal, 1989, Mol. Cell. Biol. 9:2279-2283).

In various aspects, the fusion protein comprises an immunoglobulin or antibody. The antibody may be any type of antibody, i.e., immunoglobulin, known in the art. In illustrative embodiments, the antibody is an antibody of class or isotype IgA, IgD, IgE, IgG, or IgM. In illustrative embodiments, the antibody described herein comprises one or more alpha, delta, epsilon, gamma, and/or mu heavy chains. In illustrative embodiments, the antibody described herein comprises one or more kappa or light chains. In illustrative aspects, the antibody is an IgG antibody and optionally is one of the four human subclasses: IgG1, IgG2, IgG3 and IgG4. Also, the antibody in some embodiments is a monoclonal antibody. In other embodiments, the antibody is a polyclonal antibody. In some embodiments, the antibody is structurally similar to or derived from a naturally-occurring antibody, e.g., an antibody isolated and/or purified from a mammal, e.g., mouse, rabbit, goat, horse, chicken, hamster, human, and the like. In this regard, the antibody may be considered as a mammalian antibody, e.g., a mouse antibody, rabbit antibody, goat antibody, horse antibody, chicken antibody, hamster antibody, human antibody, and the like. In illustrative aspects, the antibody comprises sequence of only mammalian antibodies.

In illustrative aspects, the fusion protein comprises a fragment of an immunoglobulin or antibody. Antibody fragments include, but are not limited to, the F(ab′)₂ fragment which may be produced by pepsin digestion of the antibody molecule; the Fab′ fragments which may be generated by reducing the disulfide bridges of the F(ab′)₂ fragment, and the two Fab′ fragments which may be generated by treating the antibody molecule with papain and a reducing agent. In exemplary aspects, the fusion protein comprises an Fc fragment of an antibody.

DNAs encoding immunoglobulin light or heavy chain constant regions are known or readily available from cDNA libraries. See, for example, Adams et al., Biochemistry 1980, 19:2711-2719; Gough et al., Biochemistry 1980 19:2702-2710; Dolby et al., Proc Natl Acad Sci USA 1980, 77:6027-6031; Rice et al., Proc Natl Acad Sci USA 1982, 79:7862-7865; Falkner et al., Nature 1982, 298:286-288; and Morrison et al., Ann Rev Immunol 1984, 2:239-256.

In some embodiments, a gp96 peptide can be fused to the hinge, CH2 and CH3 domains of murine IgG1 (Bowen et al., J Immunol 1996, 156:442-449). This region of the IgG1 molecule contains three cysteine residues that normally are involved in disulfide bonding with other cysteines in the Ig molecule. Since none of the cysteines are required for the peptide to function as a tag, one or more of these cysteine residues can be substituted by another amino acid residue, such as, for example, serine.

In illustrative aspects, the fusion protein comprises an Fc fragment of an IgG1 antibody. In illustrative aspects, the Fc fragment comprises the amino acid sequence of SEQ ID NO: 5.

In exemplary aspects, the fusion protein comprises a gp96 chaperone protein fused to a Fc fragment of an IgG1 antibody. In illustrative aspects, the fusion protein comprises the amino acid sequence of SEQ ID NO: 8.

A nucleic acid encoding a gp96-Ig fusion sequence can be produced using the methods described in U.S. Pat. No. 8,685,384, which is incorporated herein by reference in its entirety. In some embodiments, the gp96 portion of a gp96-Ig fusion protein can contain all or a portion of a wild type gp96 sequence (e.g., the human sequence set forth herein). For example, a secretable gp96-Ig fusion protein can include the first 799 amino acids of the human gp96 sequence provided herein, such that it lacks the C-terminal KDEL sequence. Alternatively, the gp96 portion of the fusion protein can have an amino acid sequence that contains one or more substitutions, deletions, or additions as compared to any known wild type amino acid sequences of gp96 or a gp96 amino acid sequence disclosed herein.

In various embodiments, the gp96-Ig fusion protein and/or the coronavirus protein or an antigenic portion thereof, further comprises a linker. In various embodiments, the linker may be derived from naturally-occurring multi-domain proteins or are empirical linkers as described, for example, in Chichili et al., (2013), Protein Sci. 22(2):153-167, Chen et al., (2013), Adv Drug Deliv Rev. 65(10):1357-1369, the entire contents of which are hereby incorporated by reference. In some embodiments, the linker may be designed using linker designing databases and computer programs such as those described in Chen et al., (2013), Adv Drug Deliv Rev. 65(10):1357-1369 and Crasto et. al., (2000), Protein Eng. 13(5):309-312, the entire contents of which are hereby incorporated by reference. In some embodiments, the linker is a synthetic linker such as PEG. In other embodiments, the linker is a polypeptide. In some embodiments, the linker is less than about 100 amino acids long. For example, the linker may be less than about 100, about 95, about 90, about 85, about 80, about 75, about 70, about 65, about 60, about 55, about 50, about 45, about 40, about 35, about 30, about 25, about 20, about 19, about 18, about 17, about 16, about 15, about 14, about 13, about 12, about 11, about 10, about 9, about 8, about 7, about 6, about 5, about 4, about 3, or about 2 amino acids long. In some embodiments, the linker is flexible. In another embodiment, the linker is rigid. In various embodiments, the linker is substantially comprised of glycine and serine residues (e.g. about 30%, or about 40%, or about 50%, or about 60%, or about 70%, or about 80%, or about 90%, or about 95%, or about 97% glycines and serines).

In various embodiments, the linker is a hinge region of an antibody (e.g., of IgG, IgA, IgD, and IgE, inclusive of subclasses (e.g. IgG1, IgG2, IgG3, and IgG4, and IgA1 and IgA2)). The hinge region, found in IgG, IgA, IgD, and IgE class antibodies, acts as a flexible spacer, allowing the Fab portion to move freely in space. In contrast to the constant regions, the hinge domains are structurally diverse, varying in both sequence and length among immunoglobulin classes and subclasses. For example, the length and flexibility of the hinge region varies among the IgG subclasses. The hinge region of IgG1 encompasses amino acids 216-231 and, because it is freely flexible, the Fab fragments can rotate about their axes of symmetry and move within a sphere centered at the first of two inter-heavy chain disulfide bridges. IgG2 has a shorter hinge than IgG1, with 12 amino acid residues and four disulfide bridges. The hinge region of IgG2 lacks a glycine residue, is relatively short, and contains a rigid poly-proline double helix, stabilized by extra inter-heavy chain disulfide bridges. These properties restrict the flexibility of the IgG2 molecule. IgG3 differs from the other subclasses by its unique extended hinge region (about four times as long as the IgG1 hinge), containing 62 amino acids (including 21 prolines and 11 cysteines), forming an inflexible poly-proline double helix. In IgG3, the Fab fragments are relatively far away from the Fc fragment, giving the molecule a greater flexibility. The elongated hinge in IgG3 is also responsible for its higher molecular weight compared to the other subclasses. The hinge region of IgG4 is shorter than that of IgG1 and its flexibility is intermediate between that of IgG1 and IgG2. The flexibility of the hinge regions reportedly decreases in the order IgG3>IgG1>IgG4>IgG2.

Additional illustrative linkers include, but are not limited to, linkers having the sequence LE, GGGGS, (GGGGS)ₙ (n=1-4), (Gly)₈, (Gly)₆, (EAAAK)ₙ (n=1-3), A(EAAAK)ₙA (n=2-5), AEAAAKEAAAKA, A(EAAAK)₄ALEA(EAAAK)₄A, PAPAP, KESGSVSSEQLAQFRSLD, EGKSSGSGSESKST, GSAGSAAGSGEF, and (XP)ₙ, with X designating any amino acid, e.g., Ala, Lys, or Glu.

In various embodiments, the linker may be functional. For example, without limitation, the linker may function to improve the folding and/or stability, improve the expression, improve the pharmacokinetics, and/or improve the bioactivity of the present compositions. In another example, the linker may function to target the compositions to a particular cell type or location.

Host Cells

Also provided by the present invention is a host cell comprising any one of the expression vector systems described herein. As used herein, the term “host cell” refers to any type of cell that can contain the inventive expression vector system. The host cell can be a eukaryotic cell, e.g., plant, animal, fungi, or algae, or can be a prokaryotic cell, e.g., bacteria or protozoa. The host cell can be a cultured cell or a primary cell, i.e., isolated directly from an organism, e.g., a human. The host cell can be an adherent cell or a suspended cell, i.e., a cell that grows in suspension. In illustrative aspects, the host cell is a mammalian host cell. In illustrative aspects, the host cell is a human host cell. In illustrative aspects, the human host cell is an NIH 3T3 cell or an HEK 293 cell. The presently disclosed host cells are not limited to just these two types of cells, however, and may be any cell type described herein. For example, the cells that can be used include, without limitation, epithelial cells, endothelial cells, keratinocytes, fibroblasts, muscle cells, hepatocytes; blood cells such as T lymphocytes, B lymphocytes, monocytes, macrophages, neutrophils, eosinophils, megakaryocytes, or granulocytes, various stem or progenitor cells, such as hematopoietic stem or progenitor cells (e.g., as obtained from bone marrow), umbilical cord blood, peripheral blood, fetal liver, etc., and tumor cells (e.g., human tumor cells). The choice of cell type can be determined by one of skill in the art. In various embodiments, the cells are irradiated.

Also provided by the present invention is a population of cells comprising at least one host cell described herein. The population of cells can be a heterogeneous population comprising the host cell comprising any of the recombinant expression vectors described, in addition to at least one other cell. Alternatively, the population of cells can be a substantially homogeneous population, in which the population comprises mainly host cells (e.g., consisting essentially of) comprising the expression vector(s). The population also can be a clonal population of cells, in which all cells of the population are clones of a single host cell comprising the recombinant expression vector(s), such that all cells of the population comprise the recombinant expression vector(s). In one embodiment of the invention, the population of cells is a clonal population comprising host cells comprising the expression vector(s) as described herein. In illustrative aspects, the cell population of the present invention is one wherein at least 50% of the cells are host cells as described herein. In illustrative aspects, the cell population of the present invention is one wherein at least 60%, at least 70%, at least 80% or at least 90% or more of the cells are host cells as described herein.

Compositions

The present invention also provides a composition comprising an expression vector system or a host cell or a population of cells, as described herein, and an excipient, carrier, or diluent. In exemplary aspects, the composition is a pharmaceutical composition. In illustrative aspects, the composition may comprise virus particles containing the vector expression system. In illustrative aspects, the composition is a sterile composition. In some embodiments, the composition is suitable for administration to a human. In illustrative aspects, the composition comprises a unit dose of host cells. In some embodiments, the unit dose is about 10⁵, about 10⁶, about 10⁷, about 10⁸, about 10⁹, about 10¹⁰, about 10¹¹, about 10¹², about 10¹³, about 10¹⁴, about 10¹⁵, or more host cells transfected with the expression vector system. In some embodiments, the composition comprises at least or about 10⁶ cells transfected with the expression vector system.

The pharmaceutical composition can comprise any pharmaceutically acceptable ingredient, including, for example, acidifying agents, additives, adsorbents, aerosol propellants, air displacement agents, alkalizing agents, anticaking agents, anticoagulants, antimicrobial preservatives, antioxidants, antiseptics, bases, binders, buffering agents, chelating agents, coating agents, coloring agents, desiccants, detergents, diluents, disinfectants, disintegrants, dispersing agents, dissolution enhancing agents, dyes, emollients, emulsifying agents, emulsion stabilizers, fillers, film forming agents, flavor enhancers, flavoring agents, flow enhancers, gelling agents, granulating agents, humectants, lubricants, mucoadhesives, ointment bases, ointments, oleaginous vehicles, organic bases, pastille bases, pigments, plasticizers, polishing agents, preservatives, sequestering agents, skin penetrants, solubilizing agents, solvents, stabilizing agents, suppository bases, surface active agents, surfactants, suspending agents, sweetening agents, therapeutic agents, thickening agents, tonicity agents, toxicity agents, viscosity-increasing agents, water-absorbing agents, water-miscible cosolvents, water softeners, or wetting agents.

The pharmaceutical compositions may be formulated to achieve a physiologically compatible pH. In some embodiments, the pH of the pharmaceutical composition may be at least 5, at least 5.5, at least 6, at least 6.5, at least 7, at least 7.5, at least 8, at least 8.5, at least 9, at least 9.5, at least 10, or at least 10.5 up to and including pH 11, depending on the formulation and route of administration, for example between 4 and 7, or 4.5 and 5.5. In illustrative embodiments, the pharmaceutical compositions may comprise buffering agents to achieve a physiological compatible pH. The buffering agents may include any compounds capable of buffering at the desired pH such as, for example, phosphate buffers (e.g., PBS), triethanolamine, Tris, bicine, TAPS, tricine, HEPES, TES, MOPS, PIPES, cacodylate, MES, acetate, citrate, succinate, histidine or other pharmaceutically acceptable buffers.

The present invention therefore provides compositions including pharmaceutical compositions containing an expression vector system or a cell containing the expression vector system as described herein, in combination with a physiologically and pharmaceutically acceptable carrier. In various embodiments, the physiologically and pharmaceutically acceptable carrier can include any of the well-known components useful for immunization. The carrier can facilitate or enhance an immune response to an antigen administered in a vaccine. The cell formulations can contain buffers to maintain a preferred pH range, salts or other components that present an antigen to an individual in a composition that stimulates an immune response to the antigen. The physiologically acceptable carrier also can contain one or more adjuvants that enhance the immune response to an antigen. Pharmaceutically acceptable carriers include, for example, pharmaceutically acceptable solvents, suspending agents, or any other pharmacologically inert vehicles for delivering compounds to a subject. Pharmaceutically acceptable carriers can be liquid or solid, and can be selected with the planned manner of administration in mind so as to provide for the desired bulk, consistency, and other pertinent transport and chemical properties, when combined with one or more therapeutic compounds and any other components of a given pharmaceutical composition. Typical pharmaceutically acceptable carriers include, without limitation: water, saline solution, binding agents (e.g., polyvinylpyrrolidone or hydroxypropyl methylcellulose); fillers (e.g., lactose or dextrose and other sugars, gelatin, or calcium sulfate), lubricants (e.g., starch, polyethylene glycol, or sodium acetate), disintegrates (e.g., starch or sodium starch glycolate), and wetting agents (e.g., sodium lauryl sulfate). Compositions can be formulated for subcutaneous, intramuscular, or intradermal administration, or in any manner acceptable for administration.

An adjuvant refers to a substance which, when added to an immunogenic agent such as a cell containing the expression vector system of the invention, nonspecifically enhances or potentiates an immune response to the agent in the recipient host upon exposure to the mixture. Adjuvants can include, for example, oil-in-water emulsions, water-in oil emulsions, alum (aluminum salts), liposomes and microparticles, such as, polysytrene, starch, polyphosphazene and polylactide/polyglycosides.

Adjuvants can also include, for example, squalene mixtures (SAF-I), muramyl peptide, saponin derivatives, mycobacterium cell wall preparations, monophosphoryl lipid A, mycolic acid derivatives, nonionic block copolymer surfactants, Quit A, cholera toxin B subunit, polyphosphazene and derivatives, and immunostimulating complexes (ISCOMs) such as those described by Takahashi et al., Nature 1990, 344:873-875. For veterinary use and for production of antibodies in animals, mitogenic components of Freund's adjuvant (both complete and incomplete) can be used. In humans, Incomplete Freund's Adjuvant (IFA) is a useful adjuvant. Various appropriate adjuvants are well known in the art (see, for example, Warren and Chedid, CRC Critical Reviews in Immunology 1988, 8:83; and Allison and Byars, in Vaccines: New Approaches to Immunological Problems, 1992, Ellis, ed., Butterworth-Heinemann, Boston). Additional adjuvants include, for example, bacille Calmett-Guerin (BCG), DETOX (containing cell wall skeleton of Mycobacterium phlei (CWS) and monophosphoryl lipid A from Salmonella minnesota (MPL)), and the like (see, for example, Hoover et al., J Clin Oncol 1993, 11:390; and Woodlock et al., J Immunother 1999, 22:251-259).

Routes of Administration

Methods of administering cells to a subject are well-known, and include, but not limited to perfusions, infusions and injections. See, e.g., Burch et al., Clin Cancer Res 6(6): 2175-2182 (2000), Dudley et al., J Clin Oncol 26(32): 5233-5239 (2008); Khan et al., Cell Transplant 19:409-418 (2010); Gridelli et al., Liver Transpl 18:226-237 (2012)).

Methods of Use

Without being bound to a particular theory, the methods of the present invention advantageously rely on the chaperone function of the secreted fusion protein. The fusion protein chaperones the one or more SARS-CoV-2 (2019-nCoV) proteins or antigen portions thereof, which are efficiently taken up by activated antigen presenting cells (APCs). The APCs act to cross-present the 2019-nCoV proteins or antigen portions thereof via MHC I to CD8+ CTLs, whereupon an avid, antigen specific, cytotoxic CD8+ T cell response is stimulated. Without being bound to a particular theory, the expression vector systems of the present invention are advantageously capable of initiating both an innate immune response (including, e.g., activation of APCs, pro-inflammatory cytokine release, activation of NK cells), and an adaptive immune response (including, e.g., priming, activation and proliferation of antigen specific CTLs). Such dual-activation leads to successful clearance of the antigen/pathogen.

Accordingly, in various embodiments, the present invention provides a method of eliciting an immune response against a coronavirus, e.g., SARS-CoV-2 (2019-nCoV) virus, in a subject. In illustrative embodiments, the method comprises administering to the subject the expression vector as disclosed herein, or a population of cells transfected with the expression vector.

In various embodiments, the present invention provides a method of treating or preventing a SARS-CoV-2 infection. In some embodiments, the SARS-CoV-2 infection causes COVID-19 or a similar disease. The present method includes prevention or reduction of symptoms, such as fever, cough, shortness of breath, diarrhea, upper respiratory symptoms (e.g. sneezing, runny nose, sore throat), lower respiratory symptoms, and/or pneumonia.

In various embodiments, the present methods stimulate an immune response, e.g. against a coronavirus, e.g., SARS-CoV-2 virus The present invention also provides a method of treating or preventing a coronavirus infection in a subject, comprising administering to the subject the expression vector as disclosed herein, or a population of cells transfected with the expression vector.

As used herein, the term “treat,” as well as words related thereto, do not necessarily imply 100% or complete treatment. Rather, there are varying degrees of treatment of which one of ordinary skill in the art recognizes as having a potential benefit or therapeutic effect. In this respect, the methods of treating a coronavirus infection of the present invention can provide any amount or any level of treatment. Furthermore, the treatment provided by the method of the present invention may include treatment of one or more conditions or symptoms or signs of the infection, being treated. Also, the treatment provided by the methods of the present invention may encompass slowing the progression of the infection. For example, the methods can treat the infection by virtue of eliciting an immune response against coronavirus, stimulating or activating CD8+ T cells specific for coronavirus (e.g., SARS-CoV-2), to proliferate, and the like.

As used herein, the term “prevent” and words stemming therefrom encompasses inhibiting or otherwise blocking infection by coronavirus. As used herein, the term “inhibit” and words stemming therefrom may not be a 100% or complete inhibition or abrogation. Rather, there are varying degrees of inhibition of which one of ordinary skill in the art recognizes as having a potential benefit or therapeutic effect. In this respect, the presently disclosed expression vector systems or host cells may inhibit coronavirus infection to any amount or level. In illustrative embodiments, the inhibition provided by the methods of the present invention is at least or about a 10% inhibition (e.g., at least or about a 20% inhibition, at least or about a 30% inhibition, at least or about a 40% inhibition, at least or about a 50% inhibition, at least or about a 60% inhibition, at least or about a 70% inhibition, at least or about a 80% inhibition, at least or about a 90% inhibition, at least or about a 95% inhibition, at least or about a 98% inhibition).

In various embodiments, methods of the invention prevent, alleviate, and/or treat one or more symptoms associated with coronavirus infection. Illustrative symptoms that may be treated include, but are not limited to fever, cough (e.g., dry cough), shortness of breath and other breathing difficulties, fatigue, diarrhea, upper respiratory symptoms (e.g. sneezing, runny nose, sore throat), and/or pneumonia.

The present expression vector system and cells comprising the same may be administered by any route considered appropriate by a medical practitioner. Illustrative routes of administration include, for example: oral, intradermal, intramuscular, intraperitoneal, intravenous, subcutaneous, intranasal, epidural, sublingual, intranasal, intracerebral, intravaginal, transdermal, rectally, by inhalation, by electroporation, or topically. Administration can be local or systemic.

In embodiments, the expression vector system, biological cells, and compositions in accordance with the present disclosure are administered as a single dose.

In various embodiments, the expression vector system, biological cells, and compositions in accordance with the present disclosure are administered via a prime dose and one or more booster (or boosting) doses. The booster dose is administered after the initial prime dose administration. The booster dose can include the same or different variant of a coronavirus protein than a variant of a coronavirus protein administered with the prime dose.

In embodiments, the prime and booster doses include the same coronavirus protein, or a variant of a coronavirus protein or an antigenic portion thereof. The booster dose can be administered about one week, or about two weeks, or about three weeks, or about four weeks, or about five weeks, or about six weeks after administration of the prime dose. In some embodiments, the booster is administered about two weeks, or about three weeks, or about four weeks after administration of the prime dose. In some embodiments, the booster is administered in from about three weeks to about six weeks, or from about three weeks to about five weeks, or from about three weeks to about four weeks after administration of the prime dose.

In embodiments, the prime and booster doses include different variants of a coronavirus protein or an antigenic portion thereof. The booster dose can be administered about one week, or about two weeks, or about three weeks, or about four weeks, or about five weeks, or about six weeks after administration of the prime dose. In some embodiments, the booster is administered about two weeks, or about three weeks, or about four weeks after administration of the prime dose. In some embodiments, the booster is administered in from about three weeks to about six weeks, or from about three weeks to about five weeks, or from about three weeks to about four weeks after administration of the prime dose.

In some embodiments, a booster dose is administered to target a new variant of a coronavirus protein or an antigenic portion thereof. For example, a booster dose can be administered in situations when a new variant of a coronavirus protein has been discovered and/or engineered and an expression vector system, biological cell, and/or composition in accordance with the present disclosure is provided that targets that new variant. In such embodiments, the booster dose can be administered in a suitable period of time after the prime dose. The booster dose can be in the form of one, two, or more doses.

In some embodiments, the expression vector system, biological cells, and compositions in accordance with the present disclosure are administered in a multi-dose schedule.

In illustrative aspects, the method comprises intramuscular (IM) administration of the expression vector. In illustrative aspects, the method comprises electroporation or electroporation following the IM administration of expression vector. In various embodiments, electroporation is used to help deliver vectors (genes) into the cell by applying short and intense electric pulses that transiently permeabilize the cell membrane, thus allowing transport of molecules otherwise not transported through a cellular membrane. Methods for electroporating a nucleic acid construct into cells and electroporation devices for such delivery are known. See, for example, Flanagan et al. Cancer Gene Ther (2012) 18:579-586, WO 2014/066655, U.S. Pat. No. 9,020,605, the entire contents are incorporated by reference.

In exemplary aspects, DNA (50 μg) containing expression vector that contains gp96-Ig and coronavirus proteins in 50 μL of saline is injected in the tibialis anterior muscle of anesthetized wild-type C57BL/6 mice. A two-needle array electrode pair is inserted into muscle immediately after DNA delivery and the injection site is electroporated with field strength of 50 V/cm (constant) and six electric pulses of 50 ms each by using the AgilePulse in Vivo System (BTX, Harvard Apparatus).

In illustrative aspects, the method comprises subcutaneously administering the population of cells. In illustrative aspects, the method comprises subcutaneously administering the population of cells to an arm or leg of the subject.

In various embodiments, the vector or the cell can be administered to a subject one or more times (e.g., once, twice, two to four times, three to five times, five to eight times, six to ten times, eight to 12 times, or more than 12 times). A vector or a cell as provided herein can be administered one or more times per day, one or more times per week, every other week, one or more times per month, once every two to three months, once every three to six months, or once every six to 12 months. A vector or a cell can be administered over any suitable period of time, such as a period from about 1 day to about 12 months. In some embodiments, for example, the period of administration can be from about 1 day to 90 days; from about 1 day to 60 days; from about 1 day to 30 days; from about 1 day to 20 days; from about 1 day to 10 days; from about 1 day to 7 days. In some embodiments, the period of administration can be from about 1 week to 50 weeks; from about 1 week to 50 weeks; from about 1 week to 40 weeks; from about 1 week to 30 weeks; from about 1 week to 24 weeks; from about 1 week to 20 weeks; from about 1 week to 16 weeks; from about 1 week to 12 weeks; from about 1 week to 8 weeks; from about 1 week to 4 weeks; from about 1 week to 3 weeks; from about 1 week to 2 weeks; from about 2 weeks to 3 weeks; from about 2 weeks to 4 weeks; from about 2 weeks to 6 weeks; from about 2 weeks to 8 weeks; from about 3 weeks to 8 weeks; from about 3 weeks to 12 weeks; or from about 4 weeks to 20 weeks.

Embodiments that relate to methods of treatment and prevention are also envisioned to apply to medical uses and uses in manufacture of medicaments.

Method of Detection

In various embodiments, techniques are used for detecting a coronavirus in patient samples, e.g. to establish if a subject is suited for the present treatments and/or to evaluate if the present methods are beneficial. For example, in some embodiments, RT reverse transcription PCR (RT-PCR) techniques can be used.

In some embodiments, real-time RT-PCR can be used to detect coronaviruses from respiratory secretions as described, for example, in Corman et al. Detection of 2019 novel coronavirus (2019-nCoV) by real-time RT-PCR. Euro Surveill. 2020; 25(3), which is incorporated herein by reference in its entirety.

In some embodiments, one-step quantitative RT-PCR can be performed as described in Chu et al., Molecular Diagnosis of a Novel Coronavirus (2019-nCoV) Causing an Outbreak of Pneumonia, Clinical Chemistry, 31 Jan. 2020, which is incorporated herein by reference in its entirety, which targeted both the open reading frame 1 b (ORF1b) and the N regions of the viral genome based on the first sequence deposited at GenBank (MN908947).

Combination Therapy

In various embodiments, the composition, vector, or cell in accordance with embodiments of the present invention is co-administered in conjunction with additional therapeutic agent(s), including vaccines. Co-administration can be simultaneous or sequential.

In some embodiments, the additional therapeutic agent is an agent that is used to provide relief to symptoms of coronavirus infections. Such agents include remdesivir; favipiravir; galidesivir; prezcobix; lopinavir and/or ritonavir and/or arbidol; mRNA-1273; MSCs-derived exosomes; lopinavir/ritonavir and/or ribavirin and/or IFN-beta; xiyanping; anti-VEGF-A (e.g. Bevacizumab); fingolimod; carrimycin; hydroxychloroquine; darunavir and cobicistat; methylprednisolone; brilacidin; leronlimab (PRO 140); and thalidomide.

In some embodiments, the additional therapeutic agent is chloroquine, including chloroquine phosphate.

In an embodiment, the additional therapeutic agent is a composition comprising one or more HIV drugs. In some embodiments, the composition comprises a combination of one or more of lopinavir and/or ritonavir and/or arbidol.

In some embodiments, the additional therapeutic agent comprises one or more vaccines. In some embodiments, the additional therapeutic agent comprises one or more coronavirus vaccines. In some embodiments, the additional therapeutic agent comprises one or more coronavirus vaccines and/or one or more of other types of vaccines.

In some embodiments, the composition, vector, or cell in accordance with embodiments of the present disclosure, which employs a gp-96-based vaccine, may be delivered alone (e.g., as a standalone vaccine) or in combination with other vaccines that drive humoral immunity, to provide an added layer of cellular immunity. The composition, vector, or cell can be administered in combination with one or more other vaccines, e.g., without limitation, flu vaccines, SARS-CoV-2 vaccines, and other vaccines. In a combination approach, the gp96-based SARS-CoV-2 vaccine in accordance with embodiments of the present disclosure, in combination with other vaccines (including conventional vaccines), induces effective and durable immune responses.

In some embodiments, a combination of the gp96-based SARS-CoV-2 vaccine and other vaccines may boost immunity in certain types of patients, including elderly patients, patents with comorbidities, and patients with compromised immune system. The gp96-based SARS-CoV-2 vaccine enhances effect of other vaccines and by providing an added layer of T-cell immunity boost to generate an effective and long-term immune response.

In some embodiments, an additional vaccine is a coronavirus vaccine. In some embodiments, a composition, vector, or cell in accordance with embodiments of the present disclosure is administered in combination with a coronavirus vaccine either simultaneously or sequentially. In some embodiments, the coronavirus vaccine is in the exploratory, preclinical, clinical, post-clinical, or approved stage. In some embodiments, the coronavirus vaccine comprises one or more of: a live attenuated virus, an inactivated virus, a non-replicating viral vector, a replicating viral vector, a recombinant protein, a peptide, a virus-like particle, DNA, RNA, mRNA, another macromolecule, and a fragment thereof.

In some embodiments, the coronavirus vaccine is selected from mRNA-1273, AZD1222, BNT162, Ad5-nCoV, INO-4800, LV-SMENP-DC, and pathogen-specific aAPC, or a variant or derivative thereof. In some embodiments, the coronavirus vaccine comprises an mRNA vaccine encoding SARS-CoV-2 spike (S) protein, optionally LNP-encapsulated, like mRNA-1273. In some embodiments, the coronavirus vaccine comprises a viral vector vaccine expressing the S protein, optionally a viral vector (ChAdOx1—chimpanzee adenovirus Oxford 1) vaccine (ChAdOx1 nCoV-19) expressing the S protein, like AZD1222. In some embodiments, the coronavirus vaccine comprises an mRNA vaccine encoding an optimized SARS-CoV-2 receptor-binding domain (RBD), like BNT162b1. In some embodiments, the coronavirus vaccine comprises an mRNA vaccine encoding an optimized full-length S protein, like BNT162b2. In some embodiments, the coronavirus vaccine comprises Adenovirus type 5 vector that expresses a protein selected from spike surface glycoprotein, membrane glycoprotein M, envelope protein E, and nucleocapsid phosphoprotein N; optionally Adenovirus type 5 vector that expresses S protein, like Ad5-nCoV. In some embodiments, the coronavirus vaccine comprises a plasmid encoding S protein delivered by electroporation, optionally a DNA plasmid encoding S protein delivered by electroporation, like INO-4800. In some embodiments, the coronavirus vaccine comprises dendritic cells (DCs) modified with lentiviral vector expressing synthetic minigene based on domains of selected viral proteins, administered with antigen-specific cytotoxic T lymphocytes (CTLs), like LV-SMENP-DC. In some embodiments, the coronavirus vaccine comprises artificial antigen-presenting cells (aAPCs) modified with lentiviral vector expressing synthetic minigene based on domains of selected viral proteins, like pathogen-specific aAPC.

In some embodiments, the vaccine induces a CD8+ T cell response in the patient. In some embodiments, the vaccine induces the CD8+ T cell to target the immunodominant epitope of the SARS-CoV-2 spike (S) protein. In some embodiments, the vaccine induces a CD69+CD8+ T cell response in the patient. In some embodiments, the vaccine induces a CD4+ T cell response in the patient. In some embodiments, the CD4+ T cell response in the patient releases antiviral cytokines. In some embodiments, the antiviral cytokines are selected from IFNγ, INF-α, and IL-2. In some embodiments, the vaccine induces the response in a lung and/or airway passage of the patient. In some embodiments, the vaccine induces cytotoxic CD8+ T-cell effector memory cells and resident memory T-cell responses. In some embodiments, the methods further comprise administering the vaccine as a single vaccination. In some embodiments, the vaccine induces a SARS-CoV-2, Spike protein specific CD4+ Th1 T-cell response.

Subjects

In illustrative embodiments, the subject is a mammal, including, but not limited to, mammals of the order Rodentia, such as mice and hamsters, and mammals of the order Logomorpha, such as rabbits, mammals from the order Carnivora, including Felines (cats) and Canines (dogs), mammals from the order Artiodactyla, including Bovines (cows) and Swines (pigs) or of the order Perssodactyla, including Equines (horses). In some aspects, the mammals are of the order Primates, Ceboids, or Simoids (monkeys) or of the order Anthropoids (humans and apes).

In various embodiments, the mammal is a human. In some embodiments, the human is an adult aged 18 years or older. In some embodiments, the human is a child aged 17 years or less. In an embodiment, the subject is male, e.g., a male human. In another embodiment, the subject is a female subject. In illustrative embodiments, the subject is a female subject, e.g., a female human.

Patient Selection

In embodiments, methods for selecting patients who can benefit from compositions and methods in accordance with embodiments of the present disclosure are provided. In some embodiments, the compositions, cells, expression vectors, and methods employ a vaccine which can be, without limitation, a gp96/OX40L-Ig COVID-19 vaccine, and which can activate robust T-cell immunity along with humoral immunity. It should be appreciated however that a gp96-based COVID-19 vaccine in accordance with embodiments of the present disclosure can have any other T cell costimulatory fusion protein, as the vaccine is not limited to the OX40L-Ig T cell costimulatory fusion protein.

In some embodiments, the vaccine (e.g., without limitation, a gp96/OX40L-Ig COVID-19 vaccine) is useful for harnessing natural antigen presentation and T-cell activation pathways in, without limitations, elderly patients (e.g., patients over the age of 65), patients with comorbidities, and/or in patients with a compromised immune system. Accordingly, the patient can be selected for treatment in accordance with embodiments of the present disclosure based on one or more of that patient's age, the status of the patient's immune system, and based on whether or not the patient has a comorbidity. The comorbidity can be defined as the simultaneous presence of two or more chronic diseases or conditions in the patient.

As mentioned above, a composition in accordance with embodiments of the present disclosure may be used as a standalone vaccine or as a vaccine in combination with other vaccines that drive humoral immunity, to provide an added layer of cellular immunity. As shown in Table 1 below, immunity in the elderly patients' population is compromised (see Siegrist. Chapter 2, Vaccine Immunology. In: Plotkin et al., eds. Plotkin's Vaccines. Elsevier, 2018, 7th Edition:16-34), which, without wishing to be bound by the theory, may explain the heavy toll that the SARS-CoV-2 pandemic has inflicted on the aged population and in patients with comorbidities. Table 1 shows various features that elderly patients may have and that prevent effective vaccination of this patient group.

The reduction in the reservoir of robust, naïve T cells and limited effector memory T cells in the elderly patients is a problem that the present gp96/OX40L-Ig COVID-19 vaccine can address. For example, elderly patients are treated with a double dose of the flu vaccine in order to compensate for weaker immune systems. Therefore, the present compositions can significantly improve immune response in elderly patients, as well as in other patients who may have a compromised immune system, when the compositions are administered alone or in combination with another (e.g. conventional) vaccine.

Elderly patients' features

| | |
|---|---|
| Limited magnitude of antibody | Low reservoir of IgM memory cells; |
| responses to polysaccharide | weaker differentiation into plasma |
| | cells |
| Limited magnitude of antibody | Limited germinal center responses: |
| responses to proteins | suboptimal CD4 helper responses, |
| | suboptimal B-cell activation, limited |
| | FDC network development; changes |
| | in B/T cell repertoire |
| Limited quality (affinity, | Limited germinal center responses; |
| isotope) of antibodies | changes in B/T cell repertoire |
| Short persistence of antibody | Limited plasma cell survival |
| responses to proteins | |
| Limited induction of CD4/CD8 | Decline in naïve T-cell reservoir |
| responses | (accumulation of effector memory |
| | and CD8 T-cell clones) |
| Limited persistence of CD4 | Limited induction of new effector |
| responses | memory T cells (IL-2, IL-7) |

FDC, follicular dendritic cell;
Ig, immunoglobulin;
IL, interleukin.

Kits

Kits comprising host cells (or a cell population comprising the same) or expression vector systems or a composition comprising any one of the foregoing of the present invention are also provided. In illustrative aspects, the kits comprise a unit dose of cells comprising the expression vector systems of the present invention. In illustrative aspects, the kit comprises a sterile, GMP-grade unit dose of the cells. In illustrative aspects, a unit dose of cells comprises 10⁵, 10⁶, 10⁷, 10⁸, 10⁹, 10¹⁰, 10¹¹, 10¹² 10¹³, or more than 10¹⁵ cells comprising the expression vector system of the present invention.

In illustrative aspects, the unit dose of cells are packaged in an intravenous bag. In illustrative aspects, the unit dose of cells are provided in a cryogenic form. In illustrative aspects, the unit dose of cells are ready to use. In illustrative aspects, the unit dose of cells are provided in a tube, a flask, a dish, or like container.

In illustrative aspects, the cells are cryopreserved. In illustrative aspects, the cells are not frozen.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein.

All methods described herein can be performed in any suitable order unless otherwise indicated herein or otherwise clearly contradicted by context. The use of any and all examples, or illustrative language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

As used herein, all headings are simply for organization and are not intended to limit the disclosure in any manner. The content of any individual section may be equally applicable to all sections.

### EXAMPLES

### Example 1—Vector-Engineered Therapy

Vector-engineered therapy incorporating a gp96-Ig fusion protein, a T cell costimulatory fusion protein (e.g., OX40L-Ig), and/or coronavirus protein, or an antigenic portion thereof elicits a superior antigen-specific CD8+ T cell response.

A gp96-Ig expression vector was re-engineered to simultaneously co-express OX40L-Ig, ICOSL-Ig, or 4-1BBL-Ig, thus providing a costimulatory benefit without the need for additional antibody therapy. Thus, combination immunotherapy can be achieved by vector re-engineering, obviating the need for vaccine/antibody/fusion protein regimens, and importantly may limit both cost of therapy and the risk of systemic toxicity.

### Example 2—Vaccine+Costimulator Vector Re-Engineering

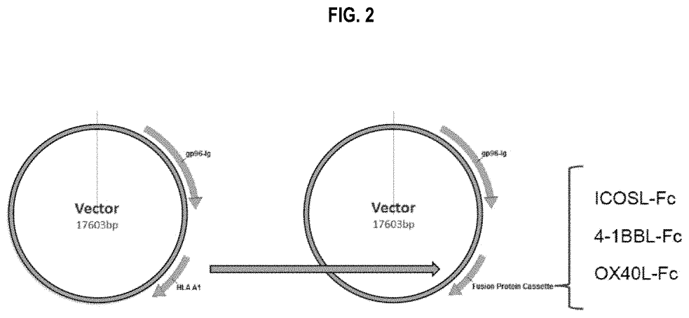

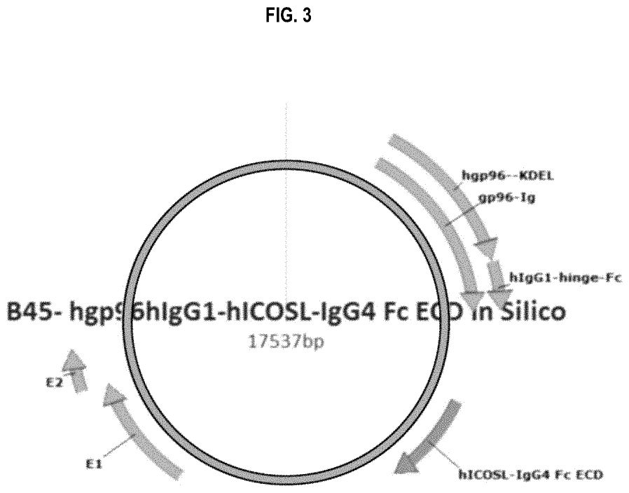

A vector re-engineering strategy was employed to incorporate vaccine and T cell costimulatory fusion proteins into a single vector. Specifically, the original gp96-Ig vector was re-engineered to generate a cell-based combination 10 product that secretes both the gp96-Ig fusion protein and various T cell costimulatory fusion proteins (FIGS. 2 and 3).

### Example 3—Generation and Testing of Gp96/OX40L-Ig, SARS-CoV-2 Vaccine

The animal model system used to test the inventive novel gp96/OX40L-Ig, SARS-CoV-2 vaccine is C57Bl/6 mice administered an immunization and rechallenge protocol, typical of vaccine development. This vaccine uses heat shock protein gp96, genetically fused to an immunoglobulin domain, which acts as a potent adjuvant that activates TLR2 and TLR4 on professional antigen presenting cells. An advantage offered by this gp96 based technology is that it allows for an antigen fused or presented in the context of gp96 to drive a potent and long-standing immune response. The technology can be used to genetically fuse the 51 and S2 capsid proteins of SARS-CoV-2 to gp96-Ig to create a potent vaccine, designed to generate protective, adaptive and humoral immunity against shared sequences of SARS-CoV-2. The gp96 protein is used to deliver multiple SARS-CoV-2 antigens to activate the immune system and thereby elicit long-lasting immune response against SARS-CoV-2 virus. Coronavirus spike proteins (S1 and S2) are being inserted using a clinically proven vector, plasmid B45. These replicates serve as a multi-copy episome and provide high levels of expression. COVID-19 capsid proteins can be incorporated into vectors that express OX40L, in addition to gp96, which will provide CD4+ T-cell help, subsequent B-cell class switching and protective antibody production.

Using S1/S2-SARS-CoV-2, expressing gp96/OX40L-Ig, mice are immunized, and CD4+ and CD8+ T-cell responses are evaluated. Primary immune responses can be measured by intracellular staining for IFN-gamma by flow cytometry following re-stimulation of isolated cells with SARS-CoV-2-spike protein overlapping peptide pools. In addition to specific evaluation the CD8+ T-cell response, the SARS-CoV-2 specific antibody responses can also be evaluated using serum samples. After establishing the best route of vaccination, memory responses in the lungs after secondary immunization can be further evaluated. Immunogenicity of gp96-Ig-OX40L-Fc that express SARS-CoV-2 antigens can be compared in head-to-head experiments with gp96-Ig-SARS-CoV-2. These experiments are optionally followed by measurement of the induction of memory CD8+ T cell responses in the lung after secondary immunizations. These studies form a backbone in establishing the efficacy of the gp96-SARS-CoV-2 vaccine. In this way, potent vaccines against nCoV are generated and then tested in humans for protective cell and humoral immunity.

### Example 4: AD100 and HEK-293 Express Gp96-Ig and Protein S

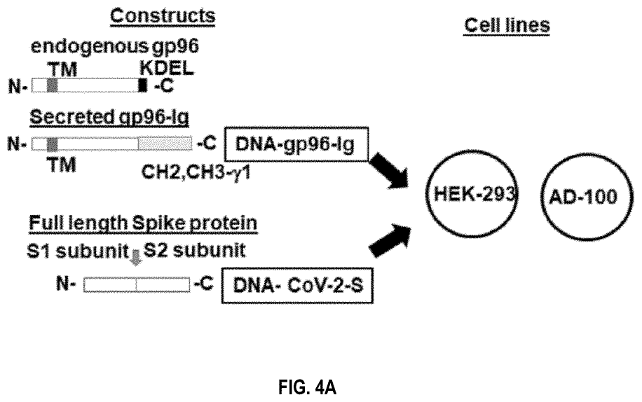

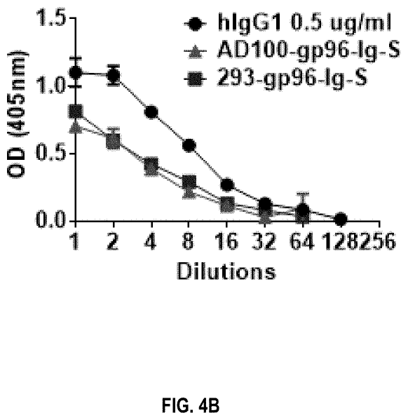

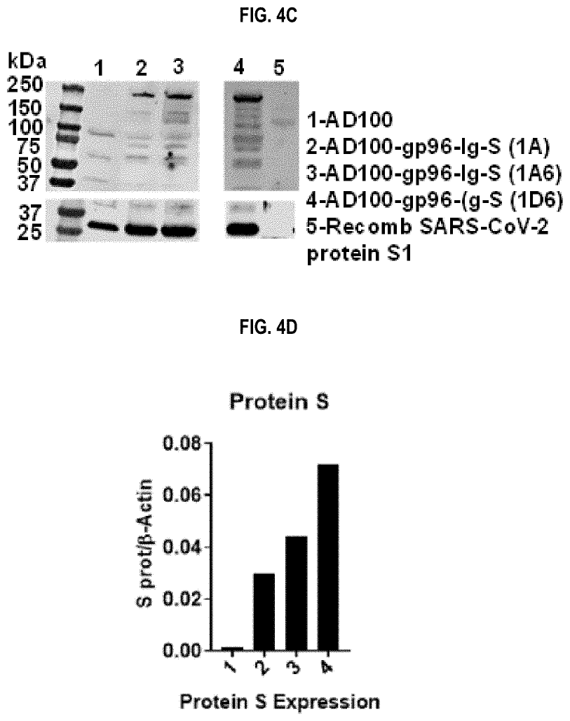

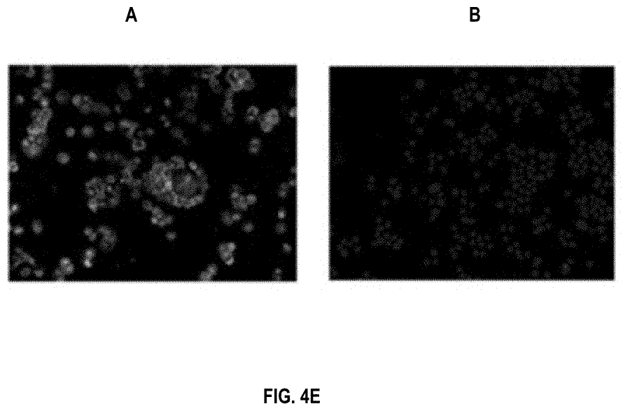

In the experiments of this example, cell based secreted heat shock protein technology was utilized to generate vaccine cells HEK-293-gp96-Ig-S and AD-100-gp96-Ig-S. The secretory form of gp96 protein (gp96-Ig) was generated by replacing the c-terminal, KDEL retention sequence of human gp96 gene, with the hinge region and constant heavy chains (CH2 and CH3) of human IgG1 (FIG. 4A). Vector pcDNA 3.1(+) has high-level, constitutive expression in mammalian cell lines and this vector was used to express SARS-CoV-2 spike (S) protein (disclosed herein as “protein S”) (FIG. 4A). cDNAs encoding the full-length SARS-CoV S glycoprotein included the Kozak sequence (A/GCCAUGG) (SEQ ID NO: 98) to optimize expression in eukaryotic cells without any other modification, containing endogenous leader sequence, transmembrane and cytosolic domain.

Vaccine cells, HEK-293-gp96-Ig-S and AD100-gp96-Ig, were generated by co-transfection of AD100 and HEK293 cells with plasmids encoding gp96-Ig (B45) and protein S (pcDNA3.1) and selection with G418 and L-histidonol. ELISA experiments confirmed that both stable transfected cell lines secreted gp96-Ig into culture supernatants at a rate of 125 ng/mL/24 h/10⁶ vaccine cells (FIG. 4B).

Expression of protein S by the vaccine cells was confirmed by analyzing vaccine cell lysates on SDS-page and blotting with anti-SARS-CoV2 S1 antibody (FIGS. 4C and 4D), and by immunofluorescence (FIG. 4E). Expression of full-length protein S (250 kDa) was observed only in AD100 transfected cell lines (lanes 2-4), and not in non-transfected AD100 cell line (lane 1). Cleavage product, protein S1 (slightly higher molecular weight of 120 kDa) was determined by detection with anti-protein S1 antibody (FIG. 4C, lanes 2-4). In addition, some additional, slightly lower molecular weight bend of 120 kDa was observed, which may represent gp96-Ig fusion protein chaperoning the protein S1 epitope. Molecular weight of gp96-Ig fusion protein was 116 kDa. Additional bends, of approximately 70 kDa, were found to be expressed only in transfected cell line. The non-transfected AD100 cell line did not express proteins molecular weight of 250 kDa or 120 kDa. However, expression of some non-specific bends of 100, 60 and 40 kDa was observed. Recombinant protein S1 120 kDa was used as a positive control in these experiments. The ratio of protein S to β-actin expression was calculated (FIG. 4D), and protein S expression was confirmed by immunofluorescence (FIG. 4E). Cytoplasmic and transmembrane distribution of protein S within AD100-gp96-Ig-S cell line was observed.

### Example 5: Secreted Gp96-Ig-S Vaccine Induces CD8+ T Cell Effector Memory and Resident Memory Responses in the Lungs

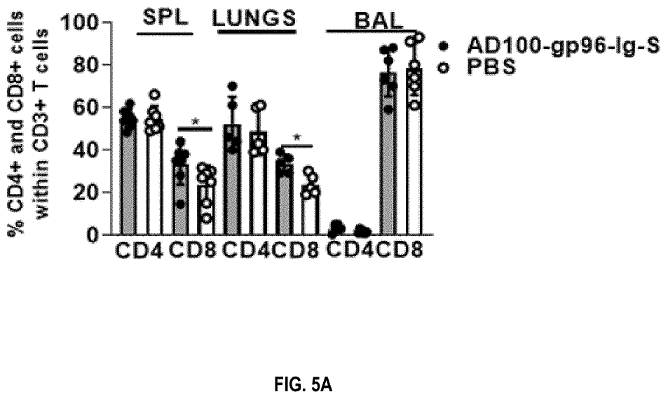

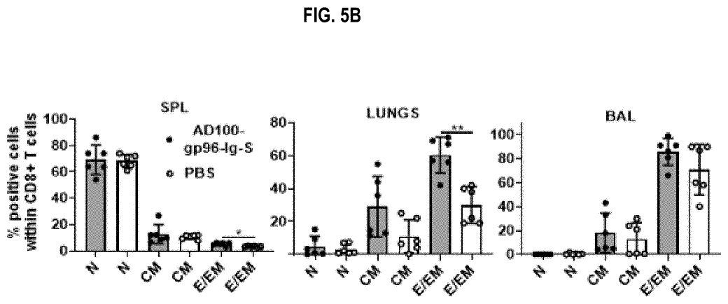

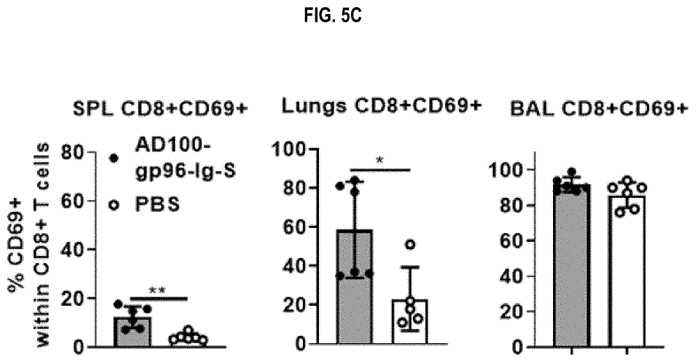

In the experiments of this example, a dose of 200 ng/ml was used to immunize mice with AD100-gp96-Ig-S vaccine. Mice were vaccinated by subcutaneous (s.c) route of administration, and after 5 days, the frequency of T cells within spleen, lungs (lung parenchyma) and bronhioalveolar lavage cells (lung airways) was determined. A significant increase in the frequencies of CD8+ T cells in the spleen and lungs was observed, but not within bronchoalveolar lavage (BAL) of vaccinated mice (FIG. 5A). Frequency of CD4+ T cells was unchanged between vaccinated and control mice in all analyzed tissues. While vaccination with gp96-Ig induces CD8+ T cell effector memory differentiation, in the experiments of this example, it was confirmed that gp96-Ig-S vaccine primes a strong effector memory CD8+ T-cell responses, as determined by analysis of CD44 and CD62L expression (FIG. 5B). While the frequency of naïve (N), CD44−CD62L+CD8 T cells and central memory (CM), CD44+CD62L+CD8+ T cells was unchanged, there was a statistically significant increase of effector memory (EM) CD44+CD62L− CD8+ T cells within the spleen and lungs (FIG. 5B). In addition, a trend of more EM CD8+ T cells within the CD8+ T cells in the BAL was observed (FIG. 5B). Resident memory T cells (RM) are distinct memory T cell subset compared to CM and EM cells that are uniquely situated in different tissues, including lungs. One of the canonical markers of tissue resident memory T cells is CD69. There was a significant increase in the frequency of CD8+CD69+ T cells in vaccinated compared to control, non-vaccinated mice in both, spleen, and lungs (FIG. 5C). Even though the frequency of CD8+CD69+ T cells was the highest in the BAL compared to spleen and lungs, it was not observed in the difference in their frequencies between vaccinated and control mice. Overall, AD100-gp96-Ig vaccine induced both, EM, and RM CD8+ T cells in the spleen and lungs.

### Example 6: Protein S Specific CD8 and CD4 Th1 T Cell Responses are Both Induced by Gp96-Ig-S Vaccine

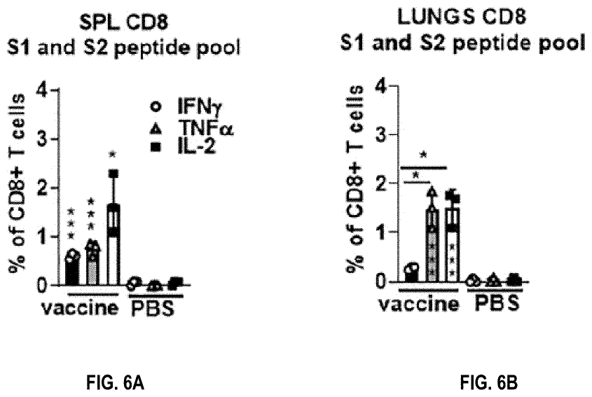

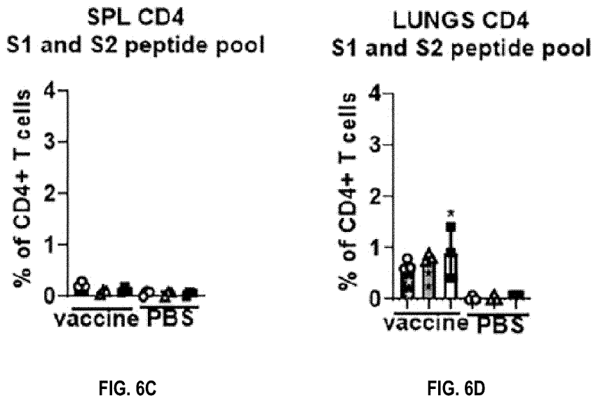

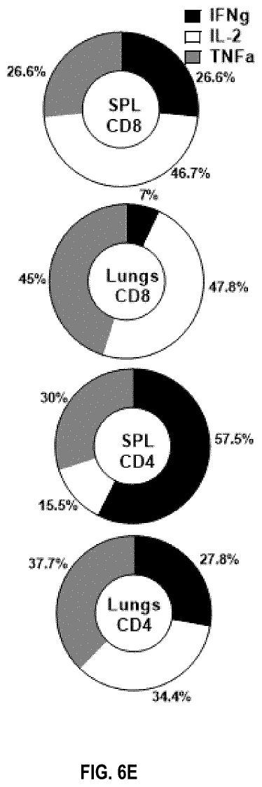

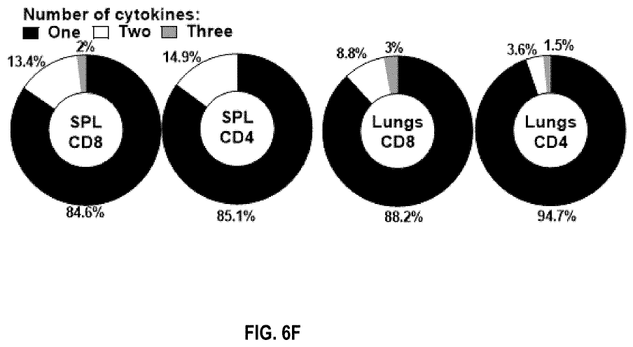

To evaluate polyepitope, protein S specific CD8+ and CD4+ T cell responses induced by gp96-Ig-S vaccination, the experiments of this example used pooled S peptides (S1+S2) and multiparameter intracellular cytokine staining assay to assess Th1 (IFNγ+, IL-2+ and TNFα+) CD8+ and CD4+ T cells (FIG. 6A). Spleen and lung cells were tested for responses to the pool of overlapping protein S peptides (S1+S2), and all of the vaccinated animals showed significantly higher magnitude of the protein S-specific T cell responses against 51 and S2 epitopes compared to the non-vaccinated controls (FIG. 6A, FIG. 6B, FIG. 6C, FIG. 6D). Increases in the vaccine induced Th1 CD8 T cell responses (IFNγ+, IL-2+ and TNFα+) was noted in both, spleen and lungs (FIG. 4A and FIG. 4B), while Th1 CD4 T cell responses (IFNγ+, IL-2+ and TNFα+) were induced only in lungs (FIG. 6C and FIG. 6D). The proportion of the protein S-specific CD8+ T cells that produce IFNγ (26.6%) was significantly reduced in the lungs (7%) while both, TNFα and IL-2 productions was increased in the lungs (45% and 47%), compared to spleen (26% and 26%) (FIG. 6E). In these experiments, the proportion of the protein S-specific CD4+ T cells that produces IFNγ was found to be higher in the spleen than in the lungs (57% in the spleen vs 27% in the lungs), while IL-2 production was higher in the lungs than in the spleen (15% in the spleen vs 34% in the lungs) (FIG. 6E). Assessment of the polyfunctionality of protein S-specific CD8+ and CD4+ T cells in the spleen and lungs revealed that the vast majority of protein 5-specific CD8+ and CD4+ T cells, irrespective of their location, synthesized only 1 cytokine (FIG. 6F). The proportion of the protein S-specific CD8+ T cells in the spleen and lungs that produced 3 cytokines at the same time was higher than for CD4+ T cells. Only a small proportion of protein S-specific CD4+ T cells in the lungs produced 2 or 3 cytokines (3.6% two cytokines and 1.5% three cytokines) (FIG. 6F).

### Example 7: Induction of SARS-CoV-2 Protein S Immunodominant Epitopes Specific CD8+ T Cells in the Lungs and Airways of Vaccinated HLA-A2-Transgenic Mice

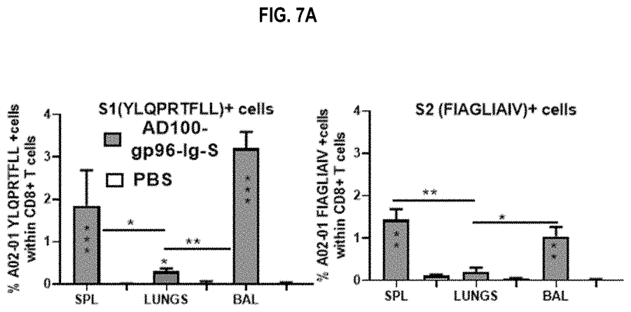

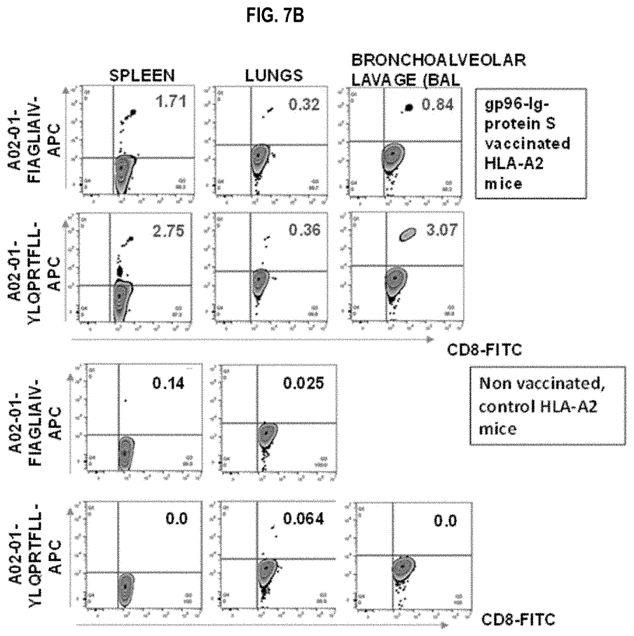

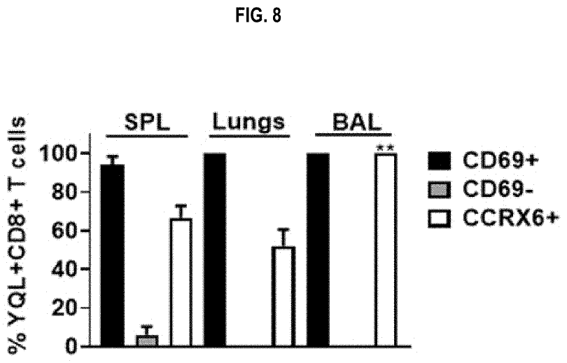

Polyfunctional SARS-CoV-2-specific memory CD8+ T cell responses generated against cognate antigens may be positively correlated with several symptom free days after infection. Therefore, it is important to develop vaccines that can elicited SARS-CoV specific CD8+ T cells. Having identified overall T cell responses to SARS-CoV protein S (FIGS. 6A-6F), the experiments of this example analyzed how gp96-Ig-S vaccine induced HLA class I specific cross presentation of immunodominant SARS-CoV2 protein S epitopes. Transgenic HLA-A 02:01 mice and HLA class I pentamers were used as probes to detect CD8+ T cells specific for two immunodominant SARS-CoV2 protein S epitopes: YLQPRTFLL (YLQ) (aa 269-277) (SEQ ID NO: 97) and FIAGLIAIV (FIA) (aa 1220-1228) (SEQ ID NO: 96) in vaccinated mice (FIGS. 7A and 7B). The experiments showed that the vaccine efficaciously induces both, YLQ+CD8 T cells, as well as FIA+CD8+ T cells in the spleen, lungs and BAL (FIGS. 7A and 7B). Interestingly, the highest magnitude of YLQ+CD8+ T cells in the BAL of vaccinated mice and the lowest frequency of YLQ+ and FIA+CD8+ T cells was observed in the lungs. Further phenotype analysis of YLQ+CD8+ T cells confirmed that these cells express the CD69 marker and CXCR6 (FIG. 8). Particularly, the experiments demonstrated that all of the YLQ+CD8+ T cells in the BAL are also CXCR6+, and the frequency of YLQ+CD8+CXCR6+ cells was significantly higher in the BAL compared to lungs.

### Example 8: Cell-Based Vaccine Methods

Generation of Vaccine Cell Lines

Human embryonic kidney (HEK)-293 cells, obtained from the American Tissue Culture Collection (ATCC #CRL-1573) and human lung adenocarcinoma cell lines (AD100), were transfected with two plasmids: B45, encoding gp96-Ig, UM and pcDNA3.1, encoding full length SARS-CoV2-protein S gene, (Genomic Sequence: NC_045512.2; NCBI Reference Sequence: YP_009724390.1 GenBank Reference Sequence: QHD43416). The B45 plasmid expressing secreted gp96-Ig has been approved by FDA and OBA for human use and is currently employed in a clinical study for the treatment of non-small cell lung cancer (NCT02117024, NCT02439450). The histidinol-selected, B45 plasmid, replicates as multi-copy episome and provides high levels of expression. SARS-CoV-2 protein S cDNA was generated by reverse transcription-PCR with primers that amplified the cDNA between the ATG codon of the leader peptide and the termination codon and cloned into the neomycin-selectable eukaryotic expression vector, pcDNA 3.1. HEK-293 and AD100 cells were simultaneously transfected with B45 and pcDNA3.1 plasmid by Lipofectamin. Transfected cells were selected with 1 mg/ml of G418 (Life Technologies, Inc.) for B45 and with 7.5 mM of L-Histidinol (Sigma Chemical Co., St. Louis, Mo.) for pcDNA 3.1). After a stable transfection cell line was established, single cell cloning by limiting dilution assay was performed and all the cell clones were first screen for gp96-Ig production and then for protein S expression. Vaccine cells sterility testing, IMPACT II PCR evaluation was performed for: Ectromelia, EDIM, LCMV, LDEV, MAV1, MAV2, mCMV, MHV, MNV, MPV, MVM, Mycoplasma pulmonis, Mycoplasma sp., Polyoma, PVM, REO3, Sendai, TMEV and all test results were found negative.

Western Blotting and ELISA

Protein expression was verified by SDS-page and Western blotting using rabbit anti-SARS-CoV-2 spike glycoprotein antibody (Abcam, ab272504) at 1/1000 dilution and secondary antibody: Peroxidase AffiniPure F(ab′)₂ Fragment Donkey Anti-Rabbit IgG (H+L) (Jackson ImmunoResearch Laboratories) at/5000 dilution) HRP conjugated anti rabbit IgG (Jackson ImmunoResarch) at 1/5000 dilution. S protein was visualized by an enhanced chemiluminescence detection system (Amersham Biosciences, Piscataway, N.J.) (FIG. 4C). Recombinant Human coronavirus SARS-CoV-2 Spike Glycoprotein 51 (Fc Chimera) (ab272105, Abcam) was used a as a positive control (loaded 2.4 ug/lane). One million cells were plated in 1 ml for 24 h and gp96-Ig production was determined in the supernatant by ELISA using anti-human IgG antibody for detection and human IgG1 as a standard (FIG. 4B).

Immunofluorescence (IF)

AD100-gp96-Ig cytospins were fixed in pure cold acetone (VWR chemicals, BDH®, Catalog #: BDH1101) for 10 minutes followed by 3 washes of 5 minutes each with PBS. The slides were left in blocking media (5% BSA in PBS) at RT for 2 hours. The following fluorescent antibodies: Anti-SARS-CoV-2 spike glycoprotein antibody—Coronavirus (ab272504) from Abcam and Donkey anti rabbit IgG FITC, BioLegend Cat #406403, were added in 1/50 and 1/100 dilutions of the antibodies combined in 5% BSA in PBS and/or Rabbit Isotype control, Abcam Ab172730 diluted 1/50 and incubated overnight at 4° C. in a dark moisture chamber. Next day slides were washed 3 times for 5 minutes with PBS and mounted with Prolong Gold antifade reagent with DAPI from Invitrogen, Catalog #36935, covered with a coverslip and allowed to cure. Sealed with nail polish and taken to the Keyence microscope for examination. The following filter cubes were used: DAPI (for nuclear stain), FITC (for protein S) and acquired on Keyance microscope (BZ-X Viewer).

Animals and Vaccination

Mice used in these experiments were colony-bred mice (C57Bl/6) and HLA-A02-01 transgenic mice (C57BL/6-Mcph1Tg(HLA-A2.1)1Enge/J, Stock No: 003475) purchased from JAX Mice, The Jackson laboratory (Farmington, Conn. USA). Homozygous mice carrying the Tg(HLA-A2.1)1Enge transgene express human class I MHC Ag HLA-A2.1. The animals were housed and handled in accordance with the standards of the Association for the Assessment and Accreditation of Laboratory Animal Care International under an IACUC approved protocol. Both, female and male mice were used at 6-10 weeks of age. Equivalent number of 293-gp96-Ig-protein S and AD100-gp96-Ig-protein S cells that produce 200 ng gp96-Ig or PBS were injected by subcutaneous (s.c.) route in C57Bl/6 and HLA-A2 transgenic mice. Mice were sacrificed 5 days after vaccination and spleen, lungs and BAL were collected and processed into single-cell suspension.

BAL and Lung Harvest and Cell Isolation

For mouse samples, spleens were collected, and tissues processed into single cell suspension. Leukocytes were isolated form spleen and cervical lymph nodes by mechanical dissociation and red blood cells were lysed by lysing solution. BAL was harvested directly from euthanized mice via insertion of a 22-gauge catheter into an incision in the trachea. HBSS was injected into trachea and aspirated 4 times. Recovered lavage fluid was collected and BAL cells were collected after centrifugation. To isolate intraparenchymal lung lymphoid cells, the lungs were flushed by 5 ml of pre-chilled HBSS into the right ventricle. When the color of the lungs changed to white, the lungs were excised avoiding the peritracheal lymph nodes. Lungs were then removed, washed in HBSS and cut into 300 mm pieces, and incubated in IMDM containing 1 mg/ml collagenase IV (Sigma) for 30 min at 37 C on a rotary agitator (approximately 60 rpm). Any remaining intact tissue was disrupted by passage through a 21-gauge needle. Tissue fragments and majority of the dead cells were removed by a 250-mm mesh screen, and cells were collected after centrifugation.

Ex Vivo Stimulation and Intracellular Cytokine Staining

Spleen and intraparenchymal lung lymphocytes from immunized and control animals were analyzed for Protein S-specific CD8+ T cell responses. 1-1.5×10⁶ cells were incubated for 20 h with two protein S peptide pools (51 and S2, homologous to vaccine insert) (JPT Peptide Technologies; PM-WCPV-S1). Peptide pools contain pools of 15-meric peptides overlapping by 11 amino acids covering the entire protein S proteins. Peptide pools were combined (S1+S2) and used at a final concentration of 1.25 ug/ml of each peptide, followed by addition of Brefeldin A (GolgiPlug; BD Bioscience) (10 ug/ml) for last 5 h or incubation. Stimulation without peptides served as background control. The results are calculated as the total number of cytokine-positive cells with background subtracted. Peptide stimulated and non-stimulated cells were first labeled with live/dead detection kit (Thermo Fisher Scientific) and then resuspended in BD Fc Block (clone 2.4G2) for 5 bmin RT prior to staining with a surface stain cocktail containing following antibodies purchased form Biolegend: CD45(clone) AF700, CD3, CD4, CD8, CD69, CXCR6, CD44, CD62L. After 30 min, cells were washed with FACS buffer then fixed and permeabilized using BD Cytofix/Perm fixation/permeabilization solution kit according to manufacturer instructions, followed by intracellular staining using cocktail of the following antibodies purchased from Biolegend: IFNg, IL-2 and TNFα. Data was collected on an Fortessa instrument (BD Biosciences). Analysis was performed using FlowJo software version 10.8 (Tree Star). First cells were gated on live cells and then lymphocytes were gated for CD3+ and progressive gating on CD8+ T cell subsets. Antigen-responding CD8 T cells (IFNγ or IL-2 or TNFα producing/expressing cells) were determined either on the total CD8+ T cell population or on CD8+ CD69+ cells. Acquisition was limited to cells expressing Alexa700 fluorochrome/CD3 at a particle cut-off size (FSC) of 3000 and 50,000 events/sample were acquired at a medium flow rate by 20-color, Fortessa flow cytometer using the FACS DIVA software. Flow data was analyzed by Flow.Jo 10 software.

HLA-A02-01 Pentamer Staining

A total of 1-2×10⁶ spleen, Bronchoalveolar Lavage (BAL) or lung cells were labelled with peptide-MHC class I pentamer-APC (ProIMmune, UK) and incubated for 15 min at 37 C. Dead cells were labelled with LIVE/DEAD Violet stain kit (Invitrogen) and then following antibody cocktail was used: CD45 (clone) AF700, CD3, CD4, CD8, CD69, CXCR6, CD44, CD62L. Cells (spleen and lung cells) that were stimulated overnight with peptide pools (as described under ex vivo stimulation and intracellular staining) were fix permeabilized with Cytofix/Perm solution (BD) and then stained for intracellular cytokines: IFNγ, IL-2 and TNFα. Cells were acquired on a Fortessa instrument, and data analyzed using FlowJo software version 10.8. Data were analyzed using forward side scatter single cell gate followed by CD45, CD3 and CD8 gating then tetramer gating within CD8 T cells positive cells. These cells were then analyzed for percentage expression of a marker using unstained and overall CD8+ population to determine the placement of the gate. Single color samples were run for compensation and FMO control samples were also applied to determine positive and negative populations, as well as channel spillover.

Statistics

All experiments were conducted independently at least three times on different days. Comparisons of flow cytometry cell frequencies for mouse studies was measured by the two-way ANOVA test with Holm-Sidak multiple-comparison test, *p<0.05, **p<0.01 and ***p<0.001 or unpaired T-tests (two-tailed) was carried out to compare between the control group and each of the experimental groups (alpha level of 0.05) using the Prism software (GraphPad software). Welch's correction was applied with unpaired T test, when P value of the F test to compare variances were 0.05. Data approximately conformed Shapiro-Wilk test and Kolmogorov-Smirnov tests for normality at 0.05 alpha level. Data were presented as mean±standard deviation in the text and in the figures. All statistical analysis was conducted using Graph Pad Prism 8 software.

### Example 9: Effect of Gp96-Based COVID-19 Vaccine Cell Lines ZVX-60 and ZVX-55 on CD8+ T Cells

Comparison of Frequency of HLA-A2-YLQ+(Pentamer+) Cells within CD8+ T Cells after Vaccination with Different Doses of ZVX-60 and ZVX-55 Vaccine Cells

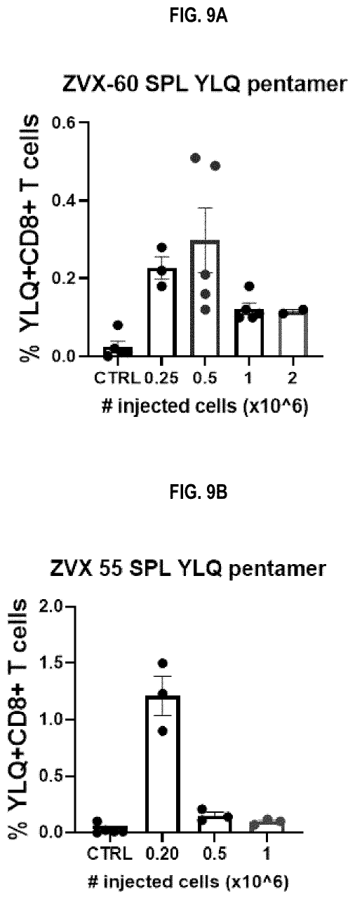

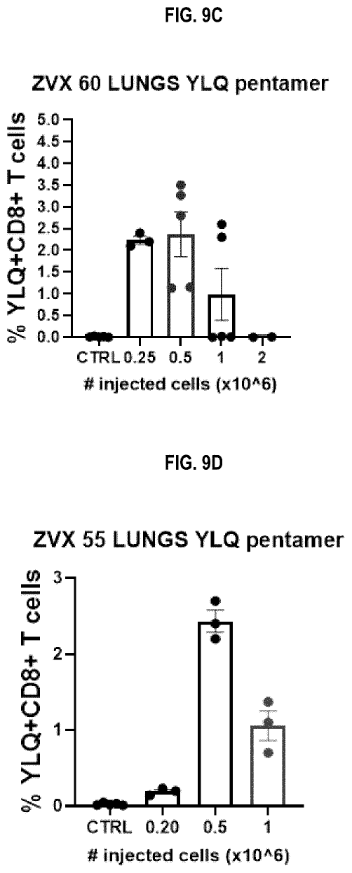

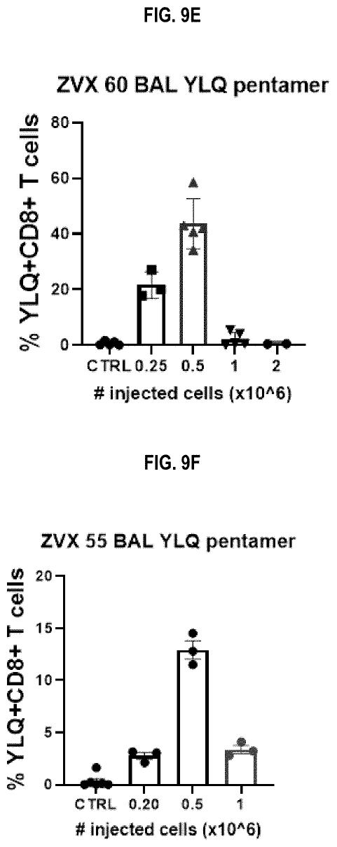

FIGS. 9A-9F show results of comparing frequency of HLA-A2.1 pentamer+ cells (YLQ+) within CD8+ T cells after vaccination with different number of ZVX-60 and ZVX-55 vaccine cells, which are SARS-CoV-2 cell-based vaccines in accordance with embodiments of the present disclosure. ZVX-60 is a SARS-CoV-2 cell-based vaccine that expresses gp96 and OX40L, along with a SARS-CoV-2 antigen; and ZVX-55 is a SARS-CoV-2 cell-based vaccine that expresses gp96, along with a SARS-CoV-2 antigen.

In FIGS. 9A-9F, bar graphs represent percentage of pentamer positive (YLQ+) cells within CD8+ T cells, as follows: ZVX-60 in spleen (“SPL”) (FIG. 9A), ZVX-55 in spleen (“SPL”) (FIG. 9B), ZVX-60 in lungs (FIG. 9C), ZVX-55 in lungs (FIG. 9D), ZVX-60 in BAL (FIG. 9E), and ZVX-55 in BAL (FIG. 9F). In FIGS. 9A, 9C, and 9E, the x-axis shows control (“CTRL”), 0.25×10⁶, 0.5×10⁶, 1×10⁶, and 2×10⁶ injected cells for ZVX-60. In FIGS. 9B, 9D, and 9F, the x-axis shows control (“CTRL”), 0.2×10⁶, 0.5×10⁶, and 1×10⁶ injected cells for ZVX-55. The data represents at least 2 technical replicates with 3-5 independent biologic replicates per group.

In this example, 5 days after the vaccination of HLA-A2 transgenic mice with different doses (number of injected vaccine cells), splenocytes, lung cells and BAL were isolated form vaccinated and control mice (PBS). Cells were stained with HLA-A2 02-01 pentamer containing YLQPRTFLL peptides, followed by surface staining for CD45, CD3, CD4, CD8, CD69, and CXCR6.

In this example, injected ZVX-60 cells produce 2000 ng/ml/10⁶ cell/24 h, while ZVX-55 produce 1200 ng/ml/10⁶ cell/24 h. The dose of 0.5×10⁶ ZVX-60 vaccine cells induced the highest frequency of pentamer+ (YLQ+) cells in all three compartments: spleen, lungs and BAL. This dose corresponds to a dose for a human of about 1000 ng of gp96-Ig. The highest frequency was observed in the BAL (40.7%), and the lowest in the spleen (0.29%). Animals vaccinated with the number of ZVX-55 vaccine cells that produce the same amount of gp96-Ig as ZVX-60 (1000 ng/ml/10⁶ cell/24 h) showed lower frequency of pentamer+cell in all three compartments compared to ZVX-60 vaccinated animals. However, decrease in the pentamer+ cells in both vaccines was not observed when vaccine dose was 1200 ng/ml for ZVX-55 and 2000 or 4000 ng/ml for ZVX-60.

Thus, ZVX-60 vaccine induced 51-specific CD8+ T cells in the spleen, lung tissue, and BAL.

Analysis of CD69 and CXCR6 Marker Expression on CD8+ T Cells after ZVX-60 Vaccination

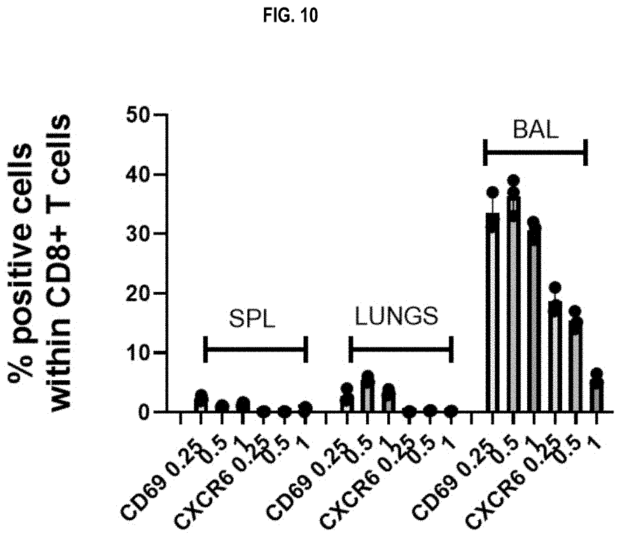

FIG. 10 illustrates results of the study of CD69 and CXCR6 marker expression on CD8+ T cells after ZVX-60 vaccination, and shows that ZVX-60 vaccine upregulates CD69 and CXCR6 markers on CD8+ T cells in the BAL. In FIG. 10, bar graphs represent percentage of marker positive cells within total CD8+ T cells for CD69 (0.25×10⁶ injected cells), CD69 (0.5×10⁶ injected cells), CD69 (1×10⁶ injected cells), CXCR6 (0.25×10⁶ injected cells), CXCR6 (0.5×10⁶ injected cells), and CXCR6 (1×10⁶ injected cells) for each of the spleen (“SPL”), lungs, and BAL. Data represent at least 2 technical replicates with 3 independent biologic replicates per group.

In this study, 5 days after the vaccination of HLA-A2 transgenic mice with different doses (number of injected vaccine cells), splenocytes, lung cells and BAL were isolated form vaccinated and control mice (PBS). Cells were stained for CD45, CD3, CD4, CD8, CD69, CXCR6.

Recently, CD69 and CXCR6 have been confirmed as core markers that define tissue resident memory (TRM) cells in the lungs. In this study, expression of CD69 and CXCR6 on total CD8+ T cells was compared in ZVX-60 vaccinated mice. The results confirmed the previous findings regarding induction of CD69 and CXCR6 on CD8+ T cells by gp96-Ig vaccination (Fisher et al., Frontiers in Immunology, 11, 26 Jan. 2021; 3740). The ZVX-60-induced CD69 and CXCR6 expression was the highest in the BAL for both doses: 0.25×10⁶ and 0.5×10⁶ injected cells, while 1×10⁶ vaccine cells induced the lowest expression of CD69 and CXCR6 on CD8 T cells.

Frequency of Different CD8+ and CD4+ T Cell Subsets after Different Doses of ZVX-60

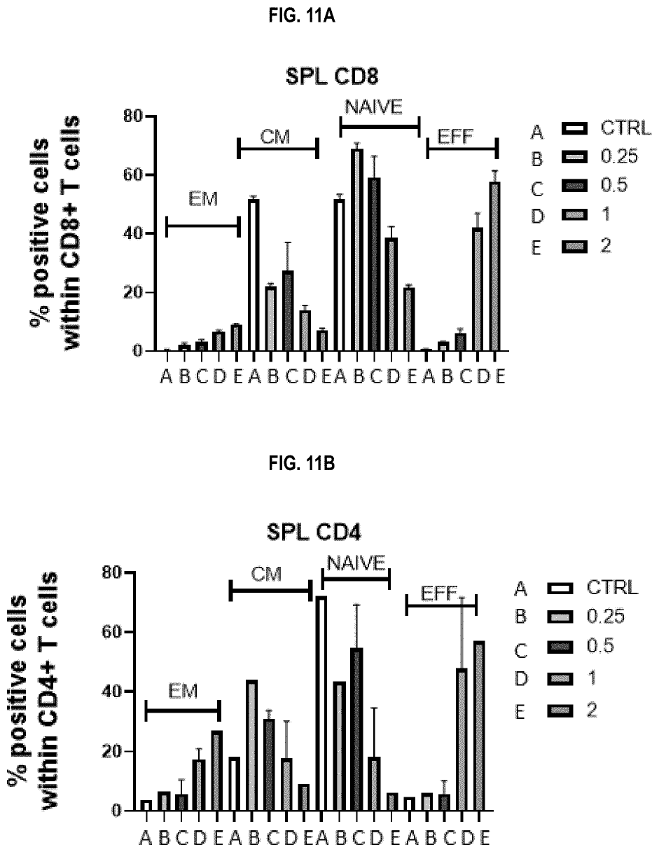

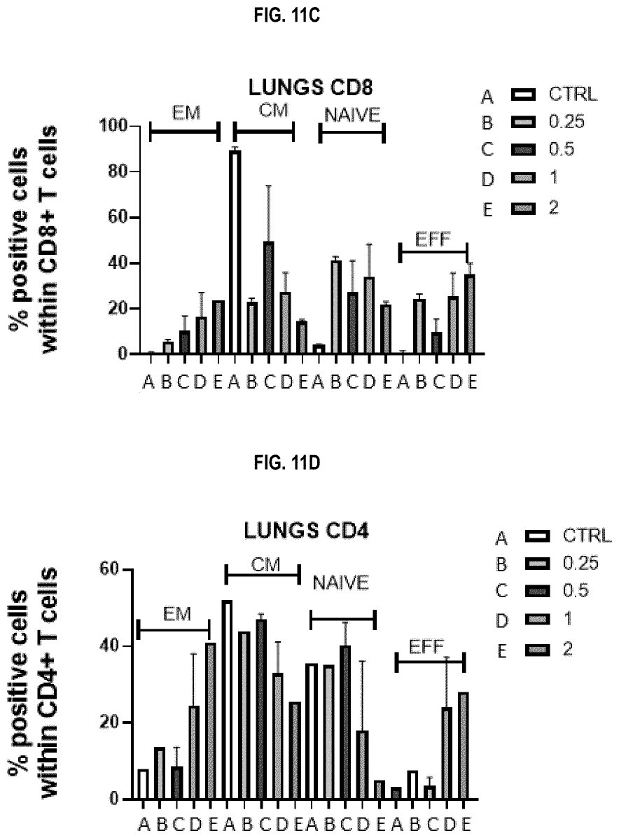

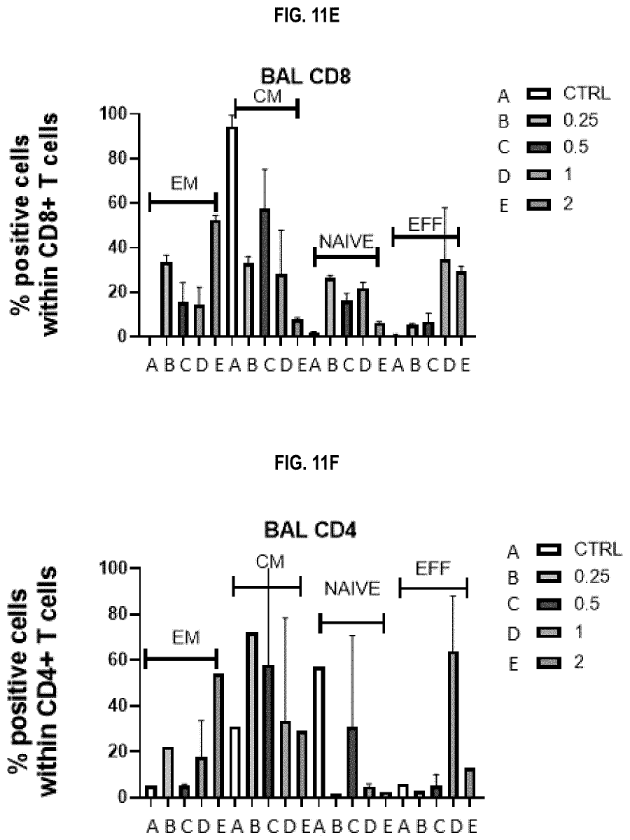

This study assessed a frequency of different CD8+ and CD4+ T cell subsets after several different doses of ZVX-60. In FIGS. 11A-11F, bar graphs represent percentage of positive cells of CD8+T and CD4+ T cell subsets: effector memory (“EM,” CD44+CD62L−), central memory (“CM,” CD44+CD62L+), naïve (“Naïve,” CD44−CD62L−); and effector (“EFF,” CD44−CD62L−) cells, within total CD8+ T or CD4+ T cells. FIGS. 11A-11F show results for the following doses of ZVX-60 vaccine cells for each of the EM, CM, Naïve, and EFF subsets: control (“CTRL”), 0.25×10⁶, 0.5×10⁶, 1×10⁶, and 2×10⁶ vaccine cells, in this order. FIG. 11A shows percentage of positive cells within CD8+ T cells in the spleen (“SPL”), FIG. 11B shows percentage of positive cells within CD4+ T cells in the spleen (“SPL”), FIG. 11C shows percentage of positive cells within CD8+ T cells in the lungs, FIG. 11D shows percentage of positive cells within CD4+ T cells in the lungs, FIG. 11E shows percentage of positive cells within CD8+ T cells in the BAL, and FIG. 11F shows percentage of positive cells within CD4+ T cells in the BAL. Data represent at least 2 technical replicates with 3-5 independent biologic replicates per group.

In this study, 5 days after the vaccination of HLA-A2 transgenic mice with different doses (number of injected vaccine cells), splenocytes, lung cells and BAL were isolated form vaccinated and control mice (PBS). Cells were stained for CD45, CD3, CD4, CD8, CD44, CD62L.

The results of this study demonstrate a dose-dependent induction of CD8+ effector cells by ZVX-60. It was determined that the dose of 0.25×10⁶ and 0.5×10⁶ ZVX-60 vaccine cells primarily induces central memory CD8+ T cells in all compartments (SPL, lungs and BAL), in striking contrast to the effect of the higher dose of ZVX-60 (1×10⁶ and 2×10⁶ cells) which induces primarily effector memory and effector CD8+ T cell phenotype. The 0.25×10⁶ and 0.5×10⁶ dose used in mice corresponds to a dose for a human in the range of from about 500 ng to about 1000 ng of gp96-Ig. Similar effect of ZVX-60 vaccine dose was observed for CD4+ T cells: low dose induced central memory, while high dose induced effector CD4+ T cell phenotype.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

The content of any individual section may be equally applicable to all sections.

### INCORPORATION BY REFERENCE

All patents and publications referenced herein are hereby incorporated by reference in their entireties.

The publications discussed herein are provided solely for their disclosure prior to the filing date of the present application. Nothing herein is to be construed as an admission that the present invention is not entitled to antedate such publication by virtue of prior invention.

As used herein, all headings are simply for organization and are not intended to limit the disclosure in any way.

What is claimed is:

1. An expression vector system comprising
(i) a nucleic acid encoding a secretable fusion protein comprising a chaperone protein and an immunoglobulin, or a fragment thereof, and
(ii) a nucleic acid encoding a T cell costimulatory fusion protein, wherein the T cell costimulatory fusion protein enhances activation of antigen-specific T cells when administered to a subject; and/or
(iii) a nucleic acid encoding a coronavirus protein, or an antigenic portion thereof, wherein each nucleic acid is operably linked to a promoter.

2. The expression vector system of claim 1, wherein the chaperone protein of the secretable fusion protein is a secretable gp96-Ig fusion protein which optionally lacks the gp96 KDEL sequence.

3. The expression vector system of claim 2, wherein the immunoglobulin comprises an Ig tag of the gp96-Ig fusion protein comprising the Fc region of human IgG1, IgG2, IgG3, IgG4, IgM, IgA, or IgE.

4. The expression vector system of claim 1, wherein the nucleic acid encoding the secretable fusion protein is operably linked to a promoter which is different from a promoter which is operably linked to the nucleic acid encoding the coronavirus protein, or an antigenic portion thereof.

5. The expression vector system of claim 4, wherein the nucleic acid encoding the secretable fusion protein is operably linked to a CMV promoter.

6. The expression vector system of any one of claims 1 to 5, wherein the nucleic acid encoding the coronavirus protein, or an antigenic portion thereof is operably linked to an Mth promoter.

7. The expression vector system of any one of claims 1 to 6, wherein the nucleic acid encoding the fusion protein and the nucleic acid encoding the coronavirus protein, or antigenic portion thereof, are present on the same expression vector.

8. The expression vector system of any one of claims 1 to 6, wherein the nucleic acid encoding the fusion protein is present on an expression vector which is different from the expression vector comprising the nucleic acid encoding the coronavirus protein, or antigenic portion thereof.

9. The expression vector system of any one of claims 1 to 8, comprising two or more nucleic acids each encoding a different coronavirus protein, or an antigenic portion thereof.

10. The expression vector system of any one of the previous claims, wherein the chaperone protein is selected from the group consisting of: gp96, Hsp70, BiP, and Grp78.

11. The expression vector system of any one of the previous claims, wherein the T cell costimulatory fusion protein is OX40L-Ig, or a portion thereof that binds to OX40.

12. The expression vector system of any one of the previous claims, wherein the T cell costimulatory fusion protein is selected from OX40L-Ig or a portion thereof that binds specifically to OX40, ICOSL-Ig or a portion thereof that binds specifically to ICOS, 4-1BBL-Ig, or a portion thereof that binds specifically to 4-1BBR, CD40L-Ig, or a portion thereof that binds specifically to CD40, CD70-Ig, or a portion thereof that binds specifically to CD27, TL1A-Ig or a portion thereof that binds specifically to TNFRSF25, or GITRL-Ig or a portion thereof that binds specifically to GITR.

13. The expression vector system of any one of the previous claims, wherein the chaperone protein comprises an amino acid sequence of any one of SEQ ID NOs: 2, 29, 30, and 31, or an amino acid sequence having at least about 90%, or at least about 95%, or at least about 97%, or at least about 98%, or at least about 99% identity thereto.

14. The expression vector system of claim 13, wherein the chaperone protein is gp96 comprising the amino acid sequence of SEQ ID NO: 2.

15. The expression vector system of any one of the previous claims, wherein the fusion protein comprises an Fc fragment of an immunoglobulin.

16. The expression vector system of claim 15, wherein the immunoglobulin is an IgG1 immunoglobulin.

17. The expression vector system of claim 15 or claim 16, wherein the Fc fragment comprises the amino acid sequence of SEQ ID NO: 5, or an amino acid sequence having at least about 90%, or at least about 95%, or at least about 97%, or at least about 98%, or at least about 99% identity thereto.

18. The expression vector system of any one of the previous claims, wherein the fusion protein comprises the amino acid sequence of SEQ ID NO: 8, or an amino acid sequence having at least about 90%, or at least about 95%, or at least about 97%, or at least about 98%, or at least about 99% identity thereto.

19. The expression vector system of any one of the previous claims, wherein the coronavirus protein is a betacoronavirus protein or an alphacoronavirus protein, optionally wherein the betacoronavirus protein is selected from a SARS-CoV-2, SARS-CoV, MERS-CoV, HCoV-HKU1, and HCoV-OC43 protein, or an antigenic fragment thereof or the alphacoronavirus protein is selected from a HCoV-NL63 and HCoV-229E protein, or an antigenic fragment thereof.

20. The expression vector system of claim 19, wherein the betacoronavirus protein is a SARS-CoV-2 protein.

21. The expression vector system of claim 20, wherein the SARS-CoV-2 protein is a variant of a SARS-CoV-2 protein.

22. The expression vector system of claim 21, wherein the SARS-CoV-2 protein comprises an amino acid encoded by a nucleic acid having a nucleotide sequence of SEQ ID NO: 46, or an antigenic fragment thereof.

23. The expression vector system of any one of the previous claims, wherein the coronavirus protein is a SARS-CoV-2 protein, or an antigenic fragment thereof selected from spike surface glycoprotein, membrane glycoprotein M, envelope protein E, and nucleocapsid phosphoprotein N.

24. The expression vector system of claim 23, wherein the spike surface glycoprotein comprises the amino acid sequence of SEQ ID NO: 37, membrane glycoprotein precursor M comprises the amino acid sequence of SEQ ID NO: 40, the envelope protein E comprises the amino acid sequence of SEQ ID NO: 39, and the nucleocapsid phosphoprotein N comprises the amino acid sequence of SEQ ID NO: 44, or an amino acid sequence having at least about 90%, or at least about 95%, or at least about 97%, or at least about 98%, or at least about 99% identity with any of the foregoing, or an antigenic fragment of any of the foregoing, or a variant of any of the foregoing.

25. The expression vector system of any one of the previous claims, further comprising a nucleic acid encoding a bovine papillomavirus (BPV) E1 protein and/or a BPV E2 protein.

26. The expression vector system of any one of the previous claims, further comprising a nucleic acid encoding a BPV E1 protein having an amino acid sequence of SEQ ID NO: 19 and/or a BPV E2 protein having an amino acid sequence of SEQ ID NO: 22 or an amino acid sequence having at least about 90%, or at least about 95%, or at least about 97%, or at least about 98%, or at least about 99% identity thereto.

27. The expression vector system of any one of the previous claims, which does not comprise a nucleic acid encoding an E5 sequence, E6 sequence, E7 sequence.

28. The expression vector system of any one of the previous claims, comprising the nucleotide sequence of SEQ ID NO: 24 or SEQ ID NO: 25.

29. A host cell comprising the expression vector system of any one of the previous claims.

30. The host cell of claim 29, which is a mammalian host cell.

31. The host cell of claim 30, which is a human host cell.

32. The host cell of claim 31, which is an NIH 3T3 cell or an HEK 293 cell.

33. A population of cells wherein at least 50% of the cells are host cells according to any one of claims 29 to 32.

34. A composition comprising an expression vector system of any one of claims 1 to 28 or a host cell of any one of claims 29 to 32, or a population of cells of claim 33, and an excipient, carrier, or diluent.

35. The composition of claim 34, which is a sterile composition.

36. The composition of claim 34 or claim 35, which is suitable for administration to a human.

37. The composition of any one of claims 34 to 36, comprising at least or about 0.5×10⁶ cells transfected with the expression vector system, optionally comprising 0.5×10⁶ cells; and/or an effective amount of cells that express and/or secrete at least or about 500-1000 ng of secretable fusion protein, optionally gp96.

38. A kit comprising an expression vector system of any one of claims 1 to 28 or a host cell of any one of claims 29 to 32, or a population of cells of claim 33, or a composition of any one of claims 34 to 37.

39. A method of eliciting an immune response against coronavirus in a subject, comprising administering to the subject the expression vector of any one of claims 1 to 28, or a population of cells transfected with the expression vector.

40. A method of treating or preventing a coronavirus infection in a subject, comprising administering to the subject the expression vector of any one of claims 1 to 30, or a population of cells transfected with the expression vector.

41. The method of claim 39 or claim 40, wherein the coronavirus is a betacoronavirus protein or an alphacoronavirus protein, optionally wherein the betacoronavirus protein is selected from a SARS-CoV-2, SARS-CoV, MERS-CoV, HCoV-HKU1, and HCoV-OC43 protein, or an antigenic fragment thereof, or the alphacoronavirus protein is selected from a HCoV-NL63 and HCoV-229E protein, or an antigenic fragment thereof.

42. The method of claim 41, wherein the betacoronavirus protein is SARS-CoV-2 protein.

43. The method of claim 42, wherein the SARS-CoV-2 protein is a variant of a SARS-CoV-2 protein.

44. The method of claim 42 or claim 43, wherein the SARS-CoV-2 protein comprises an amino acid encoded by a nucleic acid having a nucleotide sequence of SEQ ID NO: 46, or an antigenic fragment thereof.

45. A method of treating or preventing a coronavirus infection in a subject, comprising administering to the subject an expression vector comprising the nucleic acid encoding the coronavirus protein, or an antigenic portion thereof that has a sequence having at least 90% identity with a nucleic acid having a nucleotide sequence of SEQ ID NO: 46, or a fragment thereof.

46. A method of treating or preventing a coronavirus infection in a subject, comprising administering to the subject an expression vector comprising the nucleic acid encoding the coronavirus protein, or an antigenic portion thereof that has a sequence having at least 95% identity with a nucleic acid having a nucleotide sequence of SEQ ID NO: 46, or a fragment thereof.

47. An expression vector system comprising (i) a nucleic acid encoding the amino acid sequence of SEQ ID NO: 2 and (ii) a nucleic acid encoding the amino acid sequence of SEQ ID NO: 37, a nucleic acid encoding the amino acid sequence of SEQ ID NO: 40, a nucleic acid encoding the amino acid sequence of SEQ ID NO: 39, a nucleic acid encoding the amino acid sequence of SEQ ID NO: 44, or an amino acid sequence having at least about 90%, or at least about 95%, or at least about 97%, or at least about 98%, or at least about 99% identity with any of the foregoing, or an antigenic fragment of any of the foregoing; wherein each nucleic acid is operably linked to a promoter.

48. The expression vector system of claim 47, wherein SEQ ID NO: 2 lacks the terminal KDEL sequence (SEQ ID NO: 49).

49. A method of treating or preventing a coronavirus infection in a subject, comprising administering to the subject the expression vector of claim 47 or claim 48.

50. A biological cell comprising a first recombinant protein having an amino acid sequence of at least 95% sequence identity with SEQ ID NO: 2 and a second recombinant protein having an amino acid sequence of at least 95% sequence identity with the amino acid sequence of SEQ ID NO: 37, the amino acid sequence of SEQ ID NO: 40, the amino acid sequence of SEQ ID NO: 39, or the amino acid sequence of SEQ ID NO: 44, or an antigenic fragment of any of the foregoing.

51. The biological cell of claim 50, wherein the first recombinant protein has at least 97% sequence identity with SEQ ID NO: 2 and the second recombinant protein having an amino acid sequence of at least 97% sequence identity with the amino acid sequence of SEQ ID NO: 37, the amino acid sequence of SEQ ID NO: 40, the amino acid sequence of SEQ ID NO: 39, or the amino acid sequence of SEQ ID NO: 44, or an antigenic fragment of any of the foregoing.

52. The biological cell of claim 50, wherein the first recombinant protein has at least 98% sequence identity with SEQ ID NO: 2 and the second recombinant protein having an amino acid sequence of at least 98% sequence identity with the amino acid sequence of SEQ ID NO: 37, the amino acid sequence of SEQ ID NO: 40, the amino acid sequence of SEQ ID NO: 39, or the amino acid sequence of SEQ ID NO: 44, or an antigenic fragment of any of the foregoing.

53. The biological cell of any one of claims 50 to 52, wherein SEQ ID NO: 2 lacks the terminal KDEL sequence (SEQ ID NO: 49).

54. A method of treating or preventing a coronavirus infection in a subject, comprising administering to the subject the biological cell of any one of claims 50 to 53.

55. A composition comprising a biological cell comprising an expression vector system comprising one or more:
(i) a nucleic acid encoding a secretable fusion protein comprising a chaperone protein and an immunoglobulin, or a fragment thereof,
(ii) a nucleic acid encoding a T cell costimulatory fusion protein, wherein the T cell costimulatory fusion protein enhances activation of antigen-specific T cells when administered to a subject; and
(iii) a nucleic acid encoding a coronavirus protein, or an antigenic portion thereof, wherein each nucleic acid is operably linked to a promoter.

56. The composition of claim 55, wherein the chaperone protein of the secretable fusion protein is a secretable gp96-Ig fusion protein which optionally lacks the gp96 KDEL sequence.

57. The composition of claim 56, wherein the immunoglobulin comprises a Ig tag of the gp96-Ig fusion protein comprising the Fc region of human IgG1, IgG2, IgG3, IgG4, IgM, IgA, or IgE.

58. The composition of claim 55, wherein the nucleic acid encoding the secretable fusion protein is operably linked to a promoter which is different from a promoter which is operably linked to the nucleic acid encoding the coronavirus protein, or an antigenic portion thereof.

59. The composition of claim 56, wherein the nucleic acid encoding the secretable fusion protein is operably linked to a CMV promoter.

60. The composition of any one of claims 55 to 59, wherein the nucleic acid encoding the coronavirus protein, or an antigenic portion thereof, is operably linked to an Mth promoter.

61. The composition of any one of claims 55 to 60, wherein the nucleic acid encoding the secretable fusion protein and the nucleic acid encoding the coronavirus protein, or antigenic portion thereof, are present on the same expression vector.

62. The composition of any one of claims 55 to 61, wherein the nucleic acid encoding the fusion protein is present on an expression vector which is different from the expression vector comprising the nucleic acid encoding the coronavirus protein, or antigenic portion thereof.

63. The composition of any one of claims 55 to 62, comprising two or more nucleic acids each encoding a different coronavirus protein, or an antigenic portion thereof.

64. The composition of any one of claims 55 to 63, wherein the chaperone protein is selected from the group consisting of: gp96, Hsp70, BiP, and Grp78.

65. The composition of any one of claims 55 to 64, wherein the T cell costimulatory fusion protein is OX40L-Ig, or a portion thereof that binds to OX40.

66. The composition of any one of claims 55 to 65, wherein the T cell costimulatory fusion protein is selected from OX40L-Ig or a portion thereof that binds specifically to OX40, ICOSL-Ig or a portion thereof that binds specifically to ICOS, 4-1BBL-Ig, or a portion thereof that binds specifically to 4-1BBR, CD40L-Ig, or a portion thereof that binds specifically to CD40, CD70-Ig, or a portion thereof that binds specifically to CD27, TL1A-Ig or a portion thereof that binds specifically to TNFRSF25, or GITRL-Ig or a portion thereof that binds specifically to GITR.

67. The composition of any one of claims 55 to 66, wherein the chaperone protein comprises an amino acid sequence of any one of SEQ ID NOs: 2, 29, 30, and 31, or an amino acid sequence having at least about 90%, or at least about 95%, or at least about 97%, or at least about 98%, or at least about 99% identity thereto.

68. The composition of claim 67, wherein the chaperone protein is gp96 comprising the amino acid sequence of SEQ ID NO: 2.

69. The composition of any one of claims 55 to 68, wherein the fusion protein comprises an Fc fragment of an immunoglobulin.

70. The composition of claim 69, wherein the immunoglobulin is an IgG1 immunoglobulin.

71. The composition of claim 69 or claim 70, wherein the Fc fragment comprises the amino acid sequence of SEQ ID NO: 5, or an amino acid sequence having at least about 90%, or at least about 95%, or at least about 97%, or at least about 98%, or at least about 99% identity thereto.

72. The composition of any one of claims 55 to 71, wherein the fusion protein comprises the amino acid sequence of SEQ ID NO: 8, or an amino acid sequence having at least about 90%, or at least about 95%, or at least about 97%, or at least about 98%, or at least about 99% identity thereto.

73. The composition of any one of claims 55 to 72, wherein the coronavirus protein is a betacoronavirus protein or an alphacoronavirus protein, optionally wherein the betacoronavirus protein is selected from a SARS-CoV-2, SARS-CoV, MERS-CoV, HCoV-HKU1, and HCoV-OC43 protein, or an antigenic fragment thereof or the alphacoronavirus protein is selected from a HCoV-NL63 and HCoV-229E protein, or an antigenic fragment thereof.

74. The composition of claim 73, wherein the betacoronavirus protein is a SARS-CoV-2 protein.

75. The composition of claim 74, wherein the SARS-CoV-2 protein is a variant of a SARS-CoV-2 protein.

76. The composition of claim 74 or claim 75, wherein the SARS-CoV-2 protein comprises an amino acid encoded by a nucleic acid having a nucleotide sequence of SEQ ID NO: 46, or an antigenic fragment thereof.

77. The composition of any one of claims 55 to 76, wherein the coronavirus protein is a SARS-CoV-2 protein, or an antigenic fragment thereof selected from spike surface glycoprotein, membrane glycoprotein M, envelope protein E, and nucleocapsid phosphoprotein N.

78. The composition of claim 77, wherein the spike surface glycoprotein comprises the amino acid sequence of SEQ ID NO: 37, membrane glycoprotein precursor M comprises the amino acid sequence of SEQ ID NO: 40, the envelope protein E comprises the amino acid sequence of SEQ ID NO: 39, and the nucleocapsid phosphoprotein N comprises the amino acid sequence of SEQ ID NO: 44, or an amino acid sequence having at least about 90%, or at least about 95%, or at least about 97%, or at least about 98%, or at least about 99% identity with any of the foregoing, or an antigenic fragment of any of the foregoing, or a variant of any of the foregoing.

79. The composition of any one of claims 55 to 78, further comprising a nucleic acid encoding a bovine papillomavirus (BPV) E1 protein and/or a BPV E2 protein.

80. The composition of any one of claims 55 to 79, further comprising a nucleic acid encoding a BPV E1 protein having an amino acid sequence of SEQ ID NO: 19 and/or a BPV E2 protein having an amino acid sequence of SEQ ID NO: 22 or an amino acid sequence having at least about 90%, or at least about 95%, or at least about 97%, or at least about 98%, or at least about 99% identity thereto.

81. The composition of any one of claims 55 to 80, which does not comprise a nucleic acid encoding an E5 sequence, E6 sequence, E7 sequence.

82. The composition of any one of claims 55 to 81, wherein the expression vector system comprises the nucleotide sequence of SEQ ID NO: 24 or SEQ ID NO: 25.

83. The composition of any one of claims 55 to 82, which is a sterile composition.

84. The composition of any one of claims 55 to 83, which is suitable for administration to a human.

85. The composition of any one of claims 55 to 84, comprising at least or about 0.5×10⁶ cells transfected with the expression vector system, optionally comprising 0.5×10⁶ cells; and/or an effective amount of cells that express and/or secrete at least or about 500-1000 ng of secretable fusion protein, optionally gp96.

86. The composition of any one of claims 55 to 84, comprising at least 0.5×10⁶ cells transfected with the expression vector system.

87. The composition of any one of claims 55 to 84, comprising about 0.5×10⁶ cells transfected with the expression vector system.

88. The composition of any one of claims 55 to 84, comprising an effective amount of cells that express and/or secrete at least 500 ng of secretable fusion protein, optionally gp96.

89. The composition of any one of claims 55 to 84, comprising an effective amount of cells that express and/or secrete about 500 ng of secretable fusion protein, optionally gp96.

90. A method of eliciting an immune response against coronavirus in a subject, comprising administering to the subject the composition of any one of claims 55 to 89.

91. A method of treating or preventing a coronavirus infection in a subject, comprising administering to the subject the composition of any one of claims 55 to 89.

92. The method of claim 90 or claim 91, wherein the coronavirus is a betacoronavirus protein or an alphacoronavirus protein, optionally wherein the betacoronavirus protein is selected from a SARS-CoV-2, SARS-CoV, MERS-CoV, HCoV-HKU1, and HCoV-OC43 protein, or an antigenic fragment thereof, or the alphacoronavirus protein is selected from a HCoV-NL63 and HCoV-229E protein, or an antigenic fragment thereof.

93. The method of claim 92, wherein the betacoronavirus protein is SARS-CoV-2 protein.

94. The method of claim 93, wherein the SARS-CoV-2 protein is a variant of SARS-CoV-2 protein.

95. The method of claim 92 or claim 93, wherein the SARS-CoV-2 protein comprises an amino acid encoded by a nucleic acid having a nucleotide sequence of SEQ ID NO: 46, or an antigenic fragment thereof.

96. The method of any one of claims 90 to 95, wherein the composition comprises at least or about 0.5×10⁶ cells transfected with the expression vector system, optionally comprising 0.5×10⁶ cells; and/or an effective amount of cells that express and/or secrete at least or about 500-1000 ng of secretable fusion protein, optionally gp96.

97. A composition having a biological cell comprising an expression vector system, the expression vector system comprising:
(i) a nucleic acid encoding a secretable fusion protein comprising a chaperone protein and an immunoglobulin, or a fragment thereof; and/or
(ii) a nucleic acid encoding a T cell costimulatory fusion protein, wherein the T cell costimulatory fusion protein enhances activation of antigen-specific T cells when administered to a subject; and
(iii) a nucleic acid encoding a coronavirus protein, or an antigenic portion thereof, wherein each nucleic acid is operably linked to a promoter.

98. The composition of claim 97, wherein the composition comprises a single biological cell.

99. The composition of claim 97, wherein the T cell costimulatory fusion protein is optionally OX40L, and wherein the composition comprises two or more biological cells, wherein a biological cell of the two or more biological cells optionally encodes
a nucleic acid encoding a secretable fusion protein comprising a chaperone protein and an immunoglobulin, or a fragment thereof, and
a nucleic acid encoding a coronavirus protein, or an antigenic portion thereof.

100. A method of vaccinating against SARS-CoV-2 infection comprising administering a composition to a patient in need thereof, the composition having a biological cell comprising an expression vector system, the expression vector system comprising one or more:
(i) a nucleic acid encoding a secretable fusion protein comprising a chaperone protein and an immunoglobulin, or a fragment thereof,
(ii) a nucleic acid encoding a T cell costimulatory fusion protein, wherein the T cell costimulatory fusion protein enhances activation of antigen-specific T cells when administered to a subject; and
(iii) a nucleic acid encoding a coronavirus protein, or an antigenic portion thereof, wherein each nucleic acid is operably linked to a promoter.

101. The method of claim 100, wherein the chaperone protein of the secretable fusion protein is a secretable gp96-Ig fusion protein which optionally lacks the gp96 KDEL sequence.

102. The method of claim 101, wherein the immunoglobulin comprises a Ig tag of the gp96-Ig fusion protein comprising the Fc region of human IgG1, IgG2, IgG3, IgG4, IgM, IgA, or IgE.

103. The method of claim 100, wherein the nucleic acid encoding the secretable fusion protein is operably linked to a promoter which is different from a promoter which is operably linked to the nucleic acid encoding the coronavirus protein, or an antigenic portion thereof.

104. The method of claim 101, wherein the nucleic acid encoding the secretable fusion protein is operably linked to a CMV promoter.

105. The method of any one of claims 100 to 104, wherein the nucleic acid encoding the coronavirus protein, or an antigenic portion thereof, is operably linked to an Mth promoter.

106. The method of any one of claims 100 to 105, wherein the nucleic acid encoding the secretable fusion protein and the nucleic acid encoding the coronavirus protein, or antigenic portion thereof, are present on the same expression vector.

107. The method of any one of claims 100 to 106, wherein the nucleic acid encoding the fusion protein is present on an expression vector which is different from the expression vector comprising the nucleic acid encoding the coronavirus protein, or antigenic portion thereof.

108. The method of any one of claims 100 to 107, comprising two or more nucleic acids each encoding a different coronavirus protein, or an antigenic portion thereof.

109. The method of any one of claims 100 to 108, wherein the chaperone protein is selected from the group consisting of: gp96, Hsp70, BiP, and Grp78.

110. The method of any one of claims 100 to 109, wherein the T cell costimulatory fusion protein is OX40L-Ig, or a portion thereof that binds to OX40.

111. The method of any one of claims 100 to 110, wherein the T cell costimulatory fusion protein is selected from OX40L-Ig or a portion thereof that binds specifically to OX40, ICOSL-Ig or a portion thereof that binds specifically to ICOS, 4-1BBL-Ig, or a portion thereof that binds specifically to 4-1BBR, CD40L-Ig, or a portion thereof that binds specifically to CD40, CD70-Ig, or a portion thereof that binds specifically to CD27, TL1A-Ig or a portion thereof that binds specifically to TNFRSF25, or GITRL-Ig or a portion thereof that binds specifically to GITR.

112. The method of any one of claims 100 to 111, wherein the chaperone protein comprises an amino acid sequence of any one of SEQ ID NOs: 2, 29, 30, and 31, or an amino acid sequence having at least about 90%, or at least about 95%, or at least about 97%, or at least about 98%, or at least about 99% identity thereto.

113. The method of claim 112, wherein the chaperone protein is gp96 comprising the amino acid sequence of SEQ ID NO: 2.

114. The method of any one of claims 100 to 113, wherein the fusion protein comprises an Fc fragment of an immunoglobulin.

115. The method of claim 114, wherein the immunoglobulin is an IgG1 immunoglobulin.

116. The method of claim 114 or claim 115, wherein the Fc fragment comprises the amino acid sequence of SEQ ID NO: 5, or an amino acid sequence having at least about 90%, or at least about 95%, or at least about 97%, or at least about 98%, or at least about 99% identity thereto.

117. The method of any one of claims 100 to 116, wherein the fusion protein comprises the amino acid sequence of SEQ ID NO: 8, or an amino acid sequence having at least about 90%, or at least about 95%, or at least about 97%, or at least about 98%, or at least about 99% identity thereto.

118. The method of any one of claims 100 to 117, wherein the coronavirus protein is a betacoronavirus protein or an alphacoronavirus protein, optionally wherein the betacoronavirus protein is selected from a SARS-CoV-2, SARS-CoV, MERS-CoV, HCoV-HKU1, and HCoV-OC43 protein, or an antigenic fragment thereof or the alphacoronavirus protein is selected from a HCoV-NL63 and HCoV-229E protein, or an antigenic fragment thereof.

119. The method of claim 118, wherein the betacoronavirus protein is a SARS-CoV-2 protein.

120. The method of claim 119, wherein the SARS-CoV-2 protein is a variant of a SARS-CoV-2 protein.

121. The method of claim 119 or claim 120, wherein the SARS-CoV-2 protein comprises an amino acid encoded by a nucleic acid having a nucleotide sequence of SEQ ID NO: 46, or an antigenic fragment thereof.

122. The method of any one of claims 100 to 121, wherein the coronavirus protein is a SARS-CoV-2 protein, or an antigenic fragment thereof selected from spike surface glycoprotein, membrane glycoprotein M, envelope protein E, and nucleocapsid phosphoprotein N.

123. The method of claim 122, wherein the spike surface glycoprotein comprises the amino acid sequence of SEQ ID NO: 37, membrane glycoprotein precursor M comprises the amino acid sequence of SEQ ID NO: 40, the envelope protein E comprises the amino acid sequence of SEQ ID NO: 39, and the nucleocapsid phosphoprotein N comprises the amino acid sequence of SEQ ID NO: 44, or an amino acid sequence having at least about 90%, or at least about 95%, or at least about 97%, or at least about 98%, or at least about 99% identity with any of the foregoing, or an antigenic fragment of any of the foregoing, or a variant of any of the foregoing.

124. The method of any one of claims 100 to 123, further comprising a nucleic acid encoding a bovine papillomavirus (BPV) E1 protein and/or a BPV E2 protein.

125. The method of any one of claims 100 to 124, further comprising a nucleic acid encoding a BPV E1 protein having an amino acid sequence of SEQ ID NO: 19 and/or a BPV E2 protein having an amino acid sequence of SEQ ID NO: 22 or an amino acid sequence having at least about 90%, or at least about 95%, or at least about 97%, or at least about 98%, or at least about 99% identity thereto.

126. The method of any one of claims 100 to 125, which does not comprise a nucleic acid encoding an E5 sequence, E6 sequence, E7 sequence.

127. The method of any one of claims 100 to 126, wherein the expression vector system comprises the nucleotide sequence of SEQ ID NO: 24 or SEQ ID NO: 25.

128. The method of any one of claims 100 to 127, which is a sterile composition.

129. The method of any one of claims 100 to 128, which is suitable for administration to a human.

130. The method of claim 100, comprising administering the composition in combination with one or more additional vaccines.

131. The method of claim 130, wherein the one or more additional vaccines are selected from an mRNA vaccine encoding SARS-CoV-2 spike (S) protein, optionally LNP-encapsulated; a viral vector vaccine expressing the S protein, optionally a viral vector (ChAdOx1—chimpanzee adenovirus Oxford 1) vaccine (ChAdOx1 nCoV-19) expressing the S protein; an mRNA vaccine encoding an optimized SARS-CoV-2 receptor-binding domain (RBD); an mRNA vaccine encoding an optimized full-length S protein; Adenovirus type 5 vector that expresses the S protein; a plasmid encoding the S protein delivered by electroporation, optionally a DNA plasmid encoding the S protein delivered by electroporation; dendritic cells (DCs) modified with lentiviral vector expressing synthetic minigene based on domains of selected viral proteins, administered with antigen-specific cytotoxic T lymphocytes (CTLs); and artificial antigen-presenting cells (aAPCs) modified with lentiviral vector expressing synthetic minigene based on domains of selected viral proteins.

132. The method of any one of claims 100 to 131, wherein the composition induces a CD8+ T cell response in the patient.

133. The method of claim 132, wherein the composition induces the CD8+ T cell to target the immunodominant epitope of the SARS-CoV-2 spike (S) protein.

134. The method of any one of claims 100 to 131, wherein the composition induces a CD69+CD8+ T cell response in the patient.

135. The method of any one of claims 100 to 131, wherein the composition induces a CD4+ T cell response in the patient.

136. The method of claim 135, wherein the CD4+ T cell response in the patient releases antiviral cytokines.

137. The method of claim 136, wherein the antiviral cytokines are selected from IFNγ, TNF-α, and IL-2.

138. The method of any one of claims 100 to 137, wherein the composition induces the response in a lung and/or airway passage of the patient.

139. The method of any one of claims 100 to 138, wherein the composition induces cytotoxic CD8+ T-cell effector memory cells and resident memory T-cell responses.

140. The method of any one of claims 100 to 139, further comprising administering the composition as a single vaccination.

141. The method of any one of claims 100 to 131, wherein the composition induces a SARS-CoV-2, Spike protein specific CD4+ Th1 T-cell response.

142. The method of any one of claims 100 to 141, wherein the composition comprises at least or about 0.5×10⁶ cells transfected with the expression vector system, optionally comprising 0.5×10⁶ cells; and/or an effective amount of cells that express and/or secrete at least or about 500-1000 ng of secretable fusion protein, optionally gp96.

143. The expression vector system of any one of claims 1 to 28, wherein the coronavirus protein is selected from a plurality of variants of a coronavirus protein comprising B.1.1.7, B.1.351 (501Y.V2), B.1, B.1.1.28, B.1.2, CAL.20C, B.6, P.1 and P.2 variants, or antigenic fragments thereof.

144. The expression vector system of claim 22, wherein the SARS-CoV-2 protein comprises an amino acid sequence having at least one mutation relative to the amino acid sequence encoded by a nucleic acid having a nucleotide sequence of SEQ ID NO: 46 or an antigenic fragment thereof.

145. The expression vector system of claim 24, wherein the spike surface glycoprotein comprises an amino acid sequence having at least one mutation relative to the amino acid sequence of SEQ ID NO: 37 or an antigenic fragment thereof.

146. The expression vector system of claim 24, wherein the spike surface glycoprotein comprises an amino acid sequence having one or more of D614G, E484K, N501Y, K417N, S477G, and S477N mutations relative to the amino acid sequence of SEQ ID NO: 37 or an antigenic fragment thereof.

147. The composition of any one of claims 55 to 89, wherein the coronavirus protein is selected from a plurality of variants of a coronavirus protein comprising B.1.1.7, B.1.351 (501Y.V2), B.1, B.1.1.28, B.1.2, CAL.20C, B.6, P.1, and P.2 variants, or antigenic fragments thereof.

148. The composition of claim 76, wherein the SARS-CoV-2 protein comprises an amino acid sequence having at least one mutation relative to the amino acid sequence encoded by a nucleic acid having a nucleotide sequence of SEQ ID NO: 46, or an antigenic fragment thereof.

149. The composition of claim 78, wherein the spike surface glycoprotein comprises an amino acid sequence having at least one mutation relative to the amino acid sequence of SEQ ID NO: 37 or an antigenic fragment thereof.

150. The composition of claim 78, wherein the spike surface glycoprotein comprises an amino acid sequence having one or more of D614G, E484K, N501Y, K417N, S477G, and S477N mutations relative to the amino acid sequence of SEQ ID NO: 37 or an antigenic fragment thereof.

151. A method of eliciting an immune response against coronavirus in a subject, comprising administering to the subject a composition having a biological cell comprising an expression vector system, the expression vector system comprising:
(i) a nucleic acid encoding a secretable fusion protein comprising a gp96-Ig, or a fragment thereof,
(ii) a nucleic acid encoding a T cell costimulatory fusion protein, optionally OX40L, wherein the T cell costimulatory fusion protein enhances activation of antigen-specific T cells when administered to a subject; and
(iii) a nucleic acid encoding a coronavirus protein, or an antigenic portion thereof, wherein each nucleic acid is operably linked to a promoter.


SEQUENCE LISTING (98 sequences)

SEQ ID NO: 1 - DNA, 2170 bp, Artificial Sequence
  Synthetic Polymer
  CDS (26)..(2170)
ttggcaaagaattcgaagcctcgagatgatgaaacttatcatcaattcattgtataaaaataaagagattttcctgagagaactgatttcaaatgcttctgatgctttagataagataaggctaatatcactgactgatgaaaatgctctttctggaaatgaggaactaacagtcaaaattaagtgtgataaggagaagaacctgctgcatgtcacagacaccggtgtaggaatgaccagagaagagttggttaaaaaccttggtaccatagccaaatctgggacaagcgagtttttaaacaaaatgactgaagcacaggaagatggccagtcaacttctgaattgattggccagtttggtgtcggtttctattccgccttccttgtagcagataaggttattgtcacttcaaaacacaacaacgatacccagcacatctgggagtctgactccaatgaattttctgtaattgctgacccaagaggaaacactctaggacggggaacgacaattacccttgtcttaaaagaagaagcatctgattaccttgaattggatacaattaaaaatctcgtcaaaaaatattcacagttcataaactttcctatttatgtatggagcagcaagactgaaactgttgaggagcccatggaggaagaagaagcagccaaagaagagaaagaagaatctgatgatgaagctgcagtagaggaagaagaagaagaaaagaaaccaaagactaaaaaagttgaaaaaactgtctgggactgggaacttatgaatgatatcaaaccaatatggcagagaccatcaaaagaagtagaagaagatgaatacaaagctttctacaaatcattttcaaaggaaagtgatgaccccatggcttatattcactttactgctgaaggggaagttaccttcaaatcaattttatttgtacccacatctgctccacgtggtctgtttgacgaatatggatctaaaaagagcgattacattaagctctatgtgcgccgtgtattcatcacagacgacttccatgatatgatgcctaaatacctcaattttgtcaagggtgtggtggactcagatgatctccccttgaatgtttcccgcgagactcttcagcaacataaactgcttaaggtgattaggaagaagcttgttcgtaaaacgctggacatgatcaagaagattgctgatgataaatacaatgatactttttggaaagaatttggtaccaacatcaagcttggtgtgattgaagaccactcgaatcgaacacgtcttgctaaacttcttaggttccagtcttctcatcatccaactgacattactagcctagaccagtatgtggaaagaatgaaggaaaaacaagacaaaatctacttcatggctgggtccagcagaaaagaggctgaatcttctccatttgttgagcgacttctgaaaaagggctatgaagttatttacctcacagaacctgtggatgaatactgtattcaggcccttcccgaatttgatgggaagaggttccagaatgttgccaaggaaggagtgaagttcgatgaaagtgagaaaactaaggagagtcgtgaagcagttgagaaagaatttgagcctctgctgaattggatgaaagataaagcccttaaggacaagattgaaaaggctgtggtgtctcagcgcctgacagaatctccgtgtgctttggtggccagccagtacggatggtctggcaacatggagagaatcatgaaagcacaagcgtaccaaacgggcaaggacatctctacaaattactatgcgagtcagaagaaaacatttgaaattaatcccagacacccgctgatcagagacatgcttcgacgaattaaggaagatgaagatgataaaacagttttggatcttgctgtggttttgtttgaaacagcaacgcttcggtcagggtatcttttaccagacactaaagcatatggagatagaatagaaagaatgcttcgcctcagtttgaacattgaccctgatgcaaaggtggaagaagagcccgaagaagaacctgaagagacagcagaagacacaacagaagacacagagcaagacgaagatgaagaaatggatgtgggaacagatgaagaagaagaaacagcaaaggaatctacagctgaa

SEQ ID NO: 2 - protein, 803 aa, Artificial Sequence
  Synthetic Construct
MRALWVLGLCCVLLTFGSVRADDEVDVDGTVEEDLGKSREGSRTDDEVVQREEEAIQLDGLNASQIRELREKSEKFAFQAEVNRMMKLIINSLYKNKEIFLRELISNASDALDKIRLISLTDENALSGNEELTVKIKCDKEKNLLHVTDTGVGMTREELVKNLGTIAKSGTSEFLNKMTEAQEDGQSTSELIGQFGVGFYSAFLVADKVIVTSKHNNDTQHIWESDSNEFSVIADPRGNTLGRGTTITLVLKEEASDYLELDTIKNLVKKYSQFINFPIYVWSSKTETVEEPMEEEEAAKEEKEESDDEAAVEEEEEEKKPKTKKVEKTVWDWELMNDIKPIWQRPSKEVEEDEYKAFYKSFSKESDDPMAYIHFTAEGEVTFKSILFVPTSAPRGLFDEYGSKKSDYIKLYVRRVFITDDFHDMMPKYLNFVKGVVDSDDLPLNVSRETLQQHKLLKVIRKKLVRKTLDMIKKIADDKYNDTFWKEFGTNIKLGVIEDHSNRTRLAKLLRFQSSHHPTDITSLDQYVERMKEKQDKIYFMAGSSRKEAESSPFVERLLKKGYEVIYLTEPVDEYCIQALPEFDGKRFQNVAKEGVKFDESEKTKESREAVEKEFEPLLNWMKDKALKDKIEKAVVSQRLTESPCALVASQYGWSGNMERIMKAQAYQTGKDISTNYYASQKKTFEINPRHPLIRDMLRRIKEDEDDKTVLDLAVVLFETATLRSGYLLPDTKAYGDRIERMLRLSLNIDPDAKVEEEPEEEPEETAEDTTEDTEQDEDEEMDVGTDEEEETAKESTAEKDEL

SEQ ID NO: 3 - DNA, 2170 bp, Artificial Sequence
  Synthetic Polynucleotide
aaccgtttcttaagcttcggagctctactactttgaatagtagttaagtaacatatttttatttctctaaaaggactctcttgactaaagtttacgaagactacgaaatctattctattccgattatagtgactgactacttttacgagaaagacctttactccttgattgtcagttttaattcacactattcctcttcttggacgacgtacagtgtctgtggccacatccttactggtctcttctcaaccaatttttggaaccatggtatcggtttagaccctgttcgctcaaaaatttgttttactgacttcgtgtccttctaccggtcagttgaagacttaactaaccggtcaaaccacagccaaagataaggcggaaggaacatcgtctattccaataacagtgaagttttgtgttgttgctatgggtcgtgtagaccctcagactgaggttacttaaaagacattaacgactgggttctcctttgtgagatcctgccccttgctgttaatgggaacagaattttcttcttcgtagactaatggaacttaacctatgttaatttttagagcagttttttataagtgtcaagtatttgaaaggataaatacatacctcgtcgttctgactttgacaactcctcgggtacctccttcttcttcgtcggtttcttctctttcttcttagactactacttcgacgtcatctccttcttcttcttcttttctttggtttctgattttttcaacttttttgacagaccctgacccttgaatacttactatagtttggttataccgtctctggtagttttcttcatcttcttctacttatgtttcgaaagatgtttagtaaaagtttcctttcactactggggtaccgaatataagtgaaatgacgacttccccttcaatggaagtttagttaaaataaacatgggtgtagacgaggtgcaccagacaaactgcttatacctagatttttctcgctaatgtaattcgagatacacgcggcacataagtagtgtctgctgaaggtactatactacggatttatggagttaaaacagttcccacaccacctgagtctactagaggggaacttacaaagggcgctctgagaagtcgttgtatttgacgaattccactaatccttcttcgaacaagcattttgcgacctgtactagttcttctaacgactactatttatgttactatgaaaaacctttcttaaaccatggttgtagttcgaaccacactaacttctggtgagcttagcttgtgcagaacgatttgaagaatccaaggtcagaagagtagtaggttgactgtaatgatcggatctggtcatacacctttcttacttcctttttgttctgttttagatgaagtaccgacccaggtcgtcttttctccgacttagaagaggtaaacaactcgctgaagactttttcccgatacttcaataaatggagtgtcttggacacctacttatgacataagtccgggaagggcttaaactacccttctccaaggtcttacaacggttccttcctcacttcaagctactttcactcttttgattcctctcagcacttcgtcaactctttcttaaactcggagacgacttaacctactttctatttcgggaattcctgttctaacttttccgacaccacagagtcgcggactgtcttagaggcacacgaaaccaccggtcggtcatgcctaccagaccgttgtacctctcttagtactttcgtgttcgcatggtttgcccgttcctgtagagatgtttaatgatacgctcagtcttcttttgtaaactttaattagggtctgtgggcgactagtctctgtacgaagctgcttaattccttctacttctactattttgtcaaaacctagaacgacaccaaaacaaactttgtcgttgcgaagccagtcccatagaaaatggtctgtgatttcgtatacctctatcttatctttcttacgaagcggagtcaaacttgtaactgggactacgtttccaccttcttctcgggcttcttcttggacttctctgtcgtcttctgtgttgtcttctgtgtctcgttctgcttctacttctttacctacacccttgtctacttcttcttctttgtcgtttccttagatgtcgactt

SEQ ID NO: 4 - DNA, 690 bp, Artificial Sequence
  Synthetic Polymer
  CDS (7)..(690)
ggatccgtgcccagggattctggttctaagccttccatatctacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacaaaaccccgggaggagcagttcaacagcactttccgttcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatga

SEQ ID NO: 5 - protein, 227 aa, Artificial Sequence
  Synthetic Construct
VPRDSGSKPSISTVPEVSSVFIFPPKPKDVLTITLTPKVTCVVVDISKDDPEVQFSWFVDDVEVHTAQTKPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 6 - DNA, 690 bp, Artificial Sequence
  Synthetic Polynuleotide
cctaggcacgggtccctaagaccaagattcggaaggtatagatgtcagggtcttcatagtagacagaagtagaaggggggtttcgggttcctacacgagtggtaatgagactgaggattccagtgcacacaacaccatctgtagtcgttcctactagggctccaggtcaagtcgaccaaacatctactacacctccacgtgtgtcgagtctgttttggggccctcctcgtcaagttgtcgtgaaaggcaagtcagtcacttgaagggtagtacgtggtcctgaccgagttaccgttcctcaagtttacgtcccagttgtcacgtcgaaagggacgggggtagctcttttggtagaggttttggtttccgtctggcttccgaggtgtccacatgtggtaaggtggagggttcctcgtctaccggttcctatttcagtcagactggacgtactattgtctgaagaagggacttctgtaatgacacctcaccgtcaccttacccgtcggtcgcctcttgatgttcttgtgagtcgggtagtacctgtgtctaccgagaatgaagcagatgtcgttcgagttacacgtcttctcgttgaccctccgtcctttatgaaagtggacgagacacaatgtactcccggacgtgttggtggtatgactcttctcggagagggtgagaggaccatttact

SEQ ID NO: 7 - DNA, 2900 bp, Artificial Sequence
  Synthetic Polymer
  CDS (26)..(2857)
ttggcaaagaattcgaagcctcgagatgatgaaacttatcatcaattcattgtataaaaataaagagattttcctgagagaactgatttcaaatgcttctgatgctttagataagataaggctaatatcactgactgatgaaaatgctctttctggaaatgaggaactaacagtcaaaattaagtgtgataaggagaagaacctgctgcatgtcacagacaccggtgtaggaatgaccagagaagagttggttaaaaaccttggtaccatagccaaatctgggacaagcgagtttttaaacaaaatgactgaagcacaggaagatggccagtcaacttctgaattgattggccagtttggtgtcggtttctattccgccttccttgtagcagataaggttattgtcacttcaaaacacaacaacgatacccagcacatctgggagtctgactccaatgaattttctgtaattgctgacccaagaggaaacactctaggacggggaacgacaattacccttgtcttaaaagaagaagcatctgattaccttgaattggatacaattaaaaatctcgtcaaaaaatattcacagttcataaactttcctatttatgtatggagcagcaagactgaaactgttgaggagcccatggaggaagaagaagcagccaaagaagagaaagaagaatctgatgatgaagctgcagtagaggaagaagaagaagaaaagaaaccaaagactaaaaaagttgaaaaaactgtctgggactgggaacttatgaatgatatcaaaccaatatggcagagaccatcaaaagaagtagaagaagatgaatacaaagctttctacaaatcattttcaaaggaaagtgatgaccccatggcttatattcactttactgctgaaggggaagttaccttcaaatcaattttatttgtacccacatctgctccacgtggtctgtttgacgaatatggatctaaaaagagcgattacattaagctctatgtgcgccgtgtattcatcacagacgacttccatgatatgatgcctaaatacctcaattttgtcaagggtgtggtggactcagatgatctccccttgaatgtttcccgcgagactcttcagcaacataaactgcttaaggtgattaggaagaagcttgttcgtaaaacgctggacatgatcaagaagattgctgatgataaatacaatgatactttttggaaagaatttggtaccaacatcaagcttggtgtgattgaagaccactcgaatcgaacacgtcttgctaaacttcttaggttccagtcttctcatcatccaactgacattactagcctagaccagtatgtggaaagaatgaaggaaaaacaagacaaaatctacttcatggctgggtccagcagaaaagaggctgaatcttctccatttgttgagcgacttctgaaaaagggctatgaagttatttacctcacagaacctgtggatgaatactgtattcaggcccttcccgaatttgatgggaagaggttccagaatgttgccaaggaaggagtgaagttcgatgaaagtgagaaaactaaggagagtcgtgaagcagttgagaaagaatttgagcctctgctgaattggatgaaagataaagcccttaaggacaagattgaaaaggctgtggtgtctcagcgcctgacagaatctccgtgtgctttggtggccagccagtacggatggtctggcaacatggagagaatcatgaaagcacaagcgtaccaaacgggcaaggacatctctacaaattactatgcgagtcagaagaaaacatttgaaattaatcccagacacccgctgatcagagacatgcttcgacgaattaaggaagatgaagatgataaaacagttttggatcttgctgtggttttgtttgaaacagcaacgcttcggtcagggtatcttttaccagacactaaagcatatggagatagaatagaaagaatgcttcgcctcagtttgaacattgaccctgatgcaaaggtggaagaagagcccgaagaagaacctgaagagacagcagaagacacaacagaagacacagagcaagacgaagatgaagaaatggatgtgggaacagatgaagaagaagaaacagcaaaggaatctacagctgaaggatccgtgcccagggattctggttctaagccttccatatctacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacaaaaccccgggaggagcagttcaacagcactttccgttcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatgactcgacccagactagtcaaattaagccgaattctgcagat

SEQ ID NO: 8 - protein, 944 aa, Artificial Sequence
  Synthetic Construct
MMKLIINSLYKNKEIFLRELISNASDALDKIRLISLTDENALSGNEELTVKIKCDKEKNLLHVTDTGVGMTREELVKNLGTIAKSGTSEFLNKMTEAQEDGQSTSELIGQFGVGFYSAFLVADKVIVTSKHNNDTQHIWESDSNEFSVIADPRGNTLGRGTTITLVLKEEASDYLELDTIKNLVKKYSQFINFPIYVWSSKTETVEEPMEEEEAAKEEKEESDDEAAVEEEEEEKKPKTKKVEKTVWDWELMNDIKPIWQRPSKEVEEDEYKAFYKSFSKESDDPMAYIHFTAEGEVTFKSILFVPTSAPRGLFDEYGSKKSDYIKLYVRRVFITDDFHDMMPKYLNFVKGVVDSDDLPLNVSRETLQQHKLLKVIRKKLVRKTLDMIKKIADDKYNDTFWKEFGTNIKLGVIEDHSNRTRLAKLLRFQSSHHPTDITSLDQYVERMKEKQDKIYFMAGSSRKEAESSPFVERLLKKGYEVIYLTEPVDEYCIQALPEFDGKRFQNVAKEGVKFDESEKTKESREAVEKEFEPLLNWMKDKALKDKIEKAVVSQRLTESPCALVASQYGWSGNMERIMKAQAYQTGKDISTNYYASQKKTFEINPRHPLIRDMLRRIKEDEDDKTVLDLAVVLFETATLRSGYLLPDTKAYGDRIERMLRLSLNIDPDAKVEEEPEEEPEETAEDTTEDTEQDEDEEMDVGTDEEEETAKESTAEGSVPRDSGSKPSISTVPEVSSVFIFPPKPKDVLTITLTPKVTCVVVDISKDDPEVQFSWFVDDVEVHTAQTKPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 9 - DNA, 2900 bp, Artificial Sequence
  Synthetic Polynucleotide
aaccgtttcttaagcttcggagctctactactttgaatagtagttaagtaacatatttttatttctctaaaaggactctcttgactaaagtttacgaagactacgaaatctattctattccgattatagtgactgactacttttacgagaaagacctttactccttgattgtcagttttaattcacactattcctcttcttggacgacgtacagtgtctgtggccacatccttactggtctcttctcaaccaatttttggaaccatggtatcggtttagaccctgttcgctcaaaaatttgttttactgacttcgtgtccttctaccggtcagttgaagacttaactaaccggtcaaaccacagccaaagataaggcggaaggaacatcgtctattccaataacagtgaagttttgtgttgttgctatgggtcgtgtagaccctcagactgaggttacttaaaagacattaacgactgggttctcctttgtgagatcctgccccttgctgttaatgggaacagaattttcttcttcgtagactaatggaacttaacctatgttaatttttagagcagttttttataagtgtcaagtatttgaaaggataaatacatacctcgtcgttctgactttgacaactcctcgggtacctccttcttcttcgtcggtttcttctctttcttcttagactactacttcgacgtcatctccttcttcttcttcttttctttggtttctgattttttcaacttttttgacagaccctgacccttgaatacttactatagtttggttataccgtctctggtagttttcttcatcttcttctacttatgtttcgaaagatgtttagtaaaagtttcctttcactactggggtaccgaatataagtgaaatgacgacttccccttcaatggaagtttagttaaaataaacatgggtgtagacgaggtgcaccagacaaactgcttatacctagatttttctcgctaatgtaattcgagatacacgcggcacataagtagtgtctgctgaaggtactatactacggatttatggagttaaaacagttcccacaccacctgagtctactagaggggaacttacaaagggcgctctgagaagtcgttgtatttgacgaattccactaatccttcttcgaacaagcattttgcgacctgtactagttcttctaacgactactatttatgttactatgaaaaacctttcttaaaccatggttgtagttcgaaccacactaacttctggtgagcttagcttgtgcagaacgatttgaagaatccaaggtcagaagagtagtaggttgactgtaatgatcggatctggtcatacacctttcttacttcctttttgttctgttttagatgaagtaccgacccaggtcgtcttttctccgacttagaagaggtaaacaactcgctgaagactttttcccgatacttcaataaatggagtgtcttggacacctacttatgacataagtccgggaagggcttaaactacccttctccaaggtcttacaacggttccttcctcacttcaagctactttcactcttttgattcctctcagcacttcgtcaactctttcttaaactcggagacgacttaacctactttctatttcgggaattcctgttctaacttttccgacaccacagagtcgcggactgtcttagaggcacacgaaaccaccggtcggtcatgcctaccagaccgttgtacctctcttagtactttcgtgttcgcatggtttgcccgttcctgtagagatgtttaatgatacgctcagtcttcttttgtaaactttaattagggtctgtgggcgactagtctctgtacgaagctgcttaattccttctacttctactattttgtcaaaacctagaacgacaccaaaacaaactttgtcgttgcgaagccagtcccatagaaaatggtctgtgatttcgtatacctctatcttatctttcttacgaagcggagtcaaacttgtaactgggactacgtttccaccttcttctcgggcttcttcttggacttctctgtcgtcttctgtgttgtcttctgtgtctcgttctgcttctacttctttacctacacccttgtctacttcttcttctttgtcgtttccttagatgtcgacttcctaggcacgggtccctaagaccaagattcggaaggtatagatgtcagggtcttcatagtagacagaagtagaaggggggtttcgggttcctacacgagtggtaatgagactgaggattccagtgcacacaacaccatctgtagtcgttcctactagggctccaggtcaagtcgaccaaacatctactacacctccacgtgtgtcgagtctgttttggggccctcctcgtcaagttgtcgtgaaaggcaagtcagtcacttgaagggtagtacgtggtcctgaccgagttaccgttcctcaagtttacgtcccagttgtcacgtcgaaagggacgggggtagctcttttggtagaggttttggtttccgtctggcttccgaggtgtccacatgtggtaaggtggagggttcctcgtctaccggttcctatttcagtcagactggacgtactattgtctgaagaagggacttctgtaatgacacctcaccgtcaccttacccgtcggtcgcctcttgatgttcttgtgagtcgggtagtacctgtgtctaccgagaatgaagcagatgtcgttcgagttacacgtcttctcgttgaccctccgtcctttatgaaagtggacgagacacaatgtactcccggacgtgttggtggtatgactcttctcggagagggtgagaggaccatttactgagctgggtctgatcagtttaattcggcttaagacgtcta

SEQ ID NO: 10-17 - skipped (8 entries)

SEQ ID NO: 18 - DNA, 1818 bp, Artificial Sequence
  Synthetic Polymer
  CDS (1)..(1818)
atggcaaacgataaaggtagcaattgggattcgggcttgggatgctcatatctgctgactgaggcagaatgtgaaagtgacaaagagaatgaggaacccggggcaggtgtagaactgtctgtggaatctgatcggtatgatagccaggatgaggattttgttgacaatgcatcagtctttcagggaaatcacctggaggtcttccaggcattagagaaaaaggcgggtgaggagcagattttaaatttgaaaagaaaagtattggggagttcgcaaaacagcagcggttccgaagcatctgaaactccagttaaaagacggaaatcaggagcaaagcgaagattatttgctgaaaatgaagctaaccgtgttcttacgcccctccaggtacagggggagggggaggggaggcaagaacttaatgaggagcaggcaattagtcatctacatctgcagcttgttaaatctaaaaatgctacagtttttaagctggggctctttaaatctttgttcctttgtagcttccatgatattacgaggttgtttaagaatgataagaccactaatcagcaatgggtgctggctgtgtttggccttgcagaggtgttttttgaggcgagtttcgaactcctaaagaagcagtgtagttttctgcagatgcaaaaaagatctcatgaaggaggaacttgtgcagtttacttaatctgctttaacacagctaaaagcagagaaacagtccggaatctgatggcaaacatgctaaatgtaagagaagagtgtttgatgctgcagccacctaaaattcgaggactcagcgcagctctattctggtttaaaagtagtttgtcacccgctacacttaaacatggtgctttacctgagtggatacgggcgcaaactactctgaacgagagcttgcagaccgagaaattcgacttcggaactatggtgcaatgggcctatgatcacaaatatgctgaggagtctaaaatagcctatgaatatgctttggctgcaggatctgatagcaatgcacgggcttttttagcaactaacagccaagctaagcatgtgaaggactgtgcaactatggtaagacactatctaagagctgaaacacaagcattaagcatgcctgcatatattaaagctaggtgcaagctggcaactggggaaggaagctggaagtctatcctaactttttttaactatcagaatattgaattaattacctttattaatgctttaaagctctggctaaaaggaattccaaaaaaaaactgtttagcatttattggccctccaaacacaggcaagtctatgctctgcaactcattaattcattttttgggtggtagtgttttatcttttgccaaccataaaagtcacttttggcttgcttccctagcagatactagagctgctttagtagatgatgctactcatgcttgctggaggtactttgacacatacctcagaaatgcattggatggctaccctgtcagtattgatagaaaacacaaagcagcggttcaaattaaagctccacccctcctggtaaccagtaatattgatgtgcaggcagaggacagatatttgtacttgcatagtcgggtgcaaacctttcgctttgagcagccatgcacagatgaatcgggtgagcaaccttttaatattactgatgcagattggaaatctttttttgtaaggttatgggggcgtttagacctgattgacgaggaggaggatagtgaagaggatggagacagcatgcgaacgtttacatgcagcgcaagaaacacaaatgcagttgattga

SEQ ID NO: 19 - protein, 605 aa, Artificial Sequence
  Synthetic Construct
MANDKGSNWDSGLGCSYLLTEAECESDKENEEPGAGVELSVESDRYDSQDEDFVDNASVFQGNHLEVFQALEKKAGEEQILNLKRKVLGSSQNSSGSEASETPVKRRKSGAKRRLFAENEANRVLTPLQVQGEGEGRQELNEEQAISHLHLQLVKSKNATVFKLGLFKSLFLCSFHDITRLFKNDKTTNQQWVLAVFGLAEVFFEASFELLKKQCSFLQMQKRSHEGGTCAVYLICFNTAKSRETVRNLMANMLNVREECLMLQPPKIRGLSAALFWFKSSLSPATLKHGALPEWIRAQTTLNESLQTEKFDFGTMVQWAYDHKYAEESKIAYEYALAAGSDSNARAFLATNSQAKHVKDCATMVRHYLRAETQALSMPAYIKARCKLATGEGSWKSILTFFNYQNIELITFINALKLWLKGIPKKNCLAFIGPPNTGKSMLCNSLIHFLGGSVLSFANHKSHFWLASLADTRAALVDDATHACWRYFDTYLRNALDGYPVSIDRKHKAAVQIKAPPLLVTSNIDVQAEDRYLYLHSRVQTFRFEQPCTDESGEQPFNITDADWKSFFVRLWGRLDLIDEEEDSEEDGDSMRTFTCSARNTNAVD

SEQ ID NO: 20 - DNA, 1818 bp, Artificial Sequence
  Synthetic Polynucleotide
taccgtttgctatttccatcgttaaccctaagcccgaaccctacgagtatagacgactgactccgtcttacactttcactgtttctcttactccttgggccccgtccacatcttgacagacaccttagactagccatactatcggtcctactcctaaaacaactgttacgtagtcagaaagtccctttagtggacctccagaaggtccgtaatctctttttccgcccactcctcgtctaaaatttaaacttttcttttcataacccctcaagcgttttgtcgtcgccaaggcttcgtagactttgaggtcaattttctgcctttagtcctcgtttcgcttctaataaacgacttttacttcgattggcacaagaatgcggggaggtccatgtccccctccccctcccctccgttcttgaattactcctcgtccgttaatcagtagatgtagacgtcgaacaatttagatttttacgatgtcaaaaattcgaccccgagaaatttagaaacaaggaaacatcgaaggtactataatgctccaacaaattcttactattctggtgattagtcgttacccacgaccgacacaaaccggaacgtctccacaaaaaactccgctcaaagcttgaggatttcttcgtcacatcaaaagacgtctacgttttttctagagtacttcctccttgaacacgtcaaatgaattagacgaaattgtgtcgattttcgtctctttgtcaggccttagactaccgtttgtacgatttacattctcttctcacaaactacgacgtcggtggattttaagctcctgagtcgcgtcgagataagaccaaattttcatcaaacagtgggcgatgtgaatttgtaccacgaaatggactcacctatgcccgcgtttgatgagacttgctctcgaacgtctggctctttaagctgaagccttgataccacgttacccggatactagtgtttatacgactcctcagattttatcggatacttatacgaaaccgacgtcctagactatcgttacgtgcccgaaaaaatcgttgattgtcggttcgattcgtacacttcctgacacgttgataccattctgtgatagattctcgactttgtgttcgtaattcgtacggacgtatataatttcgatccacgttcgaccgttgaccccttccttcgaccttcagataggattgaaaaaaattgatagtcttataacttaattaatggaaataattacgaaatttcgagaccgattttccttaaggtttttttttgacaaatcgtaaataaccgggaggtttgtgtccgttcagatacgagacgttgagtaattaagtaaaaaacccaccatcacaaaatagaaaacggttggtattttcagtgaaaaccgaacgaagggatcgtctatgatctcgacgaaatcatctactacgatgagtacgaacgacctccatgaaactgtgtatggagtctttacgtaacctaccgatgggacagtcataactatcttttgtgtttcgtcgccaagtttaatttcgaggtggggaggaccattggtcattataactacacgtccgtctcctgtctataaacatgaacgtatcagcccacgtttggaaagcgaaactcgtcggtacgtgtctacttagcccactcgttggaaaattataatgactacgtctaacctttagaaaaaaacattccaatacccccgcaaatctggactaactgctcctcctcctatcacttctcctacctctgtcgtacgcttgcaaatgtacgtcgcgttctttgtgtttacgtcaactaact

SEQ ID NO: 21 - DNA, 567 bp, Artificial Sequence
  Synthetic Polymer
  CDS (4)..(567)
aggatggagacagcatgcgaacgtttacatgcagcgcaagaaacacaaatgcagttgattgagaaaagtagtgataagttgcaagatcatatactgtactggactgctgttagaactgagaacacactgctttatgctgcaaggaaaaaaggggtgactgtcctaggacactgcagagtaccacactctgtagtttgtcaagagagagccaagcaggccattgaaatgcagttgtctttgcaggagttaagcaaaactgagtttggggatgaaccatggtctttgcttgacacaagctgggaccgatatatgtcagaacctaaacggtgctttaagaaaggcgccagggtggtagaggtggagtttgatggaaatgcaagcaatacaaactggtacactgtctacagcaatttgtacatgcgcacagaggacggctggcagcttgcgaaggctgggctgacggaactgggctctactactgcaccatggccggtgctggacgcatttactattctcgctttggtgacgaggcagccagatttagtacaacagggcattactctgtaa

SEQ ID NO: 22 - protein, 187 aa, Artificial Sequence
  Synthetic Construct
METACERLHAAQETQMQLIEKSSDKLQDHILYWTAVRTENTLLYAARKKGVTVLGHCRVPHSVVCQERAKQAIEMQLSLQELSKTEFGDEPWSLLDTSWDRYMSEPKRCFKKGARVVEVEFDGNASNTNWYTVYSNLYMRTEDGWQLAKAGLTELGSTTAPWPVLDAFTILALVTRQPDLVQQGITL

SEQ ID NO: 23 - DNA, 567 bp, Artificial Sequence
  Synthetic Polynucleotide
tcctacctctgtcgtacgcttgcaaatgtacgtcgcgttctttgtgtttacgtcaactaactcttttcatcactattcaacgttctagtatatgacatgacctgacgacaatcttgactcttgtgtgacgaaatacgacgttccttttttccccactgacaggatcctgtgacgtctcatggtgtgagacatcaaacagttctctctcggttcgtccggtaactttacgtcaacagaaacgtcctcaattcgttttgactcaaacccctacttggtaccagaaacgaactgtgttcgaccctggctatatacagtcttggatttgccacgaaattctttccgcggtcccaccatctccacctcaaactacctttacgttcgttatgtttgaccatgtgacagatgtcgttaaacatgtacgcgtgtctcctgccgaccgtcgaacgcttccgacccgactgccttgacccgagatgatgacgtggtaccggccacgacctgcgtaaatgataagagcgaaaccactgctccgtcggtctaaatcatgttgtcccgtaatgagacatt

SEQ ID NO: 24 - DNA, 16105 bp, Artificial Sequence
  Synthetic Polynucleotide
tctagagagcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggtcgatcgaccgatcctgagaacttcagggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacaccctggtcatcatcctgcctttctctttatggttacaatgatatacactgtttgagatgaggataaaatactctgagtccaaaccgggcccctctgctaaccatgttcatgccttcttctctttcctacagctcctgggcaacgtgctggttgttgtgctgtctcatcattttggcaaagaattcgaagcctcgagatgatgaaacttatcatcaattcattgtataaaaataaagagattttcctgagagaactgatttcaaatgcttctgatgctttagataagataaggctaatatcactgactgatgaaaatgctctttctggaaatgaggaactaacagtcaaaattaagtgtgataaggagaagaacctgctgcatgtcacagacaccggtgtaggaatgaccagagaagagttggttaaaaaccttggtaccatagccaaatctgggacaagcgagtttttaaacaaaatgactgaagcacaggaagatggccagtcaacttctgaattgattggccagtttggtgtcggtttctattccgccttccttgtagcagataaggttattgtcacttcaaaacacaacaacgatacccagcacatctgggagtctgactccaatgaattttctgtaattgctgacccaagaggaaacactctaggacggggaacgacaattacccttgtcttaaaagaagaagcatctgattaccttgaattggatacaattaaaaatctcgtcaaaaaatattcacagttcataaactttcctatttatgtatggagcagcaagactgaaactgttgaggagcccatggaggaagaagaagcagccaaagaagagaaagaagaatctgatgatgaagctgcagtagaggaagaagaagaagaaaagaaaccaaagactaaaaaagttgaaaaaactgtctgggactgggaacttatgaatgatatcaaaccaatatggcagagaccatcaaaagaagtagaagaagatgaatacaaagctttctacaaatcattttcaaaggaaagtgatgaccccatggcttatattcactttactgctgaaggggaagttaccttcaaatcaattttatttgtacccacatctgctccacgtggtctgtttgacgaatatggatctaaaaagagcgattacattaagctctatgtgcgccgtgtattcatcacagacgacttccatgatatgatgcctaaatacctcaattttgtcaagggtgtggtggactcagatgatctccccttgaatgtttcccgcgagactcttcagcaacataaactgcttaaggtgattaggaagaagcttgttcgtaaaacgctggacatgatcaagaagattgctgatgataaatacaatgatactttttggaaagaatttggtaccaacatcaagcttggtgtgattgaagaccactcgaatcgaacacgtcttgctaaacttcttaggttccagtcttctcatcatccaactgacattactagcctagaccagtatgtggaaagaatgaaggaaaaacaagacaaaatctacttcatggctgggtccagcagaaaagaggctgaatcttctccatttgttgagcgacttctgaaaaagggctatgaagttatttacctcacagaacctgtggatgaatactgtattcaggcccttcccgaatttgatgggaagaggttccagaatgttgccaaggaaggagtgaagttcgatgaaagtgagaaaactaaggagagtcgtgaagcagttgagaaagaatttgagcctctgctgaattggatgaaagataaagcccttaaggacaagattgaaaaggctgtggtgtctcagcgcctgacagaatctccgtgtgctttggtggccagccagtacggatggtctggcaacatggagagaatcatgaaagcacaagcgtaccaaacgggcaaggacatctctacaaattactatgcgagtcagaagaaaacatttgaaattaatcccagacacccgctgatcagagacatgcttcgacgaattaaggaagatgaagatgataaaacagttttggatcttgctgtggttttgtttgaaacagcaacgcttcggtcagggtatcttttaccagacactaaagcatatggagatagaatagaaagaatgcttcgcctcagtttgaacattgaccctgatgcaaaggtggaagaagagcccgaagaagaacctgaagagacagcagaagacacaacagaagacacagagcaagacgaagatgaagaaatggatgtgggaacagatgaagaagaagaaacagcaaaggaatctacagctgaaggatcctgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgactcgacccagactagtcaaattaagccgaattctgcagatatccatcacactggcggccgctggaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacggatccctagagtcgaggcgatgcggcgcagcaccatggcctgaaataacctctgaaagaggaacttggttaggtaccttggtttttaaaaccagcctggagtagagcagatgggttaaggtgagtgacccctcagccctggacattcttagatgagccccctcaggagtagagaataatgttgagatgagttctgttggctaaaataatcaaggctagtctttataaaactgtctcctcttctcctagcttcgatccagagagagacctgggcggagctggtcgctgctcaggaactccaggaaaggagaagctgaggttaccacgctgcgaatgggtttacggagatagctggctttccggggtgagttctcgtaaactccagagcagcgataggccgtaatatcggggaaagcactatagggacatgatgttccacacgtcacatgggtcgtcctatccgagccagtcgtgccaaaggggcggtcccgctgtgcacactggcgctccagggagctctgcactccgcccgaaaagtgcgctcggctctgccaggacgcggggcgcgtgactatgcgtgggctggagcaaccgcctgctgggtgcaaaccctttgcgcccggactcgtccaacgactataaagagggcaggctgtcctctaagcgtcaccacgacttcaacgtcctgagtaccttctcctcacttactccgtagctccagcttcaccaccaagctcctcgacgtcgatcgcgaagctttggcccctttggccttagcgtcgaccgatcctgagaacttcagggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacaccctggtcatcatcctgcctttctctttatggttacaatgatatacactgtttgagatgaggataaaatactctgagtccaaaccgggcccctctgctaaccatgttcatgccttcttctctttcctacagctcctgggcaacgtgctggttgttgtgctgtctcatcattttggcaaagaattcctcgaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatctgcagtgctagtctcccggaactatcactctttcacagtctgctttggaaggactgggcttagtatgaaaagttaggactgagaagaatttgaaagggggctttttgtagcttgatattcactactgtcttattaccctatcataggcccaccccaaatggaagtcccattcttcctcaggatgtttaagattagcattcaggaagagatcagaggtctgctggctcccttatcatgtcccttatggtgcttctggctctgcagttattagcatagtgttaccatcaaccaccttaacttcatttttcttattcaatacctaggtaggtagatgctagattctggaaataaaatatgagtctcaagtggtccttgtcctctctcccagtcaaattctgaatctagttggcaagattctgaaatcaaggcatataatcagtaataagtgatgatagaagggtatatagaagaattttattatatgagagggtgaaatcccagcaatttgggaggctgaggcaggagaatcgcttgatcctgggaggcagaggttgcagtgagccaagattgtgccactgcattccagcccaggtgacagcatgagactccgtcacaaaaaaaaaagaaaaaaaaggggggggggggcggtggagccaagatgaccgaataggaacagctccagtactatagctcccatcgtgagtgacgcagaagacgggtgatttctgcatttccaactgaggtaccaggttcatctcacagggaagtgccaggcagtgggtgcaggacagtaggtgcagtgcactgtgcatgagccgaagcagggacgaggcatcacctcacccgggaagcacaaggggtcagggaattccctttcctagtcaaagaaaagggtgacagatggcacctggaaaatcgggtcactcccgccctaatactgcgctcttccaacaagcttgtctttggaaaatagatcaatttcccttgggaagaagatttttagcacagcaaggggcaggatgttcaactgtgagaaaacgaagaattagccaaaaaacttccagtaagcctgcaaaaaaaaaaaaaaaataaaagctaagtttctataaatgttctgtaaatgtaaaacagaaggtaagtcaactgcacctaataaaaatcacttaatagcaatgtgctgtgtcagttgtttattggaaccacacccggtacacatcctgtccagcatttgcagtgcgtgcattgaattattgtgctggctagacttcatggcgcctggcaccgaatcctgccttctcagcgaaaatgaataattgctttgttggcaagaaactaagcatcaatgggacgcgtgcaaagcaccggcggcggtagatgcggggtaagtactgaattttaattcgacctatcccggtaaagcgaaagcgacacgcttttttttcacacatagcgggaccgaacacgttataagtatcgattaggtctatttttgtctctctgtcggaaccagaactggtaaaagtttccattgcgtctgggcttgtctatcattgcgtctctatggtttttggaggattagacggggccaccagtaatggtgcatagcggatgtctgtaccgccatcggtgcaccgatataggtttggggctccccaagggactgctgggatgacagcttcatattatattgaatgggcgcataatcagcttaattggtgaggacaagctacaagttgtaacctgatctccacaaagtacgttgccggtcggggtcaaaccgtcttcggtgctcgaaaccgccttaaactacagacaggtcccagccaagtaggcggatcaaaacctcaaaaaggcgggagccaatcaaaatgcagcattatattttaagctcaccgaaaccggtaagtaaagactatgtattttttcccagtgaataattgttgttaactataaaaagcgtcatggcaaacgataaaggtagcaattgggattcgggcttgggatgctcatatctgctgactgaggcagaatgtgaaagtgacaaagagaatgaggaacccggggcaggtgtagaactgtctgtggaatctgatcggtatgatagccaggatgaggattttgttgacaatgcatcagtctttcagggaaatcacctggaggtcttccaggcattagagaaaaaggcgggtgaggagcagattttaaatttgaaaagaaaagtattggggagttcgcaaaacagcagcggttccgaagcatctgaaactccagttaaaagacggaaatcaggagcaaagcgaagattatttgctgaaaatgaagctaaccgtgttcttacgcccctccaggtacagggggagggggaggggaggcaagaacttaatgaggagcaggcaattagtcatctacatctgcagcttgttaaatctaaaaatgctacagtttttaagctggggctctttaaatctttgttcctttgtagcttccatgatattacgaggttgtttaagaatgataagaccactaatcagcaatgggtgctggctgtgtttggccttgcagaggtgttttttgaggcgagtttcgaactcctaaagaagcagtgtagttttctgcagatgcaaaaaagatctcatgaaggaggaacttgtgcagtttacttaatctgctttaacacagctaaaagcagagaaacagtccggaatctgatggcaaacatgctaaatgtaagagaagagtgtttgatgctgcagccacctaaaattcgaggactcagcgcagctctattctggtttaaaagtagtttgtcacccgctacacttaaacatggtgctttacctgagtggatacgggcgcaaactactctgaacgagagcttgcagaccgagaaattcgacttcggaactatggtgcaatgggcctatgatcacaaatatgctgaggagtctaaaatagcctatgaatatgctttggctgcaggatctgatagcaatgcacgggcttttttagcaactaacagccaagctaagcatgtgaaggactgtgcaactatggtaagacactatctaagagctgaaacacaagcattaagcatgcctgcatatattaaagctaggtgcaagctggcaactggggaaggaagctggaagtctatcctaactttttttaactatcagaatattgaattaattacctttattaatgctttaaagctctggctaaaaggaattccaaaaaaaaactgtttagcatttattggccctccaaacacaggcaagtctatgctctgcaactcattaattcattttttgggtggtagtgttttatcttttgccaaccataaaagtcacttttggcttgcttccctagcagatactagagctgctttagtagatgatgctactcatgcttgctggaggtactttgacacatacctcagaaatgcattggatggctaccctgtcagtattgatagaaaacacaaagcagcggttcaaattaaagctccacccctcctggtaaccagtaatattgatgtgcaggcagaggacagatatttgtacttgcatagtcgggtgcaaacctttcgctttgagcagccatgcacagatgaatcgggtgagcaaccttttaatattactgatgcagattggaaatctttttttgtaaggttatgggggcgtttagacctgattgacgaggaggaggatagtgaagaggatggagacagcatgcgaacgtttacatgcagcgcaagaaacacaaatgcagttgattgagaaaagtagtgataagttgcaagatcatatactgtactggactgctgttagaactgagaacacactgctttatgctgcaaggaaaaaaggggtgactgtcctaggacactgcagagtaccacactctgtagtttgtcaagagagagccaagcaggccattgaaatgcagttgtctttgcaggagttaagcaaaactgagtttggggatgaaccatggtctttgcttgacacaagctgggaccgatatatgtcagaacctaaacggtgctttaagaaaggcgccagggtggtagaggtggagtttgatggaaatgcaagcaatacaaactggtacactgtctacagcaatttgtacatgcgcacagaggacggctggcagcttgcgaaggctgggctgacggaactgggctctactactgcaccatggccggtgctggacgcatttactattctcgctttggtgacgaggcagccagatttagtacaacagggcattactctgtaagagatcaggacagagtgtatgctggtgtctcatccacctcttctgattttagagatcgcccagacggagtctgggtcgcatccgaaggacctgaaggagaccctgcaggaaaagaagccgagccagcccagcctgtctcttctttgctcggctcccccgcctgcggtcccatcagagcaggcctcggttgggtacgggacggtcctcgctcgcacccctacaattttcctgcaggctcggggggctctattctccgctcttcctccaccccgtgcagggcacggtaccggtggacttggcatcaaggcaggaagaagaggagcagtcgcccgactccacagaggaagaaccagtgactctcccaaggcgcaccaccaatgatggattccacctgttaaaggcaggagggtcatgctttgctctaatttcaggaactgctaaccaggtaaagtgctatcgctttcgggtgaaaaagaaccatagacatcgctacgagaactgcaccaccacctggttcacagttgctgacaacggtgctgaaagacaaggacaagcacaaatactgatcacctttggatcgccaagtcaaaggcaagactttctgaaacatgtaccactacctcctggaatgaacatttccggctttacagccagcttggacttctgatcactgccattgccttttcttcatctgactggtgtactatgccaaatctatgcgaccgcattataaagccgaattctgcagatatccatcacactggcggccatatggccgctatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattctcatgtttgacagcttatcatcgataagcttcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatcctgcttcatccccgtggcccgttgctcgcgtttgctggcggtgtccccggaagaaatatatttgcatgtctttagttctatgatgacacaaaccccgcccagcgtcttgtcattggcgaattcgaacacgcagatgcagtcggggcggcgcggtcccaggtccacttcgcatattaaggtgacgcgtgtggcctcgaacaccgagcgaccctgcagcgacccgcttaacagcgtcaacagcgtgccgcagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccgggagatgggggaggctaactgaaacacggaaggagacaataccggaaggaacccgcgctatgaacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaagccctgccatagccacgggccccgtgggttagggacggcggatcgcggccc

SEQ ID NO: 25 - DNA, 16105 bp, Artificial Sequence
  Synthetic Polynucleotide
agatctctcgaaccgggtaacgtatgcaacataggtatagtattatacatgtaaatataaccgagtacaggttgtaatggcggtacaactgtaactaataactgatcaataattatcattagttaatgccccagtaatcaagtatcgggtatatacctcaaggcgcaatgtattgaatgccatttaccgggcggaccgactggcgggttgctgggggcgggtaactgcagttattactgcatacaagggtatcattgcggttatccctgaaaggtaactgcagttacccacctcataaatgccatttgacgggtgaaccgtcatgtagttcacatagtatacggttcatgcgggggataactgcagttactgccatttaccgggcggaccgtaatacgggtcatgtactggaataccctgaaaggatgaaccgtcatgtagatgcataatcagtagcgataatggtaccactacgccaaaaccgtcatgtagttacccgcacctatcgccaaactgagtgcccctaaaggttcagaggtggggtaactgcagttaccctcaaacaaaaccgtggttttagttgccctgaaaggttttacagcattgttgaggcggggtaactgcgtttacccgccatccgcacatgccaccctccagatatattcgtctcgagcaaatcacttggcagtctagcggacctctgcggtaggtgcgacaaaactggaggtatcttctgtggccctggctaggtcggaggccagctagctggctaggactcttgaagtcccactcaaacccctgggaactaacaagaaagaaaaagcgataacattttaagtacaatatacctcccccgtttcaaaagtcccacaacaaatcttacccttctacagggaacatagtggtacctgggagtactattaaaacaaagaaagtgaaagatgagacaactgttggtaacagaggagaataaaagaaaagtaaaagacattgaaaaagcaatttgaaatcgaacgtaaacattgcttaaaaatttaagtgaaaacaaataaacagtctaacattcatgaaagagattagtgaaaaaaaagttccgttagtcccatataatataacatgaagtcgtgtcaaaatctcttgttaacaatattaatttactattccatcttataaagacgtatatttaagaccgaccgcacctttataagaataaccatctttgttgatgtgggaccagtagtaggacggaaagagaaataccaatgttactatatgtgacaaactctactcctattttatgagactcaggtttggcccggggagacgattggtacaagtacggaagaagagaaaggatgtcgaggacccgttgcacgaccaacaacacgacagagtagtaaaaccgtttcttaagcttcggagctctactactttgaatagtagttaagtaacatatttttatttctctaaaaggactctcttgactaaagtttacgaagactacgaaatctattctattccgattatagtgactgactacttttacgagaaagacctttactccttgattgtcagttttaattcacactattcctcttcttggacgacgtacagtgtctgtggccacatccttactggtctcttctcaaccaatttttggaaccatggtatcggtttagaccctgttcgctcaaaaatttgttttactgacttcgtgtccttctaccggtcagttgaagacttaactaaccggtcaaaccacagccaaagataaggcggaaggaacatcgtctattccaataacagtgaagttttgtgttgttgctatgggtcgtgtagaccctcagactgaggttacttaaaagacattaacgactgggttctcctttgtgagatcctgccccttgctgttaatgggaacagaattttcttcttcgtagactaatggaacttaacctatgttaatttttagagcagttttttataagtgtcaagtatttgaaaggataaatacatacctcgtcgttctgactttgacaactcctcgggtacctccttcttcttcgtcggtttcttctctttcttcttagactactacttcgacgtcatctccttcttcttcttcttttctttggtttctgattttttcaacttttttgacagaccctgacccttgaatacttactatagtttggttataccgtctctggtagttttcttcatcttcttctacttatgtttcgaaagatgtttagtaaaagtttcctttcactactggggtaccgaatataagtgaaatgacgacttccccttcaatggaagtttagttaaaataaacatgggtgtagacgaggtgcaccagacaaactgcttatacctagatttttctcgctaatgtaattcgagatacacgcggcacataagtagtgtctgctgaaggtactatactacggatttatggagttaaaacagttcccacaccacctgagtctactagaggggaacttacaaagggcgctctgagaagtcgttgtatttgacgaattccactaatccttcttcgaacaagcattttgcgacctgtactagttcttctaacgactactatttatgttactatgaaaaacctttcttaaaccatggttgtagttcgaaccacactaacttctggtgagcttagcttgtgcagaacgatttgaagaatccaaggtcagaagagtagtaggttgactgtaatgatcggatctggtcatacacctttcttacttcctttttgttctgttttagatgaagtaccgacccaggtcgtcttttctccgacttagaagaggtaaacaactcgctgaagactttttcccgatacttcaataaatggagtgtcttggacacctacttatgacataagtccgggaagggcttaaactacccttctccaaggtcttacaacggttccttcctcacttcaagctactttcactcttttgattcctctcagcacttcgtcaactctttcttaaactcggagacgacttaacctactttctatttcgggaattcctgttctaacttttccgacaccacagagtcgcggactgtcttagaggcacacgaaaccaccggtcggtcatgcctaccagaccgttgtacctctcttagtactttcgtgttcgcatggtttgcccgttcctgtagagatgtttaatgatacgctcagtcttcttttgtaaactttaattagggtctgtgggcgactagtctctgtacgaagctgcttaattccttctacttctactattttgtcaaaacctagaacgacaccaaaacaaactttgtcgttgcgaagccagtcccatagaaaatggtctgtgatttcgtatacctctatcttatctttcttacgaagcggagtcaaacttgtaactgggactacgtttccaccttcttctcgggcttcttcttggacttctctgtcgtcttctgtgttgtcttctgtgtctcgttctgcttctacttctttacctacacccttgtctacttcttcttctttgtcgtttccttagatgtcgacttcctaggacactgttttgagtgtgtacgggtggcacgggtcgtggacttgaggacccccctggcagtcagaaggagaaggggggttttgggttcctgtgggagtactagagggcctggggactccagtgtacgcaccaccacctgcactcggtgcttctgggactccagttcaagttgaccatgcacctgccgcacctccacgtattacggttctgtttcggcgccctcctcgtcatgttgtcgtgcatggcacaccagtcgcaggagtggcaggacgtggtcctgaccgacttaccgttcctcatgttcacgttccagaggttgtttcgggagggtcgggggtagctcttttggtagaggtttcggtttcccgtcggggctcttggtgtccacatgtgggacgggggtagggccctactcgactggttcttggtccagtcggactggacggaccagtttccgaagatagggtcgctgtagcggcacctcaccctctcgttacccgtcggcctcttgttgatgttctggtgcggagggcacgacctgaggctgccgaggaagaaggagatgtcgttcgagtggcacctgttctcgtccaccgtcgtccccttgcagaagagtacgaggcactacgtactccgagacgtgttggtgatgtgcgtcttctcggagagggacagaggcccatttactgagctgggtctgatcagtttaattcggcttaagacgtctataggtagtgtgaccgccggcgaccttaagtgaggagtccacgtccgacggatagtcttccaccaccgaccacaccggttacgggaccgagtgtttatggtgactctagaaaaagggagacggtttttaatacccctgtagtacttcggggaactcgtagactgaagaccgattatttcctttaaataaaagtaacgttatcacacaaccttaaaaaacacagagagtgagccttcctgtataccctcccgtttagtaaattttgtagtcttactcataaaccaaatctcaaaccgttgtatacgggtatacgaccgacggtacttgtttccaaccgatatttctccagtagtcatatactttgtcgggggacgacaggtaaggaataaggtatcttttcggaactgaactccaatctaaaaaaaatataaaacaaaacacaataaaaaaagaaattgtagggattttaaaaggaatgtacaaaatgatcggtctaaaaaggaggagaggactgatgagggtcagtatcgacagggagaagagaatacctctagggagctgcctagggatctcagctccgctacgccgcgtcgtggtaccggactttattggagactttctccttgaaccaatccatggaaccaaaaattttggtcggacctcatctcgtctacccaattccactcactggggagtcgggacctgtaagaatctactcgggggagtcctcatctcttattacaactctactcaagacaaccgattttattagttccgatcagaaatattttgacagaggagaagaggatcgaagctaggtctctctctggacccgcctcgaccagcgacgagtccttgaggtcctttcctcttcgactccaatggtgcgacgcttacccaaatgcctctatcgaccgaaaggccccactcaagagcatttgaggtctcgtcgctatccggcattatagcccctttcgtgatatccctgtactacaaggtgtgcagtgtacccagcaggataggctcggtcagcacggtttccccgccagggcgacacgtgtgaccgcgaggtccctcgagacgtgaggcgggcttttcacgcgagccgagacggtcctgcgccccgcgcactgatacgcacccgacctcgttggcggacgacccacgtttgggaaacgcgggcctgagcaggttgctgatatttctcccgtccgacaggagattcgcagtggtgctgaagttgcaggactcatggaagaggagtgaatgaggcatcgaggtcgaagtggtggttcgaggagctgcagctagcgcttcgaaaccggggaaaccggaatcgcagctggctaggactcttgaagtcccactcaaacccctgggaactaacaagaaagaaaaagcgataacattttaagtacaatatacctcccccgtttcaaaagtcccacaacaaatcttacccttctacagggaacatagtggtacctgggagtactattaaaacaaagaaagtgaaagatgagacaactgttggtaacagaggagaataaaagaaaagtaaaagacattgaaaaagcaatttgaaatcgaacgtaaacattgcttaaaaatttaagtgaaaacaaataaacagtctaacattcatgaaagagattagtgaaaaaaaagttccgttagtcccatataatataacatgaagtcgtgtcaaaatctcttgttaacaatattaatttactattccatcttataaagacgtatatttaagaccgaccgcacctttataagaataaccatctttgttgatgtgggaccagtagtaggacggaaagagaaataccaatgttactatatgtgacaaactctactcctattttatgagactcaggtttggcccggggagacgattggtacaagtacggaagaagagaaaggatgtcgaggacccgttgcacgaccaacaacacgacagagtagtaaaaccgtttcttaaggagctggtcacgtccgacggatagtctttcaccaccgaccacaccgattacgggaccgggtgttcatagtgattcgagcgaaagaacgacaggttaaagataatttccaaggaaacaagggattcaggttgatgatttgaccccctataatacttcccggaactcgtagacctaagacggattattttttgtaaataaaagtaacgttactacataaatttaataaagacttataaaatgatttttcccttacaccctccagtcacgtaaattttgtatttctttacttctcgatcaagtttggaacccttttatgtgatatagaatttgaggtactttcttccactccgacgtttgtcgattacgtgtaaccgttgtcggggactacggatacggaataagtagggagtcttttcctaagttcatctccgaactaaacctccaatttcaaaacgatacgacataaaatgtaatgaataacaaaatcgacaggagtacttacagaaaagtgatgggtaaacgaataggacgtagagagtcggaactgaggtgagtcaagagaacgaatctctatggtggaaaggggacttcacaaggaaggtacaaaatgccgctctaccaaagaggagcggaccggtgagtcggaatcaacagagacaacagaatatctccagatgaacttcttcctttttgtcccccgtaccaaactgacaggacactcgggaagaagggacggagggggtgagtgtcactgggccttagacgtcacgatcagagggccttgatagtgagaaagtgtcagacgaaaccttcctgacccgaatcatacttttcaatcctgactcttcttaaactttcccccgaaaaacatcgaactataagtgatgacagaataatgggatagtatccgggtggggtttaccttcagggtaagaaggagtcctacaaattctaatcgtaagtccttctctagtctccagacgaccgagggaatagtacagggaataccacgaagaccgagacgtcaataatcgtatcacaatggtagttggtggaattgaagtaaaaagaataagttatggatccatccatctacgatctaagacctttattttatactcagagttcaccaggaacaggagagagggtcagtttaagacttagatcaaccgttctaagactttagttccgtatattagtcattattcactactatcttcccatatatcttcttaaaataatatactctcccactttagggtcgttaaaccctccgactccgtcctcttagcgaactaggaccctccgtctccaacgtcactcggttctaacacggtgacgtaaggtcgggtccactgtcgtactctgaggcagtgttttttttttcttttttttccccccccccccgccacctcggttctactggcttatccttgtcgaggtcatgatatcgagggtagcactcactgcgtcttctgcccactaaagacgtaaaggttgactccatggtccaagtagagtgtcccttcacggtccgtcacccacgtcctgtcatccacgtcacgtgacacgtactcggcttcgtccctgctccgtagtggagtgggcccttcgtgttccccagtcccttaagggaaaggatcagtttcttttcccactgtctaccgtggaccttttagcccagtgagggcgggattatgacgcgagaaggttgttcgaacagaaaccttttatctagttaaagggaacccttcttctaaaaatcgtgtcgttccccgtcctacaagttgacactcttttgcttcttaatcggttttttgaaggtcattcggacgttttttttttttttttattttcgattcaaagatatttacaagacatttacattttgtcttccattcagttgacgtggattatttttagtgaattatcgttacacgacacagtcaacaaataaccttggtgtgggccatgtgtaggacaggtcgtaaacgtcacgcacgtaacttaataacacgaccgatctgaagtaccgcggaccgtggcttaggacggaagagtcgcttttacttattaacgaaacaaccgttctttgattcgtagttaccctgcgcacgtttcgtggccgccgccatctacgccccattcatgacttaaaattaagctggatagggccatttcgctttcgctgtgcgaaaaaaaagtgtgtatcgccctggcttgtgcaatattcatagctaatccagataaaaacagagagacagccttggtcttgaccattttcaaaggtaacgcagacccgaacagatagtaacgcagagataccaaaaacctcctaatctgccccggtggtcattaccacgtatcgcctacagacatggcggtagccacgtggctatatccaaaccccgaggggttccctgacgaccctactgtcgaagtataatataacttacccgcgtattagtcgaattaaccactcctgttcgatgttcaacattggactagaggtgtttcatgcaacggccagccccagtttggcagaagccacgagctttggcggaatttgatgtctgtccagggtcggttcatccgcctagttttggagtttttccgccctcggttagttttacgtcgtaatataaaattcgagtggctttggccattcatttctgatacataaaaaagggtcacttattaacaacaattgatatttttcgcagtaccgtttgctatttccatcgttaaccctaagcccgaaccctacgagtatagacgactgactccgtcttacactttcactgtttctcttactccttgggccccgtccacatcttgacagacaccttagactagccatactatcggtcctactcctaaaacaactgttacgtagtcagaaagtccctttagtggacctccagaaggtccgtaatctctttttccgcccactcctcgtctaaaatttaaacttttcttttcataacccctcaagcgttttgtcgtcgccaaggcttcgtagactttgaggtcaattttctgcctttagtcctcgtttcgcttctaataaacgacttttacttcgattggcacaagaatgcggggaggtccatgtccccctccccctcccctccgttcttgaattactcctcgtccgttaatcagtagatgtagacgtcgaacaatttagatttttacgatgtcaaaaattcgaccccgagaaatttagaaacaaggaaacatcgaaggtactataatgctccaacaaattcttactattctggtgattagtcgttacccacgaccgacacaaaccggaacgtctccacaaaaaactccgctcaaagcttgaggatttcttcgtcacatcaaaagacgtctacgttttttctagagtacttcctccttgaacacgtcaaatgaattagacgaaattgtgtcgattttcgtctctttgtcaggccttagactaccgtttgtacgatttacattctcttctcacaaactacgacgtcggtggattttaagctcctgagtcgcgtcgagataagaccaaattttcatcaaacagtgggcgatgtgaatttgtaccacgaaatggactcacctatgcccgcgtttgatgagacttgctctcgaacgtctggctctttaagctgaagccttgataccacgttacccggatactagtgtttatacgactcctcagattttatcggatacttatacgaaaccgacgtcctagactatcgttacgtgcccgaaaaaatcgttgattgtcggttcgattcgtacacttcctgacacgttgataccattctgtgatagattctcgactttgtgttcgtaattcgtacggacgtatataatttcgatccacgttcgaccgttgaccccttccttcgaccttcagataggattgaaaaaaattgatagtcttataacttaattaatggaaataattacgaaatttcgagaccgattttccttaaggtttttttttgacaaatcgtaaataaccgggaggtttgtgtccgttcagatacgagacgttgagtaattaagtaaaaaacccaccatcacaaaatagaaaacggttggtattttcagtgaaaaccgaacgaagggatcgtctatgatctcgacgaaatcatctactacgatgagtacgaacgacctccatgaaactgtgtatggagtctttacgtaacctaccgatgggacagtcataactatcttttgtgtttcgtcgccaagtttaatttcgaggtggggaggaccattggtcattataactacacgtccgtctcctgtctataaacatgaacgtatcagcccacgtttggaaagcgaaactcgtcggtacgtgtctacttagcccactcgttggaaaattataatgactacgtctaacctttagaaaaaaacattccaatacccccgcaaatctggactaactgctcctcctcctatcacttctcctacctctgtcgtacgcttgcaaatgtacgtcgcgttctttgtgtttacgtcaactaactcttttcatcactattcaacgttctagtatatgacatgacctgacgacaatcttgactcttgtgtgacgaaatacgacgttccttttttccccactgacaggatcctgtgacgtctcatggtgtgagacatcaaacagttctctctcggttcgtccggtaactttacgtcaacagaaacgtcctcaattcgttttgactcaaacccctacttggtaccagaaacgaactgtgttcgaccctggctatatacagtcttggatttgccacgaaattctttccgcggtcccaccatctccacctcaaactacctttacgttcgttatgtttgaccatgtgacagatgtcgttaaacatgtacgcgtgtctcctgccgaccgtcgaacgcttccgacccgactgccttgacccgagatgatgacgtggtaccggccacgacctgcgtaaatgataagagcgaaaccactgctccgtcggtctaaatcatgttgtcccgtaatgagacattctctagtcctgtctcacatacgaccacagagtaggtggagaagactaaaatctctagcgggtctgcctcagacccagcgtaggcttcctggacttcctctgggacgtccttttcttcggctcggtcgggtcggacagagaagaaacgagccgagggggcggacgccagggtagtctcgtccggagccaacccatgccctgccaggagcgagcgtggggatgttaaaaggacgtccgagccccccgagataagaggcgagaaggaggtggggcacgtcccgtgccatggccacctgaaccgtagttccgtccttcttctcctcgtcagcgggctgaggtgtctccttcttggtcactgagagggttccgcgtggtggttactacctaaggtggacaatttccgtcctcccagtacgaaacgagattaaagtccttgacgattggtccatttcacgatagcgaaagcccactttttcttggtatctgtagcgatgctcttgacgtggtggtggaccaagtgtcaacgactgttgccacgactttctgttcctgttcgtgtttatgactagtggaaacctagcggttcagtttccgttctgaaagactttgtacatggtgatggaggaccttacttgtaaaggccgaaatgtcggtcgaacctgaagactagtgacggtaacggaaaagaagtagactgaccacatgatacggtttagatacgctggcgtaatatttcggcttaagacgtctataggtagtgtgaccgccggtataccggcgatacgccacactttatggcgtgtctacgcattcctcttttatggcgtagtccgcgagaaggcgaaggagcgagtgactgagcgacgcgagccagcaagccgacgccgctcgccatagtcgagtgagtttccgccattatgccaataggtgtcttagtcccctattgcgtcctttcttgtacactcgttttccggtcgttttccggtccttggcatttttccggcgcaacgaccgcaaaaaggtatccgaggcggggggactgctcgtagtgtttttagctgcgagttcagtctccaccgctttgggctgtcctgatatttctatggtccgcaaagggggaccttcgagggagcacgcgagaggacaaggctgggacggcgaatggcctatggacaggcggaaagagggaagcccttcgcaccgcgaaagagtatcgagtgcgacatccatagagtcaagccacatccagcaagcgaggttcgacccgacacacgtgcttggggggcaagtcgggctggcgacgcggaataggccattgatagcagaactcaggttgggccattctgtgctgaatagcggtgaccgtcgtcggtgaccattgtcctaatcgtctcgctccatacatccgccacgatgtctcaagaacttcaccaccggattgatgccgatgtgatcttcctgtcataaaccatagacgcgagacgacttcggtcaatggaagcctttttctcaaccatcgagaactaggccgtttgtttggtggcgaccatcgccaccaaaaaaacaaacgttcgtcgtctaatgcgcgtctttttttcctagagttcttctaggaaactagaaaagatgccccagactgcgagtcaccttgcttttgagtgcaattccctaaaaccagtactctaatagtttttcctagaagtggatctaggaaaatttaatttttacttcaaaatttagttagatttcatatatactcatttgaaccagactgtcaatggttacgaattagtcactccgtggatagagtcgctagacagataaagcaagtaggtatcaacggactgaggggcagcacatctattgatgctatgccctcccgaatggtagaccggggtcacgacgttactatggcgctctgggtgcgagtggccgaggtctaaatagtcgttatttggtcggtcggccttcccggctcgcgtcttcaccaggacgttgaaataggcggaggtaggtcagataattaacaacggcccttcgatctcattcatcaagcggtcaattatcaaacgcgttgcaacaacggtaacgacgtccgtagcaccacagtgcgagcagcaaaccataccgaagtaagtcgaggccaagggttgctagttccgctcaatgtactagggggtacaacacgttttttcgccaatcgaggaagccaggaggctagcaacagtcttcattcaaccggcgtcacaatagtgagtaccaataccgtcgtgacgtattaagagaatgacagtacggtaggcattctacgaaaagacactgaccactcatgagttggttcagtaagactcttatcacatacgccgctggctcaacgagaacgggccgcagttgtgccctattatggcgcggtgtatcgtcttgaaattttcacgagtagtaaccttttgcaagaagccccgcttttgagagttcctagaatggcgacaactctaggtcaagctacattgggtgagcacgtgggttgactagaagtcgtagaaaatgaaagtggtcgcaaagacccactcgtttttgtccttccgttttacggcgttttttcccttattcccgctgtgcctttacaacttatgagtatgagaaggaaaaagttataataacttcgtaaatagtcccaataacagagtactcgcctatgtataaacttacataaatctttttatttgtttatccccaaggcgcgtgtaaaggggcttttcacggtggactgcagattctttggtaataatagtactgtaattggatatttttatccgcatagtgctccgggaaagcagaagttcttaagagtacaaactgtcgaatagtagctattcgaagtgcgacggcgttcgtgagtcccgcgttcccgacgatttccttcgccttgtgcatctttcggtcaggcgtctttgccacgactggggcctacttacagtcgatgacccgatagacctgttcccttttgcgttcgcgtttctctttcgtccatcgaacgtcacccgaatgtaccgctatcgatctgacccgccaaaatacctgtcgttcgcttggccttaacggtcgaccccgcgggagaccattccaacccttcgggacgtttcatttgacctaccgaaagaacggcggttcctagactaccgcgtcccctagttctaggacgaagtaggggcaccgggcaacgagcgcaaacgaccgccacaggggccttctttatataaacgtacagaaatcaagatactactgtgtttggggcgggtcgcagaacagtaaccgcttaagcttgtgcgtctacgtcagccccgccgcgccagggtccaggtgaagcgtataattccactgcgcacaccggagcttgtggctcgctgggacgtcgctgggcgaattgtcgcagttgtcgcacggcgtctagactagttctctgtcctactcctagcaaagcgtactaacttgttctacctaacgtgcgtccaagaggccggcgaacccacctctccgataagccgatactgacccgtgttgtctgttagccgacgagactacggcggcacaaggccgacagtcgcgtccccgcgggccaagaaaaacagttctggctggacaggccacgggacttacttgacgtcctgctccgtcgcgccgatagcaccgaccggtgctgcccgcaaggaacgcgtcgacacgagctgcaacagtgacttcgcccttccctgaccgacgataacccgcttcacggccccgtcctagaggacagtagagtggaacgaggacggctctttcataggtagtaccgactacgttacgccgccgacgtatgcgaactaggccgatggacgggtaagctggtggttcgctttgtagcgtagctcgctcgtgcatgagcctaccttcggccagaacagctagtcctactagacctgcttctcgtagtccccgagcgcggtcggcttgacaagcggtccgagttccgcgcgtacgggctgccgctcctagagcagcactgggtaccgctacggacgaacggcttatagtaccaccttttaccggcgaaaagacctaagtagctgacaccggccgacccacaccgcctggcgatagtcctgtatcgcaaccgatgggcactataacgacttctcgaaccgccgcttacccgactggcgaaggagcacgaaatgccatagcggcgagggctaagcgtcgcgtagcggaagatagcggaagaactgctcaagaagactcgccctgagaccccaagctttactggctggttcgctgcgggttggacggtagtgctctaaagctaaggtggcggcggaagatactttccaacccgaagccttagcaaaaggccctgcggccgacctactaggaggtcgcgcccctagagtacgacctcaagaagcgggtggggccctctaccccctccgattgactttgtgccttcctctgttatggccttccttgggcgcgatacttgccgttatttttctgtcttattttgcgtgccacaacccagcaaacaagtatttgcgccccaagccagggtcccgaccgtgagacagctatggggtggctctggggtaaccccggttatgcgggcgcaaagaaggaaaaggggtggggtggggggttcaagcccacttccgggtcccgagcgtcggttgcagccccgccgttcgggacggtatcggtgcccggggcacccaatccctgccgcctagcgccggg

SEQ ID NO: 26-28 - skipped (3 entries)

SEQ ID NO: 29 - protein, 701 aa, Homo sapiens [sequence omitted]

SEQ ID NO: 30 - protein, 653 aa, Homo sapiens
  ref <308>: DATABASE ACCESSION NUMBER: NCBI / CAA61201.1
  ref <309>: DATABASE ENTRY DATE: 2008-10-07
  ref <313>: RELEVANT RESIDUES IN SEQ ID NO: (1)..(653)
MKLSLVAAMLLLLSAARAEEEDKKEDVGTVVGIDLGTTYSCVGVFKNGRVEIIANDQGNRITPSYVAFTPEGERLIGDAAKNQLTSNPENTVFDAKRLIGRTWNDPSVQQDIKFLPFKVVEKKTKPYIQVDIGGGQTKTFAPEEISAMVLTKMKETAEAYLGKKVTHAVVTVPAYFNDAQRQATKDAGTIAGLNVMRIINEPTAAAIAYGLDKREGEKNILVFDLGGGTFDVSLLTIDNGVFEVVATNGDTHLGGEDFDQRVMEHFIKLYKKKTGKDVRKDNRAVQKLRREVEKAKALSSQHQARIEIESFYEGEDFSETLTRAKFEELNMDLFRSTMKPVQKVLEDSDLKKSDIDEIVLVGGSTRIPKIQQLVKEFFNGKEPSRGINPDEAVAYGAAVQAGVLSGDQDTGDLVLLHVCPLTLGIETVGGVMTKLIPSNTVVPTKNSQIFSTASDNQPTVTIKVYEGERPLTKDNHLLGTFDLTGIPPAPRGVPQIEVTFEIDVNGILRVTAEDKGTGNKNKITITNDQNRLTPEEIERMVNDAEKFAEEDKKLKERIDTRNELESYAYSLKNQIGDKEKLGGKLSSEDKETMEKAVEEKIEWLESHQDADIEDFKAKKKELEEIVQPIISKLYGSAGPPPTGEEDTAEKDEL

SEQ ID NO: 31 - protein, 654 aa, Homo sapiens
  ref <308>: DATABASE ACCESSION NUMBER: NCBI / NP_005338.1
  ref <309>: DATABASE ENTRY DATE: 2016-02-21
  ref <313>: RELEVANT RESIDUES IN SEQ ID NO: (1)..(654)
MKLSLVAAMLLLLSAARAEEEDKKEDVGTVVGIDLGTTYSCVGVFKNGRVEIIANDQGNRITPSYVAFTPEGERLIGDAAKNQLTSNPENTVFDAKRLIGRTWNDPSVQQDIKFLPFKVVEKKTKPYIQVDIGGGQTKTFAPEEISAMVLTKMKETAEAYLGKKVTHAVVTVPAYFNDAQRQATKDAGTIAGLNVMRIINEPTAAAIAYGLDKREGEKNILVFDLGGGTFDVSLLTIDNGVFEVVATNGDTHLGGEDFDQRVMEHFIKLYKKKTGKDVRKDNRAVQKLRREVEKAKRALSSQHQARIEIESFYEGEDFSETLTRAKFEELNMDLFRSTMKPVQKVLEDSDLKKSDIDEIVLVGGSTRIPKIQQLVKEFFNGKEPSRGINPDEAVAYGAAVQAGVLSGDQDTGDLVLLDVCPLTLGIETVGGVMTKLIPRNTVVPTKKSQIFSTASDNQPTVTIKVYEGERPLTKDNHLLGTFDLTGIPPAPRGVPQIEVTFEIDVNGILRVTAEDKGTGNKNKITITNDQNRLTPEEIERMVNDAEKFAEEDKKLKERIDTRNELESYAYSLKNQIGDKEKLGGKLSSEDKETMEKAVEEKIEWLESHQDADIEDFKAKKKELEEIVQPIISKLYGSAGPPPTGEEDTAEKDEL

SEQ ID NO: 32 - DNA, 7945 bp, Deltapapillomavirus 4
  misc_feature (1205)..(1205): n is a, c, g, or t
  ref <308>: DATABASE ACCESSION NUMBER: NCBI / NC_001522.1
  ref <309>: DATABASE ENTRY DATE: 2010-03-26
  ref <313>: RELEVANT RESIDUES IN SEQ ID NO: (1)..(7945)
gttaacaataatcacaccatcaccgttttttcaagcgggaaaaaatagccagctaactataaaaagctgctgacagaccccggttttcacatggacctgaaaccttttgcaagaaccaatccattctcagggttggattgtctgtggtgcagagagcctcttacagaagttgatgcttttaggtgcatggtcaaagactttcatgttgtaattcgggaaggctgtagatatggtgcatgtaccatttgtcttgaaaactgtttagctactgaaagaagactttggcaaggtgttccagtaacaggtgaggaagctgaattattgcatggcaaaacacttgataggctttgcataagatgctgctactgtgggggcaaactaacaaaaaatgaaaaacatcggcatgtgctttttaatgagcctttctgcaaaaccagagctaacataattagaggacgctgctacgactgctgcagacatggttcaaggtccaaatacccatagaaacttggatgattcacctgcaggaccgttgctgattttaagtccatgtgcaggcacacctaccaggtctcctgcagcacctgatgcacctgatttcagacttccgtgccatttcggccgtcctactaggaagcgaggtcccactacccctccgctttcctctcccggaaaactgtgtgcaacagggccacgtcgagtgtattctgtgactgtctgctgtggaaactgcggaaaagagctgacttttgctgtgaagaccagctcgacgtccctgcttggatttgaacaccttttaaactcagatttagacctcttgtgtccacgttgtgaatctcgcgagcgtcatggcaaacgataaaggtagcaattgggattcgggcttgggatgctcatatctgctgactgaggcagaatgtgaaagtgacaaagagaatgaggaacccggggcaggtgtagaactgtctgtggaatctgatcggtatgatagccaggatgaggattttgttgacaatgcatcagtctttcagggaaatcacctggaggtcttccaggcattagagaaaaaggcgggtgaggagcagattttaaatttgaaaagaaaagtattggggagttcgcaaaacagcagcggttccgaagcatctgaaactccagttaaaagacggaaatcaggagcaaagcgaagattatttgctgaaaangaagctaaccgtgttcttacgcccctccaggtacagggggagggggaggggaggcaagaacttaatgaggagcaggcaattagtcatctacatctgcagcttgttaaatctaaaaatgctacagtttttaagctggggctctttaaatctttgttcctttgtagcttccatgatattacgaggttgtttaagaatgataagaccactaatcagcaatgggtgctggctgtgtttggccttgcagaggtgttttttgaggcgagtttcgaactcctaaagaagcagtgtagttttctgcagatgcaaaaaagatctcatgaaggaggaacttgtgcagtttacttaatctgctttaacacagctaaaagcagagaaacagtccggaatctgatggcaaacacgctaaatgtaagagaagagtgtttgatgctgcagccagctaaaattcgaggactcagcgcagctctattctggtttaaaagtagtttgtcacccgctacacttaaacatggtgctttacctgagtggatacgggcgcaaactactctgaacgagagcttgcagaccgagaaattcgacttcggaactatggtgcaatgggcctatgatcacaaatatgctgaggagtctaaaatagcctatgaatatgctttggctgcaggatctgatagcaatgcacgggcttttttagcaactaacagccaagctaagcatgtgaaggactgtgcaactatggtaagacactatctaagagctgaaacacaagcattaagcatgcctgcatatattaaagctaggtgcaagctggcaactggggaaggaagctggaagtctatcctaactttttttaactatcagaatattgaattaattacctttattaatgctttaaagctctggctaaaaggaattccaaaaaaaaactgtttagcatttattggccctccaaacacaggcaagtctatgctctgcaactcattaattcattttttgggtggtagtgttttatcttttgccaaccataaaagtcacttttggcttgcttccctagcagatactagagctgctttagtagatgatgctactcatgcttgctggaggtactttgacacatacctcagaaatgcattggatggctaccctgtcagtattgatagaaaacacaaagcagcggttcaaattaaagctccacccctcctggtaaccagtaatattgatgtgcaggcagaggacagatatttgtacttgcatagtcgggtgcaaacctttcgctttgagcagccatgcacagatgaatcgggtgagcaaccttttaatattactgatgcagattggaaatctttttttgtaaggttatgggggcgtttagacctgattgacgaggaggaggatagtgaagaggatggagacagcatgcgaacgtttacatgtagcgcaagaaacacaaatgcagttgattgagaaaagtagtgataagttgcaagatcatatactgtactggactgctgttagaactgagaacacactgctttatgctgcaaggaaaaaaggggtgactgtcctaggacactgcagagtaccacactctgtagtttgtcaagagagagccaagcaggccattgaaatgcagttgtctttgcaggagttaagcaaaactgagtttggggatgaaccatggtctttgcttgacacaagctgggaccgatatatgtcagaacctaaacggtgctttaagaaaggcgccagggtggtagaggtggagtttgatggaaatgcaagcaatacaaactggtacactgtctacagcaatttgtacatgcgcacagaggacggctggcagcttgcgaaggctggggctgacggaactgggctctactactgcaccatggccggtgctggacgcatttactattctcgctttggtgacgaggcagccagatttagtacaacagggcattactctgtaagagatcaggacagagtgtatgctggtgtctcatccacctcttctgattttagagatcgcccagacggagtctgggtcgcatccgaaggacctgaaggagaccctgcaggaaaagaagccgagccagcccagcctgtctcttctttgctcggctcccccgcctgcggtcccatcagagcaggcctcggttgggtacgggacggtcctcgctcgcacccctacaattttcctgcaggctcggggggctctattctccgctcttcctccaccccgtgcagggcacggtaccggtggacttggcatcaaggcaggaagaagaggagcagtcgcccgactccacagaggaagaaccagtgactctcccaaggcgcaccaccaatgatggattccacctgttaaaggcaggagggtcatgctttgctctaatttcaggaactgctaaccaggtaaagtgctatcgctttcgggtgaaaaagaaccatagacatcgctacgagaactgcaccaccacctggttcacagttgctgacaacggtgctgaaagacaaggacaagcacaaatactgatcacctttggatcgccaagtcaaaggcaagactttctgaaacatgtaccactacctcctggaatgaacatttccggctttacagccagcttggacttctgatcactgccattgccttttcttcatctgactggtgtactatgccaaatctatggtttctattgttcttgggactagttgctgcaatgcaactgctgctattactgttcttactcttgttttttcttgtatactgggatcattttgagtgctcctgtacaggtctgcccttttaatgcctttacatcactggctattggctgtgtttttactgttgtgtggatttgatttgttttatatactgtatgaagttttttcatttgtgcttgtattgctgtttgtaagttttttactagagtttgtattccccctgctcagattttatatggtttaagctgcagcaataaaaatgagtgcacgaaaaagagtaaaacgtgccagtgcctatgacctgtacaggacatgcaagcaagcgggcacatgtccaccagatgtgataccaaaggtagaaggagatactatagcagataaaattttgaaatttgggggtcttgcaatctacttaggagggctaggaataggaacatggtctactggaagggttgctgcaggtggatcaccaaggtacacaccactccgaacagcagggtccacatcatcgcttgcatcaataggatccagagctgtaacagcagggacccgccccagtataggtgcgggcattcctttagacacccttgaaactcttggggccttgcgtccaggggtgtatgaggacactgtgctaccagaggcccctgcaatagtcactcctgatgctgttcctgcagattcagggcttgatgccctgtccataggtacagactcgtccacggagaccctcattactctgctagagcctgagggtcccgaggacatagcggttcttgagctgcaacccctggaccgtccaacttggcaagtaagcaatgctgttcatcagtcctctgcataccacgcccctctgcagctgcaatcgtccattgcagaaacatctggtttagaaaatatttttgtaggaggctcgggtttaggggatacaggaggagaaaacattgaactgacatacttcgggtccccacgaacaagcacgccccgcagtattgcctctaaatcacgtggcattttaaactggttcagtaaacggtactacacacaggtgcccacggaagatcctgaagtgttttcatcccaaacatttgcaaacccactgtatgaagcagaaccagctgtgcttaagggacctagtggacgtgttggactcagtcaggtttataaacctgatacacttacaacacgtagcgggacagaggtgggaccacagctacatgtcaggtactcattgagtactatacatgaagatgtagaagcaatcccctacacagttgatgaaaatacacagggacttgcattcgtacccttgcatgaagagcaagcaggttttgaggagatagaattagatgattttagtgagacacatagactgctacctcagaacacctcttctacacctgttggtagtggtgtacgaagaagcctcattccaactcaggaatttagtgcaacacggcctacaggtgttgtaacctatggctcacctgacacttactctgctagcccagttactgaccctgattctacctctcctagtctagttatcgatgacactactactacaccaatcattataattgatgggcacacagttgatttgtacagcagtaactacaccttgcatccctccttgttgaggaaacgaaaaaaacggaaacatgcctaattttttttgcagatggcgttgtggcaacaaggccagaagctgtatctccctccaacccctgtaagcaaggtgctttgcagtgaaacctatgtgcaaagaaaaagcattttttatcatgcagaaacggagcgcctgctaactataggacatccatattacccagtgtctatcggggccaaaactgttcctaaggtctctgcaaatcagtatagggtatttaaaatacaactacctgatcccaatcaatttgcactacctgacaggactgttcacaacccaagtaaagagcggctggtgtgggcagtcataggtgtgcaggtgtccagagggcagcctcttggaggtactgtaactgggcaccccacttttaatgctttgcttgatgcagaaaatgtgaatagaaaagtcaccacccaaacaacagatgacaggaaacaaacaggcctagatgctaagcaacaacagattctgttgctaggctgtacccctgctgaaggggaatattggacaacagcccgtccatgtgttactgatcgtctagaaaatggcgcctgccctcctcttgaattaaaaaacaagcacatagaagatggggatatgatggaaattgggtttggtgcagccaacttcaaagaaattaatgcaagtaaatcagatctacctcttgacattcaaaatgagatctgcttgtacccagactacctcaaaatggctgaggacgctgctggtaatagcatgttcttttttgcaaggaaagaacaggtgtatgttagacacatctggaccagagggggctcggagaaagaagcccctaccacagatttttatttaaagaataataaaggggatgccacccttaaaatacccagtgtgcattttggtagtcccagtggctcactagtctcaactgataatcaaatttttaatcggccctactggctattccgtgcccagggcatgaacaatggaattgcatggaataatttattgtttttaacagtgggggacaatacacgtggtactaatcttaccataagtgtagcctcagatggaaccccactaacagagtatgatagctcaaaattcaatgtataccatagacatatggaagaatataagctagcctttatattagagctatgctctgtggaaatcacagctcaaactgtgtcacatctgcaaggacttatgccctctgtgcttgaaaattgggaaataggtgtgcagcctcctacctcatcgatattagaggacacctatcgctatatagagtctcctgcaactaaatgtgcaagcaatgtaattcctgcaaaagaagacccttatgcagggtttaagttttggaacatagatcttaaagaaaagctttctttggacttagatcaatttcccttgggaagaagatttttagcacagcaaggggcaggatgttcaactgtgagaaaacgaagaattagccaaaaaacttccagtaagcctgcaaaaaaaaaaaaaaaataaaagctaagtttctataaatgttctgtaaatgtaaaacagaaggtaagtcaactgcacctaataaaaatcacttaatagcaatgtgctgtgtcagttgtttattggaaccacacccggtacacatcctgtccagcatttgcagtgcgtgcattgaattattgtgctggctagacttcatggcgcctggcaccgaatcctgccttctcagcgaaaatgaataattgctttgttggcaagaaactaagcatcaatgggacgcgtgcaaagcaccggcggcggtagatgcggggtaagtactgaattttaattcgacctatcccggtaaagcgaaagcgacacgcttttttttcacacatagcgggaccgaacacgttataagtatcgattaggtctatttttgtctctctgtcggaaccagaactggtaaaagtttccattgcgtctgggcttgtctatcattgcgtctctatggtttttggaggattagacggggccaccagtaatggtgcatagcggatgtctgtaccgccatcggtgcaccgatataggtttggggctccccaagggactgctgggatgacagcttcatattatattgaatgggcgcataatcagcttaattggtgaggacaagctacaagttgtaacctgatctccacaaagtacgttgccggtcggggtcaaaccgtcttcggtgctcgaaaccgccttaaactacagacaggtcccagccaagtaggcggatcaaaacctcaaaaaggcgggagccaatcaaaatgcagcattatattttaagctcaccgaaaccggtaagtaaagactatgtattttttcccagtgaataattgtt

SEQ ID NO: 33 - DNA, 7412 bp, Bos taures papillomavirus 7
  ref <308>: DATABASE ACCESSION NUMBER: NCBI / NC_007612.1
  ref <309>: DATABASE ENTRY DATE: 2011-03-25
  ref <313>: RELEVANT RESIDUES IN SEQ ID NO: (1)..(7412)
cgttatagttgtcaacaacaatcactctgtcaagtaatgacatgaccggtaggggttatattaagggaccgctttgggggttcagcacaaatggctgacgaggacgtgatattcgtggaccgactgcgagctccgtggtgtatcctttgcatgtgctgtaaaagatccctaacaaatgacgagagaaaagattttttaaataagggtttaaaaacttttaagaaatggaataatgggaagaagcgttcgtttggctgctgcgagacttgctgtgtatttttagcaaatgaagaggcagaaaaaactcgcgcagaagagattcatttagaagcagatggtgtgcagcttttttgtggagcccctttgagagatatttccatgaactgtcgctattgcttagctgtgctaactttttatgacaagtacttaaataaggagaacagactgcccttttgcctacgcaggaaaaagtggagaggcacttgtgagaagtgcctgaaagacaaaaaacagtgctgatcatgcacgatccagcattgttctcgtcctcaggagagcagcctccagaagggattgtgcttgaattgcacccacttaatacaggcaatcatttagtgactgtacctgggacgacagaggtgacttcgtcacctaggtgtcaagaggaggggccaaggttgtgcttgtattatatatgtactgtatgtgcttggtgtcagagtcacctgcgcctgagtgtgtcaacgtccgattccagccttagaaaatttcaagagcttttgtgtggtgacttgacagtcgtttgcacaccctgtgcccgaaatggcagaagataaaggtactaaaggcggtgggggaatggtcagtggttcgtggtatttggatgtggaagctgaatgtgatgagcctgacaatctttgtgacttagaagcttgttttgataagtctgacagtgatgatgatccagaattcattagtaactctgatgttgaggaggggaattcttcggaactcttacacaataatcatatgctagccaaagatggtgagcagatccaactgctaaagcgaaagtacatgtccccaagcccagataaagaattaagcccgagattagcattagtgtcaatttctgctagccactctagtaagaggaggctttttccagagacgaaggacaagcatgaagctagcaattcttctgggtcggtttcgtccacgcaggttggttcaaatagccagagctataattccgaggacttgagcattgcaattcttaaaagcaaaaatcagaaagcaacagctttagctcagtttaaagaagcctttggtgtcagctttacagatttgactaggtcatttattagcaataagacttgcactcagcactgggttgtagctgtgtttggaccgaacagtgacattttagatggcactggtacactcttagaaccccactgcaccttcttgcttaagtgcacatgctttgcagaccgtgggcctataattctgcttcttatagaatttaaagccagtaagtgtcgtgatacagtgcaaaatttattgaataatattatgagggttgagcatcatcagatgttgcttgaacctccaaaaataaggagccagcttacagctttttttttttataaaaagactatggcaggaggctgcgacgtgattggcaagttgcctgattggctgactcgcctcactgtgctcagtcaccaaggcgccacagaagcatttgagctttcgagaatggtgcagtgggcttatgacaatgacatgttagaggacagtgaaatcgcttattattatgcacagcatgcagacgtggacagcaatgcagcagcatggctcaaaactaataaccaggccaaatatgttagagactgtggtaacatggtccggctttataagcagcaggaaatgaaaaacttaaccatgtcagagtatatttacaaaaggtgctgtaaagttgaaggctcaggcgattggaagcatatttttaaattgctaaggtatcaggatgttaatatgatacagtttttaacatcttttagagacttactaagttgcaagcctaaaagacagtgtctggttatatatgggccaccagacacagggaaatcatactttttatactctttgatttccttcttaaagggaaaagtcatttcattcacaaacagcaaaagccatttttggctgcagcctttgcttaatgccaaagttgcattgctagatgatgccactaaagcttgctggaactatatggactgttatatgaggacagctttagatggaaacgcagtgtctgtagatagcaagtttaaggcaccagtgcaagtaaggctcccccctttattaatctctacaaatgtagagctcccgttactcgaagaatataagtatttgcactccagaacgatgtgctattgctttgcaaagccatgtttatatgatgacgaaggaaatcccttatttaacttaactgacagacattggaaaggctttttcctgcatttggaacaacaactaggcctcaactttagtgagaaggatgaagaagctagcggagcatttagatgcatgccaagaacagatgctggaattgattgagaaggacagtcaagaattagaggaccaaatcgactactgggacttggtcaaacgtgaaaacttgctgctgtttgcagcaaaagaggctggcctgtcacggttaggctacgagccagtgccacccaccaaagtgtcagaaggcaaagccaaaaatgcaataatgatgagtatcagcttgcagtccctgcaaagttcagaatttggtagagacccctggacactgccccagacaagccttgaggtgtttatgtctaatccctctaactgttttaaaaagaatggagaacatgtggaagtgttatttgatggggacaaaaacaaagctgtgatttttgtcaagtggggtgaagtgtatgtgcaggatttgttgggtgcttggcacaaatgtcctagccatgttgtgtacgagggtatttactataaccaccctgactacggaagaaccttttacctcaggtttgaggaagaggctgcaaagtatggagctcacaaaccatggcaggtgatgaccactaacggcacccttttgcactctcctagtgaatcctcaaactccgccgacgggtcggaggagtcagctgccccctcccccggcccctccatcgaagcgccgcagcggctttccttttggggatcgcctgcaggagggcctgaacggggacggagaagacggagtgaaacgccgaggaaacggtcttttggagaccggaggcccaggccccaaactccgttgggaggactcagacggaaacgagtccgaagaggaagaggaggaggccttggggttaaagagcttgctgaaaaagctggaggacgacttgcaggaactcctggacagactgcagaaggaggtggacacacttccacggcgcctggccactatcctgtcctaattggcaaaggaaggccaaactgtctgaagtgctggagaaatcgttttggcgtgagccataaaggtctttttctagactgttcttcaactttttcctggactcagactggggggggaagaggtgtcgatggggtcatcctcattgtatttgaaacagaacaacagttgcaaacttttgtagacactgtacacaggcctacgagcatttcattgcgcagagggggaactgttttgcgtgctggctgcttttagcgggtgcagacaggggtaggggtgtattagatcaggggcgataatcatgagtgcactggctcaaagataaggttaagggcgggttgtgggaggatatttattggggaatgcgtgcagagggtgcttgtgcaggtgtgcttatttgcagcttgctttgtatagtgggtatgcgcggtccacacatttcaactgtgttgtcactgttatgtctgctgcgacaatgtcacggagtcgggttaaacgtgcttctgcagaagatttgtaccgtcaatgccaacttggcgctgactgtcctccagatgtcaaaaataaatttgaaaacaacactgttgcagaccgcatattgaaatgggtagctgggttcttatacttaggcacattagggattgggactgggaggggcacaggggggcgaggagggtatgtgcccattggacggggccctggcaccacaacagaaattgggggcacgcgcacactgaggccagtaggccctgtagagcctattggacctggcacacccactgtcatagatgcaactccccctgtagatgtggtagagactccaatagaccccacactgactgatgtcagaccaactgacccttctgtgtttgaaccagggggggaagacattgagctggaaacactgcagcctgaggaagatgtccttgcaggctctaaccctacaactgacctgccaactgtgggagagcccaacatagatttcactgaaacctcctttacagaagtgaggccccctgtctccagaactgctgacatttcagaaacaaacctagataatgcagcctataatgcagctgtagctgagtttgcaagagaagcaaaccaagtatcagtcatctttgatgctgaagttggtgggtcagtggtggggtctgaggaatttgaattagaggaagtccccttaacaagcacacctgaaaatcctgcaaggcctgctgggagaaggagaaattggggctctatgtatcataggtttataaaacaagtacgccttggctccacctcatttagcagggcagatgtaggcggacgatttgaatttgaaaatcccgcctttgaaggggatgtaggggtgtcagaggaaatgatgcaaaccagagacttgggtgaagttgtcattgccaaaggacctgaggggagagtccgtatgagtaggttggcacgaatacctggcatgcacactagaagtggactggagcttggtgagcatgtccacctattcgctgacatgagcaccatagaagagctcccattggaggaaacaatcgaactcagcactttctccaatcctcaaggcgtattggactctgggcctgtcataatagagtctgaaattggcgccacacagggtgtggtggtcaatgagcaaaccccaaacccatttgacaatgcagacctcggcaacactgtctctgaaactgcagacttacttgaatggggagttgaggacattgaacttttggcccaggaagactataatttcacaggcggacgcctaaggcttttagatgtagaagaagctccagatattgatgactggacattggagtctccaagaaaagcttatgctgtagccacaatcaataaggacagcaaaagccaaataccagttaaaatcccagtgcatgtagacccgtcagatgtagtggttattagctacacagcagatgttagcattttctctctgtttgagcccagcttatataggaaaagaaaatatagctatctgtattgatttttttgcaggatgtggaacaactccagtaaagtttatctgccaccaacgcagcctattgcaagagtactgtcaacaaaagaatatgtccaaaccactggatactactaccatggtcagagtgaacggctcataactgttggtcatccattttacccagtttacaatgaggaaagaactaaaatagtagttccacaggtgtctgcaaatcagctcagagcattcagaatcaaactgccagaccctaacaaatttgtgtttgcagacccaaacttttataatcctgaaacacataggctggtttggctgctaaaggccattgaaattggtagaggaggcccattaggtgtaggatgcacaggccatcccttttttaacaagattgacactgaaaaccctaataaatatccaaagacagacaaggatgatcgcatgcacacatcttttgacccaaagcattgtcagatgtttgtagtaggctgcaaaccctgcatagggagtcactggggtcttgcaaagtcctgtgtggacgcgcacaatcctgatattgatgagcactgccctccaatacaactagttaattcatttattgaagatggagatatgggagatataggccttggcaatatggactttctctcattgcaagaagacaggtcttgtgcaccattagaaattgtcacaaagaaatgtaaatttcctgactttctaaaaatgcaggccgaggcctctggggactctatgtttttttatggcagaaaagagtccctatatgctaggcacatgttttctagagtgggaaaaaatggagaagagtatcctcaccctgtagagcccagcgactacatcttgccaagtgcagacgctgaagatatggacagacagtctgcagcggcccccttgtactttgctactcccagtgggtctttaaatgcaagtgacagtcagctctttaacagagcttactttctcaggaactctcagggtcccaacaatggagtgctgtggaataatgaaatgtttgtgacaaccatggataattccagaaacacaaactttacaatttccattgctcctaatcccactgctcaatatgatgccacgagaatcaagtattacatgagacatgtagaaatctatgagctgatgtttgttttagaagtgggaaaaattgaattaaatggcacagtactagctcatataaatgcaatgaatccctctgtgattgacagttggaatcttgggtttgttccaatgcccacctcaactactgaggacacatatagatttttggacagtttagctactaagtgcccagccgatgtagtgccagagaaaaaggatccgtatgacggctatagtttttgggaggtggattgcacagaaaaaatgaccatggaacttgaccagtaccccctaggacgtaaatttctagctcagcgctttacagctcgtcctcgaacgaccctaaagagaccaggtgtgagaaaaagcacagctgcaaagaagcgcaggaaatgagttgtaaatgtatgcatacttgtcatgctgcagcggttccgtatgtaaacttgtgtaaataaacttatcaattcccaccgaattcggtctgttactgcgtgttcttcgactgcacccacccataagtggtcgcacctaattcgtttggaatgctagaatgcaaccgcgcccggttggcagctcctcttaacctgcaggtgcaccagttccgagccaaatagcaagatcggatcagcccgacactaatccttccagctggcacgaaccctcggactttaatccctgaatcaataaagtcttgtctgcgaaagcagtttcggtgagtacgaccggtttggttctcactaatcttcattattc

SEQ ID NO: 34 - DNA, 7412 bp, Bos taurus papillomavirus 7; identical to SEQ ID NO: 33
  ref <308>: DATABASE ACCESSION NUMBER: NCBI / NC_007612.1
  ref <309>: DATABASE ENTRY DATE: 2011-03-25
  ref <313>: RELEVANT RESIDUES IN SEQ ID NO: (1)..(7412)

SEQ ID NO: 35 - skipped

SEQ ID NO: 36 - protein, 7096 aa, Coronavirus 2019-nCoV [sequence omitted]

SEQ ID NO: 37 - protein, 1273 aa, Coronavirus 2019-nCoV
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 38 - protein, 275 aa, Coronavirus 2019-nCoV
MDLFMRIFTIGTVTLKQGEIKDATPSDFVRATATIPIQASLPFGWLIVGVALLAVFQSASKIITLKKRWQLALSKGVHFVCNLLLLFVTVYSHLLLVAAGLEAPFLYLYALVYFLQSINFVRIIMRLWLCWKCRSKNPLLYDANYFLCWHTNCYDYCIPYNSVTSSIVITSGDGTTSPISEHDYQIGGYTEKWESGVKDCVVLHSYFTSDYYQLYSTQLSTDTGVEHVTFFIYNKIVDEPEEHVQIHTIDGSSGVVNPVMEPIYDEPTTTTSVPL

SEQ ID NO: 39 - protein, 75 aa, Coronavirus 2019-nCoV
MYSFVSEETGTLIVNSVLLFLAFVVFLLVTLAILTALRLCAYCCNIVNVSLVKPSFYVYSRVKNLNSSRVPDLLV

SEQ ID NO: 40 - protein, 222 aa, Coronavirus 2019-nCoV
MADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 41 - protein, 61 aa, Coronavirus 2019-nCoV
MFHLVDFQVTIAEILLIIMRTFKVSIWNLDYIINLIIKNLSKSLTENKYSQLDEEQPMEID

SEQ ID NO: 42 - protein, 121 aa, Coronavirus 2019-nCoV
MKIILFLALITLATCELYHYQECVRGTTVLLKEPCSSGTYEGNSPFHPLADNKFALTCFSTQFAFACPDGVKHVYQLRARSVSPKLFIRQEEVQELYSPIFLIVAAIVFITLCFTLKRKTE

SEQ ID NO: 43 - protein, 121 aa, Coronavirus 2019-nCoV
MKFLVFLGIITTVAAFHQECSLQSCTQHQPYVVDDPCPIHFYSKWYIRVGARKSAPLIELCVDEAGSKSPIQYIDIGNYTVSCLPFTINCQEPKLGSLVVRCSFYEDFLEYHDVRVVLDFI

SEQ ID NO: 44 - protein, 419 aa, Coronavirus 2019-nCoV
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 45 - protein, 38 aa, Coronavirus 2019-nCoV
MGYINVFAFPFTIYSLLLCRMNSRNYIAQVDVVNFNLT

SEQ ID NO: 46 - DNA, 29903 bp, Coronavirus 2019-nCoV
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagccagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgtttaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaaaaaa

SEQ ID NO: 47 - DNA, 2412 bp, Homo sapiens
atgagggccctgtgggtgctgggcctctgctgcgtcctgctgaccttcgggtcggtcagagctgacgatgaagttgatgtggatggtacagtagaagaggatctgggtaaaagtagagaaggatcaaggacggatgatgaagtagtacagagagaggaagaagctattcagttggatggattaaatgcatcacaaataagagaacttagagagaagtcggaaaagtttgccttccaagccgaagttaacagaatgatgaaacttatcatcaattcattgtataaaaataaagagattttcctgagagaactgatttcaaatgcttctgatgctttagataagataaggctaatatcactgactgatgaaaatgctctttctggaaatgaggaactaacagtcaaaattaagtgtgataaggagaagaacctgctgcatgtcacagacaccggtgtaggaatgaccagagaagagttggttaaaaaccttggtaccatagccaaatctgggacaagcgagtttttaaacaaaatgactgaagcacaggaagatggccagtcaacttctgaattgattggccagtttggtgtcggtttctattccgccttccttgtagcagataaggttattgtcacttcaaaacacaacaacgatacccagcacatctgggagtctgactccaatgaattttctgtaattgctgacccaagaggaaacactctaggacggggaacgacaattacccttgtcttaaaagaagaagcatctgattaccttgaattggatacaattaaaaatctcgtcaaaaaatattcacagttcataaactttcctatttatgtatggagcagcaagactgaaactgttgaggagcccatggaggaagaagaagcagccaaagaagagaaagaagaatctgatgatgaagctgcagtagaggaagaagaagaagaaaagaaaccaaagactaaaaaagttgaaaaaactgtctgggactgggaacttatgaatgatatcaaaccaatatggcagagaccatcaaaagaagtagaagaagatgaatacaaagctttctacaaatcattttcaaaggaaagtgatgaccccatggcttatattcactttactgctgaaggggaagttaccttcaaatcaattttatttgtacccacatctgctccacgtggtctgtttgacgaatatggatctaaaaagagcgattacattaagctctatgtgcgccgtgtattcatcacagacgacttccatgatatgatgcctaaatacctcaattttgtcaagggtgtggtggactcagatgatctccccttgaatgtttcccgcgagactcttcagcaacataaactgcttaaggtgattaggaagaagcttgttcgtaaaacgctggacatgatcaagaagattgctgatgataaatacaatgatactttttggaaagaatttggtaccaacatcaagcttggtgtgattgaagaccactcgaatcgaacacgtcttgctaaacttcttaggttccagtcttctcatcatccaactgacattactagcctagaccagtatgtggaaagaatgaaggaaaaacaagacaaaatctacttcatggctgggtccagcagaaaagaggctgaatcttctccatttgttgagcgacttctgaaaaagggctatgaagttatttacctcacagaacctgtggatgaatactgtattcaggcccttcccgaatttgatgggaagaggttccagaatgttgccaaggaaggagtgaagttcgatgaaagtgagaaaactaaggagagtcgtgaagcagttgagaaagaatttgagcctctgctgaattggatgaaagataaagcccttaaggacaagattgaaaaggctgtggtgtctcagcgcctgacagaatctccgtgtgctttggtggccagccagtacggatggtctggcaacatggagagaatcatgaaagcacaagcgtaccaaacgggcaaggacatctctacaaattactatgcgagtcagaagaaaacatttgaaattaatcccagacacccgctgatcagagacatgcttcgacgaattaaggaagatgaagatgataaaacagttttggatcttgctgtggttttgtttgaaacagcaacgcttcggtcagggtatcttttaccagacactaaagcatatggagatagaatagaaagaatgcttcgcctcagtttgaacattgaccctgatgcaaaggtggaagaagagcccgaagaagaacctgaagagacagcagaagacacaacagaagacacagagcaagacgaagatgaagaaatggatgtgggaacagatgaagaagaagaaacagcaaaggaatctacagctgaaaaagatgaattgtaa

SEQ ID NO: 48 - protein, 803 aa, Homo sapiens; identical to SEQ ID NO: 2

SEQ ID NO: 49 - protein, 4 aa, Homo sapiens
KDEL

SEQ ID NO: 50 - DNA, 1455 bp, Artificial Sequence
  Synthetic sequence
atgagactgggaagccctggcctgctgtttctgctgttcagcagcctgagagccgacacccaggaaaaagaagtgcgggccatggtgggaagcgacgtggaactgagctgcgcctgtcctgagggcagcagattcgacctgaacgacgtgtacgtgtactggcagaccagcgagagcaagaccgtcgtgacctaccacatcccccagaacagctccctggaaaacgtggacagccggtacagaaaccgggccctgatgtctcctgccggcatgctgagaggcgacttcagcctgcggctgttcaacgtgaccccccaggacgagcagaaattccactgcctggtgctgagccagagcctgggcttccaggaagtgctgagcgtggaagtgaccctgcacgtggccgccaatttcagcgtgccagtggtgtctgccccccacagcccttctcaggatgagctgaccttcacctgtaccagcatcaacggctaccccagacccaatgtgtactggatcaacaagaccgacaacagcctgctggaccaggccctgcagaacgataccgtgttcctgaacatgcggggcctgtacgacgtggtgtccgtgctgagaatcgccagaacccccagcgtgaacatcggctgctgcatcgagaacgtgctgctgcagcagaacctgaccgtgggcagccagaccggcaacgacatcggcgagagagacaagatcaccgagaaccccgtgtccaccggcgagaagaatgccgccacctctaagtacggccctccctgcccttcttgcccagcccctgaatttctgggcggaccctccgtgtttctgttccccccaaagcccaaggacaccctgatgatcagccggacccccgaagtgacctgcgtggtggtggatgtgtcccaggaagatcccgaggtgcagttcaattggtacgtggacggggtggaagtgcacaacgccaagaccaagcccagagaggaacagttcaacagcacctaccgggtggtgtctgtgctgaccgtgctgcaccaggattggctgagcggcaaagagtacaagtgcaaggtgtccagcaagggcctgcccagcagcatcgaaaagaccatcagcaacgccaccggccagcccagggaaccccaggtgtacacactgccccctagccaggaagagatgaccaagaaccaggtgtccctgacctgtctcgtgaagggcttctacccctccgatatcgccgtggaatgggagagcaacggccagccagagaacaactacaagaccacccccccagtgctggacagcgacggctcattcttcctgtactcccggctgacagtggacaagagcagctggcaggaaggcaacgtgttcagctgcagcgtgatgcacgaagccctgcacaaccactacacccagaagtccctgtctctgtccctgggcaaatga

SEQ ID NO: 51 - protein, 484 aa, Artificial Sequence
  Synthetic sequence
MRLGSPGLLFLLFSSLRADTQEKEVRAMVGSDVELSCACPEGSRFDLNDVYVYWQTSESKTVVTYHIPQNSSLENVDSRYRNRALMSPAGMLRGDFSLRLFNVTPQDEQKFHCLVLSQSLGFQEVLSVEVTLHVAANFSVPVVSAPHSPSQDELTFTCTSINGYPRPNVYWINKTDNSLLDQALQNDTVFLNMRGLYDVVSVLRIARTPSVNIGCCIENVLLQQNLTVGSQTGNDIGERDKITENPVSTGEKNAATSKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSSWQEGNVFSCSVMHEALHNHYTQKSLSLSLGK

SEQ ID NO: 52 - DNA, 1305 bp, Artificial Sequence
  Synthetic sequence
atgtctaagtacggccctccctgccctagctgccctgcccctgaatttctgggcggacccagcgtgttcctgttccccccaaagcccaaggacaccctgatgatcagccggacccccgaagtgacctgcgtggtggtggatgtgtcccaggaagatcccgaggtgcagttcaattggtacgtggacggcgtggaagtgcacaacgccaagaccaagcccagagaggaacagttcaacagcacctaccgggtggtgtccgtgctgaccgtgctgcaccaggattggctgagcggcaaagagtacaagtgcaaggtgtccagcaagggcctgcccagcagcatcgagaaaaccatcagcaacgccaccggccagcccagggaaccccaggtgtacacactgccccctagccaggaagagatgaccaagaaccaggtgtccctgacctgtctcgtgaagggcttctacccctccgatatcgccgtggaatgggagagcaacggccagcctgagaacaactacaagaccacccccccagtgctggacagcgacggctcattcttcctgtacagcagactgaccgtggacaagagcagctggcaggaaggcaacgtgttcagctgcagcgtgatgcacgaggccctgcacaaccactacacccagaagtccctgtctctgagcctgggcaaggcctgtccatgggctgtgtctggcgctagagcctctcctggatctgccgccagccccagactgagagagggacctgagctgagccccgatgatcctgccggactgctggatctgagacagggcatgttcgcccagctggtggcccagaacgtgctgctgatcgatggccccctgagctggtacagcgatcctggactggctggcgtgtcactgacaggcggcctgagctacaaagaggacaccaaagaactggtggtggccaaggccggcgtgtactacgtgttctttcagctggaactgcggagagtggtggccggcgaaggatccggctctgtgtctctggctctgcatctgcagcccctgagatctgctgctggcgctgctgctctggccctgacagtggacctgcctcctgcctctagcgaggccagaaacagcgcattcgggtttcaaggcagactgctgcacctgtctgccggccagagactgggagtgcatctgcacacagaggccagagccaggcacgcctggcagctgactcagggcgctacagtgctgggcctgttcagagtgacccccgagattccagccggcctgcctagccccagatccgaatga

SEQ ID NO: 53 - protein, 434 aa, Artificial Sequence
  Synthetic sequence
MSKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSSWQEGNVFSCSVMHEALHNHYTQKSLSLSLGKACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 54 - DNA, 1284 bp, Artificial Sequence
  Synthetic sequence
atgtctaagtacggccctccctgccctagctgccctgcccctgaatttctgggcggacccagcgtgttcctgttccccccaaagcccaaggacaccctgatgatcagccggacccccgaagtgacctgcgtggtggtggatgtgtcccaggaagatcccgaggtgcagttcaattggtacgtggacggcgtggaagtgcacaacgccaagaccaagcccagagaggaacagttcaacagcacctaccgggtggtgtccgtgctgaccgtgctgcaccaggattggctgagcggcaaagagtacaagtgcaaggtgtccagcaagggcctgcccagcagcatcgagaaaaccatcagcaacgccaccggccagcccagggaaccccaggtgtacacactgccccctagccaggaagagatgaccaagaaccaggtgtccctgacctgtctcgtgaagggcttctacccctccgatatcgccgtggaatgggagagcaacggccagcctgagaacaactacaagaccacccccccagtgctggacagcgacggctcattcttcctgtacagcagactgaccgtggacaagagcagctggcaggaaggcaacgtgttcagctgcagcgtgatgcacgaggccctgcacaaccactacacccagaagtccctgtctctgagcctgggcaagatcgagggccggatggatagagcccagggcgaagcctgcgtgcagttccaggctctgaagggccaggaattcgcccccagccaccagcaggtgtacgcccctctgagagccgacggcgataagcctagagcccacctgacagtcgtgcggcagacccctacccagcacttcaagaatcagttccccgccctgcactgggagcacgaactgggcctggccttcaccaagaacagaatgaactacaccaacaagtttctgctgatccccgagagcggcgactacttcatctacagccaagtgaccttccggggcatgaccagcgagtgcagcgagatcagacaggccggcagacctaacaagcccgacagcatcaccgtcgtgatcaccaaagtgaccgacagctaccccgagcccacccagctgctgatgggcaccaagagcgtgtgcgaagtgggcagcaactggttccagcccatctacctgggcgccatgtttagtctgcaagagggcgacaagctgatggtcaacgtgtccgacatcagcctggtggattacaccaaagaggacaagaccttcttcggcgcctttctgctctga

SEQ ID NO: 55 - protein, 427 aa, Artificial Sequence
  Synthetic sequence
MSKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSSWQEGNVFSCSVMHEALHNHYTQKSLSLSLGKIEGRMDRAQGEACVQFQALKGQEFAPSHQQVYAPLRADGDKPRAHLTVVRQTPTQHFKNQFPALHWEHELGLAFTKNRMNYTNKFLLIPESGDYFIYSQVTFRGMTSECSEIRQAGRPNKPDSITVVITKVTDSYPEPTQLLMGTKSVCEVGSNWFQPIYLGAMFSLQEGDKLMVNVSDISLVDYTKEDKTFFGAFLL

SEQ ID NO: 56 - DNA, 1107 bp, Artificial Sequence
  Synthetic sequence
atgtctaagtacggccctccctgccctagctgccctgcccctgaatttctgggcggacccagcgtgttcctgttccccccaaagcccaaggacaccctgatgatcagccggacccccgaagtgacctgcgtggtggtggatgtgtcccaggaagatcccgaggtgcagttcaattggtacgtggacggcgtggaagtgcacaacgccaagaccaagcccagagaggaacagttcaacagcacctaccgggtggtgtccgtgctgaccgtgctgcaccaggattggctgagcggcaaagagtacaagtgcaaggtgtccagcaagggcctgcccagcagcatcgagaaaaccatcagcaacgccaccggccagcccagggaaccccaggtgtacacactgccccctagccaggaagagatgaccaagaaccaggtgtccctgacctgtctcgtgaagggcttctacccctccgatatcgccgtggaatgggagagcaacggccagcctgagaacaactacaagaccacccccccagtgctggacagcgacggctcattcttcctgtacagcagactgaccgtggacaagagcagctggcaggaaggcaacgtgttcagctgcagcgtgatgcacgaggccctgcacaaccactacacccagaagtccctgtctctgagcctgggcaagatcgagggccggatggatcaggtgtcacacagatacccccggatccagagcatcaaagtgcagtttaccgagtacaagaaagagaagggctttatcctgaccagccagaaagaggacgagatcatgaaggtgcagaacaacagcgtgatcatcaactgcgacgggttctacctgatcagcctgaagggctacttcagtcaggaagtgaacatcagcctgcactaccagaaggacgaggaacccctgttccagctgaagaaagtgcggagcgtgaacagcctgatggtggcctctctgacctacaaggacaaggtgtacctgaacgtgaccaccgacaacaccagcctggacgacttccacgtgaacggcggcgagctgatcctgattcaccagaaccccggcgagttctgcgtgctctga

SEQ ID NO: 57 - protein, 368 aa, Artificial Sequence
  Synthetic sequence
MSKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSSWQEGNVFSCSVMHEALHNHYTQKSLSLSLGKIEGRMDQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 58 - DNA, 1588 bp, Homo sapiens
tcccaagtagctgggactacaggagcccaccaccacccccggctaattttttgtatttttagtagagacggggtttcaccgtgttagccaagatggtcttgatcacctgacctcgtgatccacccgccttggcctcccaaagtgctgggattacaggcatgagccaccgcgcccggcctccattcaagtctttattgaatatctgctatgttctacacactgttctaggtgctggggatgcaacaggggacaaaataggcaaaatccctgtccttttggggttgacattctagtgactcttcatgtagtctagaagaagctcagtgaatagtgtctgtggttgttaccagggacacaatgacaggaacattcttgggtagagtgagaggcctggggagggaagggtctctaggatggagcagatgctgggcagtcttagggagcccctcctggcatgcaccccctcatccctcaggccacccccgtcccttgcaggagcaccctggggagctgtccagagcgctgtgccgctgtctgtggctggaggcagagtaggtggtgtgctgggaatgcgagtgggagaactgggatggaccgaggggaggcgggtgaggaggggggcaaccacccaacacccaccagctgctttcagtgttctgggtccaggtgctcctggctggccttgtggtccccctcctgcttggggccaccctgacctacacataccgccactgctggcctcacaagcccctggttactgcagatgaagctgggatggaggctctgaccccaccaccggccacccatctgtcacccttggacagcgcccacacccttctagcacctcctgacagcagtgagaagatctgcaccgtccagttggtgggtaacagctggacccctggctaccccgagacccaggaggcgctctgcccgcaggtgacatggtcctgggaccagttgcccagcagagctcttggccccgctgctgcgcccacactctcgccagagtccccagccggctcgccagccatgatgctgcagccgggcccgcagctctacgacgtgatggacgcggtcccagcgcggcgctggaaggagttcgtgcgcacgctggggctgcgcgaggcagagatcgaagccgtggaggtggagatcggccgcttccgagaccagcagtacgagatgctcaagcgctggcgccagcagcagcccgcgggcctcggagccgtttacgcggccctggagcgcatggggctggacggctgcgtggaagacttgcgcagccgcctgcagcgcggcccgtgacacggcgcccacttgccacctaggcgctctggtggcccttgcagaagccctaagtacggttacttatgcgtgtagacattttatgtcacttattaagccgctggcacggccctgcgtagcagcaccagccggccccacccctgctcgcccctatcgctccagccaaggcgaagaagcacgaacgaatgtcgagagggggtgaagacatttctcaacttctcggccggagtttggctgagatcgcggtattaaatctgtgaaagaaaacaaaacaaaacaa

SEQ ID NO: 59 - protein, 426 aa, Homo sapiens
MEQRPRGCAAVAAALLLVLLGARAQGGTRSPRCDCAGDFHKKIGLFCCRGCPAGHYLKAPCTEPCGNSTCLVCPQDTFLAWENHHNSECARCQACDEQASQVALENCSAVADTRCGCKPGWFVECQVSQCVSSSPFYCQPCLDCGALHRHTRLLCSRRDTDCGTCLPGFYEHGDGCVSCPTPPPSLAGAPWGAVQSAVPLSVAGGRVGVFWVQVLLAGLVVPLLLGATLTYTYRHCWPHKPLVTADEAGMEALTPPPATHLSPLDSAHTLLAPPDSSEKICTVQLVGNSWTPGYPETQEALCPQVTWSWDQLPSRALGPAAAPTLSPESPAGSPAMMLQPGPQLYDVMDAVPARRWKEFVRTLGLREAEIEAVEVEIGRFRDQQYEMLKRWRQQQPAGLGAVYAALERMGLDGCVEDLRSRLQRGP

SEQ ID NO: 60-71 - skipped (12 entries)

SEQ ID NO: 72 - protein, 5 aa, Artificial Sequence
  Synthetic sequence
GGGGS

SEQ ID NO: 73 - protein, 5 aa, Artificial sequence
  Synthetic sequence
GGGGS

SEQ ID NO: 74 - protein, 8 aa, Artificial sequence
  Synthetic sequence
GGGGGGGG

SEQ ID NO: 75 - protein, 6 aa, Artificial sequence
  Synthetic sequence
GGGGGG

SEQ ID NO: 76 - protein, 5 aa, Artificial sequence
  Synthetic sequence
EAAAK

SEQ ID NO: 77 - protein, 7 aa, Artificial sequence
  Synthetic sequence
AEAAAKA

SEQ ID NO: 78 - protein, 12 aa, Artificial sequence
  Synthetic sequence
AEAAAKEAAAKA

SEQ ID NO: 79 - protein, 46 aa, Artificial sequence
  Synthetic sequence
AEAAAKEAAAKEAAAKEAAAKALEAEAAAKEAAAKEAAAKEAAAKA

SEQ ID NO: 80 - protein, 5 aa, Artificial sequence
  Synthetic sequence
PAPAP

SEQ ID NO: 81 - protein, 18 aa, Artificial sequence
  Synthetic sequence
KESGSVSSEQLAQFRSLD

SEQ ID NO: 82 - protein, 14 aa, Artificial sequence
  Synthetic sequence
EGKSSGSGSESKST

SEQ ID NO: 83 - protein, 12 aa, Artificial sequence
  Synthetic sequence
GSAGSAAGSGEF

SEQ ID NO: 84 - DNA, 852 bp, Homo sapiens
atggagcctcctggagactgggggcctcctccctggagatccacccccaaaaccgacgtcttgaggctggtgctgtatctcaccttcctgggagccccctgctacgccccagctctgccgtcctgcaaggaggacgagtacccagtgggctccgagtgctgccccaagtgcagtccaggttatcgtgtgaaggaggcctgcggggagctgacgggcacagtgtgtgaaccctgccctccaggcacctacattgcccacctcaatggcctaagcaagtgtctgcagtgccaaatgtgtgacccagccatgggcctgcgcgcgagccggaactgctccaggacagagaacgccgtgtgtggctgcagcccaggccacttctgcatcgtccaggacggggaccactgcgccgcgtgccgcgcttacgccacctccagcccgggccagagggtgcagaagggaggcaccgagagtcaggacaccctgtgtcagaactgccccccggggaccttctctcccaatgggaccctggaggaatgtcagcaccagaccaagtgcagctggctggtgacgaaggccggagctgggaccagcagctcccactgggtatggtggtttctctcagggagcctcgtcatcgtcattgtttgctccacagttggcctaatcatatgtgtgaaaagaagaaagccaaggggtgatgtagtcaaggtgatcgtctccgtccagcggaaaagacaggaggcagaaggtgaggccacagtcattgaggccctgcaggcccctccggacgtcaccacggtggccgtggaggagacaataccctcattcacggggaggagcccaaaccattaa

SEQ ID NO: 85 - protein, 283 aa, Homo sapiens
MEPPGDWGPPPWRSTPKTDVLRLVLYLTFLGAPCYAPALPSCKEDEYPVGSECCPKCSPGYRVKEACGELTGTVCEPCPPGTYIAHLNGLSKCLQCQMCDPAMGLRASRNCSRTENAVCGCSPGHFCIVQDGDHCAACRAYATSSPGQRVQKGGTESQDTLCQNCPPGTFSPNGTLEECQHQTKCSWLVTKAGAGTSSSHWVWWFLSGSLVIVIVCSTVGLIICVKRRKPRGDVVKVIVSVQRKRQEAEGEATVIEALQAPPDVTTVAVEETIPSFTGRSPNH

SEQ ID NO: 86 - DNA, 4900 bp, Homo sapiens
taaagtcatcaaaacaacgttatatcctgtgtgaaatgctgcagtcaggatgccttgtggtttgagtgccttgatcatgtgccctaaggggatggtggcggtggtggtggccgtggatgacggagactctcaggccttggcaggtgcgtctttcagttcccctcacacttcgggttcctcggggaggaggggctggaaccctagcccatcgtcaggacaaagatgctcaggctgctcttggctctcaacttattcccttcaattcaagtaacaggaaacaagattttggtgaagcagtcgcccatgcttgtagcgtacgacaatgcggtcaaccttagctgcaagtattcctacaatctcttctcaagggagttccgggcatcccttcacaaaggactggatagtgctgtggaagtctgtgttgtatatgggaattactcccagcagcttcaggtttactcaaaaacggggttcaactgtgatgggaaattgggcaatgaatcagtgacattctacctccagaatttgtatgttaaccaaacagatatttacttctgcaaaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcctgacacggacgcctatccagaagccagccggctggcagcccccatctgctcaatatcactgctctggataggaaatgaccgccatctccagccggccacctcaggcccctgttgggccaccaatgccaatttttctcgagtgactagaccaaatatcaagatcattttgagactctgaaatgaagtaaaagagatttcctgtgacaggccaagtcttacagtgccatggcccacattccaacttaccatgtacttagtgacttgactgagaagttagggtagaaaacaaaaagggagtggattctgggagcctcttccctttctcactcacctgcacatctcagtcaagcaaagtgtggtatccacagacattttagttgcagaagaaaggctaggaaatcattccttttggttaaatgggtgtttaatcttttggttagtgggttaaacggggtaagttagagtagggggagggataggaagacatatttaaaaaccattaaaacactgtctcccactcatgaaatgagccacgtagttcctatttaatgctgttttcctttagtttagaaatacatagacattgtcttttatgaattctgatcatatttagtcattttgaccaaatgagggatttggtcaaatgagggattccctcaaagcaatatcaggtaaaccaagttgctttcctcactccctgtcatgagacttcagtgttaatgttcacaatatactttcgaaagaataaaatagttctcctacatgaagaaagaatatgtcaggaaataaggtcactttatgtcaaaattatttgagtactatgggacctggcgcagtggctcatgcttgtaatcccagcactttgggaggccgaggtgggcagatcacttgagatcaggaccagcctggtcaagatggtgaaactccgtctgtactaaaaatacaaaatttagcttggcctggtggcaggcacctgtaatcccagctgcccaagaggctgaggcatgagaatcgcttgaacctggcaggcggaggttgcagtgagccgagatagtgccacagctctccagcctgggcgacagagtgagactccatctcaaacaacaacaacaacaacaacaacaacaacaaaccacaaaattatttgagtactgtgaaggattatttgtctaacagttcattccaatcagaccaggtaggagctttcctgtttcatatgtttcagggttgcacagttggtctctttaatgtcggtgtggagatccaaagtgggttgtggaaagagcgtccataggagaagtgagaatactgtgaaaaagggatgttagcattcattagagtatgaggatgagtcccaagaaggttctttggaaggaggacgaatagaatggagtaatgaaattcttgccatgtgctgaggagatagccagcattaggtgacaatcttccagaagtggtcaggcagaaggtgccctggtgagagctcctttacagggactttatgtggtttagggctcagagctccaaaactctgggctcagctgctcctgtaccttggaggtccattcacatgggaaagtattttggaatgtgtcttttgaagagagcatcagagttcttaagggactgggtaaggcctgaccctgaaatgaccatggatatttttctacctacagtttgagtcaactagaatatgcctggggaccttgaagaatggcccttcagtggccctcaccatttgttcatgcttcagttaattcaggtgttgaaggagcttaggttttagaggcacgtagacttggttcaagtctcgttagtagttgaatagcctcaggcaagtcactgcccacctaagatgatggttcttcaactataaaatggagataatggttacaaatgtctcttcctatagtataatctccataagggcatggcccaagtctgtctttgactctgcctatccctgacatttagtagcatgcccgacatacaatgttagctattggtattattgccatatagataaattatgtataaaaattaaactgggcaatagcctaagaaggggggaatattgtaacacaaatttaaacccactacgcagggatgaggtgctataatatgaggaccttttaacttccatcattttcctgtttcttgaaatagtttatcttgtaatgaaatataaggcacctcccacttttatgtatagaaagaggtcttttaatttttttttaatgtgagaaggaagggaggagtaggaatcttgagattccagatcgaaaatactgtactttggttgatttttaagtgggcttccattccatggatttaatcagtcccaagaagatcaaactcagcagtacttgggtgctgaagaactgttggatttaccctggcacgtgtgccacttgccagcttcttgggcacacagagttcttcaatccaagttatcagattgtatttgaaaatgacagagctggagagttttttgaaatggcagtggcaaataaataaatacttttttttaaatggaaagacttgatctatggtaataaatgattttgttttctgactggaaaaataggcctactaaagatgaatcacacttgagatgtttcttactcactctgcacagaaacaaagaagaaatgttatacagggaagtccgttttcactattagtatgaaccaagaaatggttcaaaaacagtggtaggagcaatgctttcatagtttcagatatggtagttatgaagaaaacaatgtcatttgctgctattattgtaagagtcttataattaatggtactcctataatttttgattgtgagctcacctatttgggttaagcatgccaatttaaagagaccaagtgtatgtacattatgttctacatattcagtgataaaattactaaactactatatgtctgctttaaatttgtactttaatattgtcttttggtattaagaaagatatgctttcagaatagatatgcttcgctttggcaaggaatttggatagaacttgctatttaaaagaggtgtggggtaaatccttgtataaatctccagtttagccttttttgaaaaagctagactttcaaatactaatttcacttcaagcagggtacgtttctggtttgtttgcttgacttcagtcacaatttcttatcagaccaatggctgacctctttgagatgtcaggctaggcttacctatgtgttctgtgtcatgtgaatgctgagaagtttgacagagatccaacttcagccttgaccccatcagtccctcgggttaactaactgagccaccggtcctcatggctattttaatgagggtattgatggttaaatgcatgtctgatcccttatcccagccatttgcactgccagctgggaactataccagacctggatactgatcccaaagtgttaaattcaactacatgctggagattagagatggtgccaataaaggacccagaaccaggatcttgattgctatagacttattaataatccaggtcaaagagagtgacacacactctctcaagacctggggtgagggagtctgtgttatctgcaaggccatttgaggctcagaaagtctctctttcctatagatatatgcatactttctgacatataggaatgtatcaggaatactcaaccatcacaggcatgttcctacctcagggcctttacatgtcctgtttactctgtctagaatgtccttctgtagatgacctggcttgcctcgtcacccttcaggtccttgctcaagtgtcatcttctcccctagttaaactaccccacaccctgtctgctttccttgcttatttttctccatagcattttaccatctcttacattagacatttttcttatttatttgtagtttataagcttcatgaggcaagtaactttgctttgtttcttgctgtatctccagtgcccagagcagtgcctggtatataataaatatttattgactgagtgaaaaaaaaaaaaaaaaa

SEQ ID NO: 87 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 88 - DNA, 1906 bp, Homo sapiens
ccaagtcacatgattcaggattcagggggagaatccttcttggaacagagatgggcccagaactgaatcagatgaagagagataaggtgtgatgtggggaagactatataaagaatggacccagggctgcagcaagcactcaacggaatggcccctcctggagacacagccatgcatgtgccggcgggctccgtggccagccacctggggaccacgagccgcagctatttctatttgaccacagccactctggctctgtgccttgtcttcacggtggccactattatggtgttggtcgttcagaggacggactccattcccaactcacctgacaacgtccccctcaaaggaggaaattgctcagaagacctcttatgtatcctgaaaagagctccattcaagaagtcatgggcctacctccaagtggcaaagcatctaaacaaaaccaagttgtcttggaacaaagatggcattctccatggagtcagatatcaggatgggaatctggtgatccaattccctggtttgtacttcatcatttgccaactgcagtttcttgtacaatgcccaaataattctgtcgatctgaagttggagcttctcatcaacaagcatatcaaaaaacaggccctggtgacagtgtgtgagtctggaatgcaaacgaaacacgtataccagaatctctctcaattcttgctggattacctgcaggtcaacaccaccatatcagtcaatgtggatacattccagtacatagatacaagcacctttcctcttgagaatgtgttgtccatcttcttatacagtaattcagactgaacagtttctcttggccttcaggaagaaagcgcctctctaccatacagtatttcatccctccaaacacttgggcaaaaagaaaactttagaccaagacaaactacacagggtattaaatagtatacttctccttctgtctcttggaaagatacagctccagggttaaaaagagagtttttagtgaagtatctttcagatagcaggcagggaagcaatgtagtgtggtgggcagagccccacacagaatcagaagggatgaatggatgtcccagcccaaccactaattcactgtatggtcttgatctatttcttctgttttgagagcctccagttaaaatggggcttcagtaccagagcagctagcaactctgccctaatgggaaatgaaggggagctgggtgtgagtgtttacactgtgcccttcacgggatacttcttttatctgcagatggcctaatgcttagttgtccaagtcgcgatcaaggactctctcacacaggaaacttccctatactggcagatacacttgtgactgaaccatgcccagtttatgcctgtctgactgtcactctggcactaggaggctgatcttgtactccatatgaccccacccctaggaacccccagggaaaaccaggctcggacagccccctgttcctgagatggaaagcacaaatttaatacaccaccacaatggaaaacaagttcaaagacttttacttacagatcctggacagaaagggcataatgagtctgaagggcagtcctccttctccaggttacatgaggcaggaataagaagtcagacagagacagcaagacagttaacaacgtaggtaaagaaatagggtgtggtcactctcaattcactggcaaatgcctgaatggtctgtctgaaggaagcaacagagaagtggggaatccagtctgctaggcaggaaagatgcctctaagttcttgtctctggccagaggtgtggtatagaaccagaaacccatatcaagggtgactaagcccggcttccggtatgagaaattaaacttgtatacaaaatggttgccaaggcaacataaaattataagaattc

SEQ ID NO: 89 - protein, 234 aa, Homo sapiens
MDPGLQQALNGMAPPGDTAMHVPAGSVASHLGTTSRSYFYLTTATLALCLVFTVATIMVLVVQRTDSIPNSPDNVPLKGGNCSEDLLCILKRAPFKKSWAYLQVAKHLNKTKLSWNKDGILHGVRYQDGNLVIQFPGLYFIICQLQFLVQCPNNSVDLKLELLINKHIKKQALVTVCESGMQTKHVYQNLSQFLLDYLQVNTTISVNVDTFQYIDTSTFPLENVLSIFLYSNSD

SEQ ID NO: 90 - DNA, 1629 bp, Homo sapiens
tttcctgggcggggccaaggctggggcaggggagtcagcagaggcctcgctcgggcgcccagtggtcctgccgcctggtctcacctcgctatggttcgtctgcctctgcagtgcgtcctctggggctgcttgctgaccgctgtccatccagaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcacaaatactgcgaccccaacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccccaggatcggctgagagccctggtggtgatccccatcatcttcgggatcctgtttgccatcctcttggtgctggtctttatcaaaaaggtggccaagaagccaaccaataaggccccccaccccaagcaggaaccccaggagatcaattttcccgacgatcttcctggctccaacactgctgctccagtgcaggagactttacatggatgccaaccggtcacccaggaggatggcaaagagagtcgcatctcagtgcaggagagacagtgaggctgcacccacccaggagtgtggccacgtgggcaaacaggcagttggccagagagcctggtgctgctgctgctgtggcgtgagggtgaggggctggcactgactgggcatagctccccgcttctgcctgcacccctgcagtttgagacaggagacctggcactggatgcagaaacagttcaccttgaagaacctctcacttcaccctggagcccatccagtctcccaacttgtattaaagacagaggcagaagtttggtggtggtggtgttggggtatggtttagtaatatccaccagaccttccgatccagcagtttggtgcccagagaggcatcatggtggcttccctgcgcccaggaagccatatacacagatgcccattgcagcattgtttgtgatagtgaacaactggaagctgcttaactgtccatcagcaggagactggctaaataaaattagaatatatttatacaacagaatctcaaaaacactgttgagtaaggaaaaaaaggcatgctgctgaatgatgggtatggaactttttaaaaaagtacatgcttttatgtatgtatattgcctatggatatatgtataaatacaatatgcatcatatattgatataacaagggttctggaagggtacacagaaaacccacagctcgaagagtggtgacgtctggggtggggaagaagggtctggggg

SEQ ID NO: 91 - protein, 277 aa, Homo sapiens
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 92 - DNA, 913 bp, Homo sapiens
ccagagaggggcaggctggtcccctgacaggttgaagcaagtagacgcccaggagccccgggagggggctgcagtttccttccttccttctcggcagcgctccgcgcccccatcgcccctcctgcgctagcggaggtgatcgccgcggcgatgccggaggagggttcgggctgctcggtgcggcgcaggccctatgggtgcgtcctgcgggctgctttggtcccattggtcgcgggcttggtgatctgcctcgtggtgtgcatccagcgcttcgcacaggctcagcagcagctgccgctcgagtcacttgggtgggacgtagctgagctgcagctgaatcacacaggacctcagcaggaccccaggctatactggcaggggggcccagcactgggccgctccttcctgcatggaccagagctggacaaggggcagctacgtatccatcgtgatggcatctacatggtacacatccaggtgacgctggccatctgctcctccacgacggcctccaggcaccaccccaccaccctggccgtgggaatctgctctcccgcctcccgtagcatcagcctgctgcgtctcagcttccaccaaggttgtaccattgcctcccagcgcctgacgcccctggcccgaggggacacactctgcaccaacctcactgggacacttttgccttcccgaaacactgatgagaccttctttggagtgcagtgggtgcgcccctgaccactgctgctgattagggttttttaaattttattttattttatttaagttcaagagaaaaagtgtacacacaggggccacccggggttggggtgggagtgtggtggggggtagtggtggcaggacaagagaaggcattgagctttttctttcattttcctattaaaaaatacaaaaatca

SEQ ID NO: 93 - protein, 193 aa, Homo sapiens
MPEEGSGCSVRRRPYGCVLRAALVPLVAGLVICLVVCIQRFAQAQQQLPLESLGWDVAELQLNHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWVRP

SEQ ID NO: 94 - DNA, 723 bp, Homo sapiens
atggaggagagtgtcgtacggccctcagtgtttgtggtggatggacagaccgacatcccattcacgaggctgggacgaagccaccggagacagtcgtgcagtgtggcccgggtgggtctgggtctcttgctgttgctgatgggggccgggctggccgtccaaggctggttcctcctgcagctgcactggcgtctaggagagatggtcacccgcctgcctgacggacctgcaggctcctgggagcagctgatacaagagcgaaggtctcacgaggtcaacccagcagcgcatctcacaggggccaactccagcttgaccggcagcggggggccgctgttatgggagactcagctgggcctggccttcctgaggggcctcagctaccacgatggggcccttgtggtcaccaaagctggctactactacatctactccaaggtgcagctgggcggtgtgggctgcccgctgggcctggccagcaccatcacccacggcctctacaagcgcacaccccgctaccccgaggagctggagctgttggtcagccagcagtcaccctgcggacgggccaccagcagctcccgggtctggtgggacagcagcttcctgggtggtgtggtacacctggaggctggggaggaggtggtcgtccgtgtgctggatgaacgcctggttcgactgcgtgatggtacccggtcttacttcggggctttcatggtgtga

SEQ ID NO: 95 - protein, 240 aa, Homo sapiens
MEESVVRPSVFVVDGQTDIPFTRLGRSHRRQSCSVARVGLGLLLLLMGAGLAVQGWFLLQLHWRLGEMVTRLPDGPAGSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEEVVVRVLDERLVRLRDGTRSYFGAFMV

SEQ ID NO: 96 - protein, 9 aa, Artificial Sequence
  Synthetic Polymer
FIAGLIAIV

SEQ ID NO: 97 - protein, 9 aa, Artificial Sequence
  Synthetic Polymer
YLQPRTFLL

SEQ ID NO: 98 - RNA, 8 nt, Artificial Sequence
  Synthetic Sequence
agccaugg